(19) United States
(12) Patent Application Publication
(10) Pub. No.: US 2019/0183997 A1
(43) Pub. Date: Jun. 20, 2019

(54) IDENTIFICATION AND PRODUCTION OF HIGH AFFINITY IGM ANTIBODIES AND DERIVATIVES THEREOF

(71) Applicants: UNIVERSITY OF WASHINGTON, Seattle, WA (US); SEATTLE CHILDREN'S HOSPITAL D/B/A SEATTLE CHILDREN'S RESEARCH INSTITUTE, Seattle, WA (US)

(72) Inventors: Marion PEPPER, Seattle, WA (US); Akshay KRISHNAMURTY, Seattle, WA (US); David J. RAWLINGS, Seattle, WA (US); Christopher THOUVENEL, Seattle, WA (US)

(73) Assignees: UNIVERSITY OF WASHINGTON, Seattle, WA (US); SEATTLE CHILDREN'S HOSPITAL D/B/A SEATTLE CHILDREN'S RESEARCH INSTITUTE, Seattle, WA (US)

(21) Appl. No.: 16/319,755

(22) PCT Filed: Jul. 21, 2017
(86) PCT No.: PCT/US2017/043358

§ 371 Date: Jan. 22, 2019

Related U.S. Application Data
(60) Provisional application No. 62/365,514, filed on Jul. 22, 2016.

(51) Int. Cl. A61K 39/015; C07K 14/705; C07K 14/445; G01N 33/564; C07K 16/42; C07K 16/20; G01N 33/68; A61K 39/395

(52) U.S. Cl. CPC A61K 39/015; C07K 14/705; C07K 14/445; A61K 39/395; C07K 16/42; C07K 16/205; G01N 33/68; G01N 33/564

(57) ABSTRACT

Provided herein are methods of sorting antigen-specific IgM memory B cells (MBCs), compositions and methods comprising such antigen-specific IgM MBCs, and recombinant antibody or antigen-binding fragments isolated from such antigen-specific IgM MBCs. As demonstrated herein, IgM and IgD MBCs are unique populations of cells with distinct phenotypic, functional and survival properties. Accordingly, the antigen-specific IgM MBCs and antibodies and antigen-binding fragments derived from these cells described herein are useful in therapeutic applications in vaccine strategies and treatment of infectious diseases.

### CROSS REFERENCE TO RELATED APPLICATIONS

This application claims benefit under 35 U.S.C. § 119(e) of U.S. Provisional No. 62/365,514, filed Jul. 22, 2016 the contents of which are incorporated herein by reference in their entirety.

### FIELD OF THE INVENTION

The present disclosure relates to the isolation of antigen-specific IgM memory B cells and compositions and methods related to and derived therefrom.

### BACKGROUND

Memory B cells (MBCs) induced by vaccine or infection are critical components of a protective humoral response. MBCs can persist for long periods of time and rapidly respond to subsequent infection through the production of antibody secreting cells, formation of new germinal centers (GCs) and repopulation of the memory pool (Tarlinton and Good-Jacobson, 2013). Classically defined MBCs express class-switched, somatically hypermutated B cell receptors (BCRs) after undergoing a GC reaction. These cells produce high affinity antibodies within days of a secondary challenge, making them the gold standard for vaccine development. It is now recognized that diverse MBC subsets exist in both mice and humans (Dogan et al., 2009; Klein et al., 1997; Obukhanych and Nussenzweig, 2006; Pape et al., 2011; Seifert et al., 2015).

Technical advances in tracking antigen-specific B cells have revealed that MBCs are heterogeneous. They have been shown to express either isotype switched or unswitched BCRs that have undergone various degrees of somatic hypermutation (Kaji et al., 2012; Pape et al., 2011; Toyama et al., 2002). MBC subsets also exhibit varied expression of surface markers associated with T cell interactions such as CD73, CD80 and PDL2, revealing varied developmental histories and receptor ligand interactions (Anderson et al., 2007; Taylor et al., 2012b; Tomayko et al., 2010). Importantly, these phenotypically different MBC subsets have also been associated with functional heterogeneity, although different studies have led to different conclusions. Some studies have demonstrated that unswitched MBCs preferentially enter GCs, while switched MBCs preferentially form plasmablasts (Benson et al., 2009; Dogan et al., 2009; Pape et al., 2011; Seifert et al., 2015). Other studies have shown instead that unswitched MBCs rapidly generate plasmablasts upon secondary challenge whereas switched MBCs preferentially re-enter GCs (McHeyzer-Williams et al., 2015).

### SUMMARY OF THE INVENTION

Humoral immunity comprises pre-existing antibodies expressed by long-lived plasma cells and rapidly reactive memory B cells (MBC). Recent studies of MBC development and function after protein immunization have uncovered significant MBC heterogeneity. To clarify functional roles for distinct MBC subsets during infection, the inventors have focused on the protozoan parasitic disease, malaria, which despite some progress remains difficult to prevent via vaccination. The knowledge gained from the studies described herein in regard to malaria is applicable for memory B cell-derived compositions and methods permitting the targeting of any of a wide range of additional antigens.

As described herein, in the malaria model, tetramers were generated that identify Plasmodium-specific MBCs in both humans and mice. Long-lived murine Plasmodium-specific MBCs were found to be made up of three populations: a somatically hypermutated IgM⁺ subset, a somatically hypermutated IgG⁺ MBC subset, and an unmutated IgD⁺ MBC population. Rechallenge experiments revealed that high affinity, somatically hypermutated Plasmodium-specific IgM⁺ MBCs proliferated and gave rise to antibody secreting cells that dominated the early secondary response to parasite rechallenge. IgM⁺ MBCs also gave rise to T cell-dependent IgM⁺ and IgG⁺ B220⁺CD138⁺ plasmablasts or T cell-independent B220⁻CD138⁺IgM⁺ plasma cells. Thus, even in competition with IgG⁺ MBCs, IgM⁺ MBCs are rapid, plastic, early responders to a secondary Plasmodium rechallenge and thus are novel targets for vaccine strategies.

B cells play a critical role in immune protection to the blood stage of Plasmodium infection. The protective role for antibody was first demonstrated via passive transfer of hyperimmune immunoglobulin from adults to parasitemic children (Cohen et al., 1961), resulting in a dramatic decrease in blood stage parasitemia. Little is known, however, about the cellular source of Plasmodium-specific antibodies largely due to a lack of tools to analyze Plasmodium-specific B cells. Therefore, B cell tetramers specific for the blood stage Plasmodium antigen, Merozoite Surface Protein 1 (MSP1), were generated. MSP1 is a key surface protein expressed by the parasite and is required for erythrocyte invasion (Kadekoppala and Holder, 2010). Antibodies generated against the 19 kD C-terminus region of MSP1 potently inhibit erythrocyte invasion and animals actively, or passively, immunized against MSP1 are protected against subsequent infection (Blackman et al., 1990; Hirunpetcharat et al., 1997; Moss et al., 2012). Furthermore, the acquisition of both IgG and IgM antibodies against the MSP1 C-terminus have been associated with the development of clinical immunity (al-Yaman et al., 1996; Arama et al., 2015; Branch et al., 1998; Dodoo et al., 2008; Riley et al., 1992).

As shown herein, tetramer enrichment techniques permitted the direct ex vivo visualization of rare Plasmodium-specific MBCs in malaria infected humans and mice. Detailed analyses of MSP1⁺ MBC formation and function in the rodent model of malaria, Plasmodium chabaudi, were then performed. Both isotype-switched and unswitched MBCs emerged early in infection and persisted for at least one year. MSP1⁺ MBCs were composed of three distinct subsets including: classically defined, somatically hypermutated, high population that expressed somatically hypermutated BCRs that exhibit equivalent affinity to their IgG⁺ MBC counterparts. In response to various doses of malaria rechallenge, the majority of newly formed antibody-secreting cells (ASCs) were somatically hypermutated IgM⁺ cells, despite IgM⁺ MBCs being at a numerical disadvantage at the time of challenge. Furthermore, IgM⁺ MBCs produced both IgM and IgG antibody in response to rechallenge, thereby also contributing to the IgG⁺ antibody response two days later. Collectively, these studies demonstrate that Plasmodium-specific IgM⁺ MBCs are high affinity, pluripotent early responders to malaria rechallenge that can provide a critical stop gap until IgG antibodies are generated and can be used in the development of more effective vaccine strategies. These results are applicable across the spectrum of antigens, whether derived from pathogens (bacteria, viruses, fungi, protozoa, etc.) or, for example, from tumor antigens.

Accordingly, provided herein, are methods of sorting antigen-specific IgM memory B cells comprising: contacting a biological sample obtained from a subject having had prior exposure to an antigen of interest with an agent comprising the antigen or a portion thereof; and sorting a cell population comprising IgM memory B cells based on binding to the agent comprising the antigen.

In some embodiments of these methods and all such methods described herein, the method further comprises sorting the population comprising antigen-specific IgM memory B cells using an agent specific for CD21, an agent specific for CD27, an agent specific for IgM isotype, or any combination thereof to isolate a population of IgM memory B cells specific for the antigen.

In some embodiments of these methods and all such methods described herein, the agent comprising the antigen comprises a multimer of the antigen. In some embodiments, the agent comprising the antigen comprises a dimer, trimer or tetramer of the antigen.

In some embodiments of these methods and all such methods described herein, the antigen is from an infectious organism.

In some embodiments of these methods and all such methods described herein, the method further comprises one or more steps of sequencing one or more B cell receptors (BCRs) of the cell population comprising IgM memory B cells.

In some embodiments of these methods and all such methods described herein, the method further comprises one or more steps of cloning the one or more BCRs, or antigen binding domains thereof, and expressing the one or more BCRs or antigen-binding domains thereof as one or more recombinant antigen-binding polypeptides.

In some embodiments of these methods and all such methods described herein, the biological sample comprises a blood sample.

Provided herein, in some aspects, are recombinant cells producing an antigen-binding polypeptide comprising a variable heavy chain immunoglobulin sequence, a variable light chain immunoglobulin sequence, or both, from an IgM memory B cell obtained using any of the methods described herein.

In some aspects, provided herein are recombinant antigen-binding polypeptides isolated from a recombinant cell as described herein.

In some aspects, provided herein are recombinant antigen-binding polypeptides comprising an antigen-binding domain of an IgM memory B cell receptor.

In some embodiments of these aspects and all such aspects described herein, the antigen-binding domain comprises a variable light chain sequence, a variable heavy chain sequence, or both.

In some embodiments of these aspects and all such aspects described herein, the IgM memory B cell receptor antigen-binding domain is comprised in a non-IgM isotype antibody framework. In some embodiments, the non-IgM isotype antibody framework is an IgG antibody framework.

In some embodiments of these aspects and all such aspects described herein, the IgM memory B cell receptor antigen-binding domain is a human IgM memory B cell receptor antigen binding domain.

In some embodiments of these aspects and all such aspects described herein, the IgM memory B cell is CD21+CD27+. In some embodiments of these aspects and all such aspects described herein, the recombinant antigen-binding polypeptide comprises an scFv polypeptide, a single-domain antibody construct, a chimeric antibody construct or a bispecific antibody construct.

In some embodiments of these aspects and all such aspects described herein, the polypeptide binds its antigen with a K_{D} of 10⁻⁶ nM or lower.

In some embodiments of these aspects and all such aspects described herein, the variable light chain immunoglobulin sequence, variable heavy chain immunoglobulin sequence, or both has one or more somatic mutations relative to a variable heavy chain immunoglobulin sequence or variable light chain immunoglobulin sequence from a naïve B cell. In some embodiments, the variable light chain sequence, variable heavy chain sequence, or both has one to eight somatic mutations relative to a variable heavy chain sequence or variable light chain sequence from a naïve B cell. In some embodiments, the antigen-binding domain of the IgM memory B cell receptor has fewer than 5 somatic mutations.

In some embodiments of these aspects and all such aspects described herein, the IgM memory B cell receptor antigen-binding domain specifically binds an antigen comprised or expressed by an infectious organism. In some embodiments, the infectious organism is a blood-borne pathogen. In some embodiments, the infectious organism is a virus, a bacterium, a fungus or a parasite. In some embodiments, the infectious organism is P. falciparum. In some embodiments, the antigen is P. falciparum merozoite surface protein 1 (MSP1) or apical membrane antigen 1 (AMA).

In some embodiments of these aspects and all such aspects described herein, the IgM memory B cell receptor antigen-binding domain specifically binds a tumor antigen.

Provided herein, in some aspects, are compositions comprising a population of antigen-specific IgM memory B cells bound via their B cell receptors to antigen immobilized on a solid support.

In some embodiments of these aspects and all such aspects described herein, the antigen immobilized on the solid support comprises a multimer construct comprising the antigen. In some embodiments, the multimer construct comprises a dimer, trimer or tetramer of the antigen.

In some embodiments of these aspects and all such aspects described herein, the antigen is an antigen expressed by an infectious organism. In some embodiments, the infectious organism is a blood-borne pathogen. In some embodiments, the infectious organism is a virus, a bacterium, a fungus or a parasite. In some embodiments, the infectious organism is P. falciparum. In some embodiments, the antigen is P. falciparum merozoite surface protein 1 (MSP1) or apical membrane antigen 1 (AMA).

In some embodiments of these aspects and all such aspects described herein, the IgM memory B cell receptor antigen-binding domain specifically binds a tumor antigen.

In some aspects, provided herein are populations of at least 100 recombinant antigen-binding molecules, each comprising an antigen-binding domain of an IgM memory B cell receptor, and each binding its antigen with a K_{D} of 10⁻⁶ nM or lower.

In some embodiments of these aspects and all such aspects described herein, the average frequency of somatic mutation is eight or fewer per molecule. In some embodiments, the average frequency of somatic mutation is five or fewer per molecule.

In some embodiments of these aspects and all such aspects described herein, the population binds the same antigen.

In some embodiments of these aspects and all such aspects described herein, the antigen is an antigen expressed or comprised by an infectious organism. In some embodiments, the infectious organism is a blood-borne pathogen. In some embodiments, the infectious organism is a virus, a bacterium, a fungus, or a parasite. In some embodiments, the infectious organism is P. falciparum. In some embodiments, the antigen is P. falciparum merozoite surface protein 1 (MSP1) or apical membrane antigen 1 (AMA).

In some aspects, provided herein are pharmaceutical compositions comprising any of the compositions described herein and a pharmaceutically acceptable carrier.

In some aspects, provided herein are vaccine compositions comprising a composition as described herein.

In some aspects, provided herein are methods of treating a subject in need of treatment for a disease caused by an infectious organism comprising administering a composition comprising an antigen-binding polypeptide as described herein to the subject, wherein the antigen-binding polypeptide specifically binds an antigen comprised by the infectious organism.

In some aspects, provided herein are methods of reducing the likelihood of contracting a disease caused by an infectious organism comprising administering to an individual at risk of contracting the disease a composition comprising an antigen-binding polypeptide as described herein to the subject, wherein the antigen-binding polypeptide specifically binds an antigen comprised by the infectious organism.

In some aspects, provided herein are methods of treating a subject in need of treatment for a tumor that expresses a tumor antigen comprising administering a composition comprising an antigen-binding polypeptide as described herein to the subject, wherein the antigen-binding polypeptide specifically binds the tumor antigen.

Provided herein, in some aspects, are methods of sorting Plasmodium-specific IgM memory B cells (MBCs), comprising: generating B cell tetramers specific for blood or liver stage Plasmodium antigens; providing the B cell tetramers to a biological sample obtained from a subject infected with malaria; and sorting the Plasmodium-specific IgM MBCs based on binding to the tetramers.

In some embodiments of these aspects and all such aspects described herein, the method further comprises one or more steps of sequencing the Plasmodium-specific IgM MBC B cell receptors (BCRs).

In some embodiments of these aspects and all such aspects described herein, the method further comprises one or more steps of cloning the BCRs and expressing the BCRs as recombinant antibodies.

In some embodiments of these aspects and all such aspects described herein, the subject is a mammal. In some embodiments, the subject is a human.

In some aspects, provided herein are isolated or recombinant antibody-producing B-cells produced by using any of the methods described herein. In some aspects, provided herein are recombinant antibodies produced from the isolated or recombinant antibody-producing B-cell.

In some aspects, provided herein are recombinant antibodies comprising a variable region from Plasmodium-specific memory B cells and an immunoglobulin heavy chain isotype. In some embodiments, the recombinant antibody is for the treatment of or protection from malaria infection in a subject. In some embodiments, the recombinant antibody is for vaccination against malaria. In some embodiments, the recombinant antibody is for the treatment of multi-drug resistant malaria. In some aspects, provided herein are pharmaceutical composition comprising such recombinant antibodies.

In some embodiments of these aspects and all such aspects described herein, the subject is a mammal. In some embodiments, the subject is a human.

Provided herein, in some aspects, are methods of treating malaria infection in a subject, comprising administering a therapeutically effective amount of a recombinant antibody as described herein. In some embodiments of these aspects and all such aspects described herein, the subject is a mammal. In some embodiments, the subject is a human. In some embodiments, the subject is immunocompromised.

Provided herein, in some aspects, are methods of treating multi-drug resistant malaria in a subject, comprising administering a therapeutically effective amount of a recombinant antibody as described herein. In some embodiments of these aspects and all such aspects described herein, the subject is a mammal. In some embodiments, the subject is a human. In some embodiments, the subject is immunocompromised.

Provided herein, in some aspects, are methods of preventing malaria infection in a subject, comprising administering a pharmaceutically effective amount of a recombinant antibody as described herein. In some embodiments of these aspects and all such aspects described herein, the subject is a mammal. In some embodiments, the subject is a human. In some embodiments, the subject is immunocompromised.

In some embodiments of these aspects and all such aspects described herein, the recombinant antibody is administered in an amount effective to provide short-term protection against a malaria infection. In some embodiments, the short-term protection is at least about 2 months. In some embodiments, the short-term protection is at least about 3 months.

Provided herein, in some aspects, are methods for assessing an effective vaccine strategy for malaria infection in a subject comprising: generating B cell tetramers specific for blood or liver stage Plasmodium antigens; providing the B cell tetramers to a biological sample obtained from the subject; and sorting or enumerating the Plasmodium-specific IgM MBCs based on binding to the tetramers. In some embodiments of these aspects and all such aspects described herein, the method further comprises a step of sequencing the Plasmodium-specific IgM MBC B cell receptors (BCRs). In some embodiments of these aspects and all such aspects described herein, the subject is a mammal. In some embodiments, the subject is a human. In some embodiments, the subject is immunocompromised.

Provided herein, in some aspects, are recombinant antibodies or antigen-binding fragments thereof that specifically bind to the malarial antigen apical membrane antigen 1 (AMA) and comprises heavy chain complimentarity determining regions (CDRs) selected from the group consisting of:
a. a heavy chain CDR1 having the amino acid sequence of SEQ ID NO: 27; a heavy chain CDR2 having the amino acid sequence of SEQ ID NO: 28; and a heavy chain CDR3 having the amino acid sequence of SEQ ID NO: 29;
b. a heavy chain CDR1 having the amino acid sequence of SEQ ID NO: 37; a heavy chain CDR2 having the amino acid sequence of SEQ ID NO: 38; and a heavy chain CDR3 having the amino acid sequence of SEQ ID NO: 39;
c. a heavy chain CDR1 having the amino acid sequence of SEQ ID NO: 47; a heavy chain CDR2 having the amino acid sequence of SEQ ID NO: 48; and a heavy chain CDR3 having the amino acid sequence of SEQ ID NO: 49;
d. a heavy chain CDR1 having the amino acid sequence of SEQ ID NO: 57; a heavy chain CDR2 having the amino acid sequence of SEQ ID NO: 58; and a heavy chain CDR3 having the amino acid sequence of SEQ ID NO: 59;
e. a heavy chain CDR1 having the amino acid sequence of SEQ ID NO: 77; a heavy chain CDR2 having the amino acid sequence of SEQ ID NO: 78; and a heavy chain CDR3 having the amino acid sequence of SEQ ID NO: 79;
f. a heavy chain CDR1 having the amino acid sequence of SEQ ID NO: 107; a heavy chain CDR2 having the amino acid sequence of SEQ ID NO: 108; and a heavy chain CDR3 having the amino acid sequence of SEQ ID NO: 109;
g. a heavy chain CDR1 having the amino acid sequence of SEQ ID NO: 137; a heavy chain CDR2 having the amino acid sequence of SEQ ID NO: 138; and a heavy chain CDR3 having the amino acid sequence of SEQ ID NO: 139; and
h. a heavy chain CDR1 having the amino acid sequence of SEQ ID NO: 147; a heavy chain CDR2 having the amino acid sequence of SEQ ID NO: 148; and a heavy chain CDR3 having the amino acid sequence of SEQ ID NO: 149.

Provided herein, in some aspects, are recombinant antibodies or antigen-binding fragments thereof that specifically bind to the malarial antigen apical membrane antigen 1 (AMA) and comprises light chain complimentarity determining regions (CDRs) selected from the group consisting of:
a. a light chain CDR1 having the amino acid sequence of SEQ ID NO: 32; a light chain CDR2 having the amino acid sequence of SEQ ID NO: 33; and a light chain CDR3 having the amino acid sequence of SEQ ID NO: 34;
b. a light chain CDR1 having the amino acid sequence of SEQ ID NO: 42; a light chain CDR2 having the amino acid sequence of SEQ ID NO: 43; and a light chain CDR3 having the amino acid sequence of SEQ ID NO: 44;
c. a light chain CDR1 having the amino acid sequence of SEQ ID NO: 52; a light chain CDR2 having the amino acid sequence of SEQ ID NO: 53; and a light chain CDR3 having the amino acid sequence of SEQ ID NO: 54;
d. a light chain CDR1 having the amino acid sequence of SEQ ID NO: 62; a light chain CDR2 having the amino acid sequence of SEQ ID NO: 63; and a light chain CDR3 having the amino acid sequence of SEQ ID NO: 64;
e. a light chain CDR1 having the amino acid sequence of SEQ ID NO: 82; a light chain CDR2 having the amino acid sequence of SEQ ID NO: 83; and a light chain CDR3 having the amino acid sequence of SEQ ID NO: 84;
f. a light chain CDR1 having the amino acid sequence of SEQ ID NO: 112; a light chain CDR2 having the amino acid sequence of SEQ ID NO: 113; and a light chain CDR3 having the amino acid sequence of SEQ ID NO: 114;
g. a light chain CDR1 having the amino acid sequence of SEQ ID NO: 142; a light chain CDR2 having the amino acid sequence of SEQ ID NO: 143; and a light chain CDR3 having the amino acid sequence of SEQ ID NO: 144; and
h. a light chain CDR1 having the amino acid sequence of SEQ ID NO: 152; a light chain CDR2 having the amino acid sequence of SEQ ID NO: 153; and a light chain CDR3 having the amino acid sequence of SEQ ID NO: 154.

Provided herein, in some aspects, are recombinant antibodies or antigen-binding fragments thereof that specifically bind to the malarial antigen apical membrane antigen 1 (AMA) and comprises heavy and light chain complimentarity determining regions (CDRs) selected from the group consisting of:
a. a heavy chain CDR1 having the amino acid sequence of SEQ ID NO: 27; a heavy chain CDR2 having the amino acid sequence of SEQ ID NO: 28; a heavy chain CDR3 having the amino acid sequence of SEQ ID NO: 29; a light chain CDR1 having the amino acid sequence of SEQ ID NO: 32; a light chain CDR2 having the amino acid sequence of SEQ ID NO: 33; and a light chain CDR3 having the amino acid sequence of SEQ ID NO: 34;
b. a heavy chain CDR1 having the amino acid sequence of SEQ ID NO: 37; a heavy chain CDR2 having the amino acid sequence of SEQ ID NO: 38; a heavy chain CDR3 having the amino acid sequence of SEQ ID NO: 39; a light chain CDR1 having the amino acid sequence of SEQ ID NO: 42; a light chain CDR2 having the amino acid sequence of SEQ ID NO: 43; and a light chain CDR3 having the amino acid sequence of SEQ ID NO: 44;
c. a heavy chain CDR1 having the amino acid sequence of SEQ ID NO: 47; a heavy chain CDR2 having the amino acid sequence of SEQ ID NO: 48; a heavy chain CDR3 having the amino acid sequence of SEQ ID NO: 49; a light chain CDR1 having the amino acid sequence of SEQ ID NO: 52; a light chain CDR2 having the amino acid sequence of SEQ ID NO: 53; and a light chain CDR3 having the amino acid sequence of SEQ ID NO: 54;
d. a heavy chain CDR1 having the amino acid sequence of SEQ ID NO: 57; a heavy chain CDR2 having the amino acid sequence of SEQ ID NO: 58; a heavy chain CDR3 having the amino acid sequence of SEQ ID NO: 59; a light chain CDR1 having the amino acid sequence of SEQ ID NO: 62; a light chain CDR2 having the amino acid sequence of SEQ ID NO: 63; and a light chain CDR3 having the amino acid sequence of SEQ ID NO: 64;
e. a heavy chain CDR1 having the amino acid sequence of SEQ ID NO: 77; a heavy chain CDR2 having the amino acid sequence of SEQ ID NO: 78; a heavy chain CDR3 having the amino acid sequence of SEQ ID NO: 79; a light chain CDR1 having the amino acid sequence of SEQ ID NO: 82; a light chain CDR2 having the amino acid sequence of SEQ ID NO: 83; and a light chain CDR3 having the amino acid sequence of SEQ ID NO: 84;
f. a heavy chain CDR1 having the amino acid sequence of SEQ ID NO: 107; a heavy chain CDR2 having the amino acid sequence of SEQ ID NO: 108; a heavy chain CDR3 having the amino acid sequence of SEQ ID NO: 109; a light chain CDR1 having the amino acid sequence of SEQ ID NO: 112; a light chain CDR2 having the amino acid sequence of SEQ ID NO: 113; and a light chain CDR3 having the amino acid sequence of SEQ ID NO: 114;
g. a heavy chain CDR1 having the amino acid sequence of SEQ ID NO: 137; a heavy chain CDR2 having the amino acid sequence of SEQ ID NO: 138; a heavy chain CDR3 having the amino acid sequence of SEQ ID NO: 139; a light chain CDR1 having the amino acid sequence of SEQ ID NO: 142; a light chain CDR2 having the amino acid sequence of SEQ ID NO: 143; and a light chain CDR3 having the amino acid sequence of SEQ ID NO: 144; and
h. a heavy chain CDR1 having the amino acid sequence of SEQ ID NO: 147; a heavy chain CDR2 having the amino acid sequence of SEQ ID NO: 148; a heavy chain CDR3 having the amino acid sequence of SEQ ID NO: 149; a light chain CDR1 having the amino acid sequence of SEQ ID NO: 152; a light chain CDR2 having the amino acid sequence of SEQ ID NO: 153; and a light chain CDR3 having the amino acid sequence of SEQ ID NO: 154.

Provided herein, in some aspects, are recombinant antibodies or antigen-binding fragments thereof that specifically bind to the malarial antigen Merozoite Surface Protein 1 (MSP1) and comprises heavy chain complimentarity determining regions (CDRs) selected from the group consisting of:
a. a heavy chain CDR1 having the amino acid sequence of SEQ ID NO: 67; a heavy chain CDR2 having the amino acid sequence of SEQ ID NO: 68; and a heavy chain CDR3 having the amino acid sequence of SEQ ID NO: 69;
b. a heavy chain CDR1 having the amino acid sequence of SEQ ID NO: 87; a heavy chain CDR2 having the amino acid sequence of SEQ ID NO: 88; and a heavy chain CDR3 having the amino acid sequence of SEQ ID NO: 89;
c. a heavy chain CDR1 having the amino acid sequence of SEQ ID NO: 97; a heavy chain CDR2 having the amino acid sequence of SEQ ID NO: 98; and a heavy chain CDR3 having the amino acid sequence of SEQ ID NO: 99;
d. a heavy chain CDR1 having the amino acid sequence of SEQ ID NO: 117; a heavy chain CDR2 having the amino acid sequence of SEQ ID NO: 118; and a heavy chain CDR3 having the amino acid sequence of SEQ ID NO: 119; and
e. a heavy chain CDR1 having the amino acid sequence of SEQ ID NO: 127; a heavy chain CDR2 having the amino acid sequence of SEQ ID NO: 128; and a heavy chain CDR3 having the amino acid sequence of SEQ ID NO: 129.

Provided herein, in some aspects, are recombinant antibodies or antigen-binding fragments thereof that specifically bind to the malarial antigen Merozoite Surface Protein 1 (MSP1) and comprises light chain complimentarity determining regions (CDRs) selected from the group consisting of:
a. a light chain CDR1 having the amino acid sequence of SEQ ID NO: 72; a light chain CDR2 having the amino acid sequence of SEQ ID NO: 73; and a light chain CDR3 having the amino acid sequence of SEQ ID NO: 74;
b. a light chain CDR1 having the amino acid sequence of SEQ ID NO: 92; a light chain CDR2 having the amino acid sequence of SEQ ID NO: 93; and a light chain CDR3 having the amino acid sequence of SEQ ID NO: 94;
c. a light chain CDR1 having the amino acid sequence of SEQ ID NO: 102; a light chain CDR2 having the amino acid sequence of SEQ ID NO: 103; and a light chain CDR3 having the amino acid sequence of SEQ ID NO: 104;
d. a light chain CDR1 having the amino acid sequence of SEQ ID NO: 122; a light chain CDR2 having the amino acid sequence of SEQ ID NO: 123; and a light chain CDR3 having the amino acid sequence of SEQ ID NO: 124; and
e. a light chain CDR1 having the amino acid sequence of SEQ ID NO: 132; a light chain CDR2 having the amino acid sequence of SEQ ID NO: 133; and a light chain CDR3 having the amino acid sequence of SEQ ID NO: 134.

Provided herein, in some aspects, are recombinant antibodies or antigen-binding fragments thereof that specifically bind to the malarial antigen Merozoite Surface Protein 1 (MSP1) and comprises heavy and light chain complimentarity determining regions (CDRs) selected from the group consisting of:
a. a heavy chain CDR1 having the amino acid sequence of SEQ ID NO: 67; a heavy chain CDR2 having the amino acid sequence of SEQ ID NO: 68; a heavy chain CDR3 having the amino acid sequence of SEQ ID NO: 69; a light chain CDR1 having the amino acid sequence of SEQ ID NO: 72; a light chain CDR2 having the amino acid sequence of SEQ ID NO: 73; and a light chain CDR3 having the amino acid sequence of SEQ ID NO: 74;
b. a heavy chain CDR1 having the amino acid sequence of SEQ ID NO: 87; a heavy chain CDR2 having the amino acid sequence of SEQ ID NO: 88; a heavy chain CDR3 having the amino acid sequence of SEQ ID NO: 89; a light chain CDR1 having the amino acid sequence of SEQ ID NO: 92; a light chain CDR2 having the amino acid sequence of SEQ ID NO: 93; and a light chain CDR3 having the amino acid sequence of SEQ ID NO: 94;
c. a heavy chain CDR1 having the amino acid sequence of SEQ ID NO: 97; a heavy chain CDR2 having the amino acid sequence of SEQ ID NO: 98; a heavy chain CDR3 having the amino acid sequence of SEQ ID NO: 99; a light chain CDR1 having the amino acid sequence of SEQ ID NO: 102; a light chain CDR2 having the amino acid sequence of SEQ ID NO: 103; and a light chain CDR3 having the amino acid sequence of SEQ ID NO: 104;
d. a heavy chain CDR1 having the amino acid sequence of SEQ ID NO: 117; a heavy chain CDR2 having the amino acid sequence of SEQ ID NO: 118; a heavy chain CDR3 having the amino acid sequence of SEQ ID NO: 119; a light chain CDR1 having the amino acid sequence of SEQ ID NO: 122; a light chain CDR2 having the amino acid sequence of SEQ ID NO: 123; and a light chain CDR3 having the amino acid sequence of SEQ ID NO: 124; and
e. a heavy chain CDR1 having the amino acid sequence of SEQ ID NO: 127; a heavy chain CDR2 having the amino acid sequence of SEQ ID NO: 128; a heavy chain CDR3 having the amino acid sequence of SEQ ID NO: 129; a light chain CDR1 having the amino acid sequence of SEQ ID NO: 132; a light chain CDR2 having the amino acid sequence of SEQ ID NO: 133; and a light chain CDR3 having the amino acid sequence of SEQ ID NO: 134.

In some aspects, described herein are methods of sorting Plasmodium-specific IgM memory B cells (MBCs), including the steps of: generating B cell tetramers specific for blood or liver stage Plasmodium antigens; providing the B cell tetramers to a biological sample obtained from a subject infected with malaria; and sorting the Plasmodium-specific IgM MBCs based on binding to the tetramers. In some embodiments, the method further includes a step of sequencing the Plasmodium-specific IgM MBC B cell receptors (BCRs). In some embodiments, the method further includes a step of cloning the BCRs and expressing the BCRs as recombinant antibodies.

In other aspects, described herein are isolated or recombinant antibody-producing B-cells produced by the aforementioned method. In some embodiments, described herein are recombinant antibodies produced from the isolated or recombinant antibody-producing B-cell.

In other aspects, described herein are recombinant antibodies, including a variable region from Plasmodium-specific memory B cells and any heavy chain isotype. In some embodiments, the recombinant antibodies are for the treatment of or protection from malaria infection in a subject. In some embodiments, the recombinant antibodies are for vaccination against malaria. In some embodiments, the recombinant antibodies are for the treatment of multi-drug resistant malaria.

In other aspects, provided herein are pharmaceutical compositions comprising any of the aforementioned recombinant antibodies.

In other aspects, provided herein are methods of treating or preventing malaria infection in a subject by administering a therapeutically effective amount of any of the aforementioned recombinant antibodies. In some embodiments, the malaria infection is a multi-drug resistant malaria infection. In some embodiments, the recombinant antibody provides short-term protection against a malaria infection. In some embodiments, the short-term protection is at least about 2 months, or at least about 3 months.

In other aspects, provided herein are methods for assessing an effective vaccine strategy for malaria infection in a subject, including generating B cell tetramers specific for blood or liver stage Plasmodium antigens; providing the B cell tetramers to a biological sample obtained from the subject; and sorting the Plasmodium-specific IgM MBCs based on binding to the tetramers. In some embodiments, the method further includes a step of sequencing the Plasmodium-specific IgM MBC B cell receptors (BCRs).

In some embodiments of the aspects described herein, the subject is a mammal. Preferably, the subject is a human. In some embodiments of the aspects described herein, the subject is immunocompromised.

### Definitions

Unless otherwise defined herein, scientific and technical terms used in connection with the present application shall have the meanings that are commonly understood by those of ordinary skill in the art to which this disclosure belongs. It should be understood that this invention is not limited to the particular methodology, protocols, and reagents, etc., described herein and as such can vary. The terminology used herein is for the purpose of describing particular embodiments only, and is not intended to limit the scope of the present invention, which is defined solely by the claims. Definitions of common terms in immunology, and molecular biology can be found in The Merck Manual of Diagnosis and Therapy, 19th Edition, published by Merck Sharp & Dohme Corp., 2011 (ISBN 978-O-911910-19-3); Robert S. Porter et al. (eds.), The Encyclopedia of Molecular Cell Biology and Molecular Medicine, published by Blackwell Science Ltd., 1999-2012 (ISBN 9783527600908); and Robert A. Meyers (ed.), Molecular Biology and Biotechnology: a Comprehensive Desk Reference, published by VCH Publishers, Inc., 1995 (ISBN 1-56081-569-8); Immunology by Werner Luttmann, published by Elsevier, 2006; Janeway's Immunobiology, Kenneth Murphy, Allan Mowat, Casey Weaver (eds.), Taylor & Francis Limited, 2014 (ISBN 0815345305, 9780815345305); Lewin's Genes XI, published by Jones & Bartlett Publishers, 2014 (ISBN-1449659055); Michael Richard Green and Joseph Sambrook, Molecular Cloning: A Laboratory Manual, 4^{th} ed., Cold Spring Harbor Laboratory Press, Cold Spring Harbor, N.Y., USA (2012) (ISBN 1936113414); Davis et al., Basic Methods in Molecular Biology, Elsevier Science Publishing, Inc., New York, USA (2012) (ISBN 044460149X); Laboratory Methods in Enzymology: DNA, Jon Lorsch (ed.) Elsevier, 2013 (ISBN 0124199542); Current Protocols in Molecular Biology (CPMB), Frederick M. Ausubel (ed.), John Wiley and Sons, 2014 (ISBN 047150338X, 9780471503385), Current Protocols in Protein Science (CPPS), John E. Coligan (ed.), John Wiley and Sons, Inc., 2005; and Current Protocols in Immunology (CPI) (John E. Coligan, ADA M Kruisbeek, David H Margulies, Ethan M Shevach, Warren Strobe, (eds.) John Wiley and Sons, Inc., 2003 (ISBN 0471142735, 9780471142737), the contents of which are all incorporated by reference herein in their entireties.

An “increase” or “decrease” refers to a statistically significant increase or decrease respectively. For the avoidance of doubt, an increase or decrease will be at least 10% relative to a reference, such as at least 10%, at least 20%, at least 30%, at least 40%, at least 50%, at least 60%, at least 70%, at least 80%, at least 90%, at least 95%, at least 97%, at least 98%, or more, up to and including at least 100% or more, inclusive, in the case of an increase, for example, at least 2-fold, at least 3-fold, at least 4-fold, at least 5-fold, at least 6-fold, at least 7-fold, at least 8-fold, at least 9-fold, at least 10-fold, at least 50-fold, at least 100-fold, or more.

As used herein the term “comprising” or “comprises” is used in reference to compositions, methods, and respective component(s) thereof, that are essential to the method or composition, yet open to the inclusion of unspecified elements, whether essential or not.

As used herein the term “consisting essentially of” refers to those elements required for a given embodiment. The term permits the presence of elements that do not materially affect the basic and novel or functional characteristic(s) of that embodiment.

The term “consisting of” refers to compositions, methods, and respective components thereof as described herein, which are exclusive of any element not recited in that description of the embodiment.

As used in this specification and the appended claims, the singular forms “a,” “an,” and “the” include plural references unless the context clearly dictates otherwise. Thus for example, references to “the method” includes one or more methods, and/or steps of the type described herein and/or which will become apparent to those persons skilled in the art upon reading this disclosure and so forth. Similarly, the word “or” is intended to include “and” unless the context clearly indicates otherwise. Although methods and materials similar or equivalent to those described herein can be used in the practice or testing of this disclosure, suitable methods and materials are described below. The abbreviation, “e.g.” is derived from the Latin exempli gratia, and is used herein to indicate a non-limiting example. Thus, the abbreviation “e.g.” is synonymous with the term “for example.”

Other than in the operating examples, or where otherwise indicated, all numbers expressing quantities of ingredients or reaction conditions used herein should be understood as modified in all instances by the term “about.” The term “about” when used in connection with percentages can mean±1%.

The term “statistically significant” or “significantly” refers to statistical significance and generally means a two standard deviation (2SD) difference, above or below a reference value. Additional definitions are provided in the text of individual sections below.

### BRIEF DESCRIPTION OF THE DRAWINGS

FIGS. 1A-1E. Plasmodium-specific B cells expand early and persist and detection and kinetics of MSP1⁺ B cells. FIG. 1A. Splenic B cells identified by gating on all B220⁺ and CD138⁺ cell after excluding CD3⁺F4/80⁺ non-B cells and enrichment with MSP1 and Decoy tetramers. FIG. 1B. Representative plots show MSP1⁺ B cells from (left) uninfected mice or (right) mice 8 days post-infection (p.i.). FIG. 1C. Total number of MSP1⁺ B cells from individual uninfected or 8 days post-infection (p.i.) mice. Data is combined from 2 independent experiments with 5-6 mice per group. Line indicates mean **p<0.01. FIG. 1D. Kinetics of MSP1⁺ B cells (left Y-axis) and percent parasitemia (right Y-axis) over 20 days p.i. FIG. 1E. Total MSP1⁺ B cells over 340 days p.i. For 1D and 1E, each data point shows mean±SEM with 3-8 mice per time point from at least two independent experiments.

FIGS. 2A-2B. MSP1⁺ B cell fates emerge and expand early after infection along with effector B cells and MBCs persist. FIG. 2A. Gating scheme and representative plots of MSP1⁺ B cells to identify CD138+ plasmablasts and naïve/memory B cells. CD138+ cells (top row) and CD138− memory/naïve cells, precursor cells, and GC B cells (bottom row) over 265 days post-infection. FIG. 2B. Total MSP1⁺ MBCs/naïve, plasmablasts, and GC (germinal center) B cells over 340 days post-infection. Each data point shows mean±SEM with 3-8 mice per time point from at least 2 independent experiments.

FIGS. 3A-3E. MSP1⁺ MBCs are heterogeneous. FIG. 3A. Representative plot of MSP1⁺ MBCs and isotype of MSP1⁺CD38⁺ MBCs to identify IgD⁺, IgM⁺, and swIg⁺ MBCs 100 days p.i. FIG. 3B. Total number of MSP1⁺IgD⁺, IgM⁺, and swIg⁺ MBCs from day 40 to 340 p.i. Each data point shows mean±SEM with 3-8 mice per time point from at least 2 independent experiments. FIG. 3C. Representative plots showing expression of CD73 and CD80 on MSP1⁺ naïve B cells or IgD⁺, IgM⁺, and swIg⁺ MBCs 100 days p.i. FIG. 3D. Somatically hypermutated IgM+ and swIg+ MBCs are clonally related. Number of mutations in the heavy chain (VH) or light chain (VL) of individual MSP1⁺ naïve B cells or CD73⁻CD80⁻IgD⁺, CD73⁺CD80⁺IgM⁺, or CD73⁺CD80⁺swIg⁺ MBCs 100 days p.i. Each dot indicates a single cell. Line indicates mean. Data combined from 3 independent experiments. ***p<0.001 FIG. 3E. ELISA of serially diluted MSP1-specific IgM⁺ and swIg⁺ mAbs. Each line represents a single clone. OD450, optical density at 450 nm.

FIGS. 4A-4D. Rapid expansion of MSP1⁺ B cells after parasite rechallenge. FIG. 4A. Representative plots identifying MSP1⁺ B cells in memory mice (mice previously infected 12-16 weeks prior) rechallenged with 1×10⁷ unRBCs or iRBCs and analyzed 3 or 5 days later. FIG. 4B. Total number of MSP1⁺ B cells in A. Data combined from 2 independent experiments with 6-9 mice per group. Line indicates mean. **p<0.01,***p<0.001. FIG. 4C. Representative plots of B220 by CD138 on MSP1⁺ B cells in memory mice rechallenged with 1×10⁷ iRBCs analyzed 3 or 5 days later. FIG. 4D. Total number of MSP1⁺ B220⁺CD138⁻, B220⁻CD138⁺, and B220⁺CD138⁺ cells in C. Data combined from 2 independent experiments with 6-9 mice per group. Line indicates mean. *p<0.05, **p<0.01,***p<0.001.

FIGS. 5A-5D. Early secondary antibody response is IgM-dominant. FIG. 5A. Representative plots of intracellular Ig(H+L) (Ig) and CD138 expression of MSP1⁺ B cells. Bottom row shows B220 by IgM expression of MSP1⁺Ig⁺CD138⁺ cells in memory mice pre-challenge or 3 or 5 days post-challenge with 1×10⁷ iRBCs. FIG. 5B. Total number of all IgM⁺ and IgM⁻ MSP1⁺ CD138Ig⁺ cells in A. Data combined from 2 independent experiments with 3-6 mice per group. Line indicates mean. **p<0.01 FIG. 5C. MSP1-19 IgM and IgG ELISA from serum of individual memory mice prechallenge and 3 or 5 days post challenge. OD450, optical density at 450 nm. Each dilution point shows mean±SEM. Graphs represent combined data from 3 independent experiments with 3 mice per group. *p<0.05, **p<0.01 FIG. 5D. ELISPOT on MSP1⁺ IgD⁺, IgM⁺, and swIg⁺ MBCs sorted from memory mice 2 days post challenge. Data compiled from 5 mice in 2 independent experiments. Error bars show SD.

FIGS. 6A-6C. iRBC challenge dose or timing does not impact secondary IgM response. FIG. 6A. Representative plots of B220 and IgM expression on MSP1⁺Ig⁺CD138⁺ cells in memory mice (12-16 weeks post primary infection) pre challenge or 3 days post challenge with 1×10³ or 1×10⁵. FIG. 6B. Total number of all IgM⁺ and IgM⁻ MSP1⁺ CD138⁺Ig⁺ cells in 6A. Data combined from 2 independent experiments with 3-4 mice per group. Line indicates mean. *p<0.05 FIG. 6C. Representative plots of B220 and IgM expression on MSP1⁺Ig⁺CD138⁺ cells (left) and total number of all IgM⁺ and IgM⁻ MSP1⁺CD138⁺Ig⁺ cells (right) in memory mice 36 weeks p.i. prior to challenge or 3 days post challenge with 1×10⁷ iRBCs. Data combined from 2 independent experiments with 4-5 mice per group. Line indicates mean. *p<0.05

FIGS. 7A-7C. Requirements for secondary IgM⁺ MBC responses. FIG. 7A. Representative plots of MSP1⁺ B cell B220 and CD138 expression after iRBC rechallenge +/− CD4 depletion (GK1.5) FIG. 7B. Total number of MSP1⁺ B220⁺CD138⁺ cells and B220⁻CD138⁺ cells in 7A. Data combined from 2 independent experiments with 3-4 mice per group. Line indicates mean. **p<0.01 FIG. 7C. Representative plots of B220 and IgM expression on MSP1⁺Ig⁺CD138⁺ cells in 7A.

FIGS. 8A-8B. MSP1-specific B cells bind tetramer and expand in an antigen specific manner. FIG. 8A. Representative plots to identify MSP1⁺ B cells from spleens of naïve MD4-Rag2^{-/-} mice. FIG. 8B. CD45.2⁺ MD4− Rag^{-/-} splenocytes were transferred into CD45.1 WT B6 mice and infected with 1×10⁶ iRBCs the following day. 8 days post infection recipient mice spleens were enriched with Decoy and MSP1 B cell tetramers to identify MSP1⁺ B cells from either CD45.1 WT recipient cells or donor CD45.2⁺ MD4-Rag^{-/-} cells.

FIGS. 9A-9B. Measurement of parasitemia by flow cytometry. FIG. 9A. Representative gating scheme to identify P.chabaudi iRBCs gated as Ter119⁺CD45⁻Hoechst⁺ in 1 ul of blood from a WT mouse 8 days post infection. FIG. 9B. Course of parasitemia in WT mice measured by flow cytometry after infection with 1×10⁶ iRBCs over the course of 40 days. Each data point shows mean±SEM with 3-8 mice per timepoint from at least two independent experiments.

FIGS. 10A-10D. Splenic histology and serum antibody analysis reflect cellular kinetics during early, acute phase of infection. FIG. 10A. Representative plots of CD38 and CD138 expression (top row) and IgM and IgD expression (bottom row) on CD138⁺ cells of MSP1⁺ B cells on days 8, 20, and 100 post infection with 1×10⁶ Pc iRBCs. FIG. 10B. Representative plots of CD38 and GL7 expression (top row) and IgM and IgD expression (bottom row) on GL7⁺ cells of MSP1⁺ CD138⁻ cells on days 8, 20, and 100 post infection with 1×10⁶ Pc iRBCs. FIG. 10C. Representative splenic sections from mice 8 or 20 days post infection with 1×10⁶ Pc iRBCs stained with CD4 (red), B220 (blue), and IgD (green). Composite pictures generated by stitching together multiple 10× images over large area of spleen. Scale bars, 100 μm. FIG. 10D. Serum antibody analysis by ELISA for MSP1-specific IgM, IgG1, IgG2b, IgG2c, and IgG3 from individual mice on days 8 and 20 post infection with 1×10⁶ Pc iRBCs. Each dilution point shows mean±SEM. Graphs represent combined data from 3 independent experiments with 3 mice per group.

FIGS. 11A-11C. Human Plasmodium-specific MBCs are phenotypically heterogeneous. CD19⁺ human B cells identified in US and Mali PBMC after excluding CD3⁺CD14⁺CD16⁺ non-B cells and enrichment with PfMSP1, PfAMA1 and Decoy tetramers. FIG. 11A. Representative plots of PfMSP1/AMA1⁺ B cells and CD21/CD27 expression. FIG. 11B. Total PfMSP1/AMA1⁺ B cells and MBCs per 6×10⁷ PBMC in US and Mali PBMC. Data is combined from 3 independent experiments with 7 samples per group. Line indicates mean. *p<0.05 FIG. 11C. Representative plot of IgM and IgD expression on CD27⁺CD21⁺ memory B cells from Mali sample.

FIGS. 12A-12B. Naïve MSP1⁺ B cells do not differentiate or secrete antibody after primary infection with 1×10⁷ iRBCs. FIG. 12A. Representative plots identifying MSP1⁺ B cells (top row) and B220 and CD138 expression (bottom row) in uninfected naïve mice or naïve mice 3 or 5 days post primary infection with 1×10⁷ iRBCs injected i.v. FIG. 12B. Total number of MSP1⁺ B cells in uninfected naïve mice or naïve mice 3 or 5 days post primary infection with 1×10⁷ iRBCs injected i.v. Data is combined from 2 independent experiments with 3-5 mice per group.

FIGS. 13A-13C. Phenotype of newly formed proliferating MSP1-specific B cells after a secondary infection. FIG. 13A. Columns 1 and 2: Ki67 expression on B220^{+/-}MSP1⁺ B cells in memory mice pre or post iRBC challenge (day 3). Column 3: representative plots of B220 and CD138 expression (top) and CD38 and GL7 expression (middle) of Ki67⁺ cells in rechallenged memory mice. FIG. 13B. Representative plots of IgM and IgD expression on Ki67⁺B220⁺ PBs (top), precursors (middle), and MBCs (bottom). Adjacent graph shows percentage of indicated isotypes of each population. Bar graphs represents data combined from 2 independent experiments with 6 mice per group. Error bars show SD. **p<0.01 FIG. 13C. Number of mutations in the heavy chain (VH) of individual MSP1⁺ IgM⁺ MBCs 100 days p.i. prior to challenge and MSP1IgM⁺B220⁺CD138⁺ plasmablasts from memory mice 3 days after challenge with 1×10⁷ iRBCs. Each dot indicates a single cell. Line indicates mean. Data combined from 3 independent experiments.

FIG. 14. Mouse and human surface and intracellular antibodies for staining Plasmodium-specific B cells. For murine and human samples, the following surface antibodies were used in various combinations (purchased from BD Biosciences, Ebioscience, or Biolegend). For intracellular staining (ICS), cells were fixed and permed with BD Cytofix/Cytoperm and washed and stained in BD perm buffer.

FIG. 15. Somatically hypermutated IgM+ and swIg+ MBCs are clonally related. Pie charts representing total number of unique VH genes used within MSP1-specific naïve B cells or IgD+IgM+, or swig+ MBCs from mice 100 days p.i. Each slice and colors represent a specific VH gen. Similar colors between subsets denotes shared genes.

FIG. 16. Schematic showing exemplary model of memory B cell development.

FIG. 17. Schematic showing exemplary model of memory B cell development.

FIG. 18. Both IgG⁺ and IgM⁺Plasmodium-specific MBCs exist in the blood of individuals from endemic regions.

FIG. 19. Representative FACS sorting of Plasmodium falciparum-specific IgG and IgM MBCs from malaria-exposed humans. Single IgM+ and IgG+ MBCs were FACS sorted from 9 individuals and were used for sequencing and cloning, using combined Tmr for both P. falciparum AMA and MSP-1.

FIG. 20. Total Number of FACS-sorted Single Memory B Cells isolated from peripheral blood samples collected from nine human subjects living in P. falciparum malaria endemic regions in Mali.

FIG. 21. Summary of HC and LC sequences analyzed—numbers are for total sequences from human subjects in P. falciparum malaria endemic region in Mali.

FIG. 22. Summary of HC and LC sequences analyzed—sequences filtered to remove short/mixed/poor reads and non-functional BCRs.

FIG. 23. Total number of IgM and IgG MBC BCRs cloned and recombinant mAbs expressed from human subjects in P. falciparum malaria endemic region in Mali.

FIG. 24. Both IgG+ and IgM+Plasmodium-specific MBCs are somatically hypermutated in humans, as also shown herein in murine models.

FIG. 25. Recombinant BCRs (derived from IgG+ and IgM+Plasmodium-specific MBCs), expressed as IgG1, exhibit antigen specificity (recognizing either P. falciparum AMA and MSP-1 proteins). Both IgM and IgG derived mAbs bind with high affinity.

FIG. 26. Recombinant BCRs from identical clone, expressed as IgM vs. IgG, reveal enhanced avidity for IgM.

FIG. 27. Total Number of FACS-sorted Single Memory B Cells isolated from peripheral blood samples collected from nine human subjects living in P. falciparum malaria endemic regions in Mali. The number of BCRs isolated from each subject are listed at the far right of the figure.

FIG. 28. CryoEM images of recombinant IgM pentamers and hexamers from clone AIP2-B2.

### DETAILED DESCRIPTION

Provided herein are compositions and methods comprising antigen-specific IgM memory B cells (MBCs) and recombinant antibody or antigen-binding fragments isolated from such antigen-specific IgM MBCs. As demonstrated herein, IgM⁺ and IgD⁺ MBCs are unique populations of cells with distinct phenotypic, functional and survival properties. Furthermore, these studies demonstrate that antigen-specific IgM⁺ MBCs express high affinity, somatically hypermutated BCRs and rapidly respond to produce antibodies prior to IgG⁺ MBCs. In addition, as shown herein, IgM⁺ MBCs are high affinity, rapid, plastic, early responders that can initiate the secondary response. Accordingly, antigen-specific IgM MBCs and antibodies and antigen-binding fragments derived from these cells have significant therapeutic applications in vaccine strategies and treatment of infectious diseases.

Accordingly, provided herein, in some aspects are methods of isolating or sorting antigen-specific IgM memory B cells (MBCs) comprising: (i) contacting a biological sample obtained from a subject having had prior exposure to an antigen of interest with an comprising the antigen or a portion thereof of the antigen; and (ii) isolating or sorting a cell population comprising IgM MBCs based on binding to the agent comprising the antigen. The high affinity, antigen-specific IgM binding domain from such IgM MBCs can be used to prepare, for example, high affinity recombinant antibodies or antigen-binding polypeptide constructs for therapeutic and/or diagnostic purposes, as described herein.

As shown herein, antigen-specific IgM memory B cells are a subset of B cells expressing antigen-specific, high affinity IgM molecules. B cells collectively refer to a subset of lymphocytes having an antigen-specific receptor termed an immunoglobulin or B cell receptor (BCR). Mature B cells differentiate into plasma cells, which produce antibodies, and memory B cells. A “B cell progenitor” is a cell that can develop into a mature B cell. B cell progenitors include stem cells, early pro-B cells, late pro-B cells, large pre-B cells, small pre-B cells, and immature B cells and transitional B cells. Immature B cells can develop into mature B cells, which can produce immunoglobulins (e.g., IgA, IgG or IgM). Mature B cells have acquired surface IgM and IgD, are capable of responding to antigen, and express characteristic markers such as CD21 and CD23 (CD23^{hi CD}21^{hi} cells). Common biological sources of B cells and B cell progenitors include bone marrow, peripheral blood, spleen and lymph nodes.

B cells that encounter antigen for the first time are known as “naive” B cells and have cell-surface IgM and IgD expression. A mature plasma cell secretes immunoglobulins in response to a specific antigen. A memory B cell is a B cell that initiates a unique differentiation program and also undergoes affinity selection and somatic hypermutation (with or without isotype switching) that is generally found during a secondary immune response (a subsequent antigen exposure following a primary exposure), but can also be detected during a primary antigen response. The development of memory B cells typically takes place in germinal centers (GC) of lymphoid follicles where antigen-driven lymphocytes undergo somatic hypermutation and affinity selection. Typically, memory B cells also express high affinity antigen-specific immunoglobulin (B cell receptor) on their cell surface. Further, memory B cells generally express cell-surface CD27 and CD20 as well.

Antigen-specific IgM memory B cells (MBCs), as used herein, refer to a sub-population of B cells expressing cell-surface IgM that are high affinity, have undergone somatic hypermutation, and can rapidly respond to produce antibodies. Cell-surface expression molecules such as CD21, CD27, or both CD21 and CD27 can also be used to identify such MBCs. Other cell-surface molecules that can be used to identify such MBCs include CD73, CD80, or both CD73 and CD80.

The phrase “agent comprising the antigen,” in regard to the compositions and methods described herein, refers to any agent comprising all or a part of an antigen of interest to which an IgM memory B cell specific for that antigen of interest specifically binds. Thus, for example, an agent comprising malarial MSP1 (merozoite surface protein 1) is an agent that can be specifically bound by an IgM memory B cell specific for MSP1 and not to unrelated polypeptides, such as other malarial antigen. Such agents include, but are not limited to, portions or active fragments thereof of recombinant proteins, fusion proteins, peptides, aptamers, avimers, multimers, tetramers, small molecules, and protein-binding derivatives, as well as antibodies, such as anti-idiotypic antibodies that specifically bind to the variable region of the cell-surface IgM expressed by an IgM memory B cell (such “antibodies” includes antigen-binding portions of antibodies such as epitope- or antigen-binding peptides, paratopes, functional CDRs; recombinant antibodies; chimeric antibodies; tribodies; midibodies; or antigen-binding derivatives, analogs, variants, portions, or fragments thereof).

The terms “selectively binds,” “specifically binds,” or “specific for” refer, with respect to an antigen of interest, such as MSP1 or apical membrane antigen 1 (AMA), among others, to the preferential association of an IgM memory B cell, in whole or part, with a cell or tissue bearing that antigen, or an epitope thereof, and not to cells or tissues or samples lacking that antigen, with a K_{D} of 10⁻⁵ M (10000 nM) or less, e.g., 10⁻⁶ M or less, 10⁻⁷ M or less, 10⁻⁸ M or less, 10⁻⁹ M or less, 10⁻¹⁰ M or less, 10⁻¹¹M or less, or 10⁻¹² M or less. It is, of course, recognized that a certain degree of non-specific interaction can occur between a molecule and a non-target cell or tissue. Nevertheless, specific binding can be distinguished as mediated through specific recognition of the antigen. Specific binding results in a much stronger association between the agent comprising an antigen of interest and IgM memory B cells specific for the antigen, or a portion thereof, than between the agent and IgM memory B cells lacking such specificity. Specific binding typically results in greater than 2-fold, such as greater than 5-fold, greater than 10-fold, or greater than 100-fold increase in binding of an IgM memory B cell specific for that antigen of interest to a cell or tissue bearing it, as compared to a cell or tissue lacking it. Specificity of binding can be assayed, for example, by competition assays using the antigen of interest, in comparison to competition with one or more unrelated or different antigens. A variety of immunoassay formats are appropriate for selecting agents, such as multimers, like tetramers, antibodies, or other ligands that specifically bind a given IgM memory B cell. Specific binding can be influenced by, for example, the affinity and avidity of the polypeptide agent and the concentration of polypeptide agent. The person of ordinary skill in the art can determine appropriate conditions under which the agents described herein selectively bind the IgM memory B cells using any suitable methods, such as titration of an agent in a suitable cell binding assay.

As shown herein, one way of identifying IgM memory B cells having cell-surface IgM specific for an antigen of interest, is to use a multimeric form of the antigen of interest, i.e., multimeric antigen complexes, in order to increase the binding avidity of the IgM memory B cells having antigen-specific, cell-surface IgM. Accordingly, in some embodiments of the aspects described herein, the agent comprising an antigen refers to a multimer comprising two or more monomer units of an antigen of interest, i.e., a dimer, a trimer, a tetramer, a pentamer, etc. In some embodiments of the aspects described herein, the agent comprising an antigen refers to a multimer comprising four monomer units of an antigen of interest, i.e., a tetramer. A “tetramer,” as used herein, refers to an agent comprised of four monomer units each comprising all or a portion of the antigen of interest. Such tetramer agents enable sensitive identification and isolation of IgM memory B cells specific for the antigen of interest by flow cytometry, or other methods known in the art, despite their low frequency.

Subjects from which IgM memory B cells can be derived or isolated for use in the compositions and methods described herein include any subject that can be exposed to an antigen of interest and from whom IgM memory B cells can subsequently be identified and isolated. Accordingly, in some embodiments, a “subject” refers to a mammal, including, but not limited to, a human or non-human mammal, such as a rodent, including mice and rats, bovine, equine, canine, ovine, feline, or non-human primate. In some embodiments of these aspects and all such aspects described herein, the subject is a human. The term “patient” can be used interchangeably with subject in the compositions and methods described herein.

In order to obtain memory B cells expressing an IgM or IgG receptor directed to an antigen of interest, the subject from which such memory B cells are derived should have had a primary infection with or been previously exposed to a sufficient amount of the infectious organism from which the antigen of interest or portion thereof is derived, to have generated a memory B cell response or memory B cell population. In regard to a biological sample being obtained from “a subject having had prior exposure to an antigen of interest,” such a subject has previously or currently been exposed to or infected with an infectious organism or pathogen known to express an antigen of interest. For example, in the case of malarial infections, a subject previously having had malaria or been exposed to P. falciparum is one who has had prior exposure to any antigen expressed or produced by P. falciparum, such as MSP1 or AMA, such that a population of memory B cells was generated in the subject.

Biological samples, as used herein, refer to any biological sample obtained from a subject from which B cells or B cell progenitor cells can be isolated and include bone marrow, spleen, lymph node, blood, e.g., peripheral blood, urine, saliva, cerebrospinal fluid, tissue biopsies or samples, surgical specimens, fine needle aspirates, autopsy material, and the like. Most often, the biological sample has been removed from a subject, but the term “biological sample” can also refer to cells or tissue analyzed in vivo, i.e., without removal from the subject. In some embodiments of the aspects described herein, a biological sample refers to a sample isolated from a subject, such as a peripheral blood sample, which is then further processed, for example, by cell sorting (e.g., magnetic sorting or FACS), to obtain a population of antigen-specific IgM memory B cells. In other embodiments of the aspects described herein, a biological sample comprising IgM memory B cells refers to an in vitro or ex vivo culture of expanded antigen-specific IgM memory B cells. In some embodiments of the aspects described herein, the biological sample comprises a peripheral blood sample. Alternatively, it is contemplated that MBCs expressing high affinity BCRs for an antigen of interest can be induced by, for example, administering an antigen of interest, e.g., a specific polypeptide or other antigenic fragment, to a subject. Such a subject would have had prior exposure to the antigen of interest, as defined herein.

In some embodiments of the aspects described herein, the methods comprise sorting the population comprising IgM MBCs using a combination of agents specific for CD21, CD27, and IgM isotype to isolate a population of IgM MBCs.

In some embodiments of the aspects described herein, the antigen of interest is from an infectious organism.

An “infectious organism” refers to any organism, particularly microscopic organisms, that can infect a subject and lead to an infectious disease or disorder. Examples of infectious organisms or pathogens include, but are not limited to, viruses, bacteria, protozoa, mycoplasma, and fungi. Infectious diseases can impact any bodily system, be acute (short-acting) or chronic/persistent (long-acting), occur with or without fever, strike any age group, and overlap with other infectious organisms.

The compositions and methods described herein are useful against persistent infections, in some embodiments. A “persistent infection,” as used herein, refers to an infection in which the infectious agent (such as a virus, mycoplasma, bacterium, parasite, or fungus) is not cleared or eliminated from the infected host, even after the induction of an immune response. Persistent infections can be chronic infections, latent infections, or slow infections. A “latent infection” is characterized by the lack of demonstrable infectious virus between episodes of recurrent disease. “Chronic infection” is characterized by the continued presence of infectious virus following the primary infection and can include chronic or recurrent disease. “Slow infection” is characterized by a prolonged incubation period followed by progressive disease. Unlike latent and chronic infections, slow infection may not begin with an acute period of viral multiplication. While acute infections are relatively brief (lasting a few days to a few weeks) and resolved from the body by the immune system, persistent infections can last for example, for months, years, or even a lifetime. These infections may also recur frequently over a long period of time, involving stages of silent and productive infection without cell killing or even producing excessive damage to the host cells. Persistent infections often involve stages of both silent and productive infection without rapidly killing or even producing excessive damage of the host cells. During persistent viral infections, the viral genome can be either stably integrated into the cellular DNA or maintained episomally. Persistent infection occurs with viruses such as human T-Cell leukemia viruses, Epstein-Barr virus, cytomegalovirus, herpesviruses, varicella-zoster virus, measles, papovaviruses, prions, hepatitis viruses, adenoviruses, parvoviruses and papillomaviruses, among others.

Causative infectious agents for persistent infections can be detected in the host (such as inside specific cells of infected individuals) even after the immune response has resolved, using standard techniques. Mammals are diagnosed as having a persistent infection according to any standard method known in the art and described, for example, in U.S. Pat. Nos. 6,368,832, 6,579,854, and 6,808,710 and U.S. Patent Application Publication Nos. 20040137577, 20030232323, 20030166531, 20030064380, 20030044768, 20030039653, 20020164600, 20020160000, 20020110836, 20020107363, and 20020106730, all of which are hereby incorporated by reference in their entireties.

In some embodiments, the compositions and methods described herein are contemplated for use against viral infections, i.e., when the antigen of interest comprises a viral antigen of interest. Non-limiting examples of infectious viruses include: Retroviridae (for example, HIV); Picornaviridae (for example, polio viruses, hepatitis A virus; enteroviruses, human coxsackie viruses, rhinoviruses, echoviruses); Calciviridae (such as strains that cause gastroenteritis); Togaviridae (for example, equine encephalitis viruses, rubella viruses); Flaviridae (for example, dengue viruses, encephalitis viruses, yellow fever viruses, West Nile virus, Zika virus); Coronaviridae (for example, coronaviruses); Rhabdoviridae (for example, vesicular stomatitis viruses, rabies viruses); Filoviridae (for example, ebola viruses); Paramyxoviridae (for example, parainfluenza viruses, mumps virus, measles virus, respiratory syncytial virus); Orthomyxoviridae (for example, influenza viruses); Bungaviridae (for example, Hantaan viruses, bunga viruses, phleboviruses and Nairo viruses); Arena viridae (hemorrhagic fever viruses); Reoviridae (e.g., reoviruses, orbiviurses and rotaviruses); Birnaviridae; Hepadnaviridae (Hepatitis B virus); Parvoviridae (parvoviruses); Papovaviridae (papilloma viruses, polyoma viruses); Adenoviridae (most adenoviruses); Herpesviridae (herpes simplex virus (HSV) 1 and HSV-2, varicella zoster virus, cytomegalovirus (CMV), herpes viruses); Poxviridae (variola viruses, vaccinia viruses, pox viruses); and Iridoviridae (such as African swine fever virus); and unclassified viruses (for example, the etiological agents of Spongiform encephalopathies, the agent of delta hepatitis (thought to be a defective satellite of hepatitis B virus), the agents of non-A, non-B hepatitis (class 1=internally transmitted; class 2=parenterally transmitted (i.e., Hepatitis C); Norwalk and related viruses, and astroviruses). Some viral diseases occur after immunosuppression due to re-activation of viruses already present in the recipient. Examples of persistent viral infections include, but are not limited to, cytomegalovirus (CMV) pneumonia, enteritis and retinitis; Epstein-Barr virus (EBV) lymphoproliferative disease; chicken pox/shingles (caused by varicella zoster virus, VZV); HSV-1 and -2 mucositis; HSV-6 encephalitis, BK-virus hemorrhagic cystitis; viral influenza; pneumonia from respiratory syncytial virus (RSV); AIDS (caused by HIV); and hepatitis A, B or C. In some embodiments, the compositions and methods described herein are contemplated for use against viral infections caused by enteroviruses, Flaviridae, for example, dengue viruses, encephalitis viruses, yellow fever viruses, West Nile virus, Zika and virus; Filoviridae, for example, ebola viruses; Orthomyxoviridae, for example, influenza viruses; Arena viridae, for example, hemorrhagic fever viruses; and Reoviridae, e.g., reoviruses, orbiviurses and rotaviruses.

In some embodiments, the compositions and methods described herein are contemplated for use against bacterial infections, i.e., when the antigen of interest comprises an antigen of interest derived from bacteria. Non-limiting examples of infectious bacteria include: E. coli, Psuedomonas aeruginosa, Helicobacter pyloris, Borelia burgdorferi, Legionella pneumophilia, Mycobacteria sps (such as. M. tuberculosis, M. avium, M. intracellulare, M. kansaii, M. gordonae), Staphylococcus aureus, Neisseria gonorrhoeae, Neisseria meningitidis, Listeria monocytogenes, Streptococcus pyogenes (Group A Streptococcus), Streptococcus agalactiae (Group B Streptococcus), Streptococcus (viridans group), Streptococcus faecalis, Streptococcus epidermidis, Streptococcus bovis, Streptococcus (anaerobic sps.), Streptococcus pneumoniae, pathogenic Campylobacter sp., Enterococcus sp., Haemophilus influenzae, Bacillus anthracis, corynebacterium diphtheriae, corynebacterium sp., Erysipelothrix rhusiopathiae, Clostridium perfringens, Clostridium tetani, Enterobacter aerogenes, Klebsiella pneumoniae, Brucella abortus, Pasteurella multocida, Bacteroides sp., Fusobacterium nucleatum, Streptobacillus moniliformis, Treponema pallidium, Treponema pertenue, Leptospira, Nocadia brasiliensis, Borrelia hermsii, Borrelia burgdorferi, and Actinomyces israelli. In some embodiments, the compositions and methods described herein are contemplated for use against bacterial infections caused by E. coli, Psuedomonas aeruginosa, M tuberculosis, Group B Streptococcus, Streptococcus epidermidis, Streptococcus pneumoniae, Haemophilus influenzae, Bacillus anthracia, Erysipelothrix rhusiopathiae, Klebsiella pneumoniae, Brucella abortus, Nocadia brasiliensis, Borrelia hermsii, and Borrelia burgdorferi.

In some embodiments, the compositions and methods described herein are contemplated for use against fungal infections, i.e., when the antigen of interest comprises an antigen of interest derived from a fungus. Non-limiting examples of fungal infections include but are not limited to: aspergillosis; thrush (caused by Candida albicans); cryptococcosis (caused by Cryptococcus); and histoplasmosis. Thus, examples of infectious fungi include, but are not limited to, Cryptococcus neoformans, Histoplasma capsulatum, Coccidioides immitis, Blastomyces dermatitidis, Pneumocystis carinii, Chlamydia trachomatis, and Candida albicans. In some embodiments, the compositions and methods described herein are contemplated for use against fungal infections caused by Candida albicans, Cryptococcus neoformans, and Pneumocystis carinii.

In some embodiments, the compositions and methods described herein are contemplated for use against other infectious organisms, i.e., when the antigen of interest comprises an antigen of interest derived from other infectious organisms, such as protozoan parasites. Other infectious organisms, such as protozoan parasites, include Plasmodium falciparum, exemplified herein, Shistosoma mansoni, Trypanosoma cruzi, Trichinella spiralis, Strongyloides ratti, and Toxoplasma gondii, among others. In some embodiments, the compositions and methods described herein are contemplated for use against infections caused by Plasmodium falciparum, Shistosoma mansoni, Trypanosoma cruzi, Trichinella spiralis, and Strongyloides ratti, among others.

In regard to antibodies and antigen-binding fragments being specific for an “antigen of interest,” such as an antigen derived from an infectious organism, an “antigen” is a molecule that is bound by a binding site comprised by the variable region of an immunoglobulin-related or derived polypeptide agent, such as an antibody or antibody fragment or BCR, or antigen-binding fragment thereof. Typically, antigens are bound by antibody ligands and are capable of raising or causing an antibody immune response in vivo. An antigen can be a polypeptide, protein, nucleic acid or other molecule. In the case of conventional antibodies and fragments thereof, the binding site as defined by the variable loops (L1, L2, L3 and H1, H2, H3) is capable of binding to the antigen. The term “antigenic determinant” refers to an epitope on the antigen recognized by an antigen-binding molecule, and more particularly, by the antigen-binding site of said molecule.

The term “epitope” is a region or portion of an antigen that is bound by a binding protein, and includes any polypeptide determinant capable of specific binding to an immunoglobulin or T-cell receptor. In certain embodiments, epitope determinants include chemically active surface groupings of molecules such as amino acids, sugar side chains, phosphoryl, or sulfonyl, and, in certain embodiments, may have specific three dimensional structural characteristics, and/or specific charge characteristics. An epitope can be determined by obtaining an X-ray crystal structure of an antibody:antigen complex and determining which residues on the antigen are within a specified distance of residues on the antibody of interest, wherein the specified distance is, 5 Å or less, e.g., 5 Å, 4 Å, 3 Å, 2 Å, 1 Å or any distance in between. In some embodiments, an “epitope” can be formed on a polypeptide both from contiguous amino acids, or noncontiguous amino acids juxtaposed by tertiary folding of a protein. Epitopes formed from contiguous amino acids are typically retained on exposure to denaturing solvents, whereas epitopes formed by tertiary folding are typically lost on treatment with denaturing solvents. An epitope typically includes at least 3, and more usually, at least 5, about 9, or about 8-10 amino acids in a unique spatial conformation. An “epitope” includes the unit of structure conventionally bound by an immunoglobulin VH/VL pair. Epitopes define the minimum binding site for an antibody, and thus represent the target of specificity of an antibody. In the case of a single domain antibody, an epitope represents the unit of structure bound by a variable domain in isolation. The terms “antigenic determinant” and “epitope” can also be used interchangeably herein. In certain embodiments, epitope determinants include chemically active surface groupings of molecules such as amino acids, sugar side chains, phosphoryl, or sulfonyl, and, in certain embodiments, may have specific three dimensional structural characteristics, and/or specific charge characteristics.

Accordingly, in some aspects, provided herein are methods of sorting Plasmodium-specific IgM memory B cells (MBCs), comprising: contacting a biological sample obtained from a subject infected with or having been vaccinated against malaria with a tetramer comprising a Plasmodium antigen; and sorting a cell population comprising Plasmodium-specific IgM MBCs based on binding to the tetramer.

In some embodiments of these aspects and all such aspects described herein, the method further comprises generating tetramers comprising blood or liver stage Plasmodium antigens prior to the contacting step. Non-limiting examples of such blood or liver stage Plasmodium antigens include MSP-1 and AMA.

In some embodiments of the aspects described herein, the methods further comprise a step of sequencing one or more BCRs, or at least the antigen-binding domains thereof, expressed by the cell population comprising IgM MBCs. In some embodiments of these aspects and all such aspects described herein, the methods further comprise a step of sequencing Plasmodium-specific IgM MBC BCRs.

In some embodiments of the aspects described herein, the methods further comprise a step of cloning the one or more BCRs and expressing the one or more BCRs as recombinant antibodies or antigen-binding fragments thereof.

As demonstrated herein, antigen-specific IgM memory B cells are a sub-population of B cells expressing cell-surface IgM that are high affinity, have undergone somatic hypermutation, and can rapidly respond, upon subsequent exposure to the same antigen, to produce high-affinity, secreted antibodies. Upon isolation and cloning of such antigen-specific IgM memory B cells, high affinity sequences corresponding to variable heavy and variable light chain sequences, as well as corresponding CDRs, can be obtained and used to generate novel antibodies and antigen-binding fragments thereof, and recombinant cells that produce such novel antibodies and antigen-binding fragments thereof. Antigen-specific IgM antibodies selected for cloning and sequencing typically have a high binding affinity for the antigen of interest, for example, typically having a K_{D} value between 10⁻⁷ M to 10⁻¹⁰ M, or better.

Accordingly, also provided herein, in some aspects, are recombinant cells producing an antigen-binding polypeptide comprising a variable heavy chain immunoglobulin sequence, a variable light chain immunoglobulin sequence, or both, from an IgM memory B cell obtained using any of the methods described herein.

In some aspects, provided herein are recombinant antigen-binding polypeptides isolated from any of the recombinant cells described herein.

In some aspects, provided herein are recombinant antigen-binding polypeptides comprising an antigen-binding domain of an IgM memory B cell receptor.

The term “antigen-binding polypeptide” refers to a polypeptide that specifically binds to a desired antigen of interest and that is an Ig-like protein comprising one or more of the antigen binding domains described herein linked to a linker or an immunoglobulin constant domain. A binding protein can be, in some embodiments, a dual variable domain (DVD-Ig) binding protein. A “linker polypeptide” comprises two or more amino acid residues joined by peptide bonds and are used to link one or more antigen binding portions. Such linker polypeptides are well known in the art (see e.g., Holliger et al. (1993) Proc. Natl. Acad. Sci. USA 90: 6444-6448; Poljak (1994) Structure 2: 1121-1123). An immunoglobulin constant domain refers to a heavy or light chain constant domain. Human IgG heavy chain and light chain constant domain amino acid sequences are known in the art, (e.g., see SEQ ID NO: 197, 198, 199 and 200 of US Application 2016/0200813, which is incorporated herein in its entirety by reference for representative examples). In various embodiments, the binding proteins and antibodies disclosed herein can comprise any of the constant domains of SEQ ID NO: 197, 198, 199 and 200 of US Application 2016/0200813.

As known to those of skill in the art, the term “antibody” broadly refers to any immunoglobulin (Ig) molecule and immunologically active portions of immunoglobulin molecules (i.e., molecules that contain an antigen binding site that immunospecifically bind an antigen) comprised of four polypeptide chains, two heavy (H) chains and two light (L) chains, or any functional fragment, mutant, variant, or derivation thereof, which retains the essential epitope binding features of an Ig molecule. Such mutant, variant, or derivative antibody formats are known in the art. Non-limiting embodiments of which are discussed below, and include but are not limited to a variety of forms, including full length antibodies and antigen-binding portions thereof; including, for example, an immunoglobulin molecule, a monoclonal antibody, a chimeric antibody, a CDR-grafted antibody, a human antibody, a humanized antibody, a single chain antibody, a Fab, a F(ab′), a F(ab′)2, a Fv antibody, fragments produced by a Fab expression library, a disulfide linked Fv, a scFv, a single domain antibody (dAb), a diabody, a multispecific antibody, a dual specific antibody, an anti-idiotypic antibody, a bispecific antibody, a functionally active epitope-binding fragment thereof, bifunctional hybrid antibodies (e.g., Lanzavecchia et al., Eur. J. Immunol. 17, 105 (1987)) and single chains (e.g., Huston et al., Proc. Natl. Acad. Sci. U.S.A., 85, 5879-5883 (1988) and Bird et al., Science 242, 423-426 (1988), which are incorporated herein by reference) and/or antigen-binding fragments of any of the above (See, generally, Hood et al., Immunology, Benjamin, N.Y., 2ND ed. (1984), Harlow and Lane, Antibodies. A Laboratory Manual, Cold Spring Harbor Laboratory (1988) and Hunkapiller and Hood, Nature, 323, 15-16 (1986), which are incorporated herein by reference). Antibodies also refer to immunoglobulin molecules and immunologically active portions of immunoglobulin molecules, i.e., molecules that contain antigen or target binding sites or “antigen-binding fragments.”

The antibody or immunoglobulin molecules described herein can be of any type (e.g., IgG, IgE, IgM, IgD, IgA and IgY), class (e.g., IgG1, IgG2, IgG3, IgG4, IgA1 and IgA2) or subclass of immunoglobulin molecule, as is understood by one of skill in the art. Furthermore, in humans, the light chain can be a kappa chain or a lambda chain. As shown herein, high affinity CDRs from IgM+ memory B cells can be used to construct or derive other recombinant antibodies having those CDRs, but different class types, for example.

In some embodiments of the compositions and methods described herein, the antigen-binding domain comprises a variable light chain sequence, a variable heavy chain sequence, or both.

As understood by those of skill in the art, in a full-length antibody, each heavy chain is comprised of a heavy chain variable domain (abbreviated herein as HCVR or VH) and a heavy chain constant region. The heavy chain constant region is comprised of three domains: CH1, CH2, and CH3. Each light chain is comprised of a light chain variable domain (abbreviated herein LCVR as VL) and a light chain constant region. The light chain constant region is comprised of one domain, CL. The VH and VL regions can be further subdivided into regions of hypervariability, termed complementarity determining regions (CDRs), interspersed with regions that are more conserved, termed framework regions (FR). Each VH and VL is composed of three CDRs and four FRs, arranged from amino-terminus to carboxy-terminus in the following order: FR1, CDR1, FR2, CDR2, FR3, CDR3, FR4. This structure is well-known to those skilled in the art. The chains are usually linked to one another via disulfide bonds.

As used herein, the term “Complementarity Determining Regions” (“CDRs”), i.e., CDR1, CDR2, and CDR3) refers to the amino acid residues of a heavy or light chain variable domain the presence of which are necessary for specific antigen binding. Each variable domain typically has three CDR regions identified as CDR1, CDR2 and CDR3. Each complementarity determining region can comprise amino acid residues from a “complementarity determining region” as defined by Kabat (i.e., about residues 24-34 (L1), 50-56 (L2) and 89-97 (L3) in the light chain variable domain and 31-35 (H1), 50-65 (H2) and 95-102 (H3) in the heavy chain variable domain; Kabat et al., Sequences of Proteins of Immunological Interest, 5th Ed. Public Health Service, National Institutes of Health, Bethesda, Md. (1991)) and/or those residues from a “hypervariable loop” (i.e., about residues 26-32 (L1), 50-52 (L2) and 91-96 (L3) in the light chain variable domain and 26-32 (H1), 53-55 (H2) and 96-101 (H3) in the heavy chain variable domain; Chothia and Lesk J. Mol. Biol. 196:901-917 (1987)). In some instances, a complementarity determining region can include amino acids from both a CDR region defined according to Kabat and a hypervariable loop. The term “CDR set” as used herein refers to a group of three CDRs that occur in a single heavy or light chain variable region capable of binding the antigen. The exact boundaries of these CDRs have been defined differently according to different systems. The system described by Kabat (Kabat et al, Sequences of Proteins of Immunological Interest (National Institutes of Health, Bethesda, Md. (1987) and (1991)) not only provides an unambiguous residue numbering system applicable to any variable region of an antibody, but also provides precise residue boundaries defining the three CDRs. These CDRs may be referred to as Kabat CDRs. Chothia and coworkers (Chothia & Lesk, J. Mol. Biol, 196:901-917 (1987) and Chothia et al., Nature 342:877-883 (1989)) found that certain sub-portions within Kabat CDRs adopt nearly identical peptide backbone conformations, in spite of great diversity at the level of amino acid sequence. These sub-portions were designated as L1, L2 and L3 or H1, H2 and H3 where the “L” and the “H” designates the light chain and the heavy chains regions, respectively. These regions may be referred to as Chothia CDRs, which have boundaries that overlap with Kabat CDRs. Other boundaries defining CDRs overlapping with the Kabat CDRs have been described by Padlan (FASEB). 9:133-139 (1995)) and MacCallum (J Mol Biol 262(5):732-45 (1996)). Still other CDR boundary definitions may not strictly follow one of the above systems, but will nonetheless overlap with the Kabat CDRs, although they may be shortened or lengthened in light of prediction or experimental findings that particular residues or groups of residues or even entire CDRs do not significantly impact antigen binding. CDRs can also be described as comprising amino acid residues from a “complementarity determining region” as defined by the IMGT, in some embodiments. The compositions and methods used herein may utilize CDRs defined according to any of these systems, although preferred embodiments use IMGT defined CDRs. Nonetheless. The boundaries of the CDRs are clear in reference to either of these numbering conventions.

An immunoglobulin constant (C) domain refers to a heavy (CH) or light (CL) chain constant domain. Murine and human IgG heavy chain and light chain constant domain amino acid sequences are known in the art. With respect to the heavy chain, in some embodiments of the aspects described herein, the heavy chain of an antibody described herein can be an alpha (α), delta (Δ), epsilon (ε), gamma (γ) or mu (μ) heavy chain. In some embodiments of the aspects described herein, the heavy chain of an antibody described can comprise a human alpha (α), delta (Δ), epsilon (ε), gamma (γ) or mu (μ) heavy chain. Non-limiting examples of human constant region sequences have been described in the art, e.g., see U.S. Pat. No. 5,693,780 and Kabat E A et al., (1991) supra.

Accordingly, in embodiments of the compositions and methods described herein, the IgM memory B cell receptor antigen-binding domain or CDRs derived from the IgM memory B cell receptor antigen-binding domain is comprised in a non-IgM isotype acceptor antibody framework.

As used herein, the terms “donor” and “donor antibody” refer to an antibody providing one or more CDRs. In an exemplary embodiment, the donor antibody is an antibody from a species different from the antibody from which the framework regions are obtained or derived. In some embodiments, the donor antibody is of a different isotype than the acceptor antibody. In the context of a humanized antibody, the term “donor antibody” refers to a non-human antibody providing one or more CDRs.

As used herein, the terms “acceptor” and “acceptor antibody” refer to the antibody providing or nucleic acid sequence encoding at least 80%, at least 85%, at least 90%, at least 95%, at least 98%, or 100% of the amino acid sequences of one or more of the framework regions. In some embodiments, the term “acceptor” refers to the antibody amino acid providing or nucleic acid sequence encoding the constant region(s). In yet another embodiment, the term “acceptor” refers to the antibody amino acid providing or nucleic acid sequence encoding one or more of the framework regions and the constant region(s). In a specific embodiment, the term “acceptor” refers to a human antibody amino acid or nucleic acid sequence that provides or encodes at least 80%, preferably, at least 85%, at least 90%, at least 95%, at least 98%, or 100% of the amino acid sequences of one or more of the framework regions. In accordance with this embodiment, an acceptor may contain at least 1, at least 2, at least 3, least 4, at least 5, or at least 10 amino acid residues that does (do) not occur at one or more specific positions of a human antibody. An acceptor framework region and/or acceptor constant region(s) may be, e.g., derived or obtained from a germline antibody gene, a mature antibody gene, a functional antibody (e.g., antibodies well known in the art, antibodies in development, or antibodies commercially available).

Human heavy chain and light chain acceptor sequences are known in the art. In some embodiments, the human heavy chain and light chain acceptor sequences are selected from the sequences listed from V-base (found on the worldwide web at vbase.mrc-cpe.cam.ac.uk/) or from IMGT™ the international IMMUNOGENETICS INFORMATION SYSTEM™ (found on the worldwide web at imgt.cines.fr/textes/IMGTrepertoire/LocusGenes/). In another embodiment of the technology disclosed herein, the human heavy chain and light chain acceptor sequences are selected from the sequences described in Table 3 and Table 4 of U.S. Patent Publication No. 2011/0280800, incorporated by reference herein in their entireties.

As used herein, the term “germline antibody gene” or “germline antibody gene fragment” refers to immunoglobulin-encoding nucleic acid sequence encoded by non-lymphoid cells that have not undergone the maturation process that leads to genetic rearrangement and mutation for expression of a particular immunoglobulin. (See, e.g., Shapiro et al. (2002) Crit. Rev. Immunol. 22(3): 183-200; Marchalonis et al. (2001) Adv. Exp. Med. Biol. 484:13-30). One of the advantages provided by embodiments that use germline antibody sequences, e.g., for one or more constant domains, stems from the recognition that germline antibody genes are more likely than mature antibody genes to conserve essential amino acid sequence structures characteristic of individuals in the species, hence less likely to be recognized as from a foreign source when used therapeutically in that species.

As used herein, the term “key” residues refers to certain residues within the variable domain that have more impact on the binding specificity and/or affinity of an antibody, in particular a humanized antibody, than others. A key residue includes, but is not limited to, one or more of the following: a residue that is adjacent to a CDR, a potential glycosylation site (can be either N- or O-glycosylation site), a rare residue, a residue capable of interacting with the antigen, a residue capable of interacting with a CDR, a canonical residue, a contact residue between heavy chain variable domain and light chain variable domain, a residue within the Vernier zone, and a residue in the region that overlaps between the Chothia definition of a variable heavy chain CDR/and the Kabat definition of the first heavy chain framework.

The term “humanized antibody” refers to antibodies that comprise heavy and light chain variable domain sequences from a non-human species (e.g., a mouse) but in which at least a portion of the VH and/or VL sequence has been altered to be more “human-like”, i.e., more similar to human germline variable sequences. Accordingly, “humanized” antibodies are a form of a chimeric antibody, that are engineered or designed to comprise minimal sequence derived from non-human immunoglobulin. For the most part, humanized antibodies are human immunoglobulins (recipient or acceptor antibody) in which residues from a hypervariable region of the recipient are replaced by residues from a hypervariable region of a non-human species (donor antibody) such as mouse, rat, rabbit or nonhuman primate having the desired specificity, affinity, and capacity. In some instances, Fv framework region (FR) residues of the human immunoglobulin are replaced by corresponding non-human residues. Furthermore, humanized antibodies can comprise residues which are not found in the recipient antibody or in the donor antibody. These modifications are made to further refine antibody performance. In general, the humanized antibody will comprise substantially all of at least one, and typically two, variable domains, in which all or substantially all of the hypervariable loops correspond to those of a non-human immunoglobulin and all or substantially all of the FR regions are those of a human immunoglobulin sequence. The humanized antibody optionally also will comprise at least a portion of an immunoglobulin constant region (Fc), typically that of a human immunoglobulin. For further details, see Jones et al., Nature 321:522-525 (1986); Riechmann et al., Nature 332:323-329 (1988); and Presta, Curr. Op. Struct. Biol. 2:593-596 (1992). As used herein, a “composite human antibody” or “deimmunized antibody” are specific types of engineered or humanized antibodies designed to reduce or eliminate T cell epitopes from the variable domains.

The compositions and methods described herein can, in some embodiments, comprise “antigen-binding fragments” or “antigen-binding portions” of an antibody. The term “antigen-binding fragment” of an antibody refers to one or more fragments of an antibody that retain the ability to specifically bind to an antigen. Antigen-binding functions of an antibody can be performed by fragments of a full-length antibody. Such antibody fragment embodiments may also be incorporated in bispecific, dual specific, or multi-specific formats such as a dual variable domain (DVD-Ig) format; specifically binding to two or more different antigens. Non-limiting examples of antigen-binding fragments encompassed within the term “antigen-binding portion” of an antibody include (i) a Fab fragment, a monovalent fragment consisting of the VL, VH, CL, and CH1 domains; (ii) a F(ab′)2 fragment, a bivalent fragment comprising two Fab fragments linked by a disulfide bridge at the hinge region; (iii) a Fd fragment consisting of the VH and CH1 domains; (iv) a Fv fragment consisting of the VL and VH domains of a single arm of an antibody, (v) a dAb fragment (Ward et al. (1989) Nature, 341: 544-546; PCT Publication No. WO 90/05144), which comprises a single variable domain; and (vi) an isolated complementarity determining region (CDR). Furthermore, although the two domains of the Fv fragment, VL and VH, are coded for by separate genes, they can be joined, using recombinant methods, by a synthetic linker that enables them to be made as a single protein chain in which the VL and VH regions pair to form monovalent molecules (known as single chain Fv (scFv); see, for example, Bird et al. (1988) Science 242: 423-426; and Huston et al. (1988) Proc. Natl. Acad. Sci. USA 85: 5879-5883). Such single chain antibodies are also intended to be encompassed within the term “antigen-binding portion” of an antibody. Other forms of single chain antibodies, such as diabodies are also encompassed. Diabodies are bivalent, bispecific antibodies in which VH and VL domains are expressed on a single polypeptide chain, but using a linker that is too short to allow for pairing between the two domains on the same chain, thereby forcing the domains to pair with complementary domains of another chain and creating two antigen binding sites (see, for example, Holliger et al. (1993) Proc. Natl. Acad. Sci. USA 90: 6444-6448; Poljak (1994) Structure 2: 1121-1123); Kontermann and Dubel eds., Antibody Engineering, Springer-Verlag, N.Y. (2001), p. 790 (ISBN 3-540-41354-5). In addition single chain antibodies also include “linear antibodies” comprising a pair of tandem Fv segments (VH-CH1-VH-CH1) which, together with complementary light chain polypeptides, form a pair of antigen binding regions (Zapata et al. (1995) Protein Eng. 8(10): 1057-1062; and U.S. Pat. No. 5,641,870).

The term “Fc region” is used to define the C-terminal region of an immunoglobulin heavy chain, which may be generated by papain digestion of an intact antibody. The Fc region may be a native sequence Fc region or a variant Fc region. The Fc region of an immunoglobulin generally comprises two constant domains, a CH2 domain, and a CH3 domain, and optionally comprises a CH4 domain. Replacements of amino acid residues in the Fc portion to alter antibody effector function are known in the art (U.S. Pat. Nos. 5,648,260 and 5,624,821). The Fc portion of an antibody mediates several important effector functions, for example, cytokine induction, antibody-dependent cell cytotoxicity (ADCC), phagocytosis, complement dependent cytotoxicity (CDC), and half-life/clearance rate of antibody and antigen-antibody complexes. In some cases these effector functions are desirable for therapeutic antibody but in other cases might be unnecessary or even deleterious, depending on the therapeutic objectives. Certain human IgG isotypes, particularly IgG1 and IgG3, mediate ADCC and CDC via binding to Fcγ receptors and complement C1q, respectively. Neonatal Fc receptors (FcRn) are the critical components determining the circulating half-life of antibodies. In still another embodiment at least one amino acid residue is replaced in the constant region of the antibody, for example the Fc region of the antibody, such that effector functions of the antibody are altered.

The DNA sequences encoding the antibodies or antigen-binding fragments that specifically bind an antigen of interest described herein, e.g., antibodies or antigen-binding fragments specifically binding a malarial or other antigen of interest, can also be modified, for example, by substituting the coding sequence for human heavy- and light-chain constant domains or framework regions in place of the homologous murine sequences (U.S. Pat. No. 4,816,567; Morrison, et al., Proc. Natl. Acad. Sci. USA, 81:6851 (1984)), or by covalently joining to the immunoglobulin coding sequence all or part of the coding sequence for a non-immunoglobulin polypeptide, as also described elsewhere herein.

Such non-immunoglobulin polypeptides can be substituted for the constant domains of an antibody, or they can be substituted for the variable domains of one antigen-binding site of an antibody to create a chimeric bivalent antibody comprising one antigen-binding site having specificity for one antigen of interest and another antigen-binding site having specificity for a different antigen of interest.

Also provided herein, in some aspects, are humanized antibodies and antigen-binding fragments for use in the compositions and methods described herein. Humanized forms of non-human (e.g., murine) antibodies refer to chimeric antibodies that contain minimal sequence derived from non-human immunoglobulin. For the most part, humanized antibodies are human immunoglobulins (recipient antibody) in which residues from a hypervariable region of the recipient are replaced by residues from a hypervariable region of a non-human species (donor antibody) such as mouse, rat, rabbit or nonhuman primate having the desired specificity, affinity, and capacity. In some embodiments, Fv framework region (FR) residues of the human immunoglobulin can be replaced by corresponding non-human residues. Furthermore, humanized antibodies can comprise residues that are not found in the recipient antibody or in the donor antibody. These modifications are made to further refine antibody performance. In general, a humanized antibody can comprise substantially all of at least one, and typically two, variable domains, in which all or substantially all of the hypervariable loops correspond to those of a non-human immunoglobulin and all or substantially all of the FR regions are those of a human immunoglobulin sequence. The humanized antibody optionally also can comprise at least a portion of an immunoglobulin constant region (Fc), typically that of a human immunoglobulin. For further details, see Jones et al., Nature 321:522-525 (1986); Riechmann et al., Nature 332:323-329 (1988); and Presta, Curr. Op. Struct. Biol. 2:593-596 (1992).

A humanized antibody has one or more amino acid residues introduced into it from a source which is non-human. These non-human amino acid residues are often referred to as “import” residues, which are typically taken from an “import” variable domain. Humanization can be essentially performed following the method of Winter and co-workers (Jones et al., Nature, 321:522-525 (1986); Riechmann et al., Nature, 332:323-327 (1988); Verhoeyen et al., Science, 239:1534-1536 (1988)), by substituting rodent CDRs or CDR sequences for the corresponding sequences of a human antibody. Accordingly, such humanized antibodies are chimeric antibodies (U.S. Pat. No. 4,816,567) where substantially less than an intact human variable domain has been substituted by the corresponding sequence from a non-human species. In practice, humanized antibodies are typically human antibodies in which some CDR residues and possibly some FR residues are substituted by residues from analogous sites in rodent antibodies.

While applicable to the variable domains and CDRs of any immunoglobulin-type polypeptide expressed by an MBC, CDRs of murine MBCs exemplified herein can be used to generate humanized antibody constructs. Accordingly, in some embodiments, humanized antibodies comprising one or more variable domains comprising one or more CDRs encoded by the variable heavy chain sequences of SEQ ID NOs: 1, 3, 5, 7, 9, 11, 13, 15, 17, 19, 21, and 23, and/or one or more CDRs encoded by the variable light chain sequences of SEQ ID NOs: 2, 4, 6, 8, 10, 12, 14, 16, 19, 20, 22, and 24, are provided. Accordingly, in some embodiments of the aspects provided herein, the CDR sequences encoded by the variable heavy chain sequences of SEQ ID NOs: 1, 3, 5, 7, 9, 11, 13, 15, 17, 19, 21, and 23, and/or the CDRs encoded by the variable light sequences of SEQ ID NOs: 2, 4, 6, 8, 10, 12, 14, 16, 19, 20, 22, and 24 can be used to generate, for example, CDR-grafted, chimeric, humanized, or composite human antibodies or antigen-binding fragments, as described elsewhere herein. As understood by one of ordinary skill in the art, any variant, CDR-grafted, chimeric, humanized, or composite antibodies or antigen-binding fragments derived from any of these sequences will maintain the ability to immunospecifically bind the antigen of interest, such that the variant, CDR-grafted, chimeric, humanized, or composite antibody or antigen-binding fragment thereof has at least 50%, at least 60%, at least 70%, at least 80%, at least 90%, at least 95%, at least 100%, or any amount greater than the binding affinity to the antigen of interest relative to the original antibody from which it is derived.

In some embodiments of the aspects described herein, the antibody or antigen-binding fragment thereof comprises one, two, three, or four of the framework regions of a heavy chain variable region sequence which is at least 75%, 80%, 85%, 90%, 95% or 100% identical to one, two, three or four of the framework regions of the heavy chain variable region sequence from which it is derived. In some embodiments of the aspects described herein, the heavy chain variable framework region that is derived from said amino acid sequence consists of said amino acid sequence but for the presence of up to 10 amino acid substitutions, deletions, and/or insertions, preferably up to 10 amino acid substitutions. In some embodiments of the aspects described herein, the heavy chain variable framework region that is derived from said amino acid sequence consists of said amino acid sequence with 1, 2, 3, 4, 5, 6, 7, 8, 9 or 10 amino acid residues being substituted for an amino acid found in an analogous position in a corresponding non-human, primate, or human heavy chain variable framework region. In some embodiments of the aspects described herein, the antibody or antigen-binding fragment further comprises one, two, three or all four V_{H} framework regions derived from the V_{H} of a human or primate antibody. The primate or human heavy chain framework region of the antibody selected for use with the heavy chain CDR sequences described herein, can have, for example, at least 70% identity with a heavy chain framework region of the non-human parent antibody. Preferably, the primate or human antibody selected can have the same or substantially the same number of amino acids in its heavy chain complementarity determining regions encoded by the variable heavy chain sequences of SEQ ID NOs: 1, 3, 5, 7, 9, 11, 13, 15, 17, 19, 21, and 23. In some embodiments of the aspects described herein, the primate or human heavy chain framework region amino acid residues are from a natural primate or human antibody heavy chain framework region having at least 75% identity, at least 80% identity, at least 85% identity (or more) with the heavy chain framework regions of any of the antibodies described herein. In specific embodiments, the antibody or antigen-binding fragment further comprises one, two, three or all four V_{H} framework regions derived from a human heavy chain variable subfamily (e.g., one of subfamilies 1 to 7).

In some such embodiments of the aspects described herein, the antibody or antigen-binding fragment thereof comprises one, two, three or four of the framework regions of a light chain variable region sequence which is at least 75%, 80%, 85%, 90%, 95%, or 100% identical to one, two, three or four of the framework regions of the light chain variable region sequence from which it is derived. In some embodiments of the aspects described herein, the light chain variable framework region that is derived from said amino acid sequence consists of said amino acid sequence but for the presence of up to 10 amino acid substitutions, deletions, and/or insertions, preferably up to 10 amino acid substitutions. In some embodiments of the aspects described herein, the light chain variable framework region that is derived from said amino acid sequence consists of said amino acid sequence with 1, 2, 3, 4, 5, 6, 7, 8, 9 or 10 amino acid residues being substituted for an amino acid found in an analogous position in a corresponding non-human, primate, or human light chain variable framework region. In some embodiments of the aspects described herein, the antibody or antigen-binding fragment further comprises one, two, three or all four V_{L} framework regions derived from the V_{L} of a human or primate antibody. The primate or human light chain framework region of the antibody selected for use with the light chain CDR sequences described herein, can have, for example, at least 70% identity with a light chain framework region of the non-human parent antibody. The primate or human antibody selected can have the same or substantially the same number of amino acids in its light chain CDRs to that of the light chain complementarity determining regions encoded by the variable light chain sequences of SEQ ID NOs: 2, 4, 6, 8, 10, 12, 14, 16, 19, 20, 22, and 24. In some embodiments of the aspects described herein, the primate or human light chain framework region amino acid residues are from a natural primate or human antibody light chain framework region having at least 75% identity, at least 80% identity, at least 85% identity (or more) with the light chain framework regions of any of the antibodies described herein. In some embodiments, the antibody or antigen-binding fragment further comprises one, two, three or all four V_{L} framework regions derived from a human light chain variable kappa subfamily. In some embodiments, the antibody or antigen-binding fragment further comprises one, two, three or all four VL framework regions derived from a human light chain variable lambda subfamily.

In some embodiments of the aspects described herein, the position of one or more CDRs along the V_{H} (e.g., CDR1, CDR2, or CDR3) and/or VL (e.g., CDR1, CDR2, or CDR3) region of an antibody described herein can vary, i.e., be shorter or longer, by one, two, three, four, five, or six amino acid positions so long as immunospecific binding to the antigen of interest is maintained (e.g., substantially maintained, for example, at least 50%, at least 60%, at least 70%, at least 80%, at least 90%, at least 95% of the binding of the original antibody from which it is derived). For example, in some embodiments, the position defining a CDR can vary, i.e., be shorter or longer, by shifting the N-terminal and/or C-terminal boundary of the CDR by one, two, three, four, five, or six amino acids, relative to the CDR position of any one of the antibodies described herein, so long as immunospecific binding to the antigen of interest is maintained (e.g., substantially maintained, for example, at least 50%, at least 60%, at least 70%, at least 80%, at least 90%, at least 95% of the binding of the original antibody from which it is derived). In other embodiments, the length of one or more CDRs along the V_{H} (e.g., CDR1, CDR2, or CDR3) and/or V_{L} (e.g., CDR1, CDR2, or CDR3) region of an antibody described herein can vary (e.g., be shorter or longer) by one, two, three, four, five, or more amino acids, so long as immunospecific binding to the antigen of interest is maintained (e.g., substantially maintained, for example, at least 50%, at least 60%, at least 70%, at least 80%, at least 90%, at least 95% of the binding of the original antibody from which it is derived).

In some embodiments of the aspects described herein, one, two or more mutations (e.g., amino acid substitutions) are introduced into the Fc region of any of the antibodies described herein or a fragment thereof (e.g., CH2 domain (residues 231-340 of human IgG1) and/or CH3 domain (residues 341-447 of human IgG1) and/or the hinge region, with numbering according to the Kabat numbering system (e.g., the EU index in Kabat)) to alter one or more functional properties of the antibody, such as serum half-life, complement fixation, Fc receptor binding and/or antigen-dependent cellular cytotoxicity.

In some embodiments of the aspects described herein, one, two or more mutations (e.g., amino acid substitutions) are introduced into the hinge region of the Fc region (CH1 domain) such that the number of cysteine residues in the hinge region are altered (e.g., increased or decreased) as described in, e.g., U.S. Pat. No. 5,677,425. The number of cysteine residues in the hinge region of the CH1 domain can be altered to, e.g., facilitate assembly of the light and heavy chains, or to alter (e.g., increase or decrease) the stability of the antibody.

In some embodiments of the aspects described herein, one, two or more mutations (e.g., amino acid substitutions) are introduced into the Fc region of an antibody described herein or an antigen-binding fragment thereof (e.g., CH2 domain (residues 231-340 of human IgG1) and/or CH3 domain (residues 341-447 of human IgG1) and/or the hinge region, with numbering according to the Kabat numbering system (e.g., the EU index in Kabat)) to increase or decrease the affinity of the antibody for an Fc receptor (e.g., an activated Fc receptor) on the surface of an effector cell. Mutations in the Fc region of an antibody or fragment thereof that decrease or increase the affinity of an antibody for an Fc receptor and techniques for introducing such mutations into the Fc receptor or fragment thereof are known to one of skill in the art. Examples of mutations in the Fc receptor of an antibody that can be made to alter the affinity of the antibody for an Fc receptor are described in, e.g., Smith P et al., (2012) PNAS 109: 6181-6186, U.S. Pat. No. 6,737,056, and International Publication Nos. WO 02/060919; WO 98/23289; and WO 97/34631, which are incorporated herein by reference.

In some embodiments of the aspects described herein, one, two or more amino acid mutations (i.e., substitutions, insertions or deletions) are introduced into an IgG constant domain, or FcRn-binding fragment thereof (preferably an Fc or hinge-Fc domain fragment) to alter (e.g., decrease or increase) half-life of the antibody in vivo. See, e.g., International Publication Nos. WO 02/060919; WO 98/23289; and WO 97/34631; and U.S. Pat. Nos. 5,869,046, 6,121,022, 6,277,375 and 6,165,745 for examples of mutations that will alter (e.g., decrease or increase) the half-life of an antibody in vivo.

In some embodiments of the aspects described herein, one, two or more amino acid mutations (i.e., substitutions, insertions or deletions) are introduced into an IgG constant domain, or FcRn-binding fragment thereof (preferably an Fc or hinge-Fc domain fragment) to decrease the half-life of the antibody in vivo. In some embodiments of the aspects described herein, one, two or more amino acid mutations (i.e., substitutions, insertions or deletions) are introduced into an IgG constant domain, or FcRn-binding fragment thereof (preferably an Fc or hinge-Fc domain fragment) to increase the half-life of the antibody in vivo. In some embodiments of the aspects described herein, the antibodies can have one or more amino acid mutations (e.g., substitutions) in the second constant (CH2) domain (residues 231-340 of human IgG1) and/or the third constant (CH3) domain (residues 341-447 of human IgG1), with numbering according to the EU index in Kabat (Kabat E A et al., (1991) supra). In some embodiments of the aspects described herein, the constant region of the IgG1 of an antibody or antigen-binding fragment thereof described herein comprises a methionine (M) to tyrosine (Y) substitution in position 252, a serine (S) to threonine (T) substitution in position 254, and a threonine (T) to glutamic acid (E) substitution in position 256, numbered according to the EU index as in Kabat. See U.S. Pat. No. 7,658,921, which is incorporated herein by reference. This type of mutant IgG, referred to as “YTE mutant” has been shown to display fourfold increased half-life as compared to wild-type versions of the same antibody (see Dall'Acqua W F et al., (2006) J Biol Chem 281: 23514-24). In some embodiments of the aspects described herein, an antibody or antigen-binding fragment thereof comprises an IgG constant domain comprising one, two, three or more amino acid substitutions of amino acid residues at positions 251-257, 285-290, 308-314, 385-389, and 428-436, numbered according to the EU index as in Kabat.

In some embodiments of the aspects described herein, one, two or more amino acid substitutions are introduced into an IgG constant domain Fc region to alter the effector function(s) of the antibody. For example, one or more amino acids selected from amino acid residues 234, 235, 236, 237, 297, 318, 320 and 322, numbered according to the EU index as in Kabat, can be replaced with a different amino acid residue such that the antibody has an altered affinity for an effector ligand but retains the antigen-binding ability of the parent antibody. The effector ligand to which affinity is altered can be, for example, an Fc receptor or the Cl component of complement. This approach is described in further detail in U.S. Pat. Nos. 5,624,821 and 5,648,260. In some embodiments of the aspects described herein, the deletion or inactivation (through point mutations or other means) of a constant region domain can reduce Fc receptor binding of the circulating antibody thereby increasing, for example, tumor localization. See, e.g., U.S. Pat. Nos. 5,585,097 and 8,591,886 for a description of mutations that delete or inactivate the constant domain and thereby increase tumor localization. In some embodiments of the aspects described herein, one or more amino acid substitutions may be introduced into the Fc region of an antibody described herein to remove potential glycosylation sites on Fc region, which may reduce Fc receptor binding (see, e.g., Shields R L et al., (2001) J Biol Chem 276: 6591-604). In some embodiments of the aspects described herein, one or more of the following mutations in the constant region of an antibody described herein can be made: an N297 Å substitution; an N297Q substitution; a L235 Å substitution and a L237 Å substitution; a L234 Å substitution and a L235 Å substitution; a E233P substitution; a L234V substitution; a L235 Å substitution; a C236 deletion; a P238 Å substitution; a D265 Å substitution; a A327Q substitution; or a P329 Å substitution, numbered according to the EU index as in Kabat. In some embodiments of the aspects described herein, an antibody or antigen-binding fragment thereof described herein comprises the constant domain of an IgG1 with an N297Q or N297 Å amino acid substitution.

In some embodiments of the aspects described herein, one or more amino acids selected from amino acid residues 329, 331 and 322 in the constant region of an antibody described herein, numbered according to the EU index as in Kabat, can be replaced with a different amino acid residue such that the antibody has altered Clq binding and/or reduced or abolished complement dependent cytotoxicity (CDC). This approach is described in further detail in U.S. Pat. No. 6,194,551 (Idusogie et al). In some embodiments of the aspects described herein, one or more amino acid residues within amino acid positions 231 to 238 in the N-terminal region of the CH2 domain of an antibody described herein are altered to thereby alter the ability of the antibody to fix complement. This approach is described further in International Publication No. WO 94/29351. In some embodiments of the aspects described herein, the Fc region of an antibody described herein is modified to increase the ability of the antibody to mediate antibody dependent cellular cytotoxicity (ADCC) and/or to increase the affinity of the antibody for an Fcγ receptor by mutating one or more amino acids (e.g., introducing amino acid substitutions) at the following positions: 238, 239, 248, 249, 252, 254, 255, 256, 258, 265, 267, 268, 269, 270, 272, 276, 278, 280, 283, 285, 286, 289, 290, 292, 293, 294, 295, 296, 298, 301, 303, 305, 307, 309, 312, 315, 320, 322, 324, 326, 327, 329, 330, 331, 333, 334, 335, 337, 338, 340, 360, 373, 376, 378, 382, 388, 389, 398, 414, 416, 419, 430, 434, 435, 437, 438 or 439, numbered according to the EU index as in Kabat. This approach is described further in International Publication No. WO 00/42072.

In some embodiments of the aspects described herein, an antibody described herein comprises the constant region of an IgG4 antibody and the serine at amino acid residue 228 of the heavy chain, numbered according to the EU index as in Kabat, is substituted for proline.

Antibodies with reduced fucose content have been reported to have an increased affinity for Fc receptors, such as, e.g., FcγRIIIa. Accordingly, in certain embodiments, the antibodies or antigen-binding fragments thereof described herein have reduced fucose content or no fucose content. Such antibodies can be produced using techniques known to one skilled in the art. For example, the antibodies can be expressed in cells deficient or lacking the ability of fucosylation. In a specific example, cell lines with a knockout of both alleles of α1,6-fucosyltransferase can be used to produce antibodies with reduced fucose content. The POTELLIGENTR™ system (Lonza) is an example of such a system that can be used to produce antibodies with reduced fucose content. Alternatively, antibodies or antigen-binding fragments with reduced fucose content or no fucose content can be produced by, e.g.: (i) culturing cells under conditions which prevent or reduce fucosylation; (ii) posttranslational removal of fucose (e.g., with a fucosidase enzyme); (iii) posttranslational addition of the desired carbohydrate, e.g., after recombinant expression of a non-glycosylated glycoprotein; or (iv) purification of the glycoprotein so as to select for antibodies or antigen-binding fragments thereof which are not fucsoylated. See, e.g., Longmore G D & Schachter H (1982) Carbohydr Res 100: 365-92 and Imai-Nishiya H et al., (2007) BMC Biotechnol. 7: 84 for methods for producing antibodies or antigen-binding fragments thereof with no fucose content or reduced fucose content.

In some embodiments of the aspects described herein, antibodies or antigen-binding fragments thereof described herein have an increased affinity for CD32B (also known as FcγRIIB or FCGR2B), e.g., as compared to an antibody with a wild-type Fc region, e.g., an IgG1 Fc. In some embodiments of the aspects described herein, the antibodies or antigen-binding fragments thereof described herein have a selectively increased affinity for CD32B (FcγRIIB) over both CD32 Å (FcγRIIA) and CD16 (FcγRIIIA). Sequence alterations that result in increased affinity for CD32B are provided, for example, in Mimoto et al., Protein Engineering, Design & Selection 10: 589-598 (2013), Chu et al., Molecular Immunology 45: 3926-3933 (2008), and Strohl, Current Opinion in Biology 20: 685-691 (2009), each of which is herein incorporated by reference in its entirety. In some embodiments of the aspects described herein, the antibody or antigen-binding fragment with an increased affinity for CD32B comprises a heavy chain constant region, e.g., an IgG1 constant region, or fragment thereof comprising a mutation selected from the group consisting of: G236D, P238D, S239D, S267E, L328F, L328E, an arginine inserted after position 236, and combinations thereof, numbered according to EU index (Kabat et al., Sequences of Proteins of Immunological Interest, U.S. Department of Health and Human Services, Bethesda (1991)). In some embodiments of the aspects described herein, the antibody or antigen-binding fragment with an increased affinity for CD32B comprises a heavy chain constant region, e.g., an IgG1 constant region, or fragment thereof comprising S267E and L328F substitutions. In some embodiments of the aspects described herein, the antibody or antigen-binding fragment with an increased affinity for CD32B comprises a heavy chain constant region, e.g., an IgG1 constant region, or fragment thereof comprising P238D and L328E substitutions. In some embodiments of the aspects described herein, the antibody or antigen-binding fragment with an increased affinity for CD32B comprises a heavy chain constant region, e.g., an IgG1 constant region, or fragment thereof comprising a P238D substitution and substitution selected from the group consisting of E233D, G237D, H268D, P271G, A330R, and combinations thereof. In some embodiments of the aspects described herein, the antibody or antigen-binding fragment with an increased affinity for CD32B comprises a heavy chain constant region, e.g., an IgG1 constant region, or fragment thereof comprising P238D, E233D, G237D, H268D, P271G, and A330R substitutions. In some embodiments of the aspects described herein, the antibody or antigen-binding fragment with an increased affinity for CD32B comprises a heavy chain constant region, e.g., an IgG1 constant region, or fragment thereof comprising G236D and S267E. In some embodiments of the aspects described herein, the antibody or antigen-binding fragment with an increased affinity for CD32B comprises a heavy chain constant region, e.g., an IgG1 constant region, or fragment thereof comprising S239D and S267E. In some embodiments of the aspects described herein, the antibody or antigen-binding fragment with an increased affinity for CD32B comprises a heavy chain constant region, e.g., an IgG1 constant region, or fragment thereof comprising S267E and L328F. In some embodiments of the aspects described herein, the antibody or antigen-binding fragment with an increased affinity for CD32B comprises a heavy chain constant region, e.g., an IgG1 constant region, or fragment thereof comprising an arginine inserted after position 236 and L328R.

The term “CDR-grafted antibody” refers to antibodies which comprise heavy and light chain variable region sequences from one species, but in which the sequences of one or more of the CDR regions of V_{H} and/or V_{L} are replaced with CDR sequences of another species, such as antibodies having human heavy and light chain variable regions in which one or more of the human CDRs (e.g., CDR3) has been replaced with mouse CDR sequences. CDR-grafted antibodies described herein comprise heavy and light chain variable region sequences from a human antibody wherein one or more of the CDR regions of V_{H} and/or V_{L} are replaced with CDR sequences of the non-human antibodies described herein, such as one or more CDRs encoded by the variable heavy chain sequences of SEQ ID NOs: 1, 3, 5, 7, 9, 11, 13, 15, 17, 19, 21, and 23, and/or one or more CDRs encoded by the variable light chain sequences of SEQ ID NOs: 2, 4, 6, 8, 10, 12, 14, 16, 19, 20, 22, and 24.

In some embodiments of these aspects and all such aspects described herein, the IgM memory B cell receptor antigen-binding domain is a human IgM memory B cell receptor antigen binding domain. For example, provided herein are amino acid sequences for heavy chain antigen binding domains derived from malarial antigen-specific human IgM memory B cells (SEQ ID NOs: 26, 36, 46, 56, 66, 76, 86, 96, 106, 116, 126, 136, and 146) and amino acid sequences for light chain antigen binding domains derived from malarial antigen-specific human IgM memory B cells (SEQ ID NOs: 31, 41, 51, 61, 71, 81, 91, 101, 111, 121, 131, 141, and 151).

In some embodiments of these aspects and all such aspects described herein, the IgM memory B cell is CD21+CD27+.

In some embodiments of these aspects and all such aspects described herein, the non-IgM isotype antibody framework is an IgG antibody framework.

In some embodiments of these aspects and all such aspects described herein, the recombinant antigen-binding polypeptide comprises an scFv polypeptide, a single-domain antibody construct, a chimeric antibody construct or a bispecific antibody construct.

In some embodiments of these aspects and all such aspects described herein, the polypeptide binds its antigen with a K_{D} of 10⁻⁷ nM or lower.

As shown herein, sequencing of IgM receptors derived from IgM+ memory B cells demonstrates that these cells, like conventional IgG+ memory B cells, undergo somatic hypermutation in their variable heavy and light chains relative to germline variable heavy and light chains sequences. As known to those of skill in the art, “somatic hypermutation” is a cellular mechanism by which B cell receptors are diversified to increase affinity of a B cell receptor for its cognate antigen. Somatic hypermutation involves a programmed process of introducing point mutations into the variable regions of immunoglobulin genes, thereby increasing antibody diversity, and then using further positive selection to select antibodies that bind with higher affinity to the antigen. Somatic hypermutation has been estimated to expand the ultimate scope of antibody diversity 10 to 100-fold or more. Typically, an antigen-binding domain derived from an IgM memory B cell receptor for use in the compositions and methods described herein has undergone at least one or more, at least two or more, at least three or more, at least four or more, at least five or more, at least six or more, at least seven or more, but less than eight, somatic hypermutations relative to germline variable heavy and light chains sequences. Typically, an antigen-binding domain derived from an IgM memory B cell receptor for use in the compositions and methods described herein has undergone fewer than five somatic hypermutations, i.e., between one to five somatic hypermutations, between one to four somatic hypermutations, and between one to three somatic hypermutations.

In some embodiments of these aspects and all such aspects described herein, the variable light chain immunoglobulin sequence, variable heavy chain immunoglobulin sequence, or both has one or more somatic mutations relative to a variable heavy chain immunoglobulin sequence or variable light chain immunoglobulin sequence from a naïve B cell.

In some embodiments of these aspects and all such aspects described herein, the variable light chain sequence, variable heavy chain sequence, or both has one to eight somatic mutations relative to a variable heavy chain sequence or variable light chain sequence from a naïve B cell.

In some embodiments of these aspects and all such aspects described herein, the antigen-binding domain of the IgM memory B cell receptor has fewer than five somatic mutations.

In some embodiments of these aspects and all such aspects described herein, the IgM memory B cell receptor antigen-binding domain specifically binds an antigen comprised or expressed by an infectious organism.

In some embodiments of these aspects and all such aspects described herein, the infectious organism is a blood-borne pathogen.

In some embodiments of these aspects and all such aspects described herein, the infectious organism is a virus, a bacterium, a fungus or a parasite.

In some embodiments of these aspects and all such aspects described herein, the infectious organism is P. falciparum.

In some embodiments of these aspects and all such aspects described herein, the antigen is a blood stage malaria surface antigen or a sporozoite stage surface antigen. In some such embodiments, the blood stage malaria surface antigen or sporozoite stage surface antigen is selected from P. falciparum merozoite surface protein 1 (MSP1), AMA, and CSP (circumsporozoite protein).

In some embodiments of these aspects and all such aspects described herein, the IgM memory B cell receptor antigen-binding domain specifically binds a tumor antigen.

Non-limiting examples of tumor antigens to which an IgM memory B cell receptor antigen-binding domain can specifically bind include Acute myelogenous leukemia Wilms tumor 1 (WT1), preferentially expressed antigen of melanoma (PRAME), PR1, proteinase 3, elastase, cathepsin G, Chronic myelogenous WT1, Myelodysplastic syndrome WT1, Acute lymphoblastic leukemia PRAME, Chronic lymphocytic leukemia Survivin, Non-Hodgkin's lymphoma Survivin, Multiple myeloma New York esophagus 1 (NY-Esol), Malignant melanoma MAGE, MART-1/Melan-A, Tyrosinase, GP100, Breast cancer WT1, herceptin, Lung cancer WT1, Prostate-specific antigen (PSA), prostatic acid phosphatase, (PAP) Carcinoembryonic antigen (CEA), mucins (e.g., MUC-1), and Renal cell carcinoma (RCC) Fibroblast growth factor 5 (FGF-5).

Provided herein, in some aspects, are compositions comprising a population of antigen-specific IgM memory B cells bound via their B cell receptors to antigen immobilized on a solid support. A “solid support” for use in immobilizing, restraining, or capturing a population of antigen-specific IgM memory B cells can be any suitable solid support to which an antigen of interest can be attached or bound, and includes, for example, glass (e.g., a glass slide), plastic, chips, pins, filters, beads (e.g., magnetic beads, polystyrene beads, etc.), paper, membrane (e.g., nylon, nitrocellulose, polyvinylidene fluoride (PVDF), etc.), fiber bundles, or any other suitable substrate. The antigen of interest generally can be immobilized or restrained on the solid support via covalent or noncovalent interactions (e.g., ionic bonds, hydrophobic interactions, hydrogen bonds, Van der Waals forces, dipole-dipole bonds).

In some embodiments of these aspects and all such aspects described herein, the antigen immobilized on the solid support comprises a multimer construct comprising the antigen.

In some embodiments of these aspects and all such aspects described herein, the multimer construct comprises a dimer, trimer, or tetramer of the antigen.

In some embodiments of these aspects and all such aspects described herein, the antigen is an antigen expressed by an infectious organism.

In some embodiments of these aspects and all such aspects described herein, the infectious organism is a blood-borne pathogen.

In some embodiments of these aspects and all such aspects described herein, the infectious organism is a virus, a bacterium, a fungus or a parasite.

In some embodiments of these aspects and all such aspects described herein, the infectious organism is P. falciparum.

In some embodiments of these aspects and all such aspects described herein, the antigen is a blood stage malaria surface antigen or a sporozoite stage surface antigen. In some such embodiments, the blood stage malaria surface antigen or sporozoite stage surface antigen is selected from P. falciparum merozoite surface protein 1 (MSP1), AMA, and CSP.

In some embodiments of these aspects and all such aspects described herein, the IgM memory B cell receptor antigen-binding domain specifically binds a tumor antigen.

Provided herein, in some aspects, are populations comprising at least 100 recombinant antigen-binding molecules, each comprising an antigen-binding domain of an IgM memory B cell receptor, and each binding its antigen with a K_{D} of 10⁻⁷ nM or lower. The recombinant antigen-binding molecules of the population can be derived from BCR antigen-binding sequences of a population of antigen-specific MBCs isolated as described herein. In some embodiments, the average frequency of somatic mutation is eight or fewer per molecule for the population. In some embodiments, the average frequency of somatic mutation is five or fewer per molecule for the population. In some embodiments, the population binds the same antigen. In some embodiments, the population binds the same antigen, but different epitopes on the same antigen.

In some embodiments of these aspects and all such aspects described herein, the antigen is an antigen expressed or comprised by an infectious organism.

In some embodiments of these aspects and all such aspects described herein, the infectious organism is a blood-borne pathogen.

In some embodiments of these aspects and all such aspects described herein, the infectious organism is a virus, a bacterium, a fungus or a parasite.

In some embodiments of these aspects and all such aspects described herein, the infectious organism is P. falciparum.

In some embodiments of these aspects and all such aspects described herein, the antigen is P. falciparum MSP1.

In some aspects, provided herein are pharmaceutical compositions comprising any of the antibody or BCR-derived compositions described herein, and a pharmaceutically acceptable carrier.

In some aspects, provided herein are vaccine compositions comprising any of the compositions described herein.

Provided herein, in some aspects are recombinant antibodies or antigen-binding fragments thereof that specifically bind to the malarial antigen AMA and comprise heavy chain complimentarity determining regions (CDRs) selected from the group consisting of:
a. a heavy chain CDR1 having the amino acid sequence of SEQ ID NO: 27; a heavy chain CDR2 having the amino acid sequence of SEQ ID NO: 28; and a heavy chain CDR3 having the amino acid sequence of SEQ ID NO: 29;
b. a heavy chain CDR1 having the amino acid sequence of SEQ ID NO: 37; a heavy chain CDR2 having the amino acid sequence of SEQ ID NO: 38; and a heavy chain CDR3 having the amino acid sequence of SEQ ID NO: 39;
c. a heavy chain CDR1 having the amino acid sequence of SEQ ID NO: 47; a heavy chain CDR2 having the amino acid sequence of SEQ ID NO: 48; and a heavy chain CDR3 having the amino acid sequence of SEQ ID NO: 49;
d. a heavy chain CDR1 having the amino acid sequence of SEQ ID NO: 57; a heavy chain CDR2 having the amino acid sequence of SEQ ID NO: 58; and a heavy chain CDR3 having the amino acid sequence of SEQ ID NO: 59;
e. a heavy chain CDR1 having the amino acid sequence of SEQ ID NO: 77; a heavy chain CDR2 having the amino acid sequence of SEQ ID NO: 78; and a heavy chain CDR3 having the amino acid sequence of SEQ ID NO: 79;
f. a heavy chain CDR1 having the amino acid sequence of SEQ ID NO: 107; a heavy chain CDR2 having the amino acid sequence of SEQ ID NO: 108; and a heavy chain CDR3 having the amino acid sequence of SEQ ID NO: 109;
g. a heavy chain CDR1 having the amino acid sequence of SEQ ID NO: 137; a heavy chain CDR2 having the amino acid sequence of SEQ ID NO: 138; and a heavy chain CDR3 having the amino acid sequence of SEQ ID NO: 139; and
h. a heavy chain CDR1 having the amino acid sequence of SEQ ID NO: 147; a heavy chain CDR2 having the amino acid sequence of SEQ ID NO: 148; and a heavy chain CDR3 having the amino acid sequence of SEQ ID NO: 149.

In some aspects, provided herein are recombinant antibodies or antigen-binding fragments thereof that specifically bind to the malarial antigen AMA and comprise light chain complimentarity determining regions (CDRs) selected from the group consisting of:
a. a light chain CDR1 having the amino acid sequence of SEQ ID NO: 32; a light chain CDR2 having the amino acid sequence of SEQ ID NO: 33; and a light chain CDR3 having the amino acid sequence of SEQ ID NO: 34;
b. a light chain CDR1 having the amino acid sequence of SEQ ID NO: 42; a light chain CDR2 having the amino acid sequence of SEQ ID NO: 43; and a light chain CDR3 having the amino acid sequence of SEQ ID NO: 44;
c. a light chain CDR1 having the amino acid sequence of SEQ ID NO: 52; a light chain CDR2 having the amino acid sequence of SEQ ID NO: 53; and a light chain CDR3 having the amino acid sequence of SEQ ID NO: 54;
d. a light chain CDR1 having the amino acid sequence of SEQ ID NO: 62; a light chain CDR2 having the amino acid sequence of SEQ ID NO: 63; and a light chain CDR3 having the amino acid sequence of SEQ ID NO: 64;
e. a light chain CDR1 having the amino acid sequence of SEQ ID NO: 82; a light chain CDR2 having the amino acid sequence of SEQ ID NO: 83; and a light chain CDR3 having the amino acid sequence of SEQ ID NO: 84;
f. a light chain CDR1 having the amino acid sequence of SEQ ID NO: 112; a light chain CDR2 having the amino acid sequence of SEQ ID NO: 113; and a light chain CDR3 having the amino acid sequence of SEQ ID NO: 114;
g. a light chain CDR1 having the amino acid sequence of SEQ ID NO: 142; a light chain CDR2 having the amino acid sequence of SEQ ID NO: 143; and a light chain CDR3 having the amino acid sequence of SEQ ID NO: 144; and
h. a light chain CDR1 having the amino acid sequence of SEQ ID NO: 152; a light chain CDR2 having the amino acid sequence of SEQ ID NO: 153; and a light chain CDR3 having the amino acid sequence of SEQ ID NO: 154.

In some aspects, provided herein are recombinant antibodies or antigen-binding fragments thereof that specifically bind to the malarial antigen AMA and comprise heavy and light chain complimentarity determining regions (CDRs) selected from the group consisting of:
a. a heavy chain CDR1 having the amino acid sequence of SEQ ID NO: 27; a heavy chain CDR2 having the amino acid sequence of SEQ ID NO: 28; a heavy chain CDR3 having the amino acid sequence of SEQ ID NO: 29; a light chain CDR1 having the amino acid sequence of SEQ ID NO: 32; a light chain CDR2 having the amino acid sequence of SEQ ID NO: 33; and a light chain CDR3 having the amino acid sequence of SEQ ID NO: 34;
b. a heavy chain CDR1 having the amino acid sequence of SEQ ID NO: 37; a heavy chain CDR2 having the amino acid sequence of SEQ ID NO: 38; a heavy chain CDR3 having the amino acid sequence of SEQ ID NO: 39; a light chain CDR1 having the amino acid sequence of SEQ ID NO: 42; a light chain CDR2 having the amino acid sequence of SEQ ID NO: 43; and a light chain CDR3 having the amino acid sequence of SEQ ID NO: 44;
c. a heavy chain CDR1 having the amino acid sequence of SEQ ID NO: 47; a heavy chain CDR2 having the amino acid sequence of SEQ ID NO: 48; a heavy chain CDR3 having the amino acid sequence of SEQ ID NO: 49; a light chain CDR1 having the amino acid sequence of SEQ ID NO: 52; a light chain CDR2 having the amino acid sequence of SEQ ID NO: 53; and a light chain CDR3 having the amino acid sequence of SEQ ID NO: 54;
d. a heavy chain CDR1 having the amino acid sequence of SEQ ID NO: 57; a heavy chain CDR2 having the amino acid sequence of SEQ ID NO: 58; a heavy chain CDR3 having the amino acid sequence of SEQ ID NO: 59; a light chain CDR1 having the amino acid sequence of SEQ ID NO: 62; a light chain CDR2 having the amino acid sequence of SEQ ID NO: 63; and a light chain CDR3 having the amino acid sequence of SEQ ID NO: 64;
e. a heavy chain CDR1 having the amino acid sequence of SEQ ID NO: 77; a heavy chain CDR2 having the amino acid sequence of SEQ ID NO: 78; a heavy chain CDR3 having the amino acid sequence of SEQ ID NO: 79; a light chain CDR1 having the amino acid sequence of SEQ ID NO: 82; a light chain CDR2 having the amino acid sequence of SEQ ID NO: 83; and a light chain CDR3 having the amino acid sequence of SEQ ID NO: 84;
f. a heavy chain CDR1 having the amino acid sequence of SEQ ID NO: 107; a heavy chain CDR2 having the amino acid sequence of SEQ ID NO: 108; a heavy chain CDR3 having the amino acid sequence of SEQ ID NO: 109; a light chain CDR1 having the amino acid sequence of SEQ ID NO: 112; a light chain CDR2 having the amino acid sequence of SEQ ID NO: 113; and a light chain CDR3 having the amino acid sequence of SEQ ID NO: 114;
g. a heavy chain CDR1 having the amino acid sequence of SEQ ID NO: 137; a heavy chain CDR2 having the amino acid sequence of SEQ ID NO: 138; a heavy chain CDR3 having the amino acid sequence of SEQ ID NO: 139; a light chain CDR1 having the amino acid sequence of SEQ ID NO: 142; a light chain CDR2 having the amino acid sequence of SEQ ID NO: 143; and a light chain CDR3 having the amino acid sequence of SEQ ID NO: 144; and
h. a heavy chain CDR1 having the amino acid sequence of SEQ ID NO: 147; a heavy chain CDR2 having the amino acid sequence of SEQ ID NO: 148; a heavy chain CDR3 having the amino acid sequence of SEQ ID NO: 149; a light chain CDR1 having the amino acid sequence of SEQ ID NO: 152; a light chain CDR2 having the amino acid sequence of SEQ ID NO: 153; and a light chain CDR3 having the amino acid sequence of SEQ ID NO: 154.

Provided herein, in some aspects are recombinant antibodies or antigen-binding fragments thereof that specifically bind to the malarial antigen MSP1 and comprise heavy chain complimentarity determining regions (CDRs) selected from the group consisting of:
a. a heavy chain CDR1 having the amino acid sequence of SEQ ID NO: 67; a heavy chain CDR2 having the amino acid sequence of SEQ ID NO: 68; and a heavy chain CDR3 having the amino acid sequence of SEQ ID NO: 69;
b. a heavy chain CDR1 having the amino acid sequence of SEQ ID NO: 87; a heavy chain CDR2 having the amino acid sequence of SEQ ID NO: 88; and a heavy chain CDR3 having the amino acid sequence of SEQ ID NO: 89;
c. a heavy chain CDR1 having the amino acid sequence of SEQ ID NO: 97; a heavy chain CDR2 having the amino acid sequence of SEQ ID NO: 98; and a heavy chain CDR3 having the amino acid sequence of SEQ ID NO: 99;
d. a heavy chain CDR1 having the amino acid sequence of SEQ ID NO: 117; a heavy chain CDR2 having the amino acid sequence of SEQ ID NO: 118; and a heavy chain CDR3 having the amino acid sequence of SEQ ID NO: 119; and
e. a heavy chain CDR1 having the amino acid sequence of SEQ ID NO: 127; a heavy chain CDR2 having the amino acid sequence of SEQ ID NO: 128; and a heavy chain CDR3 having the amino acid sequence of SEQ ID NO: 129;

In some aspects, provided herein are recombinant antibodies or antigen-binding fragments thereof that specifically bind to the malarial antigen MSP1 and comprise light chain complimentarity determining regions (CDRs) selected from the group consisting of:
a. a light chain CDR1 having the amino acid sequence of SEQ ID NO: 72; a light chain CDR2 having the amino acid sequence of SEQ ID NO: 73; and a light chain CDR3 having the amino acid sequence of SEQ ID NO: 74;
b. a light chain CDR1 having the amino acid sequence of SEQ ID NO: 92; a light chain CDR2 having the amino acid sequence of SEQ ID NO: 93; and a light chain CDR3 having the amino acid sequence of SEQ ID NO: 94;
c. a light chain CDR1 having the amino acid sequence of SEQ ID NO: 102; a light chain CDR2 having the amino acid sequence of SEQ ID NO: 103; and a light chain CDR3 having the amino acid sequence of SEQ ID NO: 104;
d. a light chain CDR1 having the amino acid sequence of SEQ ID NO: 122; a light chain CDR2 having the amino acid sequence of SEQ ID NO: 123; and a light chain CDR3 having the amino acid sequence of SEQ ID NO: 124; and
e. a light chain CDR1 having the amino acid sequence of SEQ ID NO: 132; a light chain CDR2 having the amino acid sequence of SEQ ID NO: 133; and a light chain CDR3 having the amino acid sequence of SEQ ID NO: 134;

In some aspects, provided herein are recombinant antibodies or antigen-binding fragments thereof that specifically bind to the malarial antigen MSP1 and comprise heavy and light chain complimentarity determining regions (CDRs) selected from the group consisting of:
a. a heavy chain CDR1 having the amino acid sequence of SEQ ID NO: 67; a heavy chain CDR2 having the amino acid sequence of SEQ ID NO: 68; a heavy chain CDR3 having the amino acid sequence of SEQ ID NO: 69; a light chain CDR1 having the amino acid sequence of SEQ ID NO: 72; a light chain CDR2 having the amino acid sequence of SEQ ID NO: 73; and a light chain CDR3 having the amino acid sequence of SEQ ID NO: 74;
b. a heavy chain CDR1 having the amino acid sequence of SEQ ID NO: 87; a heavy chain CDR2 having the amino acid sequence of SEQ ID NO: 88; a heavy chain CDR3 having the amino acid sequence of SEQ ID NO: 89; a light chain CDR1 having the amino acid sequence of SEQ ID NO: 92; a light chain CDR2 having the amino acid sequence of SEQ ID NO: 93; and a light chain CDR3 having the amino acid sequence of SEQ ID NO: 94;
c. a heavy chain CDR1 having the amino acid sequence of SEQ ID NO: 97; a heavy chain CDR2 having the amino acid sequence of SEQ ID NO: 98; a heavy chain CDR3 having the amino acid sequence of SEQ ID NO: 99; a light chain CDR1 having the amino acid sequence of SEQ ID NO: 102; a light chain CDR2 having the amino acid sequence of SEQ ID NO: 103; and a light chain CDR3 having the amino acid sequence of SEQ ID NO: 104;
d. a heavy chain CDR1 having the amino acid sequence of SEQ ID NO: 117; a heavy chain CDR2 having the amino acid sequence of SEQ ID NO: 118; a heavy chain CDR3 having the amino acid sequence of SEQ ID NO: 119; a light chain CDR1 having the amino acid sequence of SEQ ID NO: 122; a light chain CDR2 having the amino acid sequence of SEQ ID NO: 123; and a light chain CDR3 having the amino acid sequence of SEQ ID NO: 124;
e. a heavy chain CDR1 having the amino acid sequence of SEQ ID NO: 127; a heavy chain CDR2 having the amino acid sequence of SEQ ID NO: 128; a heavy chain CDR3 having the amino acid sequence of SEQ ID NO: 129; a light chain CDR1 having the amino acid sequence of SEQ ID NO: 132; a light chain CDR2 having the amino acid sequence of SEQ ID NO: 133; and a light chain CDR3 having the amino acid sequence of SEQ ID NO: 134;

In some aspects, provided herein are recombinant antibodies produced from any of the isolated or recombinant antibody-producing B-cells described herein.

In some aspects, provided herein are recombinant antibodies comprising a variable region from a Plasmodium-specific memory B cell and an immunoglobulin light chain isotype.

In some embodiments of these aspects and all such aspects described herein, the recombinant antibody is for the treatment of or protection from malaria infection in a subject.

In some embodiments of these aspects and all such aspects described herein, the recombinant antibody is for vaccination against malaria.

In some embodiments of these aspects and all such aspects described herein, the recombinant antibody is for the treatment of multi-drug resistant malaria.

In some embodiments of these aspects and all such aspects described herein, the subject is a mammal.

In some embodiments of these aspects and all such aspects described herein, the subject is a human.

### Pharmaceutical Compositions and Methods of Treatment

Provided herein, in some aspects, are pharmaceutical compositions comprising any of the recombinant antibodies described herein.

For the clinical use of the methods described herein, administration of antibodies or antigen-binding fragments thereof described herein can include formulation into pharmaceutical compositions or pharmaceutical formulations for parenteral administration, e.g., intravenous; mucosal, e.g., intranasal; ocular, or other mode of administration. In some embodiments, the antibodies or antigen-binding fragments thereof described herein can be administered along with any pharmaceutically acceptable carrier compound, material, or composition which results in an effective treatment in the subject. Thus, a pharmaceutical formulation for use in the methods described herein can contain an antibody or antigen-binding fragment thereof as described herein in combination with one or more pharmaceutically acceptable ingredients.

The phrase “pharmaceutically acceptable” refers to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio. The phrase “pharmaceutically acceptable carrier” as used herein means a pharmaceutically acceptable material, composition or vehicle, such as a liquid or solid filler, diluent, excipient, solvent, media, encapsulating material, manufacturing aid (e.g., lubricant, talc magnesium, calcium or zinc stearate, or steric acid), or solvent encapsulating material, involved in maintaining the stability, solubility, or activity of, an antibody or antigen-binding fragment thereof. Each carrier must be “acceptable” in the sense of being compatible with the other ingredients of the formulation and not injurious to the patient. The terms “excipient”, “carrier”, “pharmaceutically acceptable carrier” or the like are used interchangeably herein.

The antibodies or antigen-binding fragments thereof described herein can be specially formulated for administration of the compound to a subject in solid, liquid or gel form, including those adapted for the following: (1) parenteral administration, for example, by subcutaneous, intramuscular, intravenous or epidural injection as, for example, a sterile solution or suspension, or sustained-release formulation; (2) topical application, for example, as a cream, ointment, or a controlled-release patch or spray applied to the skin; (3) intravaginally or intrarectally, for example, as a pessary, cream or foam; (4) ocularly; (5) transdermally; (6) transmucosally; or (79) nasally. Additionally, an antibody or antigen-binding fragment thereof can be implanted into a patient or injected using a drug delivery system. See, for example, Urquhart, et al., Ann. Rev. Pharmacol. Toxicol. 24: 199-236 (1984); Lewis, ed. “Controlled Release of Pesticides and Pharmaceuticals” (Plenum Press, New York, 1981); U.S. Pat. No. 3,773,919; and U.S. Pat. No. 35 3,270,960.

Therapeutic formulations of the antibodies or antigen-binding fragments thereof described herein can be prepared for storage by mixing the antibodies or antigen-binding fragments having the desired degree of purity with optional pharmaceutically acceptable carriers, excipients or stabilizers (Remington's Pharmaceutical Sciences 16th edition, Osol, A. Ed. (1980)), in the form of lyophilized formulations or aqueous solutions. Acceptable carriers, excipients, or stabilizers are nontoxic to recipients at the dosages and concentrations employed, and include buffers such as phosphate, citrate, and other organic acids; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g. Zn-protein complexes); and/or non-ionic surfactants such as TWEEN™, PLURONICS™ or polyethylene glycol (PEG). Exemplary lyophilized antibody formulations are described in WO 97/04801, expressly incorporated herein by reference.

Optionally, but preferably, the formulations comprising the compositions described herein contain a pharmaceutically acceptable salt, typically, e.g., sodium chloride, and preferably at about physiological concentrations. Optionally, the formulations of the invention can contain a pharmaceutically acceptable preservative. In some embodiments the preservative concentration ranges from 0.1 to 2.0%, typically v/v. Suitable preservatives include those known in the pharmaceutical arts. Benzyl alcohol, phenol, m-cresol, methylparaben, and propylparaben are examples of preservatives. Optionally, the formulations of the invention can include a pharmaceutically acceptable surfactant at a concentration of 0.005 to 0.02%.

The therapeutic formulations of the compositions comprising antibodies and antigen-binding fragments thereof described herein can also contain more than one active compound as necessary for the particular indication being treated, preferably those with complementary activities that do not adversely affect each other. Alternatively, the composition can comprise a cytotoxic agent, cytokine, or growth inhibitory agent, for example. Such molecules are suitably present in combination in amounts that are effective for the purpose intended.

The active ingredients of the therapeutic formulations of the compositions comprising antibodies or antigen-binding fragments described herein can also be entrapped in microcapsules prepared, for example, by coacervation techniques or by interfacial polymerization, for example, hydroxymethylcellulose or gelatin-microcapsules and poly-(methylmethacylate) microcapsules, respectively, in colloidal drug delivery systems (for example, liposomes, albumin microspheres, microemulsions, nano-particles and nanocapsules) or in macroemulsions. Such techniques are disclosed in Remington's Pharmaceutical Sciences 16th edition, Osol, A. Ed. (1980).

In some embodiments, sustained-release preparations can be used. Suitable examples of sustained-release preparations include semipermeable matrices of solid hydrophobic polymers containing the antibodies or antigen-binding fragments in which the matrices are in the form of shaped articles, e.g., films, or microcapsule. Examples of sustained-release matrices include polyesters, hydrogels (for example, poly(2-hydroxyethyl-methacrylate), or poly(vinylalcohol)), polylactides (U.S. Pat. No. 3,773,919), copolymers of L-glutamic acid and y ethyl-L-glutamate, non-degradable ethylene-vinyl acetate, degradable lactic acid-glycolic acid copolymers such as the LUPRON DEPOT™ (injectable microspheres composed of lactic acid-glycolic acid copolymer and leuprolide acetate), and poly-D-(−)-3-hydroxybutyric acid. While polymers such as ethylene-vinyl acetate and lactic acid-glycolic acid enable release of molecules for over 100 days, certain hydrogels release proteins for shorter time periods. When encapsulated antibodies remain in the body for a long time, they can denature or aggregate as a result of exposure to moisture at 37° C., resulting in a loss of biological activity and possible changes in immunogenicity. Rational strategies can be devised for stabilization depending on the mechanism involved. For example, if the aggregation mechanism is discovered to be intermolecular S—S bond formation through thio-disulfide interchange, stabilization can be achieved by modifying sulfhydryl residues, lyophilizing from acidic solutions, controlling moisture content, using appropriate additives, and developing specific polymer matrix compositions.

The therapeutic formulations to be used for in vivo administration, such as parenteral administration, in the methods described herein can be sterile, which is readily accomplished by filtration through sterile filtration membranes, or other methods known to those of skill in the art.

Antibodies and antigen-binding fragments thereof, are formulated, dosed, and administered in a fashion consistent with good medical practice. Factors for consideration in this context include the particular disorder being treated, the particular subject being treated, the clinical condition of the individual subject, the cause of the disorder, the site of delivery of the agent, the method of administration, the scheduling of administration, and other factors known to medical practitioners. The “therapeutically effective amount” of the antibodies and antigen-binding fragments thereof to be administered are governed by such considerations, and refers to the minimum amount necessary to ameliorate, treat, or stabilize, the cancer; to increase the time until progression (duration of progression free survival) or to treat or prevent the occurrence or recurrence of an infection or tumor. The antibodies and antigen-binding fragments thereof are optionally formulated, in some embodiments, with one or more additional therapeutic agents currently used to prevent or treat the infection, for example. The effective amount of such other agents depends on the amount of antibodies and antigen-binding fragments thereof present in the formulation, the type of disorder or treatment, and other factors discussed above. These are generally used in the same dosages and with administration routes as used herein before or about from 1 to 99% of the heretofore employed dosages.

The dosage ranges for the therapeutic agents depend upon the potency, and encompass amounts large enough to produce the desired effect. The dosage should not be so large as to cause unacceptable adverse side effects. Generally, the dosage will vary with the age, condition, and sex of the patient and can be determined by one of skill in the art. The dosage can also be adjusted by the individual physician in the event of any complication. In some embodiments, the dosage ranges from 0.001 mg/kg body weight to 100 mg/kg body weight. In some embodiments, the dose range is from 5 μg/kg body weight to 100 μg/kg body weight. Alternatively, the dose range can be titrated to maintain serum levels between 1 μg/mL and 1000 μg/mL. For systemic administration, subjects can be administered a therapeutic amount, such as, e.g., 0.1 mg/kg, 0.5 mg/kg, 1.0 mg/kg, 2.0 mg/kg, 2.5 mg/kg, 5 mg/kg, 7.5 mg/kg, 10 mg/kg, 15 mg/kg, 20 mg/kg, 25 mg/kg, 30 mg/kg, 40 mg/kg, 50 mg/kg, or more. These doses can be administered by one or more separate administrations, or by continuous infusion. For repeated administrations over several days or longer, depending on the condition, the treatment is sustained until, for example, the cancer is treated, as measured by the methods described above or known in the art. However, other dosage regimens can be useful.

In some aspects, provided herein are methods of treating a subject in need of treatment for a disease caused by an infectious organism, the method comprising administering an antigen-binding compositions as described herein, wherein the antigen-binding polypeptide of the composition specifically binds an antigen comprised by the infectious organism.

In some aspects, provided herein are methods reducing the likelihood of contracting a disease caused by an infectious organism, the method comprising administering to an individual at risk of contracting the disease an antigen-binding composition as described herein, wherein the antigen-binding polypeptide specifically binds an antigen comprised by the infectious organism.

In some aspects, provided herein are methods of treating a subject in need of treatment for a tumor that expresses a tumor antigen, the method comprising administering an antigen-binding composition as described herein to the subject, wherein the antigen-binding polypeptide of the composition specifically binds the tumor antigen.

In some aspects, provided herein are isolated or recombinant antibody-producing B-cells produced by any of the methods described herein.

In some aspects, provided herein are methods of treating malaria infection in a subject, comprising administering a therapeutically effective amount of any a recombinant antibody as described herein.

In some aspects, provided herein are methods of treating multi-drug resistant malaria in a subject, comprising administering a therapeutically effective amount of a recombinant antibody as described herein.

In some aspects, provided herein are methods of preventing malaria infection in a subject, comprising administering a pharmaceutically effective amount of a recombinant antibody as described herein.

In some embodiments of these methods and all such methods described herein, the subject is a mammal. In some embodiments of these aspects and all such aspects described herein, the subject is a human.

In some embodiments of these methods and all such methods described herein, the subject is immunocompromised.

The recombinant antigen-binding polypeptides comprising an antigen-binding domain of an IgM memory B cell receptor described herein can be administered to a subject in need thereof by any appropriate route which results in an effective treatment in the subject. As used herein, the terms “administering,” and “introducing” are used interchangeably and refer to the placement of an antibody or antigen-binding fragment thereof into a subject by a method or route which results in at least partial localization of such agents at a desired site, such as a site of infection or cancer, such that a desired effect(s) is produced. An antibody or antigen-binding fragment thereof can be administered to a subject by any mode of administration that delivers the agent systemically or to a desired surface or target, and can include, but is not limited to, injection, infusion, instillation, and inhalation administration. To the extent that antibodies or antigen-binding fragments thereof can be protected from inactivation in the gut, oral administration forms are also contemplated. “Injection” includes, without limitation, intravenous, intramuscular, intra-arterial, intrathecal, intraventricular, intracapsular, intraorbital, intracardiac, intradermal, intraperitoneal, transtracheal, subcutaneous, subcuticular, intraarticular, sub capsular, subarachnoid, intraspinal, intracerebro spinal, and intrasternal injection and infusion.

The phrases “parenteral administration” and “administered parenterally” as used herein, refer to modes of administration other than enteral and topical administration, usually by injection. The phrases “systemic administration,” “administered systemically”, “peripheral administration” and “administered peripherally” as used herein refer to the administration of a therapeutic agent other than directly into a target site, tissue, or organ, such as a tumor site, such that it enters the subject's circulatory system and, thus, is subject to metabolism and other like processes. In other embodiments, the antibody or antigen-binding fragment thereof is administered locally, e.g., by direct injections, when the disorder or location of the infection permits, and the injections can be repeated periodically.

As used herein, the terms “treat,” “treatment,” “treating,” or “amelioration” refer to therapeutic treatments, wherein the object is to reverse, alleviate, ameliorate, inhibit, slow down or stop the progression or severity of a condition associated with, a disease or disorder. The term “treating” includes reducing or alleviating at least one adverse effect or symptom of a condition, disease or disorder associated with a chronic immune condition, such as, but not limited to, a chronic infection or a cancer. Treatment is generally “effective” if one or more symptoms or clinical markers are reduced. Alternatively, treatment is “effective” if the progression of a disease is reduced or halted. That is, “treatment” includes not just the improvement of symptoms or markers, but also a cessation of at least slowing of progress or worsening of symptoms that would be expected in absence of treatment. Beneficial or desired clinical results include, but are not limited to, alleviation of one or more symptom(s), diminishment of extent of disease, stabilized (i.e., not worsening) state of disease, delay or slowing of disease progression, amelioration or palliation of the disease state, and remission (whether partial or total), whether detectable or undetectable. The term “treatment” of a disease also includes providing relief from the symptoms or side-effects of the disease (including palliative treatment).

The duration of a therapy using the methods described herein will continue for as long as medically indicated or until a desired therapeutic effect (e.g., those described herein) is achieved. In certain embodiments, the administration of antibody or antigen-binding fragment described herein is continued for 1 month, 2 months, 4 months, 6 months, 8 months, 10 months, 1 year, 2 years, 3 years, 4 years, 5 years, 10 years, 20 years, or for a period of years up to the lifetime of the subject.

The term “effective amount” as used herein refers to the amount of an antibody or antigen-binding fragment thereof needed to alleviate at least one or more symptom of the disease or disorder, and relates to a sufficient amount of pharmacological composition to provide the desired effect, e.g., reduce an infectious organism or tumor load or reduce pathology or any symptom associated with or caused by the infectious organism or tumor load. The term “therapeutically effective amount” therefore refers to an amount of an antibody or antigen-binding fragment thereof using the methods as disclosed herein, that is sufficient to effect a particular effect when administered to a typical subject. An effective amount as used herein would also include an amount sufficient to delay the development of a symptom of the disease, alter the course of a symptom disease (for example but not limited to, slow the progression of a symptom of the disease), or reverse a symptom of the disease. Thus, it is not possible to specify the exact “effective amount.” However, for any given case, an appropriate “effective amount” can be determined by one of ordinary skill in the art using only routine experimentation.

Effective amounts, toxicity, and therapeutic efficacy can be determined by standard pharmaceutical procedures in cell cultures or experimental animals, e.g., for determining the LD₅₀ (the dose lethal to 50% of the population) and the ED₅₀ (the dose therapeutically effective in 50% of the population). The dosage can vary depending upon the dosage form employed and the route of administration utilized. The dose ratio between toxic and therapeutic effects is the therapeutic index and can be expressed as the ratio LD₅₀/ED₅₀. Compositions and methods that exhibit large therapeutic indices are preferred. A therapeutically effective dose can be estimated initially from cell culture assays. Also, a dose can be formulated in animal models to achieve a circulating plasma concentration range that includes the IC₅₀ (i.e., the concentration of the antibody or antigen-binding fragment thereof), which achieves a half-maximal inhibition of symptoms) as determined in cell culture, or in an appropriate animal model. Levels in plasma can be measured, for example, by high performance liquid chromatography. The effects of any particular dosage can be monitored by a suitable bioassay. The dosage can be determined by a physician and adjusted, as necessary, to suit observed effects of the treatment.

In some embodiments of these methods and all such methods described herein, the recombinant antibody is administered in an amount effective to provide short-term protection against a malaria infection.

As used herein, “short-term protection” refers to protection from an infection, such as a malarial infection, lasting at least about 2 months, at least about 3 months, at least about 4 months, at least about 5 months, at least about 6 months, at least about 7 months, at least about 8 months, at least about 9 months, at least about 10 months, at least about 11 months, or at least about 12 months.

“Alleviating a symptom of a persistent infection” is ameliorating any condition or symptom associated with the persistent infection. Alternatively, alleviating a symptom of a persistent infection can involve reducing the infectious microbial (such as viral, bacterial, fungal or parasitic) load in the subject relative to such load in an untreated control. As compared with an equivalent untreated control, such reduction or degree of prevention is at least 5%, 10%, 20%, 40%, 50%, 60%, 80%, 90%, 95%, or 100% as measured by any standard technique. Desirably, the persistent infection is completely cleared as detected by any standard method known in the art, in which case the persistent infection is considered to have been treated. A patient who is being treated for a persistent infection is one who a medical practitioner has diagnosed as having such a condition. Diagnosis may be by any suitable means. Diagnosis and monitoring may involve, for example, detecting the level of microbial load in a biological sample (for example, a tissue biopsy, blood test, or urine test), detecting the level of a surrogate marker of the microbial infection in a biological sample, detecting symptoms associated with persistent infections, or detecting immune cells involved in the immune response typical of persistent infections (for example, detection of antigen specific T cells that are anergic and/or functionally impaired). A patient in whom the development of a persistent infection is being prevented may or may not have received such a diagnosis. One in the art will understand that these patients may have been subjected to the same standard tests as described above or may have been identified, without examination, as one at high risk due to the presence of one or more risk factors (such as family history or exposure to infectious agent).

For the treatment of diseases, as described herein, the appropriate dosage of an antibody or antigen-binding fragment thereof will depend on the type of disease to be treated, as defined above, the severity and course of the disease, whether the antibody or antigen-binding fragment thereof is administered for preventive or therapeutic purposes, previous therapeutic indications, the subject's clinical history and response to the antibody or antigen-binding fragment thereof, and the discretion of the attending physician. The antibody or antigen-binding fragment thereof is suitably administered to the subject at one time or over a series of treatments. In a combination therapy regimen, the antibody or antigen-binding fragment thereof and the one or more additional therapeutic agents described herein are administered in a therapeutically effective or synergistic amount. As used herein, a therapeutically effective amount is such that co-administration of an antibody or antigen-binding fragment thereof and one or more other therapeutic agents, or administration of a composition described herein, results in reduction or inhibition of a disease or disorder as described herein. A therapeutically synergistic amount is that amount of an antibody or antigen-binding fragment thereof and one or more other therapeutic agents necessary to synergistically or significantly reduce or eliminate conditions or symptoms associated with a particular disease. In some cases, the antibody or antigen-binding fragment thereof can be co-administered with one or more additional therapeutically effective agents to give an additive effect resulting in a significantly reduction or elimination of conditions or symptoms associated with a particular disease, but with a much reduced toxicity profile due to lower dosages of one or more of the additional therapeutically effective agents.

Exemplary Antigen-Specific IgM Memory B cell Clone Sequences

Provided herein as SEQ ID NOs: 1-154 are nucleotide and corresponding amino acid sequences, and heavy and light chain CDR amino acid sequences sequenced from malarial antigen-specific memory B cell clones obtained using the methods described herein.

Clone B6-3P1 uses a V_{H} IMGT of IGHV1-64*01, a J_{H} IMGT of IGHJ4*01 and has a VH light chain nucleotide sequence of:

(SEQ ID NO: 1)

CAGGTGCAGCTGCAGCAGCCTGGGGCTGAGCTGGTAAAGCCTGGGGCTTC
AGTGAAGTTGTCCTGCAAGGCTTCTGGCTACACTTTCATCAACTACTGGA
TGCACTGGGTGAAGCAGAGGCCTGGACAAGGCCTTGAGTGGATTGGAATG
ATTCATCCTAAAAGTGGTAGCACTAACTTCAATGAGAAGTTCAAGAGCAA
GGCCACACTGACTGTAGACAAATCCTCCAGCACAGCCTACATGCAAGTCA
GCAGCCTGACATCTGAGGACTCTGCGGTCTATTATTGTGCAAAGGAAGTC
ATGGACTACTGGGGTCAAGGAACCTCAGTCACCGTCTCCTCAG.

Clone B6-3P1 uses a V_{K} IMGT of IGKV8-24*01, a J_{K} IMGT of IGKJ2*01 and has a VK light chain nucleotide sequence of:

(SEQ ID NO: 2)

GACATTGTGATGACTCAGTCTCCATCCTCCCTGGCTATGTCAGTAGGACA
GAAGGTCACTATGAACTGCAAGTCCAGTCAGAGCCTTTTAAATAGTAGAA
ATCAAAAGAATTTTTTGGCCTGGTACCAACAGAAACCAGGACAGTCTCCT
AAACTTCTGGTATACTTTGCATCCACTAGGGACTCTGGGGTCCCTGATCG
CTTCATAGGCAGTGGATCTGGGACAGATTTCACTCTTACCATCAGCAGTG
TGCAGGCTGAAGACCTGGCAGATTACTTCTGTCAGCAACATTATAGCACT
CCGTACACGTTCGGAGGGGGGACCAAGCTGGAAATAAAAC.

Clone D1-3P1 uses a V_{H} IMGT of IGHV3-6*01, a J_{H} IMGT of IGHJ4*01 and has a VH light chain nucleotide sequence of:

(SEQ ID NO: 3)

GATGTACAGCTTCAGGAGTCAGGACCTGGCCTCGTGAAACCTTCTCAGTC
TCTGTCTCTCACCTGTTCTGTCATTGGTTATTCCATCACCAGTGGTTATT
ACTGGAACTGGGTCCGGCAGTTTCCAGGAAACAAACTGGAATGGATGTCC
TACATAAACTACGATGGTAACAATAACTACAACCCTTCTCTCAAAAATCG
AATCTCCATCACTCGTGACACATCTAAGAACCAGTTTTTCCTGAAGTTGA
ATTCTGTGACTACTGAGGACACAGCCACATATTACTGTGCAAGAGGGAGG
TTTCCTTATGCTTTGGACTACTGGGGTCAAGGAACCTCAGTCACCGTCTC
CTCAG.

Clone D1-3P1 uses a V_{K} IMGT of IGKV12-46*01, a J_{K} IMGT of IGKJ1*01 and has a VK light chain nucleotide sequence of:

(SEQ ID NO: 4)

GACATCCAGATGACTCAGTCTCCAGCCTCCCTATCTGTCTCTGTGGGAGA
AACTGTCACCATCACATGTCGAGCAAGTGAGAATATTTACAGTAATTTAG
CATGGTATCAGCAGAAACAGGGAAAATCTCCTCAGCTCCTGGTCTATGAA
ACAACAAAGTTAAGAGATGGTGTGTCATCAAGGTTCAGTGGCAGTGGATC
AGGCACACAGTATTTCCTCAAGATCAACAACCTGCAGTCTGAAGATTTTG
GGAGTTATTACTGTCAACATTTTTGGGGGAGTCCGTGGACGTTCGGTGGG
GGCACCAAGCTGGAAATCAAAC.

Clone D5-3P1 uses a V_{H} IMGT of IGHV10-3*01, a J_{H} IMGT of IGHJ3*01 and has a VH light chain nucleotide sequence of:

(SEQ ID NO: 5)

GAGGTGCAGCTGGTGGAGTCTGGTGGAGGATTGGTGCAGCCTAAAGGATC
ATTGAAACTCTCATGTGCCGCCTCTGGTTTCACCTTCAATACCTATGCCA
TGCACTGGGTCCGCCAGGCTCCAGGAAAGGGTTTGGAATGGGTTGCTCGC
ATAAGAAGTAAAAGTAGTAATTATGCAACATATTATGCCGATTCAGTGAA
AGACAGATTCACCATCTCCAGAGATGATTCACAAAGCATGCTCTATCTGC
AAATGAACAACCTGAAAACTGAGGACACAGCCATGTATTACTGTGTGAGA
GAGAGTTGGGACCTCTGGTTTGCTTACTGGGGCCAAGGGACTCTGGTCAC
TGTCTCTGCA.

Clone D5-3P1 uses a V_{K} IMGT of IGKV8-27*01, a J_{K} IMGT of IGKJ1*01 and has a VK light chain nucleotide sequence of:

(SEQ ID NO: 6)

AACATTATGATGACACAGTCGCCATCATCTCTGGCTGTGTCTGCAGGAGA
AAAGGTCACTATGAGCTGTAAGTCCAGTCAAAGTGTTTTATACAGTTCAA
ATCAGAAGAACTACTTGGCCTGGTACCAGCAGAAACCAGGGCAGTCTCCT
AAATTGCTGATCTACTGGGCATCCACTAGGGAATCTGGTGTCCCTGATCG
CTTCACAGGCAGTGGATCTGGGACAGATTTTACTCTTACCATCAGCAGTG
TACAAGCTGAAGACCTGGCAGTTTATTACTGTCATCAATACCTCTCCTCG
TGGACGTTCGGTGGAGGCACCAAGCTGGAAATCAAAC.

Clone B2-3P3 uses a V_{H} IMGT of IGHV1-18*01, a J_{H} IMGT of IGHJ2*01 and has a VH light chain nucleotide sequence of:

(SEQ ID NO: 7)

GAGGTGCAGCTGCAGCAGTCTGGACCTGAGCTGGTGAAGCCTGGGGCTTC
AGTGAAGATACCCTGCAAGGCTTCTGGATACACATTCACTGACTACAACA
TGGACTGGGTGAAGCAGAGCCATGGAAAGAGCCTTGAGTGGATTGGAGAT
ATTAATCCTAACAATGGTGGTACTATCTACAACCAGAAGTTCAAGGGCAA
GGCCACATTGACTGTAGACAAGTCCTCCAGCACAGCCTACATGGAGCTCC
GCAGCCTGACATCTGAGGACACTGCAGTCTATTACTGTGCAAGAAGGAGA
TTACGACGTGAGGGGGACTTTGACTACTGGGGCCAAGGCACCACTCTCAC
AGTCTCCTCA.

Clone B2-3P3 uses a V_{H} IMGT of IGKV6-15*01, a J_{H} IMGT of IGKJ2*01 and has a VK light chain nucleotide sequence of:

(SEQ ID NO: 8)

GACATTGTGATGACTCAGTCTCAAAAATTCATGTCCACATCAGTAGGAGA
CAGGGTCAGCGTCACCTGCAAGGCCAGTCAGAATGTGAATACTAATGTAG
CCTGGTATCAACAGAAACCAGGGCAATCTCCTAAAGCACTGATTTACTCG
GCATCCTTCCGGTACAGTGGAGTCCCTGATCGCTTCACAGGCAGTGGATC
TGGGACAGATTTCACTCTCACCATCAGCAATGTGCAGTCTGAAGACTTGG
CAGAGTATTTCTGTCAGCAATATAACAGCTATCCTCTCACGTTCGGAGGG
GGGACCAAGCTGGAAATAAAAC.

Clone C3-3P3 uses a V_{H} IMGT of IGHV3-6*01, a J_{H} IMGT of IGHJ4*01 and has a VH light chain nucleotide sequence of:

(SEQ ID NO: 9)

GATGTACAGCTTCAGGAGTCAGGACCTGGCCTCGTGAAACCTTCTCAGTC
TCTGTCTCTCACCTGCTCTGTCACTGGCTACTCCATCACCAGTGGTTATT
ACTGGAACTGGATCCGGCAGTTTCCAGGAAACAAACTGGAATGGATGGGC
TACATAAGCTACGATGGTAGCAATAACTACAACCCATCTCTCAAAAATCG
AATCTCCATCACTCGTGACACATCTAAGAACCAGTTTTTCCTGAAGTTGA
ATTCTGTGACTACTGAGGACACAGCCACATATTACTGTGCAAGAGGGAAG
GGATCCTATGCTATGGACTACTGGGGTCAAGGAACCTCAGTCACCGTCTC
CTCAG.

Clone C3-3P3 uses a V_{K} IMGT of IGKV12-46*01, a J_{K} IMGT of IGKJ4*01 and has a VK light chain nucleotide sequence of:

(SEQ ID NO: 10)

GACATCCAGATGACTCAGTCTCCAGCCTCCCTATCTGTATCTGTGGGAGA
AACTGTCACCATCACATGTCGAGCAAGTGAGAATATTTACAGTAATTTAG
CATGGTATCAGCAGAAACAGGGAAAATCTCCTCAGCTCCTGGTCTATGTT
GCAACAAACTTAGCAGATGGTGTGCCATCAAGGTTCAGTGGCAGTGGATC
AGGCACACAGTATTCCCTCAAGATCAACAGCCTGCAGTCTGAAGATTTTG
GGAGTTATTACTGTCAACATTTTTGGGGTACTCCATTCACGTTCGGCTCG
GGGACAAAGTTGGAAATAAAAC.

Clone A1-3P3 uses a V_{H} IMGT of IGHV1-77*01, a J_{H} IMGT of IGHJ2*01 and has a VH light chain nucleotide sequence of:

(SEQ ID NO: 11)

CAGGTGCAGCTGAAGCAGTCTGGAGCTGAGCTGGTGAAGCCTGGGGCTTC
AGTGAAGATGTCCTGCAAGGCTTCTGGCTACACCTTCACTGACTACTATA
TAAACTGGTTGAAACAGAGGCCTGGACAGGGCCTTGAGTGGATTGGAAAG
ATTGGTCCTGGAAGTGGTAGTACTTACTACAATGAGAAGTTCAAGGACAA
GGCCACACTGACTGCAGACAAATCCTCCAGCACAGCCTACATGCAGCTCA
GCAGCCTGACATCTGAGGACTCTGCAGTCTATTTCTGTACAAGAACCTAC
TATAGTAATTACTTTGACTACTGGGGCCAAGGCACCACTCTCACAGTCTC
CTCAG.

Clone A1-3P3 uses a V_{K} IMGT of IGKV6-32*01, a J_{K} IMGT of IGKJ2*01 and has a VK light chain nucleotide sequence of:

(SEQ ID NO: 12)

AGTATTGTGATGACCCAGACTCCCAAATTCCTGCTTGTATCAGCAGGAGA
CAGGGTTACCATAACCTGCAAGGCCAGTCAGAGTGTGAATAATAATGTAG
CTTGGTACCAACAGAAGCCAGGGCAGTCTCCTAAACTGCTGATATTTTAT
GCATCCAATCGCTACACTGGAGTCCCTGATCGCTTCACTGGCAGTGGATA
TGGGACGGATTTCACCTTCACCATCAACACTGTGCAGGCTGAAGACCTGG
CAGTTTATTTCTGTCAGCAGGATTATAGTTCTCCGAACACGTTCGGAGGG
GGGACCAAGCTGGAAATAAAAC.

Clone F1-3P3 uses a V_{H} IMGT of IGHV1-64*01, a J_{H} IMGT of IGHJ2*01 and has a VH light chain nucleotide sequence of:

(SEQ ID NO: 13)

CAGGTGCAGCTGCAGCAGCCTGGGGCTGAACTGGTGAAGCCTGGGGCTTC
AGTGAAGTTGTCCTGTAAGGCTTCTGGCTACACTTTCACCTTCTACTGGA
TGCACTGGGTGAAGCAGAGGCCTGGACAAGGCCTTGAGTGGATTGGAATG
ATTCATCCTAATAGTGGTAGTACTAACTACAATGAGAAGTTCAAGAGCAA
GGCCACACTGACTGTAGACAAATCCTCCAGCACAGCCTACATGCAACTCA
GCAGCCTGACATCTGAGGACTCTGCGGTCTATTACTGTGCAAGAGGGGGG
GACTTTGACTACTGGGGCCAAGGCACCACTCTCACAGTCTCCTCAG.

Clone F1-3P3 uses a V_{H} IMGT of IGKV3-2*01, a J_{H} IMGT of IGKJ1*01 and has a VK light chain nucleotide sequence of:

(SEQ ID NO: 14)

GACATTGTGCTGACCCAATCTCCACCTTCTTTGGCTGTGTCTCTAGGGCA
GAGGGCCACCATCTCCTGCAGAGCCAGCGAAAGTGTTGATAATTTTGGCA
TTAATTTTATGAACTGGTTCCAACAGAAACCAGGACAGCCACCCAAACTC
CTCATCTATGCTGCATCCAACCAAGGATCCGGGGTCCCTGCCAGGTTTAG
TGGCAGTGGGTCTGGGACAGACTTCAGCCTCAACATCCATCCTATGGAGG
AGGATGATACTGCAATGTATTTCTGTCAGCAAAGTAAGGAGGTTCCTCGG
ACGTTCGGTGGAGGCACCAAGCTGGAAATCAAAC.

Clone F5-3P3 uses a V_{H} IMGT of IGHV3-6*01, a J_{H} IMGT of IGHJ4*01 and has a VH light chain nucleotide sequence of:

(SEQ ID NO: 15)

GATGTACAGCTTCAGGAGTCAGGACCTGGCCTCGTGAAACCTTCTCAGTC
TCTGTCTCTCACCTGCTCTGTCACTGGCTACTCCATCACCAGTGGTTATT
ACTGGAACTGGATCCGGCAGTTTCCAGGAAACAACCTGGAATGGATGGGC
TACATAAACTACGATGGTAGCAATAACTACAATCCTTCTCTCAAAAATCG
AATCTCCATCACTCGTGACACATCTAAGAACCAGTTTTTCCTGAAGTTGA
ATTCTGTGACTAGTGAGGACACAGGCACGTATTACTGTGCAAGAGGGGCC
TACAATAGTAACTGGGGGGGTGCTATGGACTGCTGGGGTCAAGGAACCTC
AGTCACCGTCTCCTCAG.

Clone F5-3P3 uses a V_{H} IMGT of IGKV12-46*01, a J_{H} IMGT of IGKJ1*01 and has a VK light chain nucleotide sequence of:

(SEQ ID NO: 16)

GACATCCAGATGACTCAGTCTCCAGCCTCCCTATCTGTATCTGTGGGAGA
AATTGTCACCATCACATGTCGAGCAAGTGAGAATATTTACAGTAATTTAG
CATGGTATCAGCAGAAACAGGGAAAATCTCCTCACCTCCTGGTCTATGCT
GCAACAAAGTTAGCAGCTGGTGTGCCATCAAGGTTCAGTGGCAGTGGATC
AGGCACGCAGTATTCCCTCAAGATCAACAGCCTGCAGTCTGAAGATTTTG
GGAGTTATTACTGTCAACATTTTTGGGGTATTCCCCCGACGTTCGGTGGA
GGCACCAAGCTGGAAATCAAAC.

Clone A3-1P2 uses a V_{H} IMGT of IGHV1-64*01, a J_{H} IMGT of IGHJ2*01 and has a VH light chain nucleotide sequence of:

(SEQ ID NO: 17)

CAGGTGCAGCTGCAGCAGCCTGGGGCTGAGCTGGTAAAGCCAGGGGCTTC
AGTGAAGTTGTCCTGCAGGGCTTCTGGCTACACTTTCACCAGCTACTGGA
TGCACTGGGTGAAGCAGAGGCCTGGACAAGGCCTTGAGTGGATTGGAATG
ATTCATCCTAAAAGTGGTAGTATTAATTACAATGAGAAGTTCAAGAGCAA
GGCCACACTGACTGTAGACAAATCCTCCAGCACAGCCTACATGCAACTCA
GCAGCCTGACATCTGAGGACTCTGCGGTCTATTACTGTGCAAGAGGTGGG
GACTTTGACTACTGGGGCCAGGGCACCACTCTCACAGTCTCCTCAG.

Clone A3-1P2 uses a V_{K} IMGT of IGKV3-2*01, a J_{K} IMGT of IGKJ1*01 and has a VK light chain nucleotide sequence of:

(SEQ ID NO: 18)

GACATTGTGCTGACCCAATCTCCAGCTTCTTTGGCTGTGTCTCTAGGGCA
GAGGGCCACCATCTCCTGCAGAGCCAGCGAAAGTGTTGATAATTATGGCA
TTAGTTTTATGAACTGGTTCCAACAGAAACCAGGACAGCCACCCAAACTC
CTCATCTATGCTGCATCCAACCAAGGATCCGGGGTCCCTGCCAGGTTTAG
TGGCAGTGGGTCTGGGACAGATTTCAGCCTCAACATCCATCCTATGGAGG
AGGATGATACTGCAATGTATTTCTGTCAGCAAAGTAGGGAAATTCCTCGG
ACGTTCGGTGGAGGCACCAAGCTGGAAATCAAAC.

Clone B3-1P2 uses a V_{H} IMGT of IGHV3-6*01, a J_{H} IMGT of IGHJ4*01 and has a VH light chain nucleotide sequence of:

(SEQ ID NO: 19)

GATGTACAGCTTCAGGAGTCAGGACCTGGCCTCGTGAAACCTTCTCAGTC
TCTGTCTCTCACCTGCTCTGTCACTGGCTACTCCATCACCAGTGGTTATT
ACTGGAACTGGATCCGGCAGTTTCCAGGAAACAAACTGGAATGGATGGGC
TACATAAGCTACGATGGTAGCAATAACTACAACCCATCTCTCAAAAATCG
AATCTCCATCACTCGTGACACATCTAAGAACCAGTTTTTCCTGAAGTTGA
ATTCTGTGACTACTGAGGACACAGCCACATATTACTGTGCAAGGGAAAGT
CCGGGCTACTATGCTATGGACTACTGGGGTCAAGGAACCTCAGTCACCGT
CTCCTCAG.

Clone B3-1P2 uses a V_{K} IMGT of IGKV12-46*01, a J_{K} IMGT of IGKJ5*01 and has a VK light chain nucleotide sequence of:

(SEQ ID NO: 20)

GACATCCAGATGACTCAGTCTCCAGCCTCCCTATCTGTATCTGTGGGAGA
AACTGTCACCATCACATGTCGAGCAAGTGAGAATATTTACAGTAATTTAG
CATGGTATCAGCAGAAACAGGGAAAATCTCCTCAGCTCCTGGTCTATGCT
GCAACAAACTTAGCAGATGGTGTGCCATCAAGGTTCAGTGGCAGTGGATC
AGGCACACAGTATTCCCTCAAGATCAACAGCCTGCAGTCTGAAGATTTTG
GGAGTTATTACTGTCAACATTTTTGGGGTACTCCGTTCACGTTCGGTGCT
GGGACCAAGCTGGAGCTGAAAC.

Clone B2-1P2 uses a V_{H} IMGT of IGHV1-64*01, a J_{H} IMGT of IGHJ2*01 and has a VH light chain nucleotide sequence of:

(SEQ ID NO: 21)

CAGGTGCAGCTGCAGCAGCCTGGGGCTGAGCTGGTAAAGCCTGGGGCTTC
AGTGAAGTTGTCCTGCAAGGCTTCTGGCTACACTTTCACCAGCTACTGGA
TGCACTGGGTGAAGCAGAGGCCTGGACAAGGCCTTGAGTGGATTGGAATG
ATTCATCCTAATAGTGGTAGTACTAACTACAATGAGAAGTTCAAGAGCAA
GGCCACACTGACTGTAGACAAATCCTCCAGCACAGCCTACATGCAACTCA
GCAGCCTGACATCTGAGGACTCTGCGGTCTATTACTGTGCAAGAGGAGGT
GACTTTGACTACTGGGGCCAAGGCACCACTCTCACAGTCTCCTCAG.

Clone B2-1P2 uses a V_{K} IMGT of IGKV3-2*01, a J_{K} IMGT of IGKJ1*01 and has a VK light chain nucleotide sequence of:

(SEQ ID NO: 22)

GACATTGTGCTGACCCAATCTCCAGCTTCTTTGGCTGTGTCTCTAGGGCA
GAGGGCCACCATCTCCTGCAGAGCCAGCGAAAGTGTTGATAATTATGGCA
TTAGTTTTATGAACTGGTTCCAACAGAAACCAGGACAGCCACCCAAACTC
CTCATCTATGCTGCATCCAACCAAGGATCCGGGGTCCCTGCCAGGTTTAG
TGGCAGTGGGTCTGGGACAGACTTCAGCCTCAACATCCATCCTATGGAGG
AGGATGATACTGCAATGTATTTCTGTCAGCAAAGTAAGGAGGTTCCTCGG
ACGTTCGGTGGAGGCACCAAGCTGGAAATCAAAC.

Clone A6B-1P2 uses a V_{H} IMGT of IGHV3-6*01, a J_{H} IMGT of IGHJ2*01 and has a VH light chain nucleotide sequence of:

(SEQ ID NO: 23)

GATGTACAGCTTCAGGAGTCAGGACCTGGCCTCGTGAAACCTTCTCAGTC
TCTGTCTCTCACCTGCTCTGTCACTGGCTACTCCATCACCAGTGGTTATT
ACTGGAACTGGATCCGGCAGTTTCCAGGAAACAAACTGGAATGGATGGGC
TACATAAGCTACGATGGTAGCAATAACTACAACCCATCTCTCAAAAATCG
AATCTCCATCACTCGTGACACATCTAAGAACCAGTTTTTCCTGAAGTTGA
ATTCTGTGACTACTGAGGACACAGCCACATATTACTGTGCAAGAGAGACT
GGGACGAGGTACTTTGACTACTGGGGCCAAGGCACCACTCTCACAGTCTC
CTCAG.

Clone A6B-1P2 uses a V_{K} IMGT of IGKV12-46*01, a J_{K} IMGT of IGKJ2*01 and has a VK light chain nucleotide sequence of:

(SEQ ID NO: 24)

GACATCCAGATGACTCAGTCTCCAGCCTCCCTATCTGTATCTGTGGGAGA
AACTGTCACCATCACATGTCGAGCAAGTGAGAATATTTACAGTAATTTAG
CATGGTATCAGCAGAAACAGGGAAAATCTCCTCAGCTCCTGGTCTATGCT
GCAACAAACTTAGCAGATGGTGTGCCATCAAGGTTCAGTGGCAGTGGATC
AGGCACACAGTATTCCCTCAAGATCAACAGCCTGCAGTCTGAAGATTTTG
GGAGTTATTACTGTCAACATTTTTGGGGTACTCCGTACACGTTCGGAGGG
GGGACCAAGCTGGAAATAAAAC.

Human malaria antigen AMA-specific IgM clone A8P1-A1 uses a V_{H} IMGT of IGHV4-31*03, a J_{H} IMGT of IGHJ3*02 and has a VH light chain nucleotide sequence of:

(SEQ ID NO: 25)

CAGGTGCAGCTGCAGGAGTCGGGCCCAGGACTGGTGAAGCCTTCACAGAC
CCTGTCCCTCACCTGCACTGTCTCTGGTGGCTCCATCAGCAGTAGTGGTT
ACTACTGGAGCTGGATCCGCCAGCACCCAGCGAAGGGCCTGGAGTGGATT
GGGTACATCTATTACAGTGGGAGCACCTATCACAACCCGTCCCTCAAGAG
TCGAGTTACCATATCAGTAGGCACGTCTAAGAACCAGTTCTCCCTGAAGC
TGAGCTCTGTGACTGCCGCGGA.

The amino acid sequence of the V_{H} domain of AMA-specific IgM clone A8P1-A1 corresponding to SEQ ID NO: 25 is:

(SEQ ID NO: 26)

QVQLQESGPGLVKPSQTLSLTCTVSGGSISSSGYYWSWIRQHPAKGLEWI
GYIYYSGSTYHNPSLKSRVTISVGTSKNQFSLKLSSVTAADTAVYYCARG
YFSGTYSGAFDIWGQGTMVTVSS.

The amino acid sequence of the complementarity determining region 1 or CDR1 of the V_{H} domain of SEQ ID NO: 26 according to the IMGT sequence numbering is: GGSISSSGYY (SEQ ID NO: 27). The amino acid sequence of the CDR2 of the V_{H} domain of SEQ ID NO: 26 according to the IMGT sequence numbering is: IYYSGST (SEQ ID NO: 28). The amino acid sequence of the CDR3 of the V_{H} domain of SEQ ID NO: 26 according to the IMGT sequence numbering is: ARGYFSGTYSGAFDI (SEQ ID NO: 29).

Human malaria antigen AMA-specific IgM clone A8P1-A1 uses a V₂, IMGT of IGLV2-23*01 and IGLV2-23*03, a J_{λ} IMGT of IGLJ3*02 and has a VL light chain nucleotide sequence of:

(SEQ ID NO: 30)

CAGTCTGCCCTGACTCAGCCTGCCTCCGTGTCTGGGTCTCCTGGACAGTC
GATCACCATCTCCTGCACTGGAACCAGCAGTGATGTTGGGAGTTATAACC
TTGTCTCCTGGTACCAACAGCACCCAGGCAAAGCCCCCAAACTCATGGTT
TATGAGGGCAGTAAACGGCCCTCAGGGCTTTCTAATCGCTTCTCTGGCTC
CAAGTCTGGCAACACGGCCTCCCTGACAATCTCTGGGCTCCAGGCTGAAG
ACGAGGCTGATTATTACTGCTGCTCATATGCAGGTAGTAGCACTTGGGTG
TTCGGCGGAGGGACCAAACT.

The amino acid sequence of the V_{L} domain of AMA-specific IgM clone A8P1-A1 corresponding to SEQ ID NO: 30 is:

(SEQ ID NO: 31)

QSALTQPASVSGSPGQSITISCTGTSSDVGSYNLVSWYQQHPGKAPKLMV
YEGSKRPSGLSNRFSGSKSGNTASLTISGLQAEDEADYYCCSYAGSSTWV
FGGGTKLTVL.

The amino acid sequence of the complementarity determining region 1 or CDR1 of the V_{L} domain of SEQ ID NO: 31 according to the IMGT sequence numbering is: SSDVGSYNL (SEQ ID NO: 32). The amino acid sequence of the CDR2 of the V_{L} domain of SEQ ID NO: 31 according to the IMGT sequence numbering is: EGS (SEQ ID NO: 33). The amino acid sequence of the CDR3 of the V_{L} domain of SEQ ID NO: 31 according to the IMGT sequence numbering is: CSYAGSSTWV (SEQ ID NO: 34).

Human malaria antigen AMA-specific IgM clone A8P1-B1 uses a V_{H} IMGT of IGHV3-11*01, a J_{H} IMGT of IGHJ4*02 and has a VH light chain nucleotide sequence of:

(SEQ ID NO: 35)

CAGGTGCAGCTGGTGGAGTCTGGGGGAGGCTTGGTCAAGCCTGGAGGGTC
CCTGAGACTCTCCTGTGCAGCCTCTGGATTCACCTTCAGTGACTACTACA
TGAGCTGGATCCGCCAGGCTCCAGGGAAGGGGCTGGAGTGGATTTCATAC
ATTAGTAGTAGTGGTAGTACCATATACTACGGAGACTCTGTGAAGGGCCG
ATTCACCATCTCCAGGGACAACGCCAAGAACTCACTGTATCTACAAATGA
ACAGCCTGAGAGCCGAGGACA.

The amino acid sequence of the V_{H} domain of AMA-specific IgM clone A8P1-B1 corresponding to SEQ ID NO: 35 is:

(SEQ ID NO: 36)

QVQLVESGGGLVKPGGSLRLSCAASGFTFSDYYMSWIRQAPGKGLEWISY
ISSSGSTIYYGDSVKGRFTISRDNAKNSLYLQMNSLRAEDTAVYYCARER
GSGSYWVDYWGQGTLVTVSS.

The amino acid sequence of the complementarity determining region 1 or CDR1 of the V_{H} domain of SEQ ID NO: 36 according to the IMGT sequence numbering is: GFTFSDYY (SEQ ID NO: 37). The amino acid sequence of the CDR2 of the V_{H} domain of SEQ ID NO: 36 according to the IMGT sequence numbering is: ISSSGSTI (SEQ ID NO: 38). The amino acid sequence of the CDR3 of the V_{H} domain of SEQ ID NO: 36 according to the IMGT sequence numbering is: ARERGSGSYWVDY (SEQ ID NO: 39).

Human malaria antigen AMA-specific IgM clone A8P1-B1 uses a V₂, IMGT of IGLV4-69*01, a IMGT of IGLJ2*01 and IGLJ3*01 and has a VL light chain nucleotide sequence of:

(SEQ ID NO: 40)

CAGCTTGTGCTGACTCAATCGCCCTCTGCCTCTGCCTCCCTGGGAGCCTC
GGTCAAGCTCACCTGCACTCTGAGCAGTGGGCACAGCAACTACGCCATCG
CATGGCATCAGCAGCAGCCAGAGAAGGGCCCTCGGTGCTTGATGAAGGTT
AACAGTGATGGCAGCCACAGCAAGGGGGACGGGATTCCTGATCGCTTCTC
AGGCTCCAGCTCTGGGGCTGAGCGCTACCTCACCATCTCCAGCCTCCAGT
CTGAGGATGAGGCTGACTATTACTGTCAGACCTGGACCACTGGCATTCGG
GTATTCGGCGGAGGGA.

The amino acid sequence of the V_{L} domain of AMA-specific IgM clone A8P1-B1 corresponding to SEQ ID NO: 40 is:

(SEQ ID NO: 41)

QLVLTQSPSASASLGASVKLTCTLSSGHSNYAIAWHQQQPEKGPRCLMKV
NSDGSHSKGDGIPDRFSGSSSGAERYLTISSLQSEDEADYYCQTWTTGIR
VFGGGTKLTVL.

The amino acid sequence of the complementarity determining region 1 or CDR1 of the V_{L} domain of SEQ ID NO: 41 according to the IMGT sequence numbering is: SGHSNYA (SEQ ID NO: 42). The amino acid sequence of the CDR2 of the V_{L} domain of SEQ ID NO: 41 according to the IMGT sequence numbering is: VNSDGSH (SEQ ID NO: 43). The amino acid sequence of the CDR3 of the V_{L} domain of SEQ ID NO: 41 according to the IMGT sequence numbering is: QTWTTGIRV (SEQ ID NO: 44).

Human malaria antigen AMA-specific IgM clone A8P1-B10 uses a V_{H} IMGT of IGHV3-15*01, a J_{H} IMGT of IGHJ4*02 and has a VH light chain nucleotide sequence of:

(SEQ ID NO: 45)

GAGGTGCAGCTGGTGGAGTCTGGGGGAGGCTTGGTAAAGCCTGGGGGGTC
CCTTAGACTCTCCTGTGTTGCCTCTGGATTCACTTTCGATAACGCCTGGA
TGAGCTGGGTCCGCCAGGCTCCAGGGAAGGGGCTGGAGTGGGTTGGCCGT
ATTAAAAGTAAAAGTGATGGTGTGACAACGGACTACGCCGCACACGTGAA
AGGCAGATTCACGATCTCAAGAGACGAATCAAAAACTCCTCTATATCTGC
AAATGAACAGCCTGAGAG.

The amino acid sequence of the V_{H} domain of AMA-specific IgM clone A8P1-B10 corresponding to SEQ ID NO: 45 is:

(SEQ ID NO: 46)

EVQLVESGGGLVKPGGSLRLSCVASGFTFDNAWMSWVRQAPGKGLEWVGR
IKSKSDGVTTDYAAHVKGRFTISRDESKTPLYLQMNSLRVEDTAMYYCTT
GGNQYSFFDSWGRGTLVTVSS.

The amino acid sequence of the complementarity determining region 1 or CDR1 of the V_{H} domain of SEQ ID NO: 46 according to the IMGT sequence numbering is: GFTFDNAW (SEQ ID NO: 47). The amino acid sequence of the CDR2 of the V_{H} domain of SEQ ID NO: 46 according to the IMGT sequence numbering is: IKSKSDGVTT (SEQ ID NO: 48). The amino acid sequence of the CDR3 of the V_{H} domain of SEQ ID NO: 46 according to the IMGT sequence numbering is: TTGGNQYSFFDS (SEQ ID NO: 49).

Human malaria antigen AMA-specific IgM clone A8P1-B10 uses a V₂, IMGT of IGLV3-9*01, a IMGT of IGLJ2*01 and IGLJ3*01 and has a VL light chain nucleotide sequence of:

(SEQ ID NO: 50)

TCCTATGAGCTGACACAGCCACTCTCAGTGTCAGTGGCCCTGGGACAGAC
GGCCAAGATTACCTGTGGGGGAGACAACATTGGAAGAAAGAATGTGCACT
GGTACCAGCAGAAGCCAGGCCAGGCCCCTGTCTTGGTCATCTATAAGGAT
CGCTACCGGCCCTCTGGGATCCCTGAGCGATTCTCTGGCTCCAACTCGGG
GAACACGGCCACCCTGACCATCAACAGAGCCCAAGGCGGGGATGACGCTG
ACTATTTCTGTCAGGTGTGGGACAGTAGCGCTGCGGGGGTCCTATTCGGC
GGAGGGACCAAGCT.

The amino acid sequence of the V_{L} domain of AMA-specific IgM clone A8P1-B10 corresponding to SEQ ID NO: 50 is:

(SEQ ID NO: 51)

SYELTQPLSVSVALGQTAKITCGGDNIGRKNVHWYQQKPGQAPVLVIYKD
RYRPSGIPERFSGSNSGNTATLTINRAQGGDDADYFCQVWDSSAAGVLFG
GGTKLTVL.

The amino acid sequence of the complementarity determining region 1 or CDR1 of the V_{L} domain of SEQ ID NO: 51 according to the IMGT sequence numbering is: NIGRKN (SEQ ID NO: 52). The amino acid sequence of the CDR2 of the V_{L} domain of SEQ ID NO: 51 according to the IMGT sequence numbering is: KDR (SEQ ID NO: 53). The amino acid sequence of the CDR3 of the V_{L} domain of SEQ ID NO: 51 according to the IMGT sequence numbering is: QVWDSSAAGVL (SEQ ID NO: 54).

Human malaria antigen AMA-specific IgG clone A8P1-D10 uses a V_{H} IMGT of IGHV4-59*01 and IGHV4-59*08, a J_{H} IMGT of IGHJ4*02 and has a VH light chain nucleotide sequence of:

(SEQ ID NO: 55)

CAGGTGCAGCTGCAGGAGTCGGGCCCAGGACTGGTGAAGCCTTCGGAGAC
CCTGTCCCTCACCTGCAGTGTCTCTGGTGACTCCATCAATTTTTACTACT
GGAACTGGATCCGGCAGTCCCCAGGGAAGGGACTGGAGTGGATTGCGTAT
GTGTCTAACCGTGGTGACAGTACGAAGTATAACCCCTCACTCGAGAGTCG
AGTCACCATTTCAAGAGAGCCGTCCAAGCGCCAGTCCTCCCTGAAACTGA
ACTCTGTGACCGCCGCGGACAC.

The amino acid sequence of the V_{H} domain of AMA-specific IgG clone A8P1-D10 corresponding to SEQ ID NO: 55 is:

(SEQ ID NO: 56)

QVQLQESGPGLVKPSETLSLTCSVSGDSINFYYWNWIRQSPGKGLEWIAY
VSNRGDSTKYNPSLESRVTISREPSKRQSSLKLNSVTAADTAVYYCALWS
SYFRGYFDYWGQGILVTVSS.

The amino acid sequence of the complementarity determining region 1 or CDR1 of the V_{H} domain of SEQ ID NO: 56 according to the IMGT sequence numbering is: GDSINFYYW (SEQ ID NO: 57). The amino acid sequence of the CDR2 of the V_{H} domain of SEQ ID NO: 56 according to the IMGT sequence numbering is: SNRGDST (SEQ ID NO: 58). The amino acid sequence of the CDR3 of the V_{H} domain of SEQ ID NO: 56 according to the IMGT sequence numbering is: ALWSSYFRGYFDY (SEQ ID NO: 59).

Human malaria antigen AMA-specific IgG clone A8P1-D10 uses a V_{κ} IMGT of IGKV3-15*01, a J_{κ} IMGT of IGKJ3*01 and has a VL light chain nucleotide sequence of:

(SEQ ID NO: 60)

GAAATAGTGATGACGCAGTCTCCAGCCACCCTGTCTGTGTCTCCAGGGGA
AAGAGTCACCCTCTCCTGCAGGGCCAGTCAGAGTGTGAGCACCAATTTAG
CCTGGTACCAGCAGAGGCCTGGCCAGGCTCCCAGGCTCCTCATCTATGCT
TCTTCCACCAGGGCCACTGGTATCCCAGCCAGGTTCAGTGGCGGTGGGTC
TGGGACAGAGTTCACTCTCACCATCAGCAGCCTGCAGTCTGAAGATTTTG
CAGTTTATTACTGTCAGCAGTATGGTCACTGGCCTCCTTACACTTTCGGC
CCTGGGACCAAAGTGGA.

The amino acid sequence of the V_{L} domain of AMA-specific IgG clone A8P1-D10 corresponding to SEQ ID NO: 60 is:

(SEQ ID NO: 61)

EIVMTQSPATLSVSPGERVTLSCRASQSVSTNLAWYQQRPGQAPRLLIYA
SSTRATGIPARFSGGGSGTEFTLTISSLQSEDFAVYYCQQYGHWPPYTFG
PGTKVDIK.

The amino acid sequence of the complementarity determining region 1 or CDR1 of the V_{L} domain of SEQ ID NO: 61 according to the IMGT sequence numbering is: QSVSTN (SEQ ID NO: 62). The amino acid sequence of the CDR2 of the V_{L} domain of SEQ ID NO: 61 according to the IMGT sequence numbering is: ASS (SEQ ID NO: 63). The amino acid sequence of the CDR3 of the V_{L} domain of SEQ ID NO: 61 according to the IMGT sequence numbering is: QQYGHWPPYT (SEQ ID NO: 64).

Human malaria antigen MSP1-specific IgM clone A8P2-B7 uses a V_{H} IMGT of IGHV4-34*01 and IGHV4-34*02, a J_{H} IMGT of IGHJ4*01 and IGHJ4*02 and has a VH light chain nucleotide sequence of:

(SEQ ID NO: 65)

CAGGTGCAGCTACAGCAGTGGGGCGCAGGACTGTTGAAGCCTTCGGAGAC
CCTGTCCCTCACGTGCGCTGTCTATGGTGGGTCCTTCAGTGGTTACTACT
GGACTTGGATCCGCCAGCCCCCAGGGAAGGGACTGGAGTGGATTGGAGAA
ATCAATAATAGTGGAAAAACCAACTACAACCCGTCCCTCAAAAGTCGAGT
CAGCATTTCAATAGACACGTCCAAGAACCAGTTTTCCCTGAAGGTGACTT
CTGTGACCGCCGCGGACACAGC.

The amino acid sequence of the V_{H} domain of MSP1-specific IgM clone A8P2-B7 corresponding to SEQ ID NO: 65 is:

(SEQ ID NO: 66)

QVQLQQWGAGLLKPSETLSLTCAVYGGSFSGYYWTWIRQPPGKGLEWIGE
INNSGKTNYNPSLKSRVSISIDTSKNQFSLKVTSVTAADTAVYYCARGPQ
QHLEPPFDYWGHGTLVTVSS.

The amino acid sequence of the complementarity determining region 1 or CDR1 of the V_{H} domain of SEQ ID NO: 66 according to the IMGT sequence numbering is: GGSFSGYY (SEQ ID NO: 67). The amino acid sequence of the CDR2 of the V_{H} domain of SEQ ID NO: 66 according to the IMGT sequence numbering is: INNSGKT (SEQ ID NO: 68). The amino acid sequence of the CDR3 of the V_{H} domain of SEQ ID NO: 66 according to the IMGT sequence numbering is: ARGPQQHLEPPFDY (SEQ ID NO: 69).

Human malaria antigen MSP1-specific IgM clone A8P2-B7 uses a V₂, IMGT of IGLV1-47*01, a J_{λ} IMGT of IGLJ3*02 and has a VL light chain nucleotide sequence of:

(SEQ ID NO: 70)

CAGTCTGTGCTGACTCAGCCACCCTCAGCGTCTGGGACCCCCGGGCAAAG
GGTCACCATCTCTTGTTCTGGAAGCAACTCCAACATCGCGACTAATTATG
TGTGCTGGTACCAGCAATACCCAGGAACGGCCCCCAAACCCCTCATCTAC
AGGACTGATCAGCGGCCCTCAGGGGTCCCTGACCGATTCTCTGGCTCCAA
GTCTGGCACCTCAGCCTCCCTGGCCATCAGTGGGCTCCGGTCCGAGGATG
AGGCTGATTATTATTGTGCAACATGGGATGACAGCCTGAGTGCCTGGGTG
TTCGGCGGAGGGACCA.

The amino acid sequence of the V_{L} domain of MSP1-specific IgM clone A8P2-B7 corresponding to SEQ ID NO: 70 is:

(SEQ ID NO: 71)

QSVLTQPPSASGTPGQRVTISCSGSNSNIATNYVCWYQQYPGTAPKPLIY
RTDQRPSGVPDRFSGSKSGTSASLAISGLRSEDEADYYCATWDDSLSAWV
FGGGTKLTVL.

The amino acid sequence of the complementarity determining region 1 or CDR1 of the V_{L} domain of SEQ ID NO: 71 according to the IMGT sequence numbering is: NSNIATNY (SEQ ID NO: 72). The amino acid sequence of the CDR2 of the V_{L} domain of SEQ ID NO: 71 according to the IMGT sequence numbering is: RTD (SEQ ID NO: 73). The amino acid sequence of the CDR3 of the V_{L} domain of SEQ ID NO: 71 according to the IMGT sequence numbering is: ATWDDSLSAWV (SEQ ID NO: 74).

Human malaria antigen AMA-specific IgM clone A8P2-E5 uses a V_{H} IMGT of IGHV1-8*02, a J_{H} IMGT of IGHJ6*03 mid has a VH light chain nucleotide sequence of:

(SEQ ID NO: 75)

CAGGTTCAGCTGGTGCAGTCTGGGACTGAAGTGAGGGAGCCTGGGGCCTC
AGTGAAGGTCTCCTGCAAGGCTTCTGGATACACCTTCACCAACTATGATA
TCAACTGGGTGCGACAGGCCACAGGACAAGGGCTTGAGTGGGTGGGATGG
ATGAACCCTAATAGTGGTGAGACAGGCTATGCACAGGAGTTCCAGGGCAG
AATCACCATTACTAGGGACACCTCCATAAGCACAATTTACATGGAGTTGA
GCAGCCTGACATCTGAGGACACGGCCGTATATTACTGTGCCAG.

The amino acid sequence of the V_{H} domain of AMA-specific IgM clone A8P2-E5 corresponding to SEQ ID NO: 75 is:

(SEQ ID NO: 76)

QVQLVQSGTEVREPGASVKVSCKASGYTFTNYDINWVRQATGQGLEWVGW
MNPNSGETGYAQEFQGRITITRDTSISTIYMELSSLTSEDTAVYYCARGG
FCTSTSCYYHYMDVWGKGTTVTVSS.

The amino acid sequence of the complementarity determining region 1 or CDR1 of the V_{H} domain of SEQ ID NO: 76 according to the IMGT sequence numbering is: GYTFTNYD (SEQ ID NO: 77). The amino acid sequence of the CDR2 of the V_{H} domain of SEQ ID NO: 76 according to the IMGT sequence numbering is: MNPNSGET (SEQ ID NO: 78). The amino acid sequence of the CDR3 of the V_{H} domain of SEQ ID NO: 76 according to the IMGT sequence numbering is: ARGGFCTSTSCYYHYMDV (SEQ ID NO: 79).

Human malaria antigen AMA-specific IgM clone A8P2-E5 uses a V_{κ} IMGT of IGKV1-5*03, a J_{κ} IMGT of IGKJ2*01 and has a VL light chain nucleotide sequence of:

(SEQ ID NO: 80)

GACATCCAGATGACCCAGTCTCCTTCCACCCTGTCTGCATCTGTAGGAGA
CAGAGTCACCATCACTTGTCGGGCCAGTCAGAGTGTTAATAGCTGGTTGG
CCTGGTATCAGCAGAAACCAGGGAAAGCCCCTAAGGTCCTGATCTATAAG
GCAACTAGTTTAGAAAGTGGGGTCCCATCAAGGTTCAGCGGCAGTGAATC
TGGGACAGAATTCACTCTCACCATCAGCAGCCTGCAGGCTGATGATTTTG
CAACTTATTACTGCCAACAGTATAATGATTTTCCGTACACTTTTGGCCGG
GGGACCAAGCTGGAGATCAAAC.

The amino acid sequence of the V_{L} domain of AMA-specific IgM clone A8P2-E5 corresponding to SEQ ID NO: 80 is:

(SEQ ID NO: 81)

DIQMTQSPSTLSASVGDRVTITCRASQSVNSWLAWYQQKPGKAPKVLIYK
ATSLESGVPSRFSGSESGTEFTLTISSLQADDFATYYCQQYNDFPYTFGR
GTKLEIK.

The amino acid sequence of the complementarity determining region 1 or CDR1 of the V_{L} domain of SEQ ID NO: 81 according to the IMGT sequence numbering is: QSVNSW (SEQ ID NO: 82). The amino acid sequence of the CDR2 of the V_{L} domain of SEQ ID NO: 81 according to the IMGT sequence numbering is: KAT (SEQ ID NO: 83). The amino acid sequence of the CDR3 of the V_{L} domain of SEQ ID NO: 81 according to the IMGT sequence numbering is: QQYNDFPYT (SEQ ID NO: 84).

Human malaria antigen MSP1-specific IgM clone A8P2-E6 uses a V_{H} IMGT of IGHV4-34*01 and IGHV4-34*02, a J_{H} IMGT of IGHJ4*02 and IGHJ5*02 and has a VH light chain nucleotide sequence of:

(SEQ ID NO: 85)

CAGGTGCAGCTACAGCAGTGGGGCGCAGGACTGTTGAAGCCTGCGGAGAC
CCTGTCCCTCACTTGCGCTGTCTATGGTGGGTCCTTCACTGGTCACTACT
GGACCTGGATCCGTCAGCCCCCTGGTAAGGGGCCGGAATGGATTGGGGAA
ATCAATCATCGTGGAGGCACCGACTACAACCCGTCCCTCAAGAGTCGAGT
CACCATTTCACTAGACACGTCCAAGAACCAGGTGTCCCTGAAACTGAGCG
CTGTGACCGCCGTAGACACG.

The amino acid sequence of the V_{H} domain of MSP1-specific IgM clone A8P2-E6 corresponding to SEQ ID NO: 85 is:

(SEQ ID NO: 86)

QVQLQQWGAGLLKPAETLSLTCAVYGGSFTGHYWTWIRQPPGKGPEWIGE
INHRGGTDYNPSLKSRVTISLDTSKNQVSLKLSAVTAVDTAVYYCARGHG
RYYYSYLDSWAQGTLVTVSS.

The amino acid sequence of the complementarity determining region 1 or CDR1 of the V_{H} domain of SEQ ID NO: 86 according to the IMGT sequence numbering is: GGSFTGHY (SEQ ID NO: 87). The amino acid sequence of the CDR2 of the V_{H} domain of SEQ ID NO: 86 according to the IMGT sequence numbering is: INHRGGT (SEQ ID NO: 88). The amino acid sequence of the CDR3 of the V_{H} domain of SEQ ID NO: 86 according to the IMGT sequence numbering is: ARGHGRYYYSYLDS (SEQ ID NO: 89).

Human malaria antigen MSP1-specific IgM clone A8P2-E6 uses a V_{κ} IMGT of IGKV1-5*03, a J_{κ} IMGT of IGKJ2*01 and IGKJ2*02, and has a VL light chain nucleotide sequence of:

(SEQ ID NO: 90)

GACATCCAGATGACCCAGTCTCCTTCCACCCTGTCTGCAGCTGTAGGAGA
CCGAGTCACCATCACTTGCCGGGCCAGTCAGGCTATTAGTCCCTGGGTGG
CCTGGTATCAGCAGAAACCAGGGAAAGCCCCTTCACTCCTTATCTATCAG
GCGTCTACTTTACAAAGTGCGGTCCCATTAAGGTTCAGCGGCAGTGGATC
TGGGACAGACTTCACTCTCACCATCAGCAGCCTGCAGCCTGAGGATTTTG
CAACTTATTTCTGCCAACAGTATGGTCGCTATTCCACTTTTGGCCAGGGG
ACCAAGCTGGAGATCAAAC.

The amino acid sequence of the V_{L} domain of MSP1-specific IgM clone A8P2-E6 corresponding to SEQ ID NO: 90 is:

(SEQ ID NO: 91)

DIQMTQSPSTLSAAVGDRVTITCRASQAISPWVAWYQQKPGKAPSLLIYQ
ASTLQSAVPLRFSGSGSGTDFTLTISSLQPEDFATYFCQQYGRYSTFGQG
TKLEIK.

The amino acid sequence of the complementarity determining region 1 or CDR1 of the V_{L} domain of SEQ ID NO: 91 according to the IMGT sequence numbering is: QAISPW (SEQ ID NO: 92). The amino acid sequence of the CDR2 of the V_{L} domain of SEQ ID NO: 91 according to the IMGT sequence numbering is: QAS (SEQ ID NO: 93). The amino acid sequence of the CDR3 of the V_{L} domain of SEQ ID NO: 91 according to the IMGT sequence numbering is: QQYGRYST (SEQ ID NO: 94).

Human malaria antigen MSP1-specific IgG clone A8P2-E12 uses a V_{H} IMGT of IGHV4-34*01, a J_{H} IMGT of IGHJ4*02 and IGHJ5*02 and has a VH light chain nucleotide sequence of:

(SEQ ID NO: 95)

CAGGTGCAGCTACAGCAGTGGGGCGCAGGACTGTTGAAGCCTGCGGAGAC
CCTGTCCCTCACCTGCGCTGTCTATGGTGGGTCCTTCACTGGTTACTACT
GGACCTGGATCCGTCAGCCCCCTGGTAAGGGGCTGGAATGGATTGGGGAG
ATCAATCATCGTGGAGGCACCGACTACAATCCGTCCCTCAAGAGTCGAGT
CACCATTTCTCTTGACACGTCCAAGAACCAGGTGTCCCTGAAACTCCGCT
CTGCGACCGCCGTAGACACGGC.

The amino acid sequence of the V_{H} domain of MSP1-specific IgM clone A8P2-E12 corresponding to SEQ ID NO: 95 is:

(SEQ ID NO: 96)

QVQLQQWGAGLLKPAETLSLTCAVYGGSFTGYYWTWIRQPPGKGLEWIGE
INHRGGTDYNPSLKSRVTISLDTSKNQVSLKLRSATAVDTAVYYCARGHG
RYYYSYLNLWAQGTLVTVSS.

The amino acid sequence of the complementarity determining region 1 or CDR1 of the V_{H} domain of SEQ ID NO: 96 according to the IMGT sequence numbering is: GGSFTGYY (SEQ ID NO: 97). The amino acid sequence of the CDR2 of the V_{H} domain of SEQ ID NO: 96 according to the IMGT sequence numbering is: INHRGGT (SEQ ID NO: 98). The amino acid sequence of the CDR3 of the V_{H} domain of SEQ ID NO: 96 according to the IMGT sequence numbering is: ARGHGRYYYSYLNL (SEQ ID NO: 99).

Human malaria antigen MSP1-specific IgM clone A8P2-E12 uses a V_{κ} IMGT of IGKV1-5*03, a J_{κ} IMGT of IGKJ2*01 and IGKJ2*02, and has a VL light chain nucleotide sequence of:

(SEQ ID NO: 100)

GACATCCAGATGACCCAGTCTCCTTCCACCCTGTCTGCATCTGTAGGAGA
CAGAGTCACCATCACTTGCCGGGCCAGTCAGGCTATTAGTCCCTGGTTGG
CCTGGTATCAGCAGAAACCAGGGAGAGCCCCTAAACTCCTGATCTATCAG
GCGTCCACTTTACAAAGTGCGGTCCCATCAAGATTCAGCGGCAGTGGATC
TGGGACAGAATTCACTCTCACCATCACCAGCCTGCAGCCTGAAGATTTTG
CAACTTATTACTGCCAACAGTATGGTCGTTATTCCACTTTTGGCCAGGGG
ACCAAGCTGGAGATCAAAC.

The amino acid sequence of the V_{L} domain of MSP1-specific IgM clone A8P2-E12 corresponding to SEQ ID NO: 100 is:

(SEQ ID NO: 101)

DIQMTQSPSTLSASVGDRVTITCRASQAISPWLAWYQQKPGRAPKLLIYQ
ASTLQSAVPSRFSGSGSGTEFTLTITSLQPEDFATYYCQQYGRYSTFGQG
TKLEIK.

The amino acid sequence of the complementarity determining region 1 or CDR1 of the V_{L} domain of SEQ ID NO: 101 according to the IMGT sequence numbering is: QAISPW (SEQ ID NO: 102). The amino acid sequence of the CDR2 of the V_{L} domain of SEQ ID NO: 101 according to the IMGT sequence numbering is: QAS (SEQ ID NO: 103). The amino acid sequence of the CDR3 of the V_{L} domain of SEQ ID NO: 101 according to the IMGT sequence numbering is: QQYGRYST (SEQ ID NO: 104).

Human malaria antigen AMA-specific IgM clone A8P3-B5 uses a V_{H} IMGT of IGHV4-61*02, a J_{H} IMGT of IGHJ6*02, and has a VH light chain nucleotide sequence of:

(SEQ ID NO: 105)

CAGGTGCAGCTGCAGGAGTCGGGCCCAGGACTGGTGAAGCCTTCACAGAC
CCTGTCCCTCACCTGCACTGTCTCTGGTGGCTCCATCAGCAGTGGTAGTT
ACTACTGGAGCTGGATCCGGCAGCCCGCCGGGAAGGGACTGGAGTGGATT
GGGCGTATCTATACCAGTGGGAGCACCAACTACAACCCCTCCCTCAAGAG
TCGAGTCACCATATCAGTAGACACGTCCAAGAACCAGTTCTCCCTGAAGC
TGAGCTCTGTGACCGCCGCAGA.

The amino acid sequence of the V_{H} domain of AMA-specific IgM clone A8P3-B5 corresponding to SEQ ID NO: 105 is:

(SEQ ID NO: 106)

QVQLQESGPGLVKPSQTLSLTCTVSGGSISSGSYYWSWIRQPAGKGLEWI
GRIYTSGSTNYNPSLKSRVTISVDTSKNQFSLKLSSVTAADTAVYYCARV
MVRGVIGSYGMDVWGQGTTVTVSS.

The amino acid sequence of the complementarity determining region 1 or CDR1 of the V_{H} domain of SEQ ID NO: 106 according to the IMGT sequence numbering is: GGSISSGSYY (SEQ ID NO: 107). The amino acid sequence of the CDR2 of the V_{H} domain of SEQ ID NO: 106 according to the IMGT sequence numbering is: IYTSGST (SEQ ID NO: 108). The amino acid sequence of the CDR3 of the V_{H} domain of SEQ ID NO: 106 according to the IMGT sequence numbering is: ARVMVRGVIGSYGMDV (SEQ ID NO: 109).

Human malaria antigen AMA-specific IgM clone A8P3-B5 uses a V₂, IMGT of IGLV3-21*02, a IMGT of IGLJ3*02, and has a VL light chain nucleotide sequence of:

(SEQ ID NO: 110)

TCCTATGTGCTGACTCAGCCACCCTCGGTGTCAGTGGCCCCAGGACAGAC
GGCCAGGATTACCTGTGGGGGAAACAACATTGGAAGTAAAAGTGTGCACT
GGTACCAGCAGAAGCCAGGCCAGGCCCCTGTGCTGGTCGTCTATGATGAT
AGCGACCGGCCCTCAGGGATCCCTGAGCGATTCTCTGGCTCCAACTCTGG
GAACACGGCCACCCTGACCATCAGCAGGGTCGAAGCCGGGGATGAGGCCG
ACTATTACTGTCAGGTGTGGGATAGTAGTAGTGATCATGAGGTGTTCGGC
GGAGGGACCAAG.

The amino acid sequence of the V_{L} domain of AMA-specific IgM clone A8P3-B5 corresponding to SEQ ID NO: 110 is:

(SEQ ID NO: 111)

SYVLTQPPSVSVAPGQTARITCGGNNIGSKSVHWYQQKPGQAPVLVVYDD
SDRPSGIPERFSGSNSGNTATLTISRVEAGDEADYYCQVWDSSSDHEVFG
GGTKLTVL.

The amino acid sequence of the complementarity determining region 1 or CDR1 of the V_{L} domain of SEQ ID NO: 111 according to the IMGT sequence numbering is: NIGSKS (SEQ ID NO: 112). The amino acid sequence of the CDR2 of the V_{L} domain of SEQ ID NO: 111 according to the IMGT sequence numbering is: DDS (SEQ ID NO: 113). The amino acid sequence of the CDR3 of the V_{L} domain of SEQ ID NO: 111 according to the IMGT sequence numbering is: QVWDSSSDHEV (SEQ ID NO: 114).

Human malaria antigen MSP1-specific IgG clone A8P3-C10 uses a V_{H} IMGT of IGHV3-23*01, IGHV3-23*04, and IGHV3-23D*01, a J_{H} IMGT of IGHJ4*02, and has a VH light chain nucleotide sequence of:

(SEQ ID NO: 115)

GAGGTGCAGCTGTTGGAGTCTGGGGGAGCCTTGGTACAGCCTGGGGGGTC
CCTGAGACTGTCCTGTGTAACCTCTGGATTCACCTTTAGCTCCTATGCCA
TGACCTGGGTCCGCCAGGCTCCAGGGAAGGGGCTGGAATGGGTCTCAGGT
ATTAGTTCCGGTGGCTTTATCACATACTACGCAGACTCCGTGAAGGGCCG
CTTCACCATCTCCAGAGACAATTCCAAGAACACAGTGTATTTGCAAATGA
ACAGCCTGAGAGCCGAGGACACG.

The amino acid sequence of the V_{H} domain of MSP1-specific IgG clone A8P3-C10 corresponding to SEQ ID NO: 115 is:

(SEQ ID NO: 116)

EVQLLESGGALVQPGGSLRLSCVTSGFTFSSYAMTWVRQAPGKGLEWVSG
ISSGGFITYYADSVKGRFTISRDNSKNTVYLQMNSLRAEDTALYYCAKGM
GSNIYVGFDYWGQGTLVTVSS.

The amino acid sequence of the complementarity determining region 1 or CDR1 of the V_{H} domain of SEQ ID NO: 116 according to the IMGT sequence numbering is: GFTFSSYA (SEQ ID NO: 117). The amino acid sequence of the CDR2 of the V_{H} domain of SEQ ID NO: 116 according to the IMGT sequence numbering is: ISSGGFIT (SEQ ID NO: 118). The amino acid sequence of the CDR3 of the V_{H} domain of SEQ ID NO: 116 according to the IMGT sequence numbering is: AKGMGSNIYVGFDY (SEQ ID NO: 119).

Human malaria antigen MSP1-specific IgG clone A8P3-C10 uses a V_{κ} IMGT of IGKV3-20*01, a J_{κ} IMGT of IGKJ1*01, and has a VL light chain nucleotide sequence of:

(SEQ ID NO: 120)

GAAATTGTGTTGACGCAGTCTCCAGGCACCCTGTCTTTGTCTCCAGGGGA
AAGAGCCACCCTCTCCTGCAGGGCCAGTCAGATTGTAAGCAGCAACTACT
TAGCCTGGTACCAGCAGAAACCTGGCCAGGCTCCCAGGCTCCTCATCTTT
GGTGCATCCAGCAGGGCCACTGGCATCCCAGACAGGTTCAGTGGCAGTGG
GTCTGACACAGACTTCACTCTCACCATCCGCAGACTGGAGTCTGAAGATT
TTGCAGTGTATTACTGTCACCAGTATGGTAGCTCACCGGGGACGTTCGGC
CAAGGGACCAAGGTGGA.

The amino acid sequence of the V_{L} domain of MSP1-specific IgG clone A8P3-C10 corresponding to SEQ ID NO: 120 is:

(SEQ ID NO: 121)

EIVLTQSPGTLSLSPGERATLSCRASQIVSSNYLAWYQQKPGQAPRLLIF
GASSRATGIPDRFSGSGSDTDFTLTIRRLESEDFAVYYCHQYGSSPGTFG
QGTKVEIK.

The amino acid sequence of the complementarity determining region 1 or CDR1 of the V_{L} domain of SEQ ID NO: 121 according to the IMGT sequence numbering is: QIVSSNY (SEQ ID NO: 122). The amino acid sequence of the CDR2 of the V_{L} domain of SEQ ID NO: 121 according to the IMGT sequence numbering is: GAS (SEQ ID NO: 123). The amino acid sequence of the CDR3 of the V_{L} domain of SEQ ID NO: 121 according to the IMGT sequence numbering is: HQYGSSPGT (SEQ ID NO: 124).

Human malaria antigen MSP1-specific IgM clone A8P3-E4 uses a V_{H} IMGT of IGHV4-59*01, a J_{H} IMGT of IGHJ4*02, and has a VH light chain nucleotide sequence of:

(SEQ ID NO: 125)

CAGGTGCAGCTGCAGGAGTCGGGCCCAGGACTGGTGAAGCCTTCGGAGAC
CCTGTCCCTCACCTGCTCTGTCTCTGGTGGCTCCATCAGTAATTCCTACG
TGAGCTGGATCCGGCAGCCCCCAGGGAAGGGACTGGAGTGGATTGGGTAT
ATCTATTACAGTGGGGGCACCAACTACAACCCCTCCCTTAAGAGTCGAGT
CACCATTTCAGTAGACACGTCCAAGAACCAGTTCTCCCTGAAGCTGAGCT
CCGTGACCGCTGCGGACACGG.

The amino acid sequence of the V_{H} domain of MSP1-specific IgM clone A8P3-E4 corresponding to SEQ ID NO: 125 is:

(SEQ ID NO: 126)

QVQLQESGPGLVKPSETLSLTCSVSGGSISNSYVSWIRQPPGKGLEWIGY
IYYSGGTNYNPSLKSRVTISVDTSKNQFSLKLSSVTAADTAVYYCARGKI
YFDYWGQGTLVTVSS.

The amino acid sequence of the complementarity determining region 1 or CDR1 of the V_{H} domain of SEQ ID NO: 126 according to the IMGT sequence numbering is: GGSISNSY (SEQ ID NO: 127). The amino acid sequence of the CDR2 of the V_{H} domain of SEQ ID NO: 126 according to the IMGT sequence numbering is: IYYSGGT (SEQ ID NO: 128). The amino acid sequence of the CDR3 of the V_{H} domain of SEQ ID NO: 126 according to the IMGT sequence numbering is: ARGKIYFDY (SEQ ID NO: 129).

Human malaria antigen MSP1-specific IgM clone A8P3-E4 uses a V_{κ} IMGT of IGKV1-5*03, a J_{κ} IMGT of IGKJ1*01, and has a VL light chain nucleotide sequence of:

(SEQ ID NO: 130)

GACATCCAGATGACCCAGTCTCCTTCCACCCTGTCTGCATCCGTAGGAGA
CAGAGTCACCATCACTTGCCGGGCCAGTCAGAGTATTAGTAGTTGGTTGG
CCTGGTATCAGCTGAAACCAGGGAAGGCCCCTAAACTCCTGATTTATAAG
GCGTCCAGTTTAGAAAGTGGGGTCCCATCGAGATTCAGCGGCAGTGGATC
TGGGACAGAATTCACTCTCACCATCAGCAGCCTGCAGCCTGGTGATTTTG
CAACTTATTACTGCCAACAGTATAATAGTTATGCTTTGGCGTTCGGCCAA
GGGACCAAGGTGGAGAT.

The amino acid sequence of the V_{L} domain of MSP1-specific IgM clone A8P3-E4 corresponding to SEQ ID NO: 130 is:

(SEQ ID NO: 131)

DIQMTQSPSTLSASVGDRVTITCRASQSISSWLAWYQLKPGKAPKLLIYK
ASSLESGVPSRFSGSGSGTEFTLTISSLQPGDFATYYCQQYNSYALAFGQ
GTKVEIK.

The amino acid sequence of the complementarity determining region 1 or CDR1 of the V_{L} domain of SEQ ID NO: 131 according to the IMGT sequence numbering is: QSISSW (SEQ ID NO: 132). The amino acid sequence of the CDR2 of the V_{L} domain of SEQ ID NO: 131 according to the IMGT sequence numbering is: KAS (SEQ ID NO: 133). The amino acid sequence of the CDR3 of the V_{L} domain of SEQ ID NO: 131 according to the IMGT sequence numbering is: QQYNSYALA (SEQ ID NO: 134).

Human malaria antigen AMA-specific IgG clone A8P3-H8 uses a V_{H} IMGT of IGHV4-39*01, a J_{H} IMGT of IGHJ4*02, and has a VH light chain nucleotide sequence of:

(SEQ ID NO: 135)

CAGCTGCAGCTGCAGGAGTCGGGCCCAGGACTGGTGAAACCTTCGGAGAC
CCTGTCCCTCACCTGCACTGTCTCTGGTGGCTCCATCAGCAGTAGTCTTT
ACTACTGGGGCTGGATCCGCCAGCCCCCAGGGAAGGGACTGGAGTGGATT
GGGAATATCTATTATAGTGGGATCACCTATTACAACCCGTCCCTCACAAG
TCGAGTCACCATATCCGTAGACACGTCCAAGGACCAGTTCTCCCTGAAGC
TGAGCTCTGTGACCGTCGCAGACACGGCTGTGTATTACTGTGCGCG.

The amino acid sequence of the V_{H} domain of AMA-specific IgG clone A8P3-H8 corresponding to SEQ ID NO: 135 is:

(SEQ ID NO: 136)

QLQLQESGPGLVKPSETLSLTCTVSGGSISSSLYYWGWIRQPPGKGLEWI
GNIYYSGITYYNPSLTSRVTISVDTSKDQFSLKLSSVTVADTAVYYCARE
ILTGDPSVGGDPFDYWGQGTLVTVSS.

The amino acid sequence of the complementarity determining region 1 or CDR1 of the V_{H} domain of SEQ ID NO: 136 according to the IMGT sequence numbering is: GGSISSSLYY (SEQ ID NO: 137). The amino acid sequence of the CDR2 of the V_{H} domain of SEQ ID NO: 136 according to the IMGT sequence numbering is: IYYSGIT (SEQ ID NO: 138). The amino acid sequence of the CDR3 of the V_{H} domain of SEQ ID NO: 136 according to the IMGT sequence numbering is: AREILTGDPSVGGDPFDY (SEQ ID NO: 139).

Human malaria antigen AMA-specific IgG clone A8P3-H8 uses a V₂, IMGT of IGLV1-51*02, a IMGT of IGLJ2*01 and IGLJ3*01, and has a VL light chain nucleotide sequence of:

(SEQ ID NO: 140)

CAGTCTGTGCTGACGCAGCCGCCCTCAGTGTCTGCGGCCCCAGGACAGAA
GGTCACCATCTCCTGCTCTGGAAGCAGCTCCAACATTGGGAATAATTATG
TTTCTTGGTATCGACAACTCCCAGGAACAGCCCCCAAACTCCTCGTCTAT
GAAAGTAATAAGCGACCCTCAGGGATTCCTGACCGATTCTCTGGCTCCAA
GTCTGCCACGTCAGCCACCCTGGGCATCACCGGACTCCAGACTGGGGACG
AGGCCGATTATTACTGCGGAACATGGGATACCAGCCTGAGTGCTGTGGTA
TTCGGCGGAGGGACCAAACTGACCGTCCTAG.

The amino acid sequence of the V_{L} domain of AMA-specific IgG clone A8P3-H8 corresponding to SEQ ID NO: 140 is:

(SEQ ID NO: 141)

QSVLTQPPSVSAAPGQKVTISCSGSSSNIGNNYVSWYRQLPGTAPKLLVY
ESNKRPSGIPDRFSGSKSATSATLGITGLQTGDEADYYCGTWDTSLSAVV
FGGGTKLTVL.

The amino acid sequence of the complementarity determining region 1 or CDR1 of the V_{L} domain of SEQ ID NO: 141 according to the IMGT sequence numbering is: SSNIGNNY (SEQ ID NO: 142). The amino acid sequence of the CDR2 of the V_{L} domain of SEQ ID NO: 141 according to the IMGT sequence numbering is: ESN (SEQ ID NO: 143). The amino acid sequence of the CDR3 of the V_{L} domain of SEQ ID NO: 141 according to the IMGT sequence numbering is: GTWDTSLSAVV (SEQ ID NO: 144).

Human malaria antigen AMA-specific IgG clone A8P2-G6 uses a V_{H} IMGT of IGHV4-38-2*02, a J_{H} IMGT of IGHJ4*02, and has a VH light chain nucleotide sequence of:

(SEQ ID NO: 145)

CAGGTGCAGCTGCAGGAGTCGGGCCCAGGACTGGTGAAGCCTTCGCAGAC
CCTGTCCCTCACCTGCACTGTCTCTAATTTCCCCATTGCCAGTTCTTACT
ACTGGAACTGGATCCGCCAGTCGCCAGAAAAGGGACTGGAATGGATTGGA
AGTGTGTATTTTAGTGGCAGCACCCACTCTAATCCGTCTTTCGCGAGTCG
AGTGAGCATGTCGGTGGACACCTCCAAGAGCCAATTCACCCTCAAGTTGA
CCTCTCTGTCCGCCGCGGACACA.

The amino acid sequence of the V_{H} domain of AMA-specific IgG clone A8P2-G6 corresponding to SEQ ID NO: 145 is:

(SEQ ID NO: 146)

QVQLQESGPGLVKPSQTLSLTCTVSNFPIASSYYWNWIRQSPEKGLEWIG
SVYFSGSTHSNPSFASRVSMSVDTSKSQFTLKLTSLSAADTAVYYCAKGD
TSRLATNFDDWGPGIQVIVSS.

The amino acid sequence of the complementarity determining region 1 or CDR1 of the V_{H} domain of SEQ ID NO: 146 according to the IMGT sequence numbering is: NFPIASSYY (SEQ ID NO: 147). The amino acid sequence of the CDR2 of the V_{H} domain of SEQ ID NO: 146 according to the IMGT sequence numbering is: VYFSGST (SEQ ID NO: 148). The amino acid sequence of the CDR3 of the V_{H} domain of SEQ ID NO: 146 according to the IMGT sequence numbering is: AKGDTSRLATNFDD (SEQ ID NO: 149).

Human malaria antigen AMA-specific IgG clone A8P2-G6 uses a V_{κ} IMGT of IGKV4-1*01, a J_{κ} IMGT of IGKJ4*01 and IGKJ4*02, and has a VL light chain nucleotide sequence of:

(SEQ ID NO: 150)

GACATCGTGATGACCCAGTCTCCAGAGACCCTGCCTGTGTCTCTGGGCGA
GAGGGCCACCATCAACTGCAAGTCCAGCCAGACTCTTTTATTTACCTCCA
ACAATAAGGACTACGTAGCTTGGTACCAGCAGAAACCAGGACAGCCTCCT
AAGTTGCTCATTTACTGGGCATCTACCCGGGAATCCGGGGTCCCTGACCG
CTTCAGTGGCAGCGGGTCTGGGACAGATTTCACTCTCACCATCAACAGCC
TGCAGGCTGAAGATGTGGCGGTTTATTATTGTCAGCAATACCTTACTACT
CCTCTTACCTTCGGCGGAG.

The amino acid sequence of the V_{L} domain of AMA-specific IgG clone A8P2-G6 corresponding to SEQ ID NO: 150 is:

(SEQ ID NO: 151)

DIVMTQSPETLPVSLGERATINCKSSQTLLFTSNNKDYVAWYQQKPGQPP
KLLIYWASTRESGVPDRFSGSGSGTDFTLTINSLQAEDVAVYYCQQYLTT
PLTFGGGTKVDIR.

The amino acid sequence of the complementarity determining region 1 or CDR1 of the V_{L} domain of SEQ ID NO: 151 according to the IMGT sequence numbering is: QTLLFTSNNKDY (SEQ ID NO: 152). The amino acid sequence of the CDR2 of the V_{L} domain of SEQ ID NO: 151 according to the IMGT sequence numbering is: WAS (SEQ ID NO: 153). The amino acid sequence of the CDR3 of the V_{L} domain of SEQ ID NO: 151 according to the IMGT sequence numbering is: QQYLTTPLT (SEQ ID NO: 154).

It should be understood that this invention is not limited to the particular methodology, protocols, and reagents, etc., described herein and as such may vary. The terminology used herein is for the purpose of describing particular embodiments only, and is not intended to limit the scope of the present invention, which is defined solely by the claims.

As used herein and in the claims, the singular forms include the plural reference and vice versa unless the context clearly indicates otherwise. The term “or” is inclusive unless modified, for example, by “either.” Other than in the operating examples, or where otherwise indicated, all numbers expressing quantities of ingredients or reaction conditions used herein should be understood as modified in all instances by the term “about.”

All patents and other publications identified are expressly incorporated herein by reference for the purpose of describing and disclosing, for example, the methodologies described in such publications that might be used in connection with the present invention. These publications are provided solely for their disclosure prior to the filing date of the present application. Nothing in this regard should be construed as an admission that the inventors are not entitled to antedate such disclosure by virtue of prior invention or for any other reason. All statements as to the date or representation as to the contents of these documents is based on the information available to the applicants and does not constitute any admission as to the correctness of the dates or contents of these documents.

Unless defined otherwise, all technical and scientific terms used herein have the same meaning as those commonly understood to one of ordinary skill in the art to which this invention pertains. Although any known methods, devices, and materials may be used in the practice or testing of the invention, the methods, devices, and materials in this regard are described herein.

This invention is further illustrated by the following examples which should not be construed as limiting. It is understood that the foregoing description and the following examples are illustrative only and are not to be taken as limitations upon the scope of the invention. Various changes and modifications to the disclosed embodiments, which will be apparent to those of skill in the art, may be made without departing from the spirit and scope of the present invention.

### Examples

### Example 1

### MSP1-Specific B Cells Expand, Differentiate and Form Memory in Response to Blood Stage Malaria Infection.

The direct ex vivo visualization of antigen-specific B cells during infection has been difficult to accomplish due to a lack of tools and techniques to track small populations of B cells. Therefore, techniques used to analyze MBC development in response to protein immunization were developed. While applicable to any desired antigen, the approach is exemplified herein examining MBC development and function in response to blood stage malaria infection in C57BL/6 mice were adopted. A phycoerythrin (PE)-conjugated B cell tetramer containing the majority of the 19 kD C-terminal portion of the MSP1 protein from P. chabaudi was generated and used with magnetic bead-based enrichment to analyze malaria-specific B cells directly ex vivo throughout all phases of the immune response.

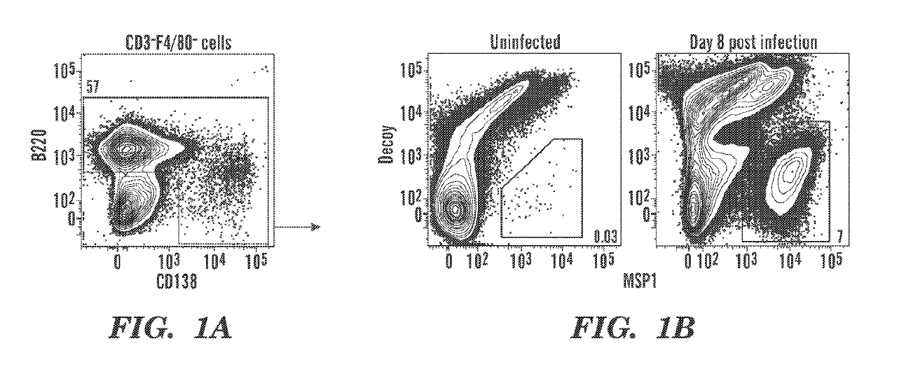

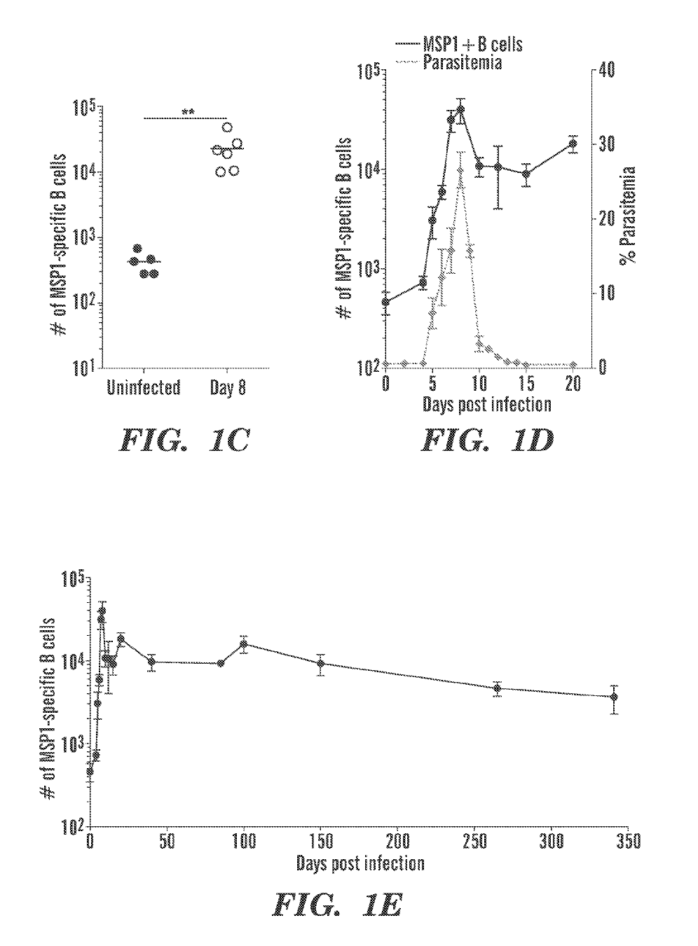

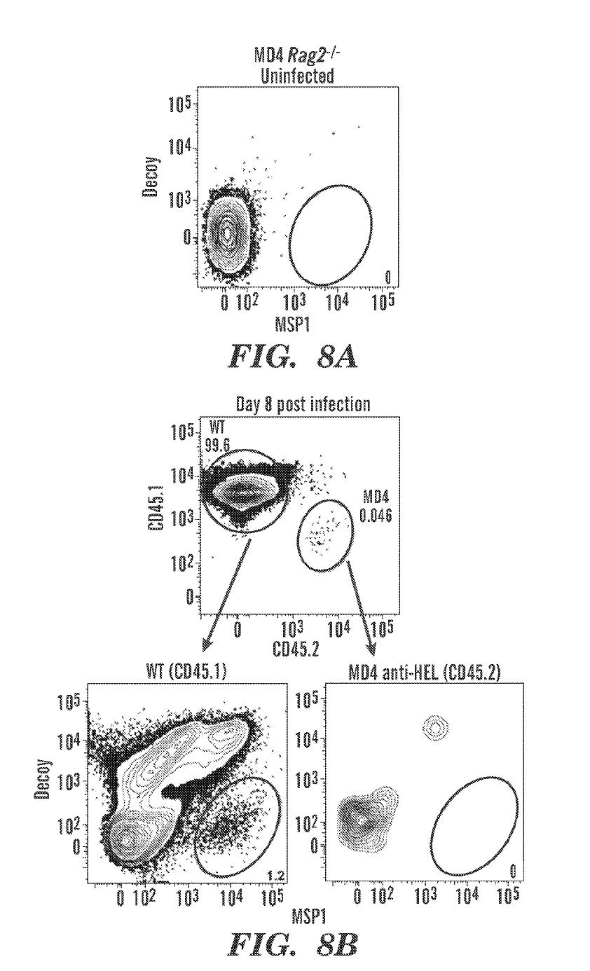

In all experiments, splenocytes were first stained with a decoy reagent and then with the MSP1 PE tetramer to exclude cells binding other components of the tetramer (Taylor et al., 2012a). Anti-PE coated magnetic beads were then used to enrich both decoy-specific and MSP1-specific B cells, which were subsequently stained with antibodies for analysis by multiparameter flow cytometry. Antibody panels were based upon gating strategies developed to visualize all stages of mature B2 B cell differentiation. After excluding non-lymphocytes and doublets, Decoy⁻MSP1⁺ B cells were identified amongst B220⁺ and B220^{low}CD138⁺ cells (identifying plasmablasts) (FIGS. 1A, 1B). In uninfected mice, there were approximately 400 MSP1⁺ B cells, while 8 days after infection with 1×10⁶ P.chabaudi iRBCs (Butler et al., 2012), the number of MSP1⁺ B cells expanded 50 fold to 23,000 cells (FIG. 1B, 1C). Control experiments demonstrated that B cells with BCRs specific for hen egg lysozyme (MD4 Rag2^{-/-} mice) did not bind the MSP1 tetramer nor were they activated non-specifically by Plasmodium 8 days post-infection after adoptive transfer into a congenic host (FIGS. 8A, 8B). Thus, rare endogenous MSP1⁺ B cells that could be identified in naïve mice, expanded in an antigen-specific manner, demonstrating the ability to stringently identify and analyze MSP1⁺ B cells throughout the course of Plasmodium infection.

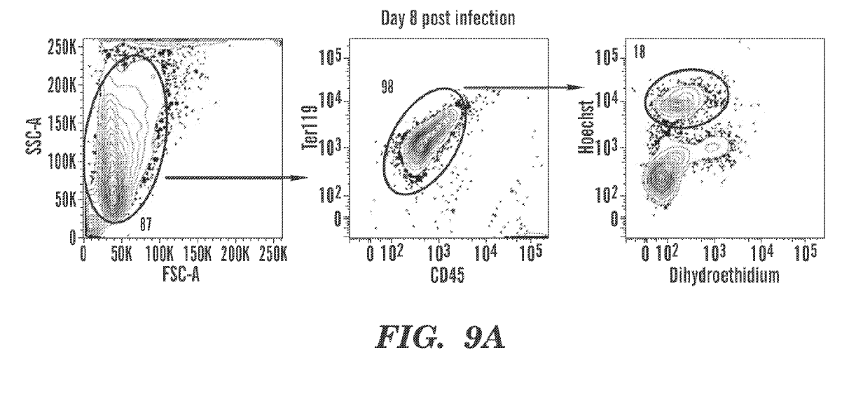

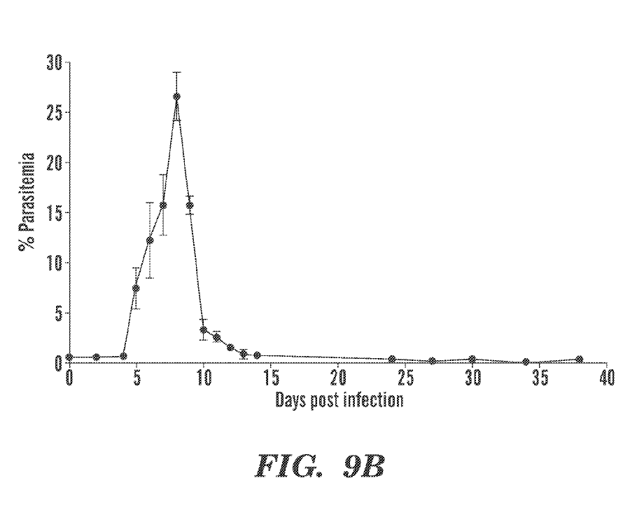

Both parasitemia and MSP1⁺ B cells were quantified in the spleens of individual mice for approximately a year after infection. Parasitemia was measured in blood samples throughout the course of infection using a flow cytometry based assay (Malleret et al., 2011; Robbiani et al., 2015) (FIG. 9A). MSP1⁺ B cells isolated from spleens of infected mice began to expand by 4 days after infection, peaked 8 days after infection, then sharply contracted, mirroring parasitemia (FIG. 1D and FIG. 9B). Variations in total MSP1⁺B cell numbers continued until day 150, although intracellular staining with the cell cycle marker Ki67 demonstrated that the vast majority (˜95%) of MSP1⁺ B cells at day 100 are quiescent. MSP1⁺ B cells persisted with a half-life of 221 days that resulted in a population of 3600 cells at 340 days post infection (FIG. 1E). MSP1⁺ B cells therefore expanded with ascending parasitemia, contracted, and then numbers fluctuated before stabilizing and slowly declining over 350 days. Importantly, these data demonstrated that long-lived, quiescent Plasmodium-specific B cells persisted and can be analyzed well after parasitemia is controlled.

MSP1-Specific B Cell Fates Emerge Early after Infection and MBCs Persist.

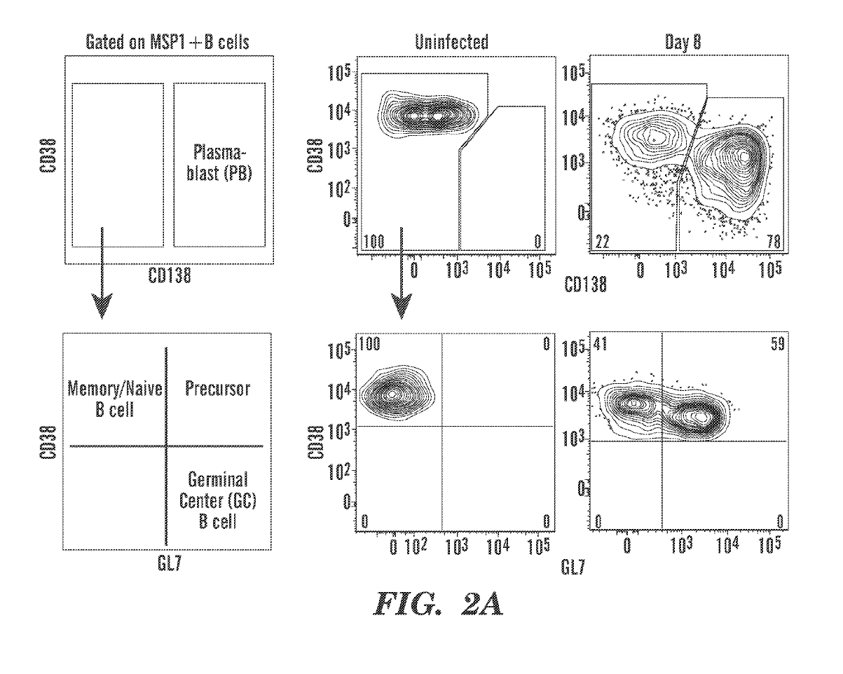

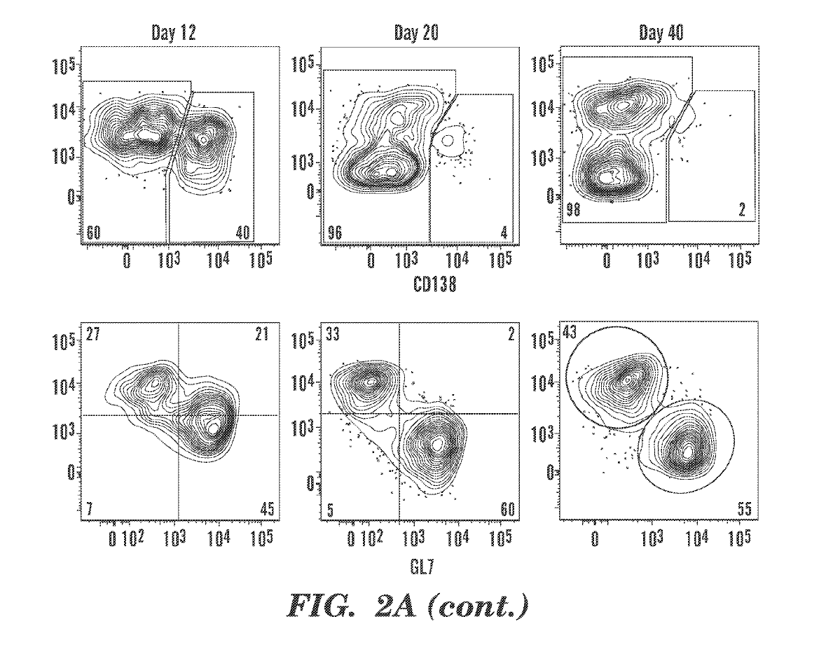

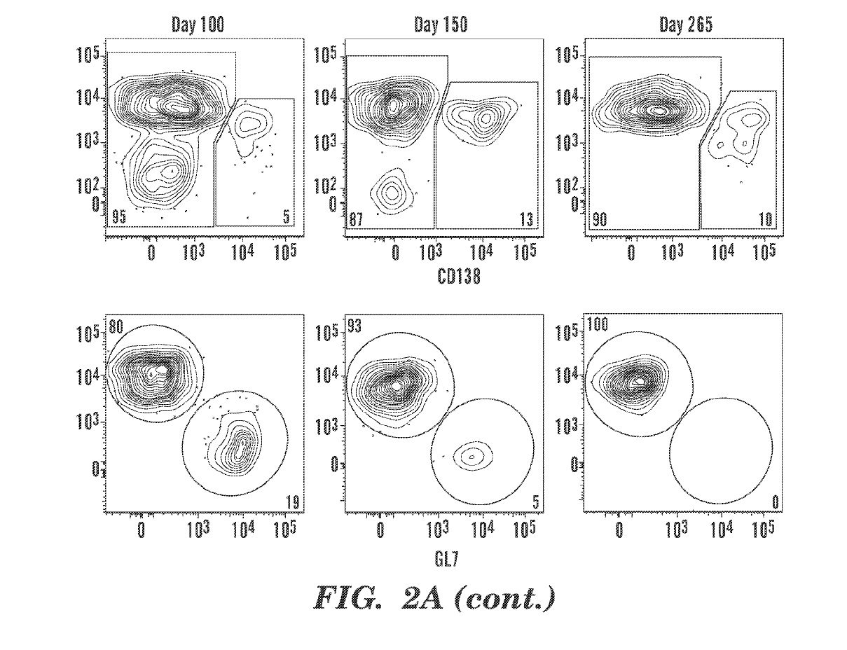

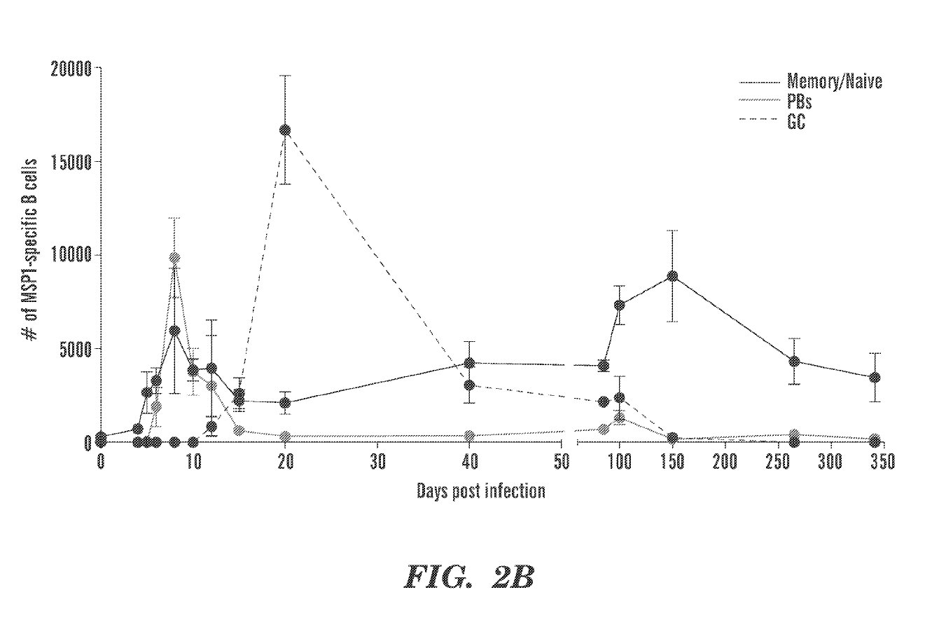

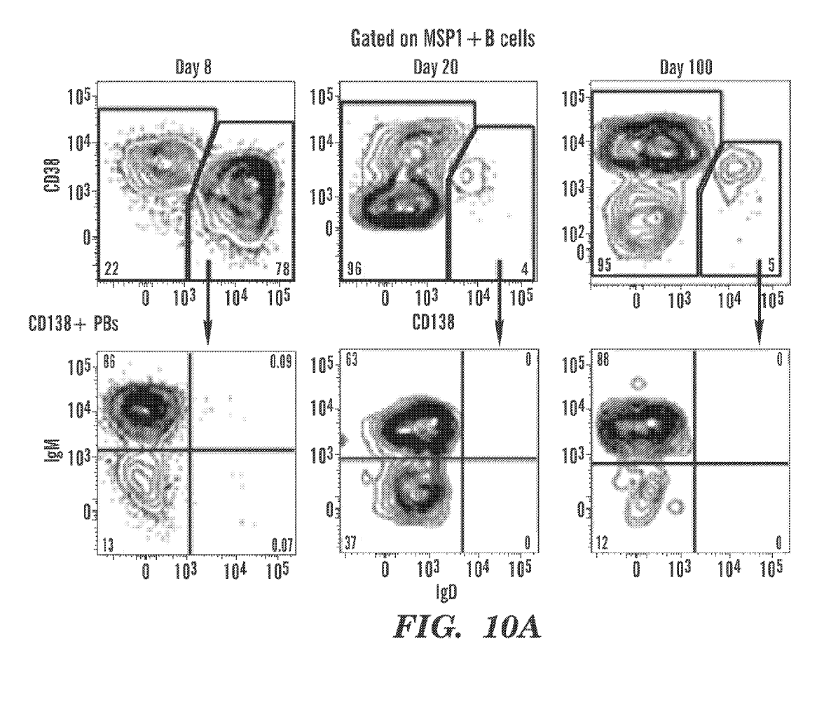

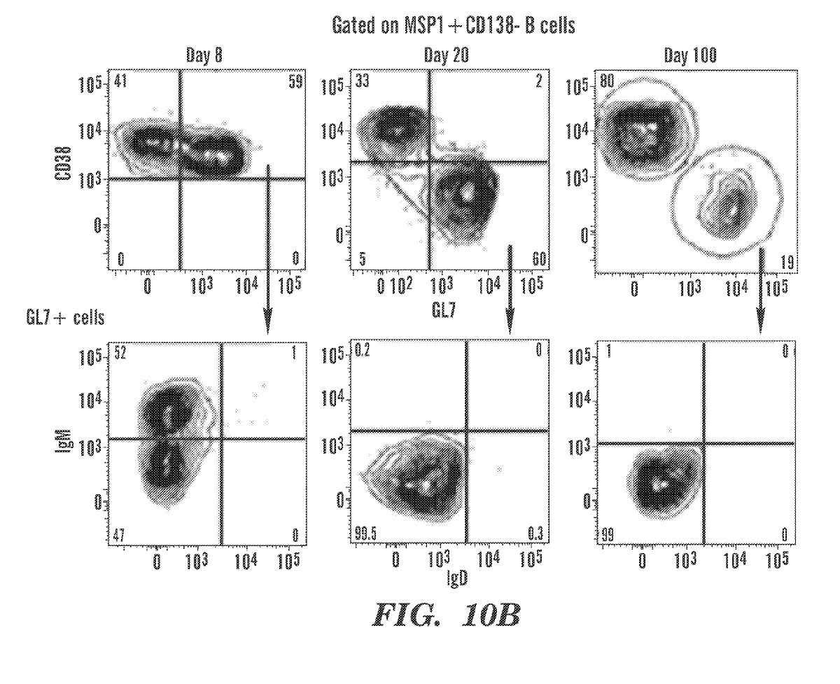

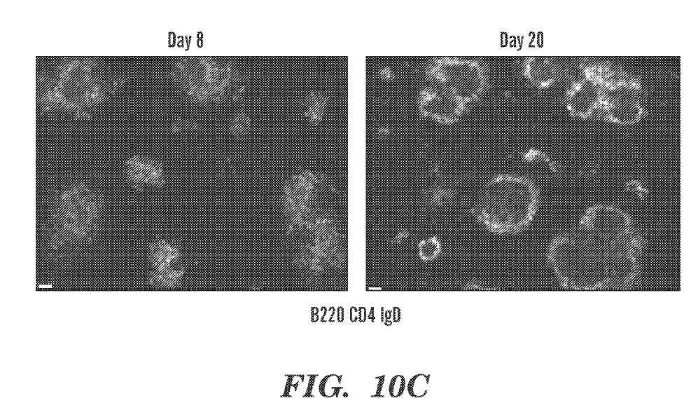

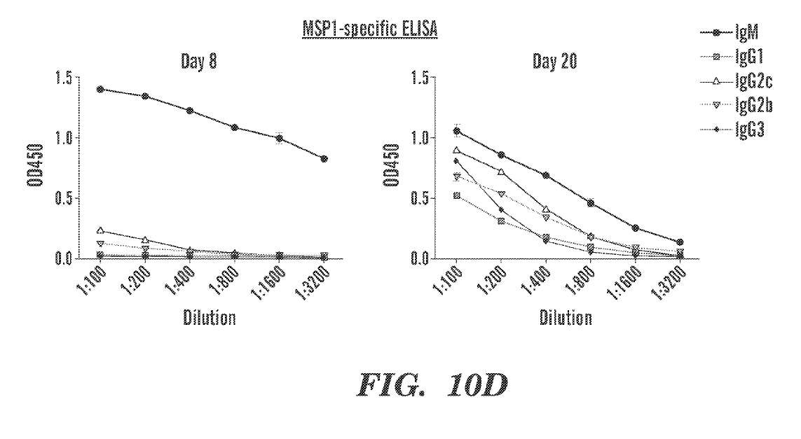

The heterogeneity of MSP1⁺ B cells was first assessed during the acute phase of the infection. Gating strategies were designed to distinguish between CD138⁺ plasmablasts (PBs), CD38⁺GL7⁺ activated precursors (Taylor et al., 2012b), CD38⁻GL7⁺ germinal center (GC) B cells, and expanded CD38⁺GL7⁻ MBC populations (FIG. 2A). Within 8 days of infection, multiple fates emerged including a dominant population of MSP1⁺ CD138⁺ PBs that primarily expressed IgM as measured by flow cytometry and serum ELISA consistent with previous reports (Achtman et al., 2003; Nduati et al., 2010) (FIGS. 2A, 10A, 10D). Several thousand MSP1⁺ B cells that retained CD38 expression, therefore resembling MBCs, were also present at day 8. The remainder of the population consisted of IgM⁺ and IgM⁻ GL7⁺CD38⁺ activated precursors which have been shown to be multipotent and capable of differentiating into GC B cells or MBCs (FIGS. 2A, 2B, 10B) (Taylor et al., 2012b). While GC responses were not present at day 8, they began to emerge at day 12, and expanded to a peak of about 15,000 MSP1⁺GL7⁺IgM⁻IgD⁻CD38⁻ cells at day 20, at which point numerous IgD⁻ germinal centers could also be found in the spleen by immunofluorescent microscopy (FIGS. 2A, 2B, 10B, 10C). This was further confirmed by the presence of various sub-classes of MSP1-specific IgG⁺ antibodies measured in the serum (FIG. 10D).

To determine which of these early fates persisted into the memory phase of the response, MSP1⁺ B cells were characterized for approximately a year using similar gating strategies described above (FIG. 2A, 2B). Although CD138⁺ PBs initially waned between days 20 to 40, a small, consistently present CD138⁺ population re-emerged around day 85 suggesting that these were splenic plasma cells (PCs), similar to recent work demonstrating that

PCs emerge after MBCs in response to protein immunization (Bortnick et al., 2012; Weisel et al., 2016). These PCs persisted at all timepoints thereafter, were still present at day 340 post infection, and were predominantly IgM⁺ (FIGS. 2A, 2B, 10A).

Enrichment techniques also facilitated the visualization of a waning GC response. MSP1⁺ GC B cells contracted by day 40 post infection and then slowly declined before eventually disappearing around 150 days post infection. Therefore, from day 50 on, the vast majority of the MSP1⁺ cells were CD38⁺GL7⁻ MBCs that remained for at least 340 days post infection (FIGS. 2A, 2B). These data demonstrate that well after parasite clearance and termination of the GC reaction, splenic MSP1⁺ B cells are predominantly comprised of an expanded population of CD38⁺ MBCs and a small but persistent CD138⁺ PC population.

### Switched and Unswitched Plasmodium-Specific MBCs can be Found in Malaria-Exposed Mice and Humans

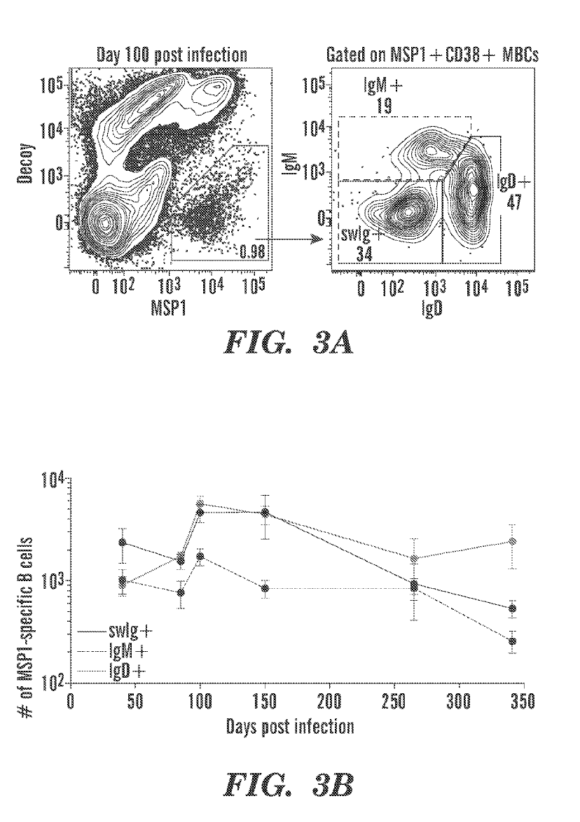

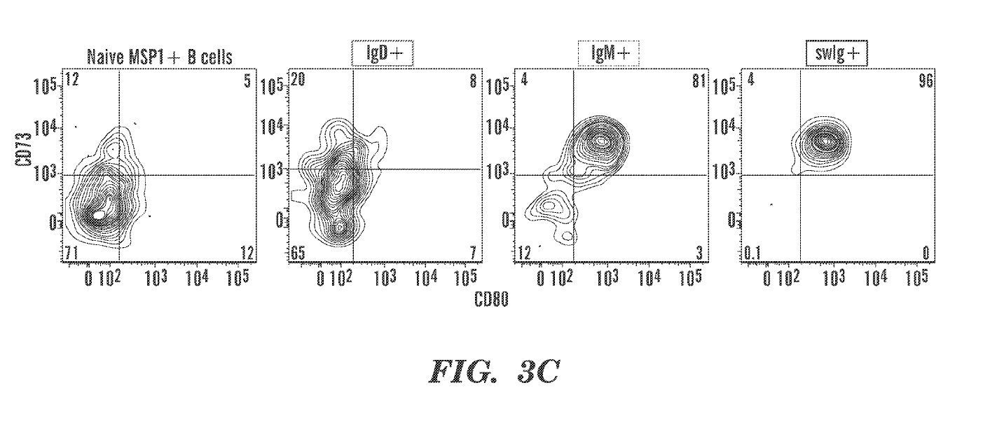

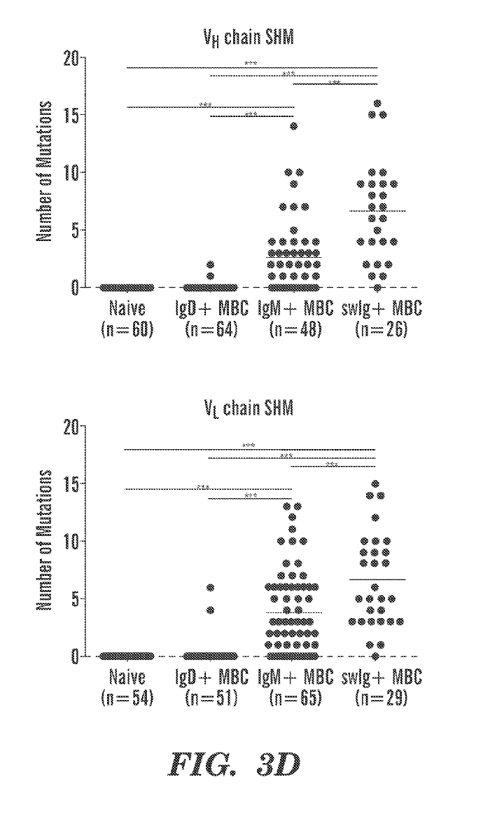

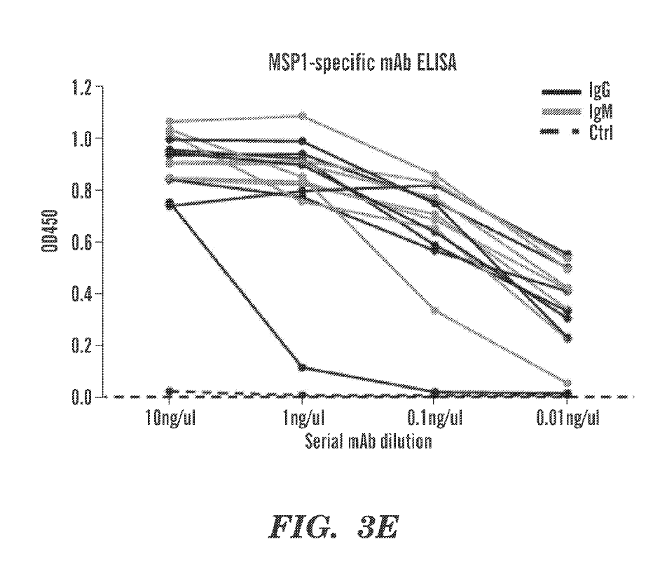

It was next important to determine if recently defined MBC subsets that emerge after protein immunization were also present in response to infection. To interrogate the diversity of the MSP1⁺ MBCs, antibodies specific for IgM and IgD were used to identify “switched” and “unswitched” MSP1⁺ B cells. Interestingly, this staining strategy identified three distinct populations of MSP1⁺ MBCs 100 days after infection: an IgM⁻IgD⁻ isotype switched population (referred to as swIg⁺), and two unswitched subsets. One subset was phenotypically IgM^{lo}IgD^{hi}g^{h} (referred to as IgD⁺) while the other subset was IgM^{hi}g^{h}IgD^{lo} (referred to as IgM⁺) (FIG. 3A). While all three populations persisted for 340 days post infection, at the latest time points IgD⁺ MBCs stably persisted whereas both the IgM⁺ and swIg⁺ MBCs declined (FIG. 3B).

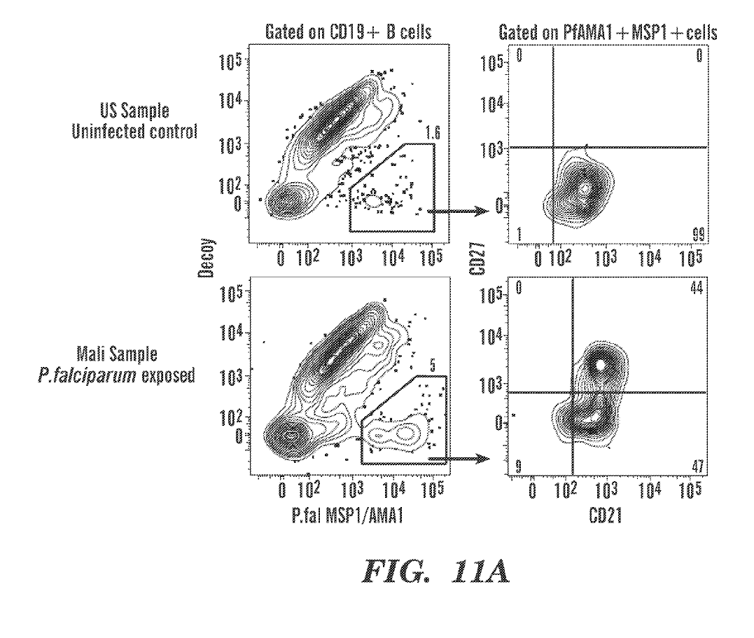

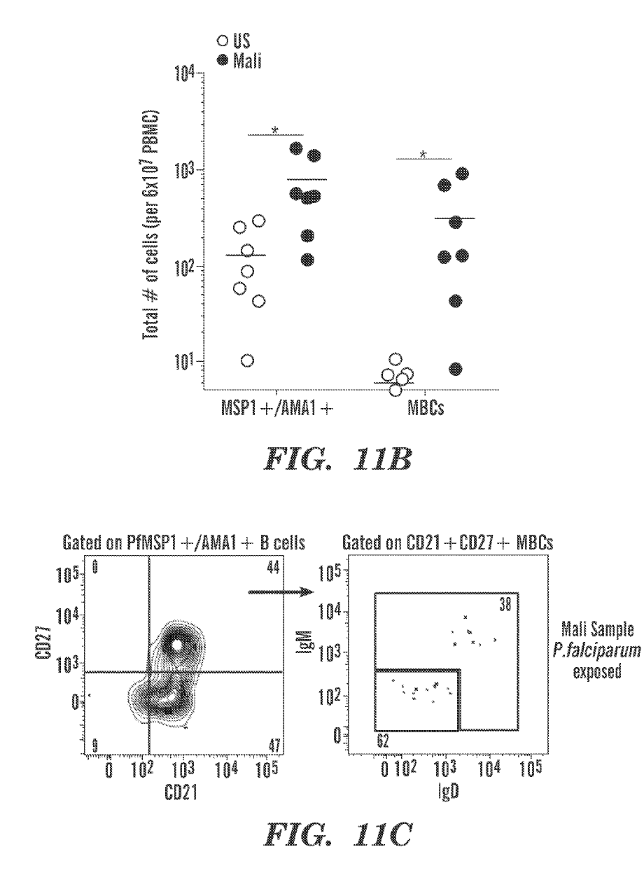

Because of the persistence of heterogeneous MBCs after malaria infection in mice, whether switched and unswitched P. falciparum-specific MBCs also occur in exposed individuals residing in an endemic area was investigated. Although IgG⁺P. falciparum-specific MBCs have been detected by ELISPOT (Weiss et al., 2012) in individuals exposed to both high (Ndungu et al., 2012; Weiss et al., 2010) and low (Clark et al., 2012; Ndungu et al., 2013; Wipasa et al., 2010) malaria transmission, it is unknown if P. falciparum infection induces antigen-specific unswitched MBCs. Antigen-specific enrichment experiments were therefore performed on peripheral blood mononuclear cells (PBMCs) collected from P.falciparum-infected Malian subjects during the malaria season (Crompton et al., 2008) or malaria-naïve U.S. subjects. To enhance the sensitivity of Plasmodium-specific cell detection (less than 20 million PBMC were available in some samples), we generated B cell tetramers using the C-terminal region of MSP1 and apical membrane antigen 1 (AMA1) from the human P. falciparum (3D7) strain. In Malian subjects, we found that approximately 40% of the P. falciparum-specific B cells in blood were CD21⁺CD27⁺ MBCs in keeping with expected frequencies of total MBCs in human blood (Kaminski et al., 2012; Klein et al., 1998; Tangye and Good, 2007) (FIG. 11A). Furthermore, there was a 6 fold increase in the total number of P. falciparum-specific B cells and a 60-fold increase amongst CD27⁺CD21⁺ MBCs compared to uninfected US controls (FIG. 11B). We further characterized the P. falciparum-specific CD27⁺ MBCs from Malian samples for their expression of BCR isotype and found that they were comprised of both switched and unswitched cells (FIG. 11C). Thus, heterogeneous populations of Plasmodium-specific MBCs are expanded in both mice and humans.

### Murine MSP1-Specific MBC Subsets are Phenotypically and Genetically Distinct

To further dissect the unique phenotypic and functional characteristics associated with distinct Plasmodium-specific MBCs, additional studies were performed in mice. Studies have demonstrated that MBC subsets display heterogeneous expression of surface markers associated with T cell interactions including CD73 and CD80 on both switched and unswitched MBCs (Anderson et al., 2007; Tomayko et al., 2010; Yates et al., 2013). Expression of these proteins was therefore examined on MSP1⁺ MBCs 100 days post infection. Again, it was found that the division of unswitched MBCs into IgM⁺ and IgD⁺ subsets largely accounted for the variability in surface marker expression. ˜81% of IgM⁺ MBCs expressed CD73 and CD80, comparable to the ˜96% of the swIg⁺ MBCs that expressed both markers, whereas only ˜8% of IgD⁺ MBCs expressed CD73 and CD80, comparable to MSP1⁺ naïve B cells (FIG. 3C). Similar to MBC diversity generated by protein immunization, phenotypically diverse Plasmodium-specific IgD⁺, IgM⁺, and swIg⁺ MBC subsets develop in response to infection. Additionally, expression of CD73 and CD80 further distinguishes IgM⁺ and IgD⁺ MBCs as two distinct, unswitched populations.

B cell expression of both CD73 and CD80 is associated with expression of activation-induced cytidine deaminase (AID) and, in some cases but not all, germinal center dependence (Anderson et al., 2007; Kaji et al., 2012; Taylor et al., 2012b; Weisel et al., 2016). Based on these observations, it was tested whether the CD73⁺CD80⁺MSP1⁺IgM⁺ MBCs represented a previously unexplained population of somatically hypermutated, unswitched MBCs identified in other immunization models (Kaji et al., 2012; Pape et al., 2011). To test this, flow cytometric sorting was used to isolate individual MSP1⁺ CD73⁻CD80⁻IgD⁺, CD73⁺CD80⁺IgM⁺, and CD73⁺CD80⁺swIg⁺ MBCs or MSP1⁺ naïve B cells. Individual BCRs were sequenced and cloned using previously described methods (Tiller et al., 2009). The relative numbers of somatic hypermutations (SHM) in both light (VH) and light (VL) chain sequences present in individual MBC subsets were calculated after comparison to BCRs from naïve MSP1⁺ B cells, which had no mutations and were identical to germline sequences. While only 3% of IgD⁺ MBC VH or VL chain sequences showed SHM, 65% of VH and 75% of VL chain sequences of CD73⁺CD80⁺IgM⁺ cells were mutated with a mean of 3 mutations in both chains (FIG. 3D). swIg⁺ MBCs were also highly mutated (97%) and displayed significantly more mutations (mean of 8) in both VH and VL chains (FIG. 3D).

Since increased levels of SHM are associated with an overall increase in BCR affinity (Chan and Brink, 2012), the affinities of IgM⁺ and swIg⁺ MBC BCRs were tested. Individual BCR variable region sequences with varying levels of somatic hypermutation from either MSP1⁺IgM⁺ or swIg⁺ MBC clones were therefore expressed as monoclonal antibodies (mAb), with human IgG constant (Fc) regions to prevent contributions to avidity by oligomerization. Antibodies were then used in dilution assays against MSP1 protein to compare affinity of the various mAbs by ELISA (Kolhatkar et al., 2015). Importantly, these studies further confirmed the specificity of MSP1-tetramer techniques, as 100% of expressed clones bound MSP1 protein, while the control PC-specific mAb did not (FIG. 3E). Furthermore, despite overall fewer mutations, individual BCRs from IgM⁺ MBCs showed comparable affinity for the MSP1 protein to swIg⁺ MBCs (FIG. 3E). These data therefore demonstrate that expression of CD73 and CD80 on both IgM⁺ and swIg⁺ MBCs is associated with increased levels of SHM, resulting in similar BCR affinities.

### Secondary Infection Induces the Rapid Proliferation and Differentiation of MSP1-Specific MBCs.

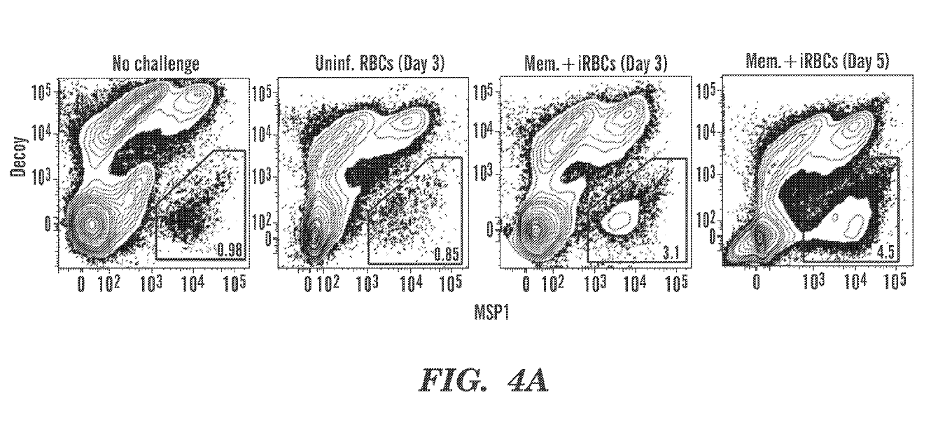

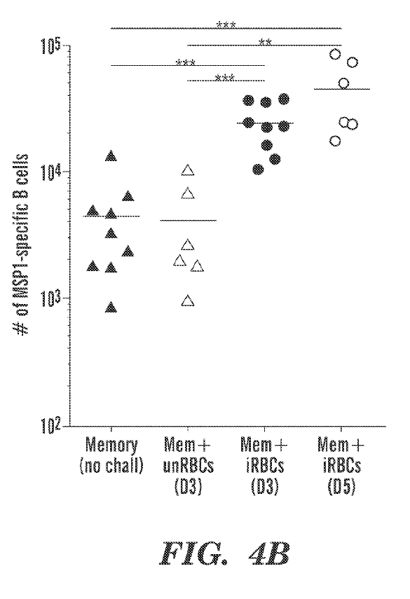

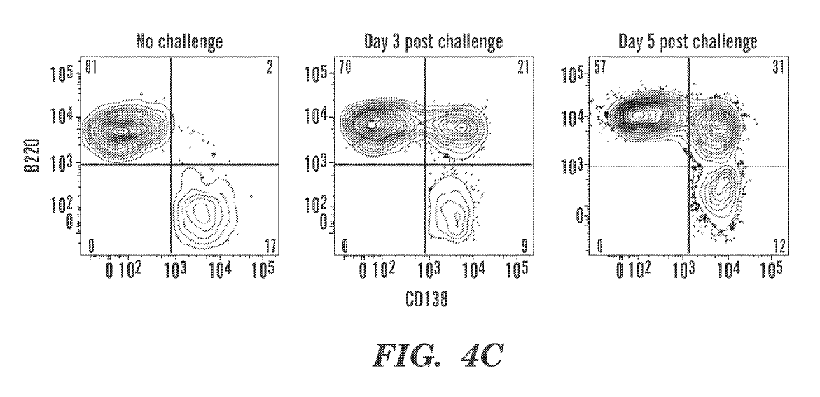

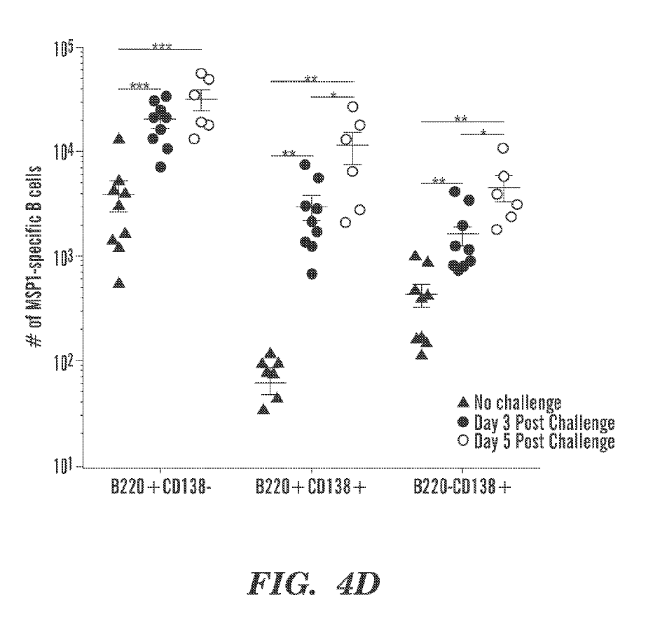

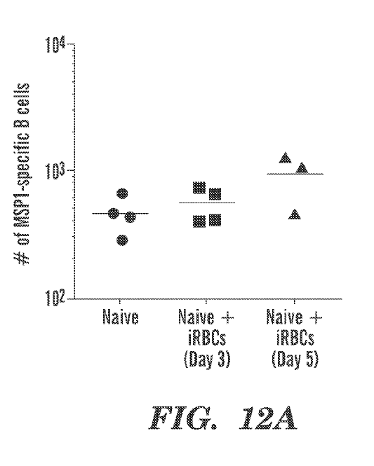

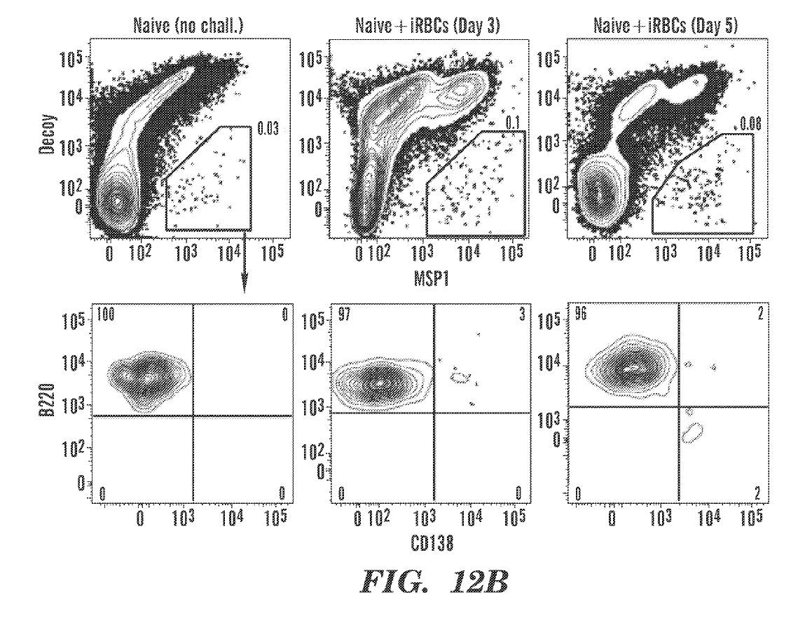

To understand how the MBCs described above function during a secondary infection, mice in the memory phase of the response were rechallenged with iRBCs. Of note, the experimental conditions used were distinct from several previous studies that utilized adoptive transfer of individual MBC populations followed by antigen rechallenge. In intact memory mice, MBC competition for antigen and T cell help, as well as the presence of pre-existing antibodies factor into the overall response, perhaps as they would in repeatedly infected humans. To replicate this, memory mice infected 12-16 weeks prior were left unchallenged or rechallenged with either 1×10⁷ uninfected RBCs (unRBCs) or iRBCs, and MSP1⁺ B cells were analyzed 3 or 5 days later. Following rechallenge with iRBCs, but not unRBCs, the total number of MSP1⁺ B cells expanded significantly on day 3 and continued to increase at day 5 compared to unchallenged memory mice (FIGS. 4A, 4B). To ascertain whether these newly formed cells were originating from MBCs or recently formed naïve cells, naïve mice were also infected with a challenge dose of 1×10⁷ iRBCs and MSP1⁺ B cells were quantified and phenotyped. In stark contrast to the logarithmic increase seen in MSP1⁺ B cells in memory mice after rechallenge, there was no significant increase in the total number of MSP1⁺ B cells in naïve mice at either 3 or 5 days after a primary infection (FIG. 12A).

It was next determined whether expanded MSP1⁺ cells in rechallenged memory mice were also differentiated. Phenotypic analyses using gating strategies described above confirmed that MSP1⁺ B cells in memory mice prior to challenge consisted of both B220⁺CD138⁻ B cells (consisting primarily of MBCs and a small, waning population of GC B cells) and B220⁻CD138⁺ PCs (FIGS. 2A-2B, 4C). Three days after iRBC challenge, a newly formed MSP1⁺B220⁺CD138⁺ population emerged and remained expanded at day 5, suggesting these were the product of recently activated MBCs (FIGS. 4C, 4D). The rapid formation of this population was unique to a memory response as we did not observe a significant B220⁺CD138⁺ population form in naïve mice three days after the same iRBC challenge (FIG. 12B). Additional quantification of the B220⁺CD138⁻B cells and B220⁻CD138⁺ PCs revealed that these populations also increased in number after rechallenge (FIG. 4D). Together, these data demonstrate that within 3 days of rechallenge, expanded and differentiated MSP1⁺ B cells form in response to a secondary infection.

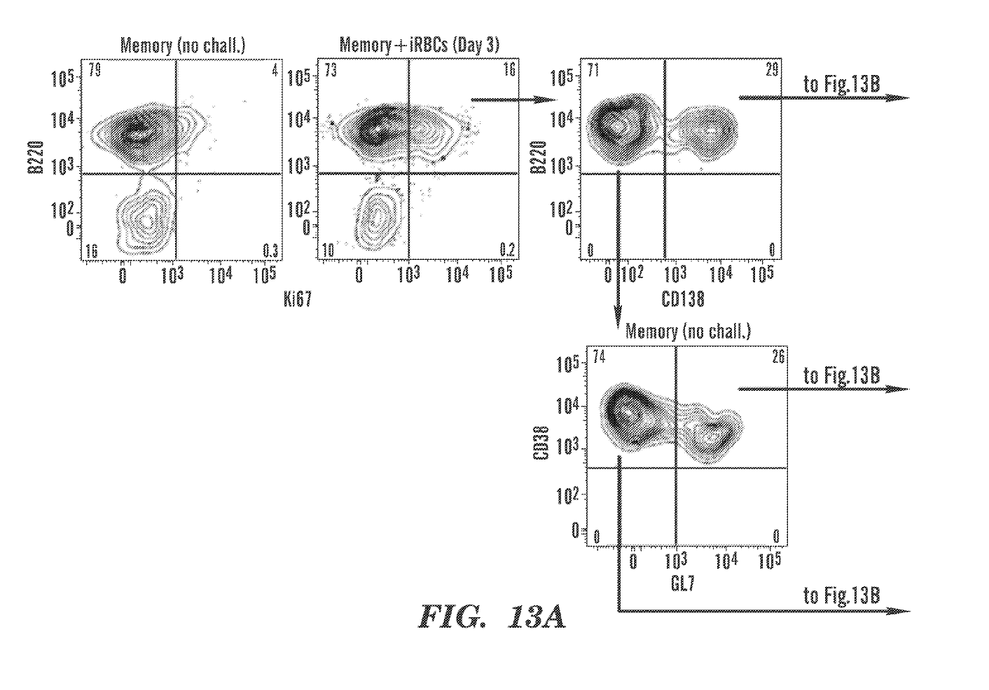

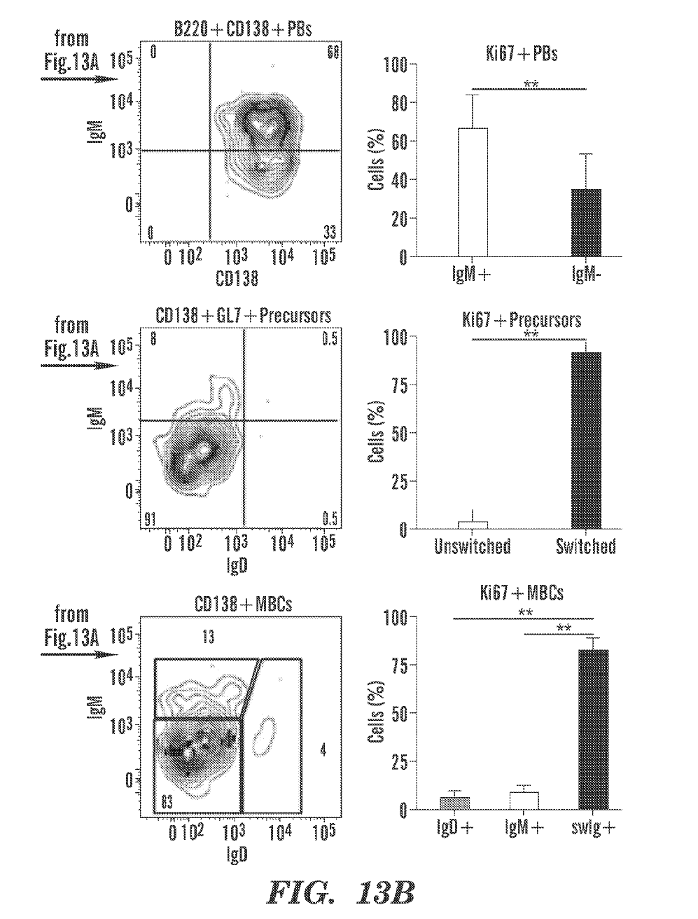

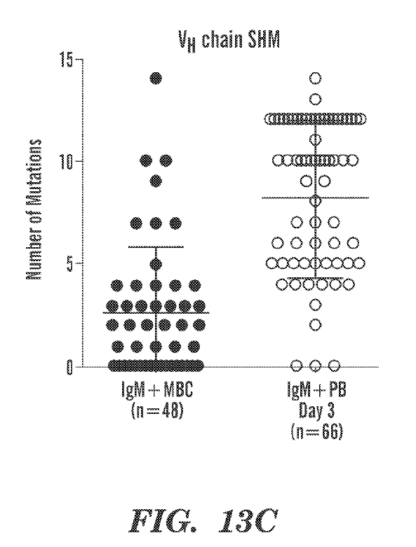

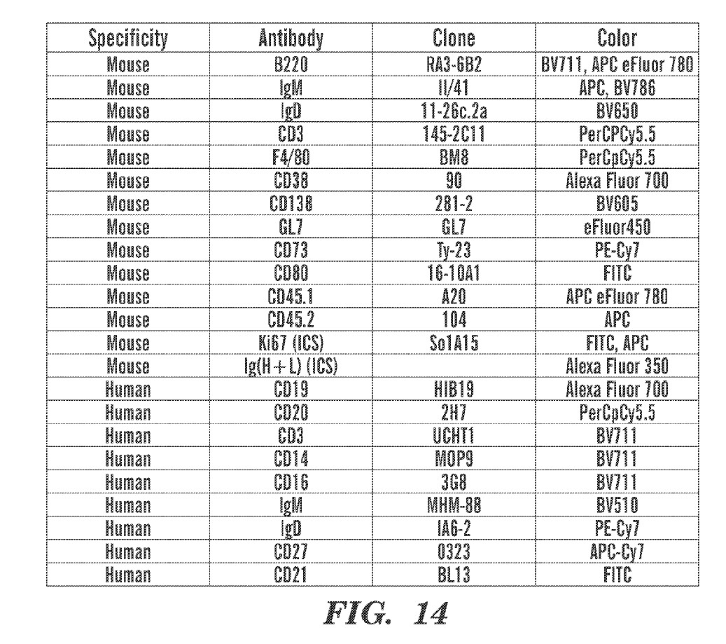

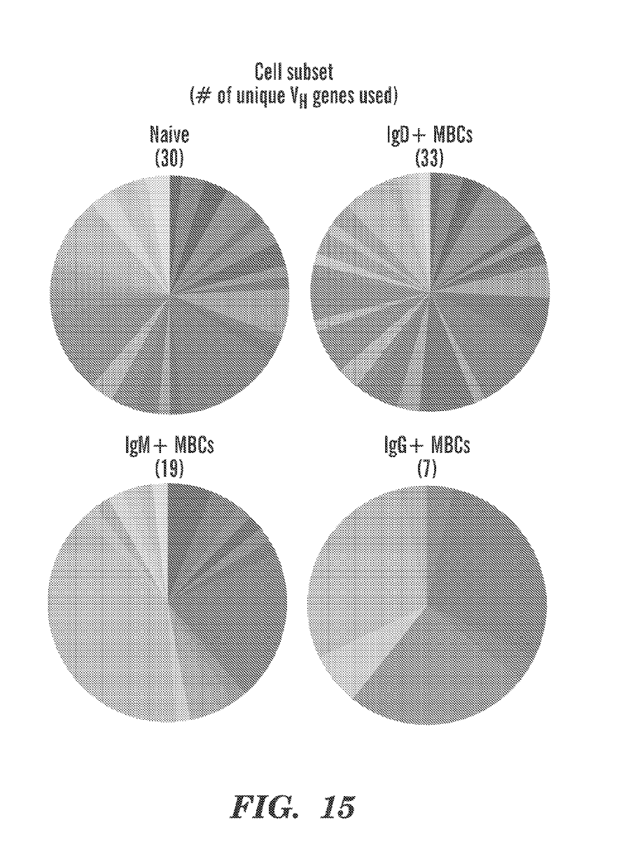

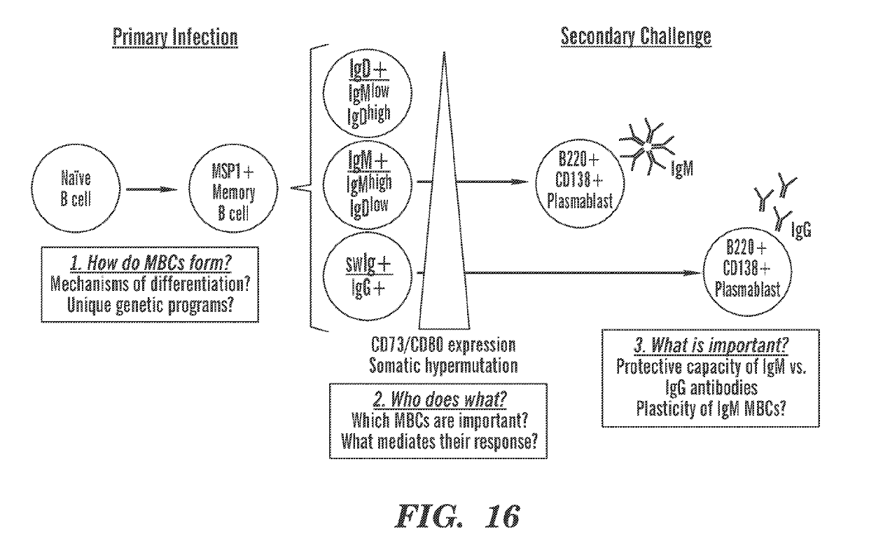

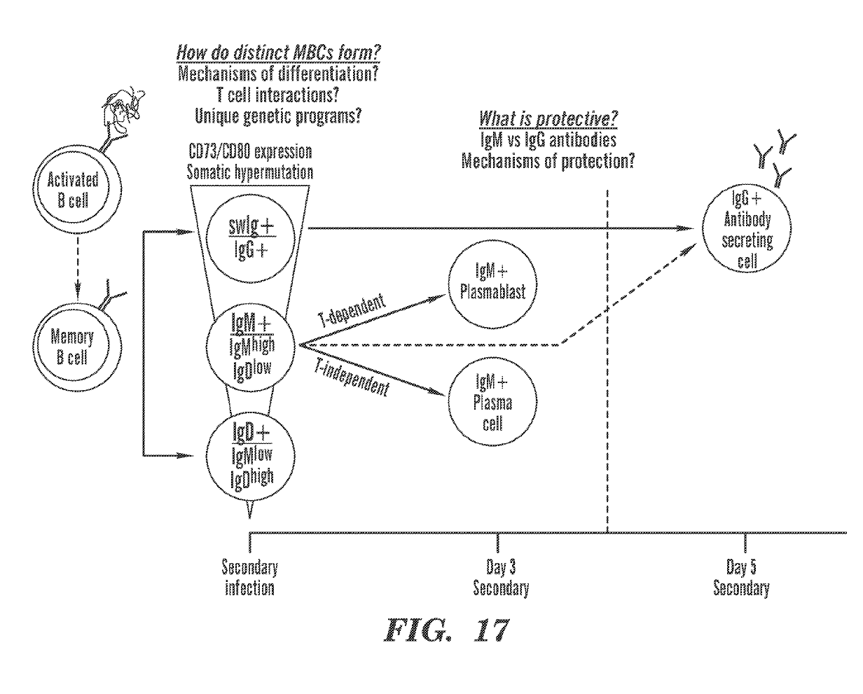

To determine what precursor populations were proliferating to produce expanded populations of MSP1⁺ B cells, Ki67 expression (which marks actively cycling cells) was compared before and after rechallenge. Prior to challenge, −4% of MSP1⁺ B cells were Ki67⁺ (FIG. 13A). Three days after rechallenge the percentage of Ki67⁺ increased to −16% of all MSP1⁺ B cells and remained restricted to the B220⁺B cells (B220⁻ PCs were Ki67⁻) (FIG. 13A). Detailed phenotypic analysis of the Ki67⁺ cells revealed that three separate MSP1⁺B220⁺ populations were proliferating: newly formed B220⁺CD138⁺ plasmablasts (PBs) (−30%), CD38⁺ MBCs (−50%) and CD38⁺GL7⁺ activated precursors (−20%) (FIG. 13A). Therefore, within three days, some MSP1⁺ MBCs had already proliferated and differentiated into PBs and CD38⁺GL7⁺ activated precursors, but many CD38⁺GL7⁻ MBCs were still proliferating but had not yet differentiated.

The isotypes of the proliferating cells were also determined to reveal precursor relationships. Surprisingly, the majority of both Ki67⁺ PBs three days after rechallenge expressed IgM despite IgM⁺ MBCs being at a numerical disadvantage to the swIg⁺ MBCs at this timepoint (FIGS. 13B, 3B). The activated precursors and MBCs were largely isotype switched (FIG. 13B). In contrast, very few of the MSP1⁺IgD⁺ MBCs were proliferating. These data demonstrate that IgM⁺ MBCs rapidly respond to secondary infection and make up the majority of the early proliferating plasmablasts.

To further discern precursor relationships for the IgM⁺ PBs, BCRs were cloned from the IgM⁺ B220⁺CD138⁺ PBs 3 days after challenge to look for somatic hypermutation. If the PBs were somatically hypermutated, it would support the idea that these cells were derived from somatically hypermutated IgM⁺ MBCs as opposed to unmutated IgD⁺ MBCs. Remarkably, 95% of newly formed IgM+PB clones (mean mutation of 8) were somatically hypermutated at levels that were comparable to MSP1⁺IgM⁺ MBCs, further establishing a precursor relationship between IgM⁺ MBCs and newly formed PBs after a secondary infection (FIG. 13C).

### The Early Secondary Antibody Response is IgM-Dominant

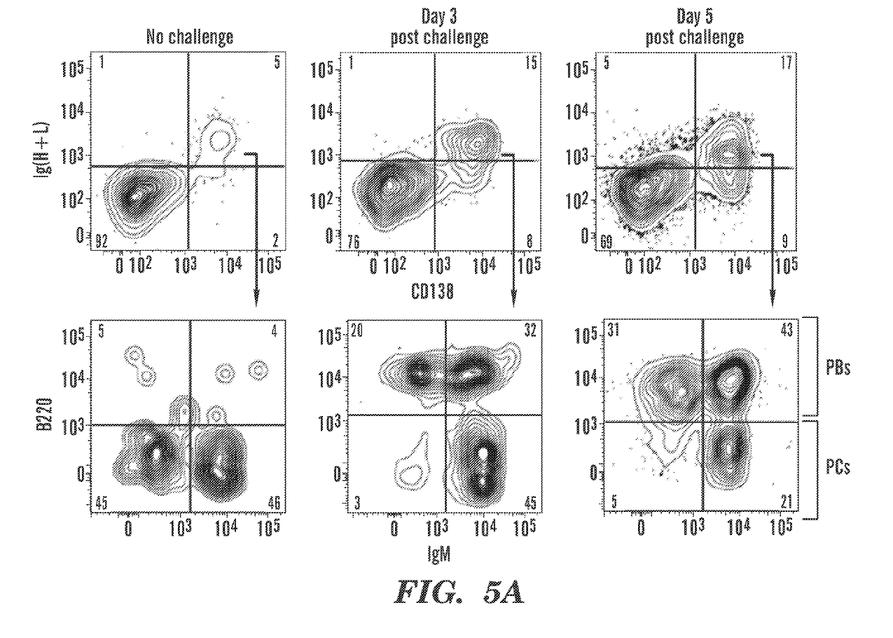

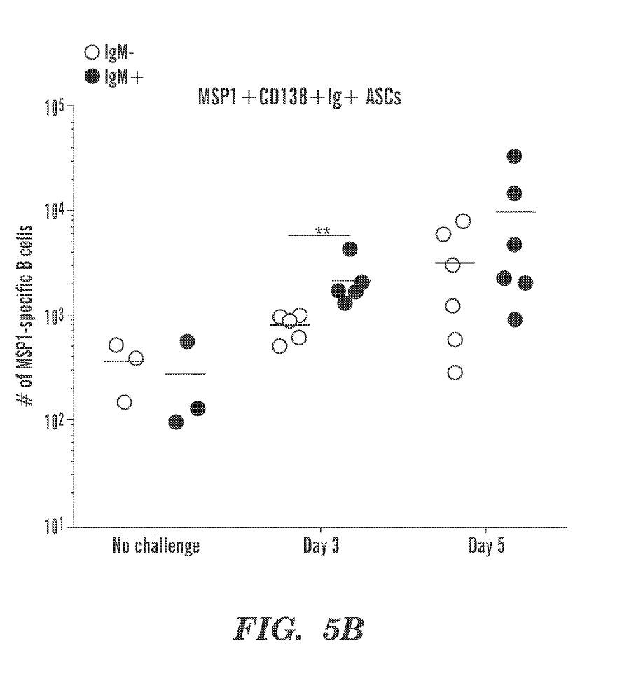

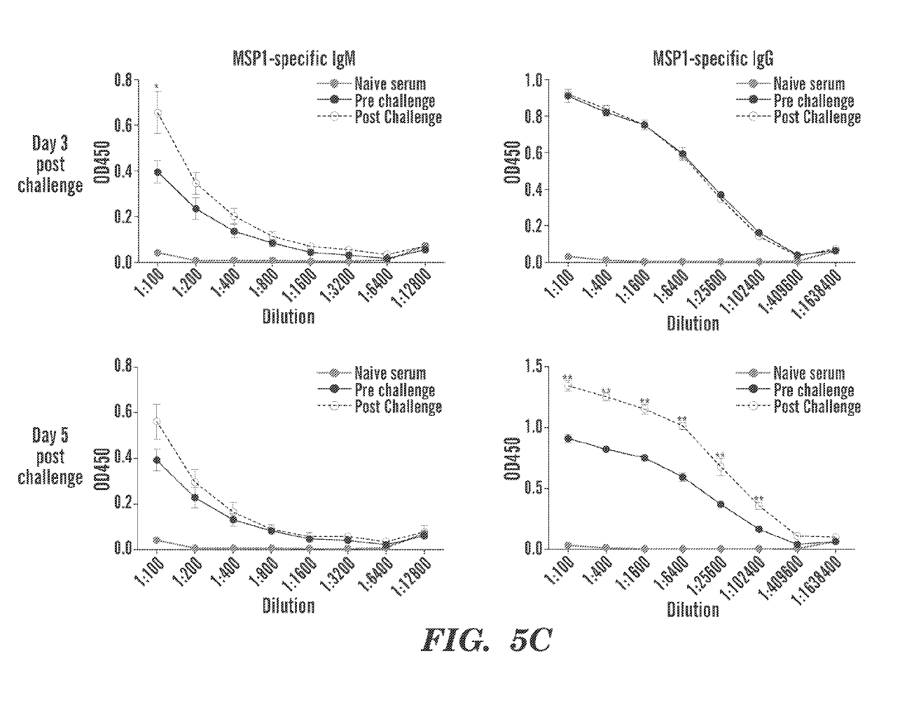

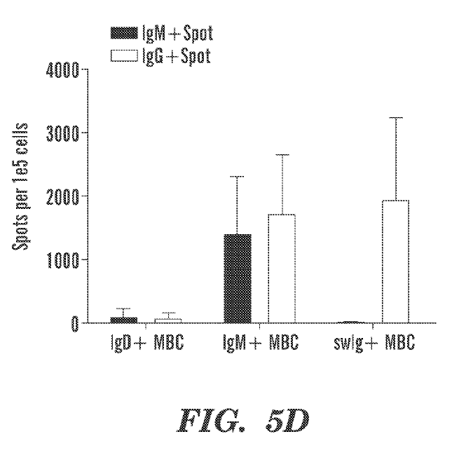

It was next asked what MSP1⁺ cells were differentiated antibody secreting cells (ASCs). Again, memory mice were rechallenged and intracellular staining for immunoglobulin light and light chain (Ig) was performed on MSP1⁺ B cells 3 or 5 days later. In memory mice analyzed prior to challenge, the only ASCs present were ˜600 B220⁻CD138⁺ PCs (which represent about 5% of the total cells) (FIG. 5A). Three days after rechallenge, approximately ˜3000 MSP1⁺ B cells (about 15%) were now making antibody, split between B220⁺CD138⁺ PBs and B220⁻CD138⁺ PCs (FIG. 5A). Now, approximately 70% of the MSP1⁺Ig⁺ ASCs were IgM⁺, while only about 30% of the ASCs were switched, resulting in significantly more IgM⁺ ASCs on day 3 than switched ASCs (FIGS. 5A, 5B). Two days later, on day 5 post challenge, IgM⁺ ASCs continued to expand, but now there was also a larger, switched antibody-secreting PB pool. Interestingly, the switched PCs stayed relatively stable at all timepoints examined (FIGS. 5A, 5B).

To confirm that intracellular antibody staining represented measurable changes of secreted antibody in vivo, MSP1-19 protein-specific ELISAs were performed on serum samples taken from individual mice before or after challenge. In conjunction with what was observed by flow cytometry, three days after infection MSP1-specific IgM antibody expression was significantly increased over pre-challenge levels while IgG antibody expression remained unchanged (FIG. 5C, top row). Two days later however, on day 5, we observed significant increases in MSP1-specific IgG antibodies, while IgM antibody levels remained elevated (FIG. 5C, bottom row). Since it was unclear if these switched PBs arose from swIg⁺ or IgM⁺ MBCs, MBCs were additionally sorted two days after rechallenge to look for IgM or IgG expression by ELISPOT after 2 days in culture. This approach revealed that, while swIg⁺ MBCs could only form IgG⁺ ASCs, IgM⁺ MBCs formed both IgM⁺ and IgG⁺ ASCs (FIG. 5D). Collectively, these data demonstrate that the secondary response is dominated by early IgM⁺ antibody expression and later IgG⁺ antibody expression. Additionally, these findings demonstrate that IgM⁺ MBCs are capable of expressing both IgM⁺ and IgG⁺ antibodies, highlighting that the IgM⁺ MBCs are rapid, plastic responders to a secondary infection.

### Secondary IgM Response is not Affected by Challenge Dose or Timing

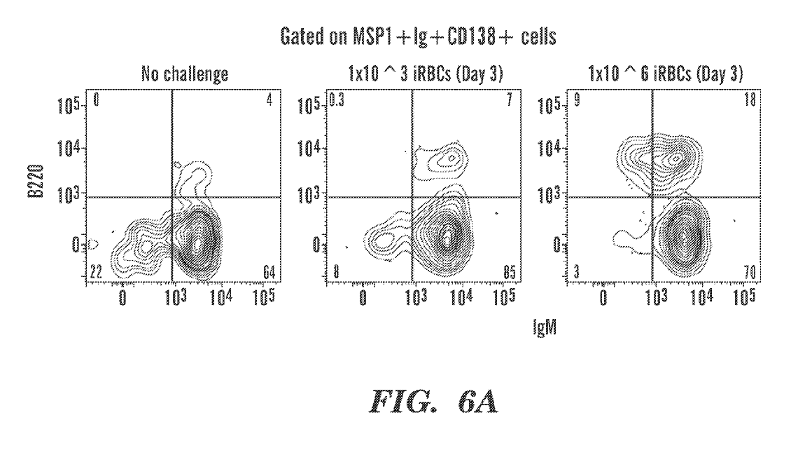

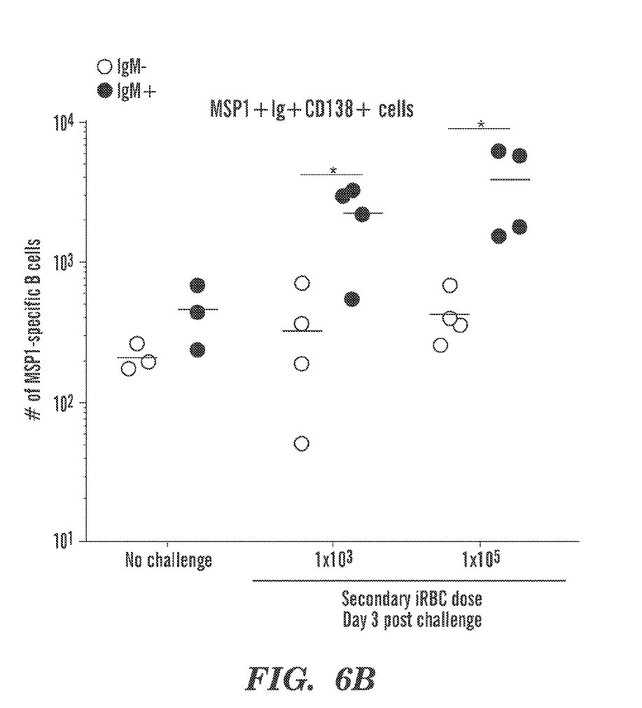

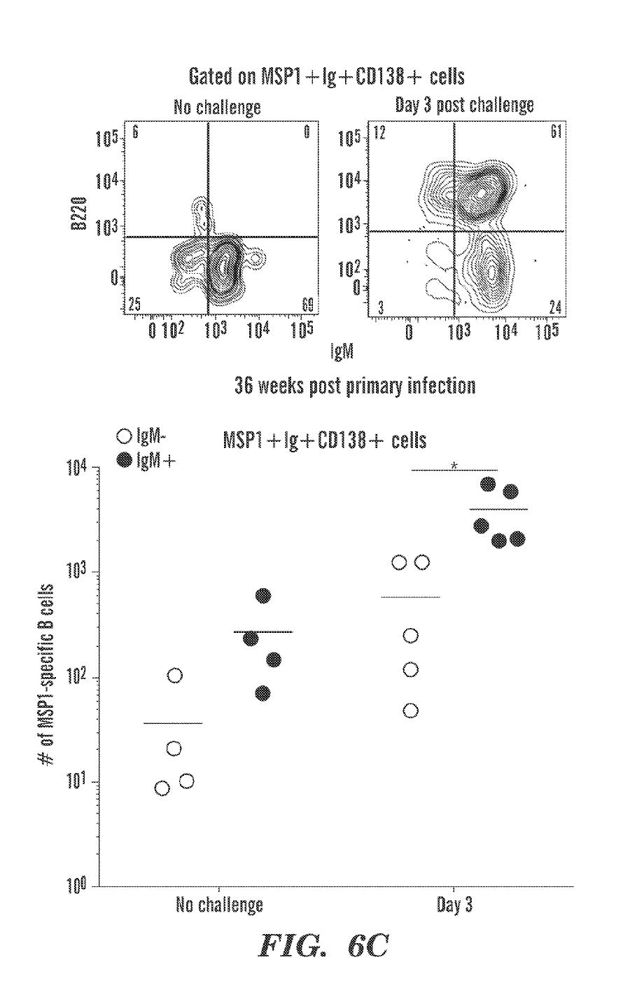

One potential cause for the early IgM dominant response after secondary challenge could be high antigen load, which could somehow preferentially activate IgM⁺ MBCs. Memory mice were therefore challenged with two lower iRBC challenge doses (1×10³ and 1×10⁵) prior to MSP1⁺ B cell analysis 3 days later. Remarkably, in both lower dose challenges, the IgM⁺ ASC response still dominated the early ASC population and even more dramatically than what was observed at the higher dose challenge (FIGS. 6A, 6B, 5B). This was especially striking given the 2.5-fold numerical disadvantage of IgM⁺ MBCs compared to swIg⁺ MBCs 100 days post-challenge (FIG. 3B).

While this ruled out dose dependent effects, it was also possible that the time of rechallenge influenced the results, for example if a germinal center was ongoing, which was the case for the 12-16 week rechallenge experiments. It was therefore tested whether the presence of an ongoing GC reaction at the time of challenge influenced the early secondary responders. Memory mice 35 weeks post infection, in which the GC reaction had ended and IgM⁺ and swIg⁺ MBCs were in equal number (FIGS. 2A-2B, 3A-3E), were therefore given a secondary challenge with 1×10⁷ iRBCs and analyzed 3 days later. As seen in mice with an ongoing GC, IgM⁺ cells were still the predominant early antibody-expressing population (FIG. 6C). Together these data indicate that despite variations in infectious dose, the presence or absence of a GC, or shifts in the numerical ratio of IgM⁺ to swIg⁺ MBCs, IgM⁺ MBCs can compete with swIg⁺ MBCs and are important early responders in a secondary Plasmodium infection.

### IgM⁺ MBCs Generate Both T-Independent and T-Dependent Antibody Secreting Effectors

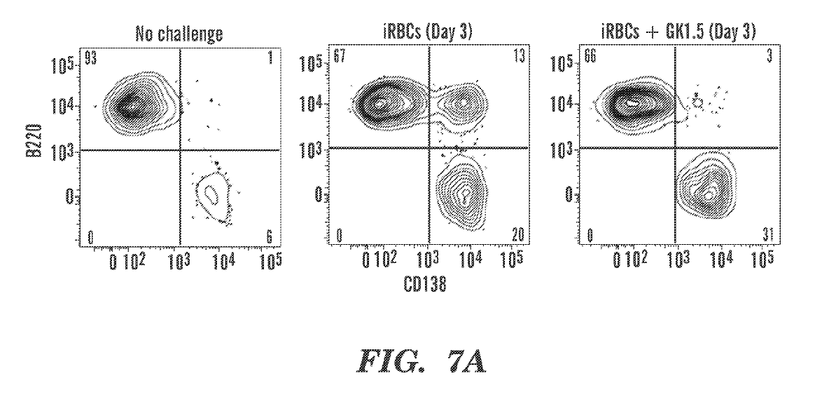

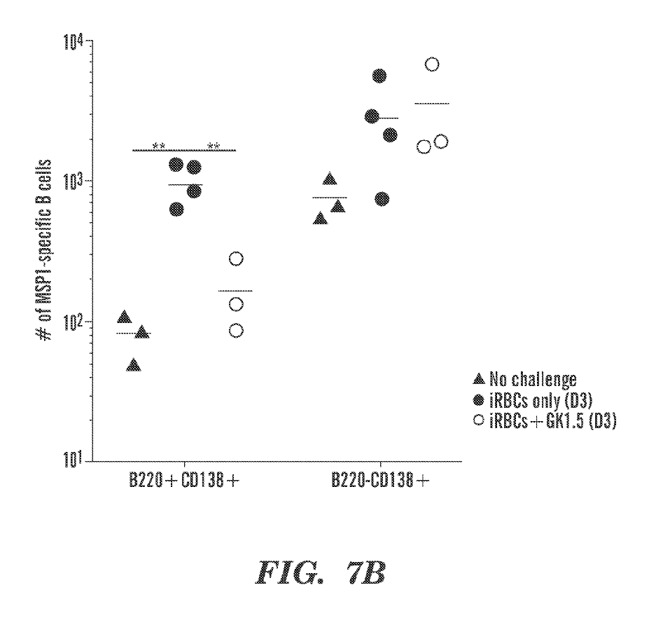

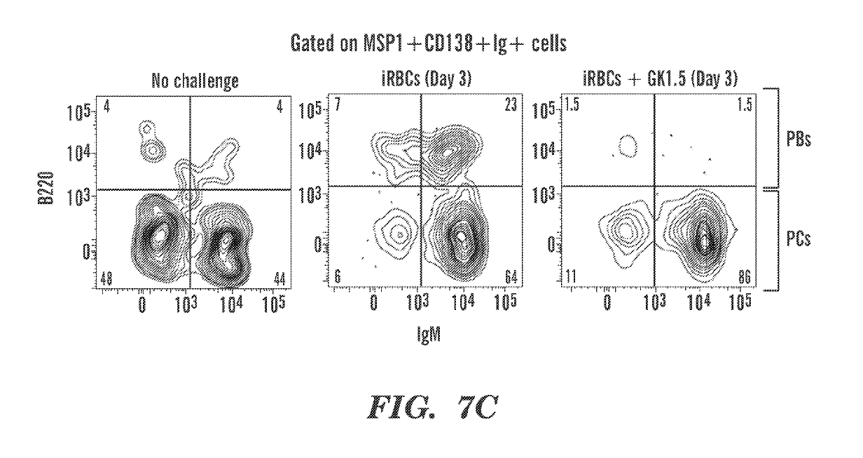

The better understand the predominant secondary IgM⁺ memory response, the mechanisms of the early IgM response were interrogated. It was hypothesized that differences in T cell dependence could allow some populations to form faster than others. To test this, mice were treated a CD4⁺ T cell depleting antibody (clone GK1.5) for two days prior to rechallenge and formation of PBs and PCs was assessed 3 days later. Strikingly, while MSP1⁺ PBs did not form in the absence of T cell help, the PCs in the GK1.5 treated animals expanded comparably to those in a T cell replete rechallenged mouse (FIGS. 7A, 7B). To assess the isotype of the responding T-independent ASCs, intracellular Ig staining was again performed. In mice depleted of T cells, more than 85% of the Ig⁺CD138⁺ ASCs expressed IgM⁺ (FIG. 7C). Therefore, the formation of both unswitched and switched PBs is T cell dependent, yet predominantly IgM⁺ expressing PCs can still form in a T cell independent manner. These data therefore indicate that IgM⁺ MBCs can form two unique ASC populations in two mechanistically distinct ways, again highlighting their plasticity.

As demonstrated herein, how MBC subsets develop and function in response to infection with a relevant pathogen was studied. To accomplish this, B cell tetramers were generated and enrichment techniques utilized to perform analyses of endogenous Plasmodium-specific B cells in malaria-exposed humans and mice. Importantly, the results presented in these studies highlight the fact that IgM⁺ and IgD⁺ MBCs are unique populations of cells with distinct phenotypic, functional and survival properties. Furthermore these studies emphasize that IgM⁺ MBCs are not low affinity cells that provide redundancy to IgG⁺ MBCs. On the contrary, Plasmodium-specific IgM⁺ MBCs express high affinity, somatically hypermutated BCRs and rapidly respond to produce antibodies prior to IgG⁺ MBCs, even in competition. Lastly, these studies reveal that a secondary memory response results in the generation of T-dependent plasmablasts and T-independent plasma cells that create multiple layers of antibody secreting cells.

In many ways, the results described herein reconcile many of the disparate findings from various studies using a variety of protein immunization strategies, BCR transgenics and isolated transfer and rechallenge techniques. Dividing unswitched cells into two populations based on differential expression of IgM and IgD, revealed that IgM⁺ MBCs were far more similar in phenotype (CD73 and CD80 expression), developmental history (evidence of somatic hypermutation), affinity, survival and function (rapid plasmablast formation) to swIg⁺ MBCs than the more naïve-like IgD⁺ MBCs. Thus either isotype, as shown by Pape et al. (Pape et al., 2011) or expression of markers associated with somatic hypermutation (Zuccarino-Catania et al., 2014) can predict MBC function, reconciling the findings of these two separate studies. While the IgD⁺ MBCs were remarkably stable, both the IgM⁺ and swIg⁺ subsets persisted with similar, and less stable kinetics as predicted by studies demonstrating a loss of somatically hypermutated B cells overtime (Gitlin et al., 2016). Without wishing to be bound or limited by theory, IgD⁺ MBCs may represent a durable, expanded memory population that provides a high number of pathogen-specific clones with kinetics similar to naïve B cells.

It was also addressed how distinct antigen-specific MBC subsets respond to a secondary infection in vivo in competition and demonstrate a hierarchy of MBC responsiveness to secondary infection. Surprisingly, at the earliest timepoints, IgM⁺ MBCs are the dominant producers of ASCs at all doses of rechallenge and timepoints examined. By 5 days post-secondary infection however, IgM antibody production did not continue to increase, while switched PBs began to produce significant amounts of antibody highlighting that this dominance is transient. Therefore unlike previous studies indicating that IgM cells do not readily form PBs perhaps due to their low affinity (Pape et al., 2011) or form plasmablasts with similar kinetics to IgG⁺ PBs (Zuccarino-Catania et al., 2014), as shown herein, IgM⁺ MBCs are high affinity, rapid, plastic early responders that can initiate the secondary response.

The results described herein may explain recent data associating the depth and breadth of Plasmodium-specific IgM antibodies with resistance to infection (Arama et al., 2015). While it was demonstrated that F(ab)s made from IgM⁺ MBCs are of comparable affinity to those sequenced from IgG⁺ MBCs, upon pentamerization of IgM antibodies, the IgM⁺ antibody avidity would be far greater than the IgG antibodies. Moreover, IgM antibodies are important mediators of complement-mediated lysis, which is important for control of blood stage infection (Boyle et al., 2015). While the importance of IgM antibodies in Plasmodium infection has been shown in murine models (Couper et al., 2005), additional studies are being performed examining the importance of IgM antibodies in human malaria infection as well as the comparison of Plasmodium-specific IgM⁺ MBCs found in the murine systems described herein to those identified in malaria-exposed humans.

Finally, the studies described herein help to clarify long-standing controversies concerning the level of T cell dependence of secondary MBC responses (Kurosaki et al., 2015). Although many studies in humans and mice have demonstrated T cell-independent activation of MBCs (Bernasconi et al., 2002; Richard et al., 2008; Von Eschen and Rudbach, 1974), later studies suggested MBCs cannot be activated by bystander inflammation (Benson et al., 2009) or without the help of T cells (Ise et al., 2014). The results described herein demonstrate that both T-dependent and T-independent processes contribute to a secondary MBC response and support recent studies demonstrating that IgM⁺ MBCs can be reactivated in a T-independent manner when transferred in isolation into T-cell depleted mice (Zuccarino-Catania et al., 2014). Specifically, secondary IgM⁺ and IgG⁺ PB formation was T-dependent, while the rapid generation of non-dividing, antibody-secreting IgM⁺ PCs was T-independent, raising many questions about the origins of these cells. Without wishing to be bound or limited by theory, the murine somatically hypermutated IgM⁺ MBCs identified in these studies could be homologous to human IgM⁺ MBCs that can mediate T-independent IgM⁺ responses to bacterial infection (Weill et al., 2009). In conclusion, the studies described herein highlight IgM⁺ MBCs as a functional, plastic, rapidly responding MBC population that should be targeted by vaccines to prevent disease.

### Experimental Procedures

### Animals

5-8 week old female C57BL/6 and B6.SJL-Ptprc^{a} Pepc^{b}/BoyJ (CD45.1⁺) mice were used for these experiments. Mice were purchased from The Jackson Laboratory and maintained/bred under specific pathogen free conditions at the University of Washington. MD4-Rag2^{-/-} mice were provided. All experiments were performed in accordance with the University of Washington Institutional Care and Use Committee guidelines.

### Plasmodium Infection

Plasmodium chabaudi chabaudi (AS) parasites were maintained as frozen blood stocks and passaged through donor mice. Primary mouse infections were initiated by intraperitoneal (i.p.) injection of 1×10⁶ iRBCs from donor mice. Secondary mouse infections were performed 12-35 weeks after primary infection using a dose of 1×10⁷ iRBCs injected intravenously (i.v.). In some cases, when indicated, secondary challenges were given at lower doses using either 1×10³ or 1×10⁵ iRBCs injected i.v.

### Tetramer Production

For murine studies, recombinant His-tagged C-terminal MSP1 protein (amino acids 4960 to 5301) from P. chabaudi (AS) was produced by Pichia pastoris and purified using a Ni-NTA agarose column as previously described (Ndungu et al., 2009). Purified P.chabaudi MSP1 protein was biotinylated and tetramerized with streptavidin-PE (Prozyme) as previously described (Taylor et al., 2012a). For human studies, AMA1 protein from P.falciparum (3D 7) (provided by Dr. Julian Rayner, Welcome Trust Sanger Institute) and MSP1-19 protein from P.falciparum (3D 7) (provided by Dr. Anthony Holder, Francis Crick Institute) were biotinylated and tetramerized as described above. Decoy reagent to gate out non-MSP1⁺B cells was made by conjugating SA-PE to AF647 using an AF647 protein labeling kit (ThermoFisher), washing and removing any unbound AF647, and incubating with an excess of an irrelevant biotinylated HIS-tagged protein, similar to what has been previously described (Taylor et al., 2012a).

### Mouse and Human Cell Enrichment and Flow Cytometry

For murine samples, splenic cell suspensions were prepared and resuspended in 200 ul in PBS containing 2% FBS and Fc block (2.4G2) and first incubated with Decoy tetramer at a concentration of 10 nM at room temperature for 10 min. MSP1-PE tetramer was added at a concentration of 10 nM and incubated on ice for 30 min. Cells were washed, incubated with anti-PE magnetic beads for 30 min on ice, and passed over magnetized LS columns (Miltenyi Biotec) to elute the bound cells as previously described (Taylor et al., 2012a). For human samples, PBMC were similarly stained and enriched using Decoy, PfAMA1 and PfMSP1 tetramers. All bound cells were stained with surface antibodies followed by intracellular antibody staining when needed. All cells were run on the LSRII (BD) and analyzed using FlowJo software (Treestar).

### Single Cell BCR Sequencing and Cloning

Single MSP MBCs were FACS sorted using an ARIAII into 96-well plates. BCRs were amplified and sequenced from the cDNA of single cells as previously described (Schwartz et al., 2014), with additional IgH primers used (Tiller et al., 2009). Amplified products were cloned and generated mAbs using previously described methods (Schwartz et al., 2014; Tiller et al., 2009).

### ELISAs

Costar 96-well EIA/RIA plates (Fisher Scientific) were coated overnight at 4° C. with 10 ug/ml of MSP1 protein. Plates were blocked with 2% BSA prior to sample incubation. For serum samples, plates were incubated with serially diluted serum from naïve or infected animals. For cloned mAbs, plates were incubated with serially diluted mAbs starting at 10 ng/W. Each sample was plated in duplicate. For serum samples, bound antibodies were detected using either IgM Biotin (II/41), IgG Biotin (Poly4053), IgG1 Biotin (A85-1), IgG2c Biotin (5.7), IgG2b Biotin (R123), or IgG3 (R40-82) followed by Streptavidin-HRP (BD). For mAbs, bound antibodies were detected with mouse anti-human IgG-HRP (SouthernBiotech). Absorbance was measured at 450 nm using an IMARK Microplate Reader (Bio-Rad). ELISPOT

96 well ELISPOT plates (Millipore) were coated overnight at 4° C. with 10 ug/ml of Ig(H+L) unlabeled antibody (Southern Biotech). Plates were blocked with 10% FBS in complete DMEM (Gibco). MSP1⁺ MBCs were sorted using a FACSARIA (BD) from memory mice 2 days after rechallenge. Cells of each MBC population were plated onto coated ELISPOT plates and incubated at 37° C. for an additional 2.5 days. Cells were washed off and secreted antibodies were detected using either IgM Biotin (II/41) or IgG Biotin (Poly4053) followed by Streptavidin-HRP (BD). Nonspecific (background) spots were determined in wells containing no cells. Spots were developed using AEC substrate (BD) and counted and analyzed using the CU ELISPOT reader and Immunospot analysis software (Cellular Technology Limited). Number of spots detected per well were used to calculate spot frequency per 1×10⁵ total cells.

### Depletion of CD4+ T Cells

For depletion of CD4⁺ T cells, GK1.5 monoclonal antibody to CD4 (rIgG2b; BioXcell) was used. One and two days prior to secondary challenge, memory mice were given an i.p. injection of 200 μg GK1.5 or isotype control diluted in PBS. Efficiency of CD4⁺ T cell depletion was monitored by checking blood of mice pre-depletion, day 1 post injection and day of challenge. Depletion was found to be greater than 98% of CD4⁺ T cells as assessed by a non-GK1.5 competing anti-CD4 clone, RM4-4.

### Statistical Analysis

Unpaired, two-tailed Student's t tests were applied to determine the statistical significance of the differences between groups with Prism (Graphpad) software. The p-values were considered significant when p<0.05 (*), p<0.01 (**), and p<0.001 (***).

### Parasitemia by Flow Cytometry

Parasitemia was measured by flow cytometry by staining 1 ul of blood with Ter119 APC eFluor780 (eBioscience), CD45 APC (BD), Hoechst33342 (Sigma), and Dihydroethidium (Sigma). Giemsa staining of thin blood smears was done in parallel.

### Human PBMC Samples

Deidentified Plasmodium-infected PBMC samples are from a cohort in Mali previously described. (Crompton et al., 2008). Uninfected control PBMC are from healthy U.S. adult donors enrolled in NIH protocol #99-CC-0168. Demographic and travel history data were not available from the anonymous U.S. donors, but prior P.falciparum exposure is unlikely.

### Immunofluorescence Staining of Spleens

Spleens from infected mice were embedded in OCT and flash frozen. 8 um sections were cut and fixed in acetone and then stained with CD4 Biotin (RM4-5), B220 Alexa Fluor 647 (RA3-6B2), and IgD Alexa Fluor 488 (11-26c.2a). Streptavidin Cy3 (Jackson Immunoresearch) was used as a secondary antibody. Images were acquired using a Nikon Eclipse 90i microscope and NIS Elements BR (Build 738) software was used for the capture of individual images for each channel Raw TIFF files were imported in Adobe Photoshop for overlay of single channel images and editing.

### Example 2

### Summary for Recombinant Human Malaria Specific Monoclonal Antibodies.

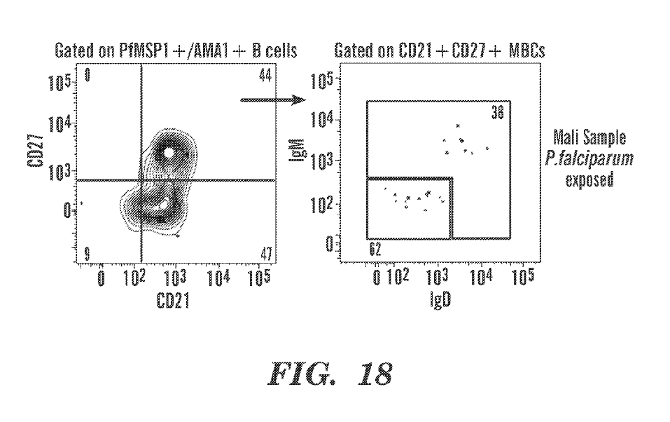

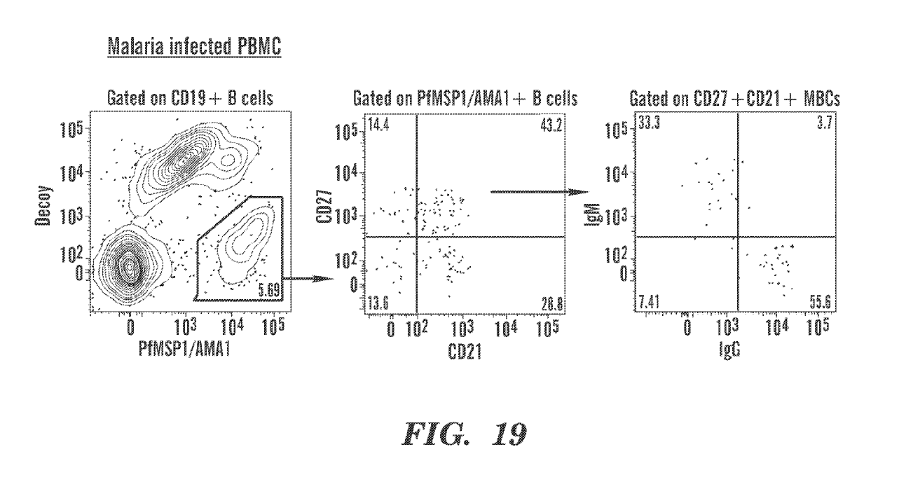

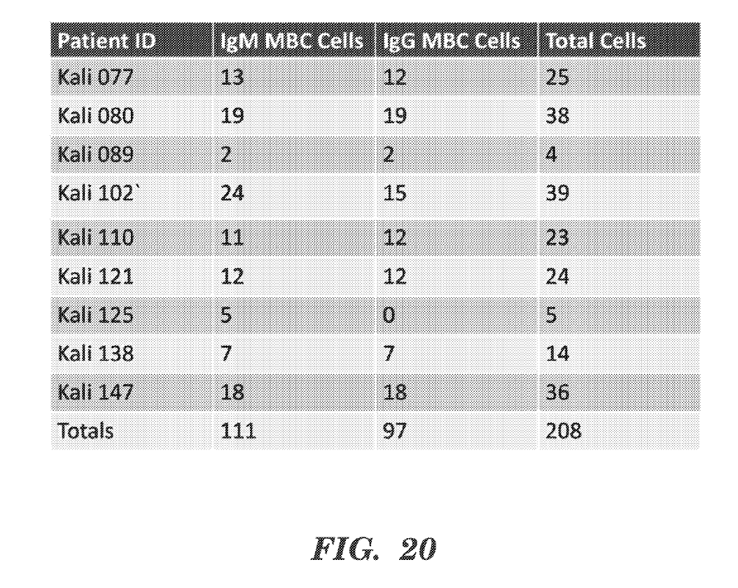

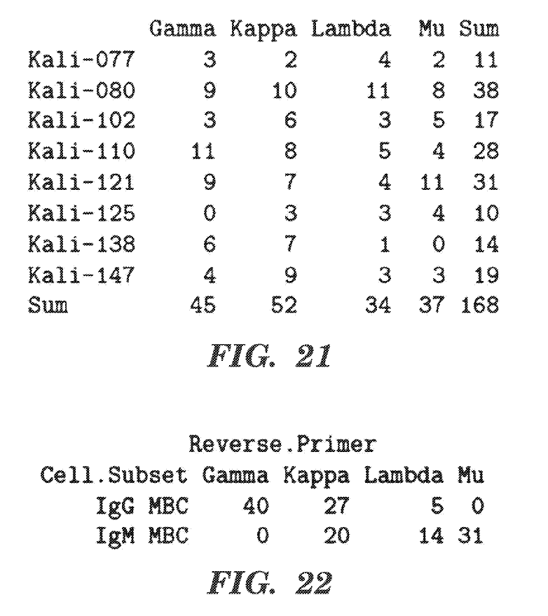

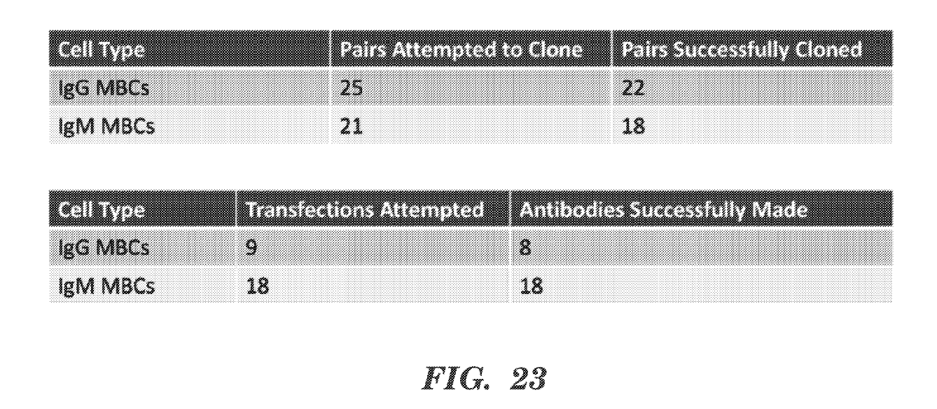

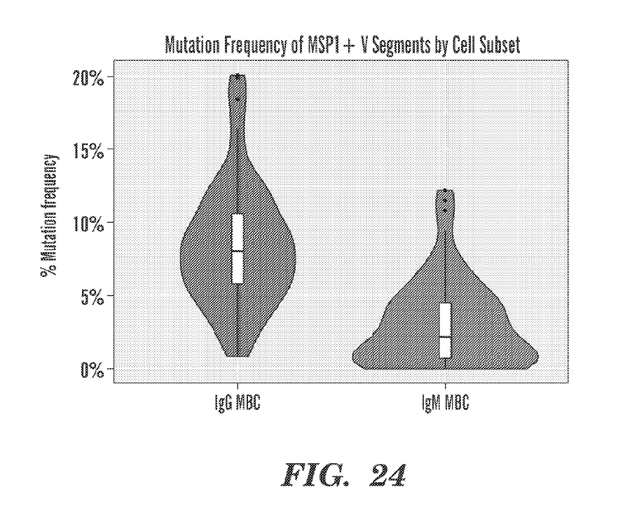

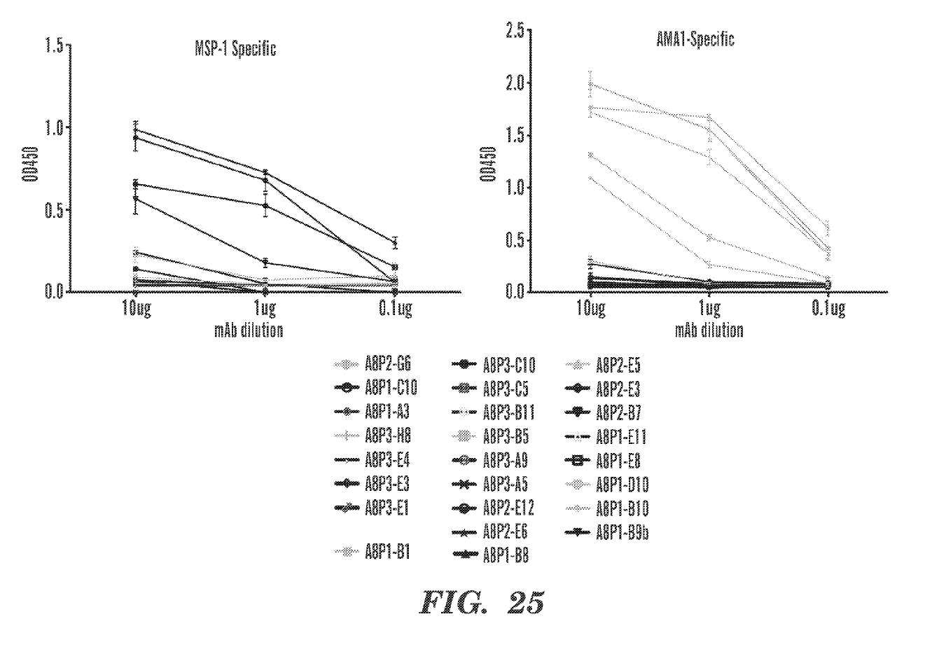

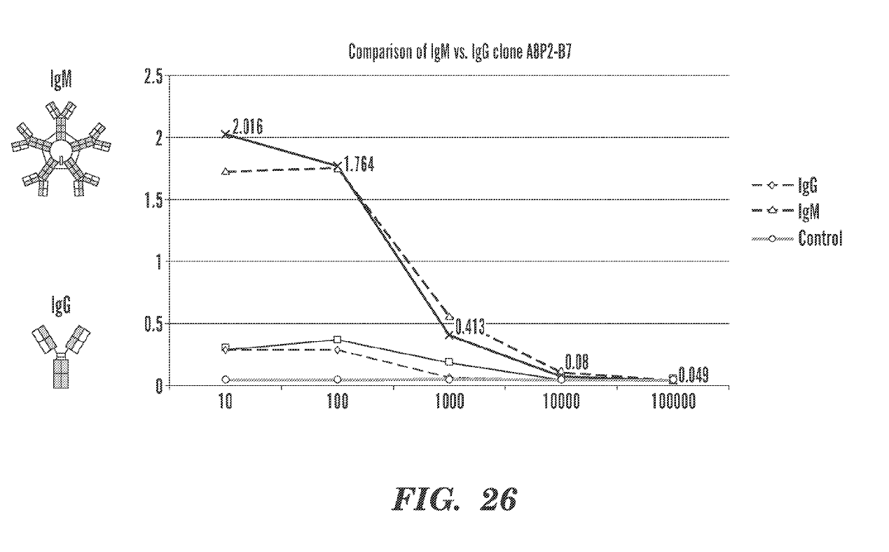

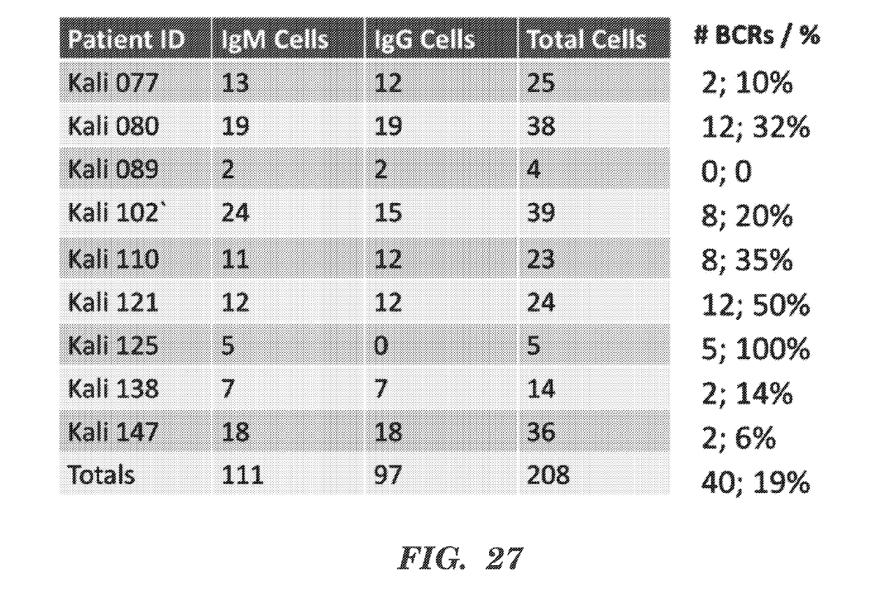

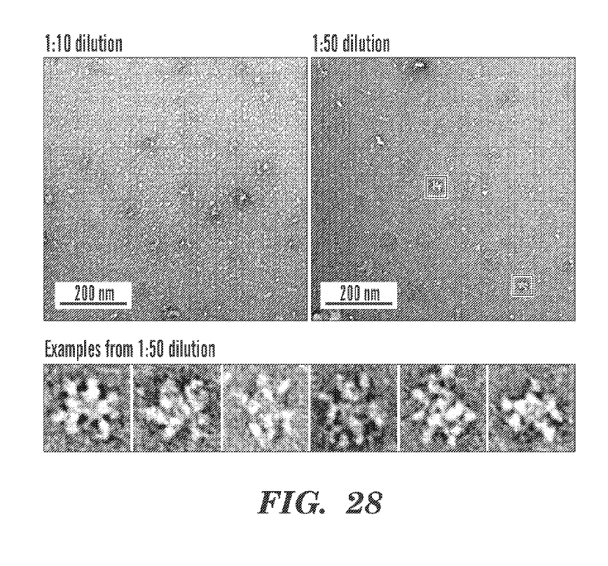

As shown in FIGS. 18-26, Plasmodium specific (AMA/MSP1 Tmr-based) FACS sorting yielded 208 (111 IgM and 97 IgG) MBCs from 9 donors (4-39 totals cells/donor). Sequence data was obtained for 168 distinct HC or LCs (from 8/9 donors; 10-38 per/donor). Full BCRs were cloned for 40 MBCs, 22 IgM and 18 IgG, and these BCRs were derived from 8/9 donors. Recombinant mAbs were expressed from 27 BCR clones, out of which 26 of the 27 expressed. The obtained mAbs were next tested by ELISA, and 20/26 were ELISA-positive for AMA or MSP1 with the positive clones obtained from 8/9 donors, ranging from 2-12 mAbs/donor. Overall yield for BCRs was 40/208 sorted MBCs=19%, with majority reactive to one of the two malarial antigens used for sorting.

### BCR Cloning Methods

Received nine plates with frozen single cells sorted partially into each plate. Created cDNA libraries using FERMENTAS MAXIMA First Strand Kit. Performed four separate PCR reactions to amplify heavy-gamma, heavy-mu, lambda, and kappa chains. Visualized products on gels. Purified and Sanger sequenced amplicons in forward and reverse.

### Analysis Methods

Quality control was performed by trimming all sequence ends with more than 1% chance of error per base. Contigs were created with forward and reverse sequences. All contigs were aligned to the human IMGT database using NCBI IgBLAST. Recorded V and J genes, functionality, and % homology to germline. Exploratory data analysis done in R.

### Sequence Summary Tables

TABLE 1
Variable Heavy Chain Segment Usage by Cell Subset

| | V.Segment | | | | | | |
|---|---|---|---|---|---|---|---|
| Cell.Subset | IGHV1-2 | IGHV1-46 | IGHV1-69 | IGHV1-8 | IGHV3-11 | IGHV3-15 | IGHV3-23 |
| IgG MBC | 2 | 0 | 0 | 1 | 0 | 6 | 0 |
| IgM MBC | 0 | 1 | 1 | 3 | 1 | 1 | 1 |

| | V.Segment | | | | | |
|---|---|---|---|---|---|---|
| Cell.Subset | IGHV3-23D | IGHV3-30 | IGHV3-30-3 | IGHV3-30-5 | IGHV3-33 | IGHV3-48 |
| IgG MBC | 1 | 2 | 0 | 1 | 1 | 1 |
| IgM MBC | 0 | 0 | 1 | 0 | 0 | 1 |

| | V.Segment | | | | | |
|---|---|---|---|---|---|---|
| Cell.Subset | IGHV3-7 | IGHV3-74 | IGHV3-9 | IGHV4-30-4 | IGHV4-31 | IGHV4-34 |
| IgG MBC | 0 | 0 | 6 | 2 | 0 | 4 |
| IgM MBC | 1 | 1 | 0 | 0 | 1 | 7 |

| | V.Segment | | | | | |
|---|---|---|---|---|---|---|
| Cell.Subset | IGHV4-38-2 | IGHV4-39 | IGHV4-4 | IGHV4-59 | IGHV4-61 | IGHV5-51 |
| IgG MBC | 3 | 3 | 1 | 2 | 3 | 1 |
| IgM MBC | 2 | 4 | 0 | 1 | 3 | 1 |

TABLE 2
IgM Cell Subset JH Segment Usage by VH Segment Usage

| | J.segment | | | | | |
|---|---|---|---|---|---|---|
| V.Seg- | | | | IGHJ4 or | | |
| ment | IGHJ1 | IGHJ3 | IGHJ4 | IGHJ5 | IGHJ5 | IGHJ6 |
| IGHV1-46 | 0 | 0 | 1 | 0 | 0 | 0 |
| IGHV1-69 | 0 | 0 | 1 | 0 | 0 | 0 |
| IGHV1-8 | 0 | 0 | 0 | 0 | 0 | 3 |
| IGHV3-11 | 0 | 0 | 1 | 0 | 0 | 0 |
| IGHV3-15 | 0 | 1 | 0 | 0 | 0 | 0 |
| IGHV3-23 | 0 | 0 | 1 | 0 | 0 | 0 |
| IGHV3-30-3 | 0 | 1 | 0 | 0 | 0 | 0 |
| IGHV3-48 | 0 | 0 | 1 | 0 | 0 | 0 |
| IGHV3-7 | 0 | 0 | 0 | 0 | 0 | 1 |
| IGHV3-74 | 0 | 0 | 0 | 0 | 0 | 1 |
| IGHV4-31 | 0 | 1 | 0 | 0 | 0 | 0 |
| IGHV4-34 | 0 | 3 | 4 | 0 | 0 | 0 |
| IGHV4-38-2 | 0 | 0 | 0 | 0 | 2 | 0 |
| IGHV4-39 | 0 | 0 | 1 | 1 | 1 | 1 |
| IGHV4-59 | 0 | 0 | 1 | 0 | 0 | 0 |
| IGHV4-61 | 0 | 0 | 1 | 0 | 1 | 1 |
| IGHV5-51 | 1 | 0 | 0 | 0 | 0 | 0 |

TABLE 3
IgG Cell Subset JH Segment Usage by VH Segment Usage

| | J.segment | | | | | | |
|---|---|---|---|---|---|---|---|
| V.Segment | IGHJ1 | IGHJ2 | IGHJ3 | IGHJ4 | IGHJ4 or IGHJ5 | IGHJ5 | IGHJ6 |
| IGHV1-2 | 0 | 0 | 0 | 0 | 0 | 0 | 2 |
| IGHV1-8 | 0 | 0 | 0 | 1 | 0 | 0 | 0 |
| IGHV3-15 | 0 | 0 | 0 | 6 | 0 | 0 | 0 |
| IGHV3-23D | 0 | 0 | 0 | 1 | 0 | 0 | 0 |
| IGHV3-30 | 0 | 0 | 0 | 2 | 0 | 0 | 0 |
| IGHV3-30-5 | 0 | 0 | 0 | 1 | 0 | 0 | 0 |
| IGHV3-33 | 0 | 0 | 1 | 0 | 0 | 0 | 0 |
| IGHV3-48 | 0 | 0 | 0 | 0 | 1 | 0 | 0 |
| IGHV3-9 | 0 | 2 | 0 | 2 | 0 | 1 | 1 |
| IGHV4-30-4 | 0 | 0 | 0 | 1 | 0 | 1 | 0 |
| IGHV4-34 | 0 | 0 | 0 | 1 | 2 | 1 | 0 |
| IGHV4-38-2 | 0 | 0 | 0 | 3 | 0 | 0 | 0 |
| IGHV4-39 | 0 | 0 | 0 | 3 | 0 | 0 | 0 |
| IGHV4-4 | 0 | 0 | 0 | 0 | 0 | 0 | 1 |
| IGHV4-59 | 0 | 0 | 0 | 2 | 0 | 0 | 0 |
| IGHV4-61 | 0 | 0 | 0 | 2 | 1 | 0 | 0 |
| IGHV5-51 | 1 | 0 | 0 | 0 | 0 | 0 | 0 |

TABLE 4
VH Usage by Individual for IgM MBCs

| | cDNA.plate | | | | | | |
|---|---|---|---|---|---|---|---|
| | Kali- | Kali- | Kali- | Kali- | Kali- | Kali- | Kali- |
| V.Segment | 077 | 080 | 102 | 110 | 121 | 125 | 147 |
| IGHV1-46 | 0 | 0 | 0 | 0 | 0 | 1 | 0 |
| IGHV1-69 | 0 | 1 | 0 | 0 | 0 | 0 | 0 |
| IGHV1-8 | 0 | 1 | 0 | 1 | 1 | 0 | 0 |
| IGHV3-11 | 0 | 0 | 0 | 0 | 1 | 0 | 0 |
| IGHV3-15 | 0 | 0 | 0 | 1 | 0 | 0 | 0 |
| IGHV3-23 | 0 | 0 | 0 | 0 | 1 | 0 | 0 |
| IGHV3-30-3 | 0 | 1 | 0 | 0 | 0 | 0 | 0 |
| IGHV3-48 | 0 | 0 | 0 | 0 | 0 | 1 | 0 |
| IGHV3-7 | 1 | 0 | 0 | 0 | 0 | 0 | 0 |
| IGHV3-74 | 0 | 0 | 0 | 0 | 1 | 0 | 0 |
| IGHV4-31 | 0 | 0 | 0 | 0 | 1 | 0 | 0 |
| IGHV4-34 | 0 | 0 | 4 | 0 | 1 | 0 | 2 |
| IGHV4-38-2 | 0 | 1 | 0 | 0 | 1 | 0 | 0 |
| IGHV4-39 | 1 | 1 | 1 | 1 | 0 | 0 | 0 |
| IGHV4-59 | 0 | 0 | 0 | 0 | 0 | 1 | 0 |
| IGHV4-61 | 0 | 2 | 0 | 0 | 0 | 1 | 0 |
| IGHV5-51 | 0 | 0 | 0 | 0 | 0 | 0 | 1 |

TABLE 5
VH Usage by Individual for IgG MBCs

| | cDNA.plate | | | | | | |
|---|---|---|---|---|---|---|---|
| | Kali- | Kali- | Kali- | Kali- | Kali- | Kali- | Kali- |
| V.Segment | 077 | 080 | 102 | 110 | 121 | 138 | 147 |
| IGHV1-2 | 0 | 0 | 0 | 2 | 0 | 0 | 0 |
| IGHV1-8 | 0 | 1 | 0 | 0 | 0 | 0 | 0 |
| IGHV3-15 | 0 | 0 | 0 | 4 | 2 | 0 | 0 |
| IGHV3-23D | 0 | 1 | 0 | 0 | 0 | 0 | 0 |
| IGHV3-30 | 0 | 0 | 0 | 0 | 2 | 0 | 0 |
| IGHV3-30-5 | 0 | 0 | 0 | 0 | 0 | 0 | 1 |
| IGHV3-33 | 0 | 1 | 0 | 0 | 0 | 0 | 0 |
| IGHV3-48 | 0 | 0 | 0 | 1 | 0 | 0 | 0 |
| IGHV3-9 | 0 | 3 | 0 | 0 | 0 | 3 | 0 |
| IGHV4-30-4 | 0 | 0 | 0 | 0 | 1 | 0 | 1 |
| IGHV4-34 | 0 | 0 | 0 | 3 | 0 | 1 | 0 |
| IGHV4-38-2 | 0 | 0 | 0 | 1 | 0 | 2 | 0 |
| IGHV4-39 | 1 | 0 | 0 | 0 | 2 | 0 | 0 |
| IGHV4-4 | 1 | 0 | 0 | 0 | 0 | 0 | 0 |
| IGHV4-59 | 0 | 0 | 2 | 0 | 0 | 0 | 0 |
| IGHV4-61 | 0 | 3 | 0 | 0 | 0 | 0 | 0 |
| IGHV5-51 | 1 | 0 | 0 | 0 | 0 | 0 | 0 |

TABLE 6
Variable Kappa Chain Segment Usage by Cell Subset

| | V.Segment | | | | | | |
|---|---|---|---|---|---|---|---|
| Cell.Subset | IGKV1-12 | IGKV1-39 | IGKV1-5 | IGKV1-8 | IGKV1D-33 | IGKV2-28 | IGKV2-30 |
| IgG MBC | 1 | 0 | 7 | 1 | 1 | 0 | 5 |
| IgM MBC | 0 | 3 | 4 | 0 | 0 | 1 | 0 |

| | V.Segment | | | | | |
|---|---|---|---|---|---|---|
| Cell.Subset | IGKV2D-29 | IGKV3-11 | IGKV3-15 | IGKV3-20 | IGKV3D-20 | IGKV4-1 |
| IgG MBC | 1 | 0 | 3 | 4 | 0 | 4 |
| IgM MBC | 0 | 1 | 1 | 5 | 1 | 4 |

TABLE 7
IgM Cell Subset JK Segment Usage by VK Segment Usage

J.segment

| V.Segment | IGKJ1 | IGKJ2 | IGKJ3 | IGKJ4 |
|---|---|---|---|---|
| IGKV1-39 | 0 | 2 | 1 | 0 |
| IGKV1-5 | 1 | 2 | 0 | 1 |
| IGKV2-28 | 0 | 1 | 0 | 0 |
| IGKV3-11 | 0 | 0 | 0 | 1 |
| IGKV3-15 | 1 | 0 | 0 | 0 |
| IGKV3-20 | 1 | 4 | 0 | 0 |
| IGKV3D-20 | 0 | 1 | 0 | 0 |
| IGKV4-1 | 3 | 1 | 0 | 0 |

TABLE 8
IgG Cell Subset JK Segment Usage by VK Segment Usage

| | J.segment | | | | |
|---|---|---|---|---|---|
| V.Segment | IGKJ1 | IGKJ2 | IGKJ3 | IGKJ4 | IGKJ5 |
| IGKV1-12 | 0 | 0 | 0 | 1 | 0 |
| IGKV1-5 | 4 | 3 | 0 | 0 | 0 |
| IGKV1-8 | 1 | 0 | 0 | 0 | 0 |
| IGKV1D-33 | 0 | 1 | 0 | 0 | 0 |
| IGKV2-30 | 0 | 0 | 4 | 0 | 1 |
| IGKV2D-29 | 0 | 0 | 0 | 1 | 0 |
| IGKV3-15 | 1 | 0 | 2 | 0 | 0 |
| IGKV3-20 | 1 | 1 | 0 | 1 | 1 |
| IGKV4-1 | 1 | 0 | 0 | 3 | 0 |

TABLE 9
VK Usage by Individual for IgM MBCs

| | cDNA.plate | | | | | | |
|---|---|---|---|---|---|---|---|
| | Kali- | Kali- | Kali- | Kali- | Kali- | Kali- | Kali- |
| V.Segment | 080 | 102 | 110 | 121 | 125 | 138 | 147 |
| IGKV1-39 | 1 | 0 | 0 | 0 | 0 | 2 | 0 |
| IGKV1-5 | 0 | 0 | 1 | 1 | 2 | 0 | 0 |
| IGKV2-28 | 0 | 0 | 1 | 0 | 0 | 0 | 0 |
| IGKV3-11 | 1 | 0 | 0 | 0 | 0 | 0 | 0 |
| IGKV3-15 | 0 | 0 | 0 | 0 | 1 | 0 | 0 |
| IGKV3-20 | 0 | 0 | 0 | 3 | 0 | 1 | 1 |
| IGKV3D-20 | 0 | 0 | 0 | 0 | 0 | 1 | 0 |
| IGKV4-1 | 0 | 3 | 0 | 1 | 0 | 0 | 0 |

TABLE 10
VK Usage by Individual for IgG MBCs

| | cDNA.plate | | | | | | |
|---|---|---|---|---|---|---|---|
| | Kali- | Kali- | Kali- | Kali- | Kali- | Kali- | Kali- |
| V.Segment | 077 | 080 | 102 | 110 | 121 | 138 | 147 |
| IGKV1-12 | 1 | 0 | 0 | 0 | 0 | 0 | 0 |
| IGKV1-5 | 0 | 4 | 0 | 3 | 0 | 0 | 0 |
| IGKV1-8 | 0 | 1 | 0 | 0 | 0 | 0 | 0 |
| IGKV1D-33 | 0 | 0 | 1 | 0 | 0 | 0 | 0 |
| IGKV2-30 | 0 | 0 | 0 | 3 | 0 | 0 | 2 |
| IGKV2D-29 | 0 | 0 | 0 | 0 | 0 | 0 | 1 |
| IGKV3-15 | 0 | 0 | 2 | 0 | 0 | 0 | 1 |
| IGKV3-20 | 1 | 1 | 0 | 0 | 0 | 1 | 1 |
| IGKV4-1 | 0 | 1 | 0 | 0 | 1 | 2 | 0 |

TABLE 11
Variable Lambda Chain Segment Usage by Cell Subset

| | V.Segment | | | | | | |
|---|---|---|---|---|---|---|---|
| Cell.Subset | IGLV1-44 | IGLV1-47 | IGLV1-51 | IGLV2-11 | IGLV2-14 | IGLV2-23 | IGLV2-8 |
| IgG MBC | 0 | 1 | 0 | 0 | 1 | 0 | 0 |
| IgM MBC | 2 | 1 | 2 | 1 | 0 | 1 | 1 |

| | V.Segment | | | | |
|---|---|---|---|---|---|
| Cell.Subset | IGLV3-21 | IGLV3-9 | IGLV4-69 | IGLV6-57 | IGLV8-61 |
| IgG MBC | 0 | 2 | 1 | 0 | 0 |
| IgM MBC | 1 | 0 | 1 | 3 | 1 |

TABLE 12
IgM Cell Subset JL Segment Usage by VL Segment Usage

| | J.segment | | |
|---|---|---|---|
| V.Segment | IGLJ1 | IGLJ2 or IGLJ3 | IGLJ3 |
| IGLV1-44 | 0 | 0 | 2 |
| IGLV1-47 | 0 | 0 | 1 |
| IGLV1-51 | 0 | 1 | 1 |
| IGLV2-11 | 1 | 0 | 0 |
| IGLV2-23 | 0 | 0 | 1 |
| IGLV2-8 | 0 | 1 | 0 |
| IGLV3-21 | 0 | 0 | 1 |
| IGLV4-69 | 0 | 1 | 0 |
| IGLV6-57 | 2 | 1 | 0 |
| IGLV8-61 | 0 | 1 | 0 |

TABLE 13
IgG Cell Subset JL Segment Usage by VL Segment Usage

J.segment

| V.Segment | IGLJ2 or IGLJ3 | IGLJ3 |
|---|---|---|
| IGLV1-47 | 0 | 1 |
| IGLV2-14 | 0 | 1 |
| IGLV3-9 | 2 | 0 |
| IGLV4-69 | 1 | 0 |

TABLE 14
VL Usage by Individual for IgM MBCs

| | cDNA.plate | | | | |
|---|---|---|---|---|---|
| V.Segment | Kali-080 | Kali-102 | Kali-110 | Kali-121 | Kali-147 |
| IGLV1-44 | 0 | 1 | 0 | 0 | 1 |
| IGLV1-47 | 0 | 0 | 0 | 0 | 1 |
| IGLV1-51 | 1 | 0 | 0 | 0 | 1 |
| IGLV2-11 | 1 | 0 | 0 | 0 | 0 |
| IGLV2-23 | 0 | 0 | 0 | 1 | 0 |
| IGLV2-8 | 0 | 0 | 1 | 0 | 0 |
| IGLV3-21 | 1 | 0 | 0 | 0 | 0 |
| IGLV4-69 | 0 | 0 | 0 | 1 | 0 |
| IGLV6-57 | 0 | 1 | 2 | 0 | 0 |
| IGLV8-61 | 1 | 0 | 0 | 0 | 0 |

TABLE 15
VL Usage by Individual for IgG MBCs

| | cDNA.plate | | |
|---|---|---|---|
| V.Segment | Kali-080 | Kali-102 | Kali-121 |
| IGLV1-47 | 0 | 1 | 0 |
| IGLV2-14 | 1 | 0 | 0 |
| IGLV3-9 | 0 | 0 | 2 |
| IGLV4-69 | 1 | 0 | 0 |

TABLE 16
Light Chain Usage by Cell Subset

Cell.Subset

| Chain | IgG MBC | IgM MBC |
|---|---|---|
| Kappa | 27 | 20 |
| Lambda | 5 | 14 |

TABLE 17
Mutation Rates (%) by Cell Subset and Chain
A tibble: 6 × 4
Groups: Cell.Subset [?]

| | Cell.Subset | Chain | Count | Mean.V.Mutation.Rate |
|---|---|---|---|---|
| | <fctr> | <fctr> | <int> | <dbl> |
| 1 | IgG MBC | Heavy | 40 | 10.285000 |
| 2 | IgG MBC | Kappa | 27 | 6.325926 |
| 3 | IgG MBC | Lambda | 5 | 4.740000 |
| 4 | IgM MBC | Heavy | 31 | 2.635484 |
| 5 | IgM MBC | Kappa | 20 | 2.645000 |
| 6 | IgM MBC | Lambda | 14 | 4.007143 |

TABLE 18
Mutation Rates (%) by Individual and Cell Subset
# A tibble: 15 × 5
# Groups: cDNA.plate [?]

| | | | | Mean.V.Mu- | |
|---|---|---|---|---|---|
| | cDNA.plate | Cell.Subset | Count | tation.Rate | SD.V.Mut.Rate |
| | <fctr> | <fctr> | <int> | <dbl> | <dbl> |
| 1 | Kali-077 | IgG MBC | 5 | 6.06000000 | 3.5990276 |
| 2 | Kali-077 | IgM MBC | 2 | 2.25000000 | 3.1819805 |
| 3 | Kali-080 | IgG MBC | 18 | 7.68333333 | 2.8226500 |
| 4 | Kali-080 | IgM MBC | 13 | 0.07692308 | 0.2047513 |
| 5 | Kali-102 | IgG MBC | 6 | 10.43333333 | 5.9634442 |
| 6 | Kali-102 | IgM MBC | 10 | 2.52000000 | 1.7806366 |
| 7 | Kali-110 | IgG MBC | 17 | 7.91764706 | 3.1800620 |
| 8 | Kali-110 | IgM MBC | 8 | 7.08750000 | 3.9678845 |
| 9 | Kali-121 | IgG MBC | 10 | 7.98000000 | 3.0684053 |
| 10 | Kali-121 | IgM MBC | 14 | 2.28571429 | 2.0635508 |
| 11 | Kali-125 | IgM MBC | 7 | 3.72857143 | 2.8726958 |
| 12 | Kali-138 | IgG MBC | 9 | 13.82222222 | 4.8640975 |
| 13 | Kali-138 | IgM MBC | 4 | 3.40000000 | 2.4372115 |
| 14 | Kali-147 | IgG MBC | 7 | 5.12857143 | 3.3179885 |
| 15 | Kali-147 | IgM MBC | 7 | 4.51428571 | 1.8068652 |

TABLE 19
Mutation Rates (%) by Individual and Cell Subset and Chain

| | cDNA.plate | Chain | Cell.Subset | Count | Mean.V.Mutation.Rate | SD.V.Mut.Rate |
|---|---|---|---|---|---|---|
| 1 | Kali-077 | Heavy | IgG MBC | 3 | 6.86666667 | 3.8423083 |
| 2 | Kali-077 | Heavy | IgM MBC | 2 | 2.25000000 | 3.1819805 |
| 3 | Kali-077 | Kappa | IgG MBC | 2 | 4.85000000 | 4.1719300 |
| 4 | Kali-080 | Heavy | IgG MBC | 9 | 8.74444444 | 3.1436886 |
| 5 | Kali-080 | Heavy | IgM MBC | 7 | 0.04285714 | 0.1133893 |
| 6 | Kali-080 | Kappa | IgG MBC | 7 | 6.78571429 | 2.3954322 |
| 7 | Kali-080 | Kappa | IgM MBC | 2 | 0.00000000 | 0.0000000 |
| 8 | Kali-080 | Lambda | IgG MBC | 2 | 6.05000000 | 0.9192388 |
| 9 | Kali-080 | Lambda | IgM MBC | 4 | 0.17500000 | 0.3500000 |
| 10 | Kali-102 | Heavy | IgG MBC | 2 | 16.20000000 | 5.2325902 |
| 11 | Kali-102 | Heavy | IgM MBC | 5 | 2.66000000 | 2.2963014 |
| 12 | Kali-102 | Kappa | IgG MBC | 3 | 8.93333333 | 3.7220066 |
| 13 | Kali-102 | Kappa | IgM MBC | 3 | 3.16666667 | 1.1239810 |
| 14 | Kali-102 | Lambda | IgG MBC | 1 | 3.40000000 | NA |
| 15 | Kali-102 | Lambda | IgM MBC | 2 | 1.20000000 | 0.2828427 |
| 16 | Kali-110 | Heavy | IgG MBC | 11 | 8.77272727 | 3.6003030 |
| 17 | Kali-110 | Heavy | IgM MBC | 3 | 5.20000000 | 2.3895606 |
| 18 | Kali-110 | Kappa | IgG MBC | 6 | 6.35000000 | 1.3707662 |
| 19 | Kali-110 | Kappa | IgM MBC | 2 | 3.30000000 | 0.1414214 |
| 20 | Kali-110 | Lambda | IgM MBC | 3 | 11.50000000 | 0.7000000 |
| 21 | Kali-121 | Heavy | IgG MBC | 7 | 9.30000000 | 2.4785749 |
| 22 | Kali-121 | Heavy | IgM MBC | 7 | 2.27142857 | 2.3178397 |
| 23 | Kali-121 | Kappa | IgG MBC | 1 | 6.50000000 | NA |
| 24 | Kali-121 | Kappa | IgM MBC | 5 | 2.52000000 | 2.3466998 |
| 25 | Kali-121 | Lambda | IgG MBC | 2 | 4.10000000 | 1.8384776 |
| 26 | Kali-121 | Lambda | IgM MBC | 2 | 1.75000000 | 0.4949747 |
| 27 | Kali-125 | Heavy | IgM MBC | 4 | 4.45000000 | 3.7027017 |
| 28 | Kali-125 | Kappa | IgM MBC | 3 | 2.76666667 | 1.3279056 |
| 29 | Kali-138 | Heavy | IgG MBC | 6 | 16.61666667 | 2.9116433 |
| 30 | Kali-138 | Kappa | IgG MBC | 3 | 8.23333333 | 1.7785762 |
| 31 | Kali-138 | Kappa | IgM MBC | 4 | 3.40000000 | 2.4372115 |
| 32 | Kali-147 | Heavy | IgG MBC | 2 | 9.20000000 | 0.5656854 |
| 33 | Kali-147 | Heavy | IgM MBC | 3 | 4.76666667 | 1.5821926 |
| 34 | Kali-147 | Kappa | IgG MBC | 5 | 3.50000000 | 2.1977261 |
| 35 | Kali-147 | Kappa | IgM MBC | 1 | 2.30000000 | NA |
| 36 | Kali-147 | Lambda | IgM MBC | 3 | 5.00000000 | 2.0952327 |

TABLE 20
Mutation Rates (%) by VH Segment and Individual

| | V.Segment | cDNA.plate | Count | Mean.V.Mutation.Rate |
|---|---|---|---|---|
| 1 | IGHV1-2 | Kali-110 | 2 | 7.300000 |
| 2 | IGHV1-46 | Kali-125 | 1 | 9.500000 |
| 3 | IGHV1-69 | Kali-080 | 1 | 0.300000 |
| 4 | IGHV1-8 | Kali-080 | 2 | 4.400000 |
| 5 | IGHV1-8 | Kali-110 | 1 | 7.800000 |
| 6 | IGHV1-8 | Kali-121 | 1 | 0.700000 |
| 7 | IGHV3-11 | Kali-121 | 1 | 1.000000 |
| 8 | IGHV3-15 | Kali-110 | 5 | 10.540000 |
| 9 | IGHV3-15 | Kali-121 | 2 | 6.350000 |
| 10 | IGHV3-23 | Kali-121 | 1 | 3.400000 |
| 11 | IGHV3-23D | Kali-080 | 1 | 7.500000 |
| 12 | IGHV3-30 | Kali-121 | 2 | 11.900000 |
| 13 | IGHV3-30-3 | Kali-080 | 1 | 0.000000 |
| 14 | IGHV3-30-5 | Kali-147 | 1 | 8.800000 |
| 15 | IGHV3-33 | Kali-080 | 1 | 9.800000 |
| 16 | IGHV3-48 | Kali-110 | 1 | 8.200000 |
| 17 | IGHV3-48 | Kali-125 | 1 | 0.700000 |
| 18 | IGHV3-7 | Kali-077 | 1 | 4.500000 |
| 19 | IGHV3-74 | Kali-121 | 1 | 7.100000 |
| 20 | IGHV3-9 | Kali-080 | 3 | 6.033333 |
| 21 | IGHV3-9 | Kali-138 | 3 | 14.266667 |
| 22 | IGHV4-30-4 | Kali-121 | 1 | 9.100000 |
| 23 | IGHV4-30-4 | Kali-147 | 1 | 9.600000 |
| 24 | IGHV4-31 | Kali-121 | 1 | 1.700000 |
| 25 | IGHV4-34 | Kali-102 | 4 | 3.000000 |
| 26 | IGHV4-34 | Kali-110 | 3 | 8.233333 |
| 27 | IGHV4-34 | Kali-121 | 1 | 1.000000 |
| 28 | IGHV4-34 | Kali-138 | 1 | 18.400000 |
| 29 | IGHV4-34 | Kali-147 | 2 | 4.950000 |
| 30 | IGHV4-38-2 | Kali-080 | 1 | 0.000000 |
| 31 | IGHV4-38-2 | Kali-110 | 1 | 1.000000 |
| 32 | IGHV4-38-2 | Kali-121 | 1 | 1.000000 |
| 33 | IGHV4-38-2 | Kali-138 | 2 | 19.250000 |
| 34 | IGHV4-39 | Kali-077 | 2 | 1.800000 |
| 35 | IGHV4-39 | Kali-080 | 1 | 0.000000 |
| 36 | IGHV4-39 | Kali-102 | 1 | 1.300000 |
| 37 | IGHV4-39 | Kali-110 | 1 | 3.100000 |
| 38 | IGHV4-39 | Kali-121 | 2 | 9.750000 |
| 39 | IGHV4-4 | Kali-077 | 1 | 5.900000 |
| 40 | IGHV4-59 | Kali-102 | 2 | 16.200000 |
| 41 | IGHV4-59 | Kali-125 | 1 | 3.200000 |
| 42 | IGHV4-61 | Kali-080 | 5 | 6.900000 |
| 43 | IGHV4-61 | Kali-125 | 1 | 4.400000 |
| 44 | IGHV5-51 | Kali-077 | 1 | 11.100000 |
| 45 | IGHV5-51 | Kali-147 | 1 | 4.400000 |

TABLE 21
Mutation Rates (%) by VH Segment and Cell Subset

| | V.Segment | Cell.Subset | Count | Mean.V.Mutation.Rate |
|---|---|---|---|---|
| 1 | IGHV1-2 | IgG MBC | 2 | 7.300000 |
| 2 | IGHV1-46 | IgM MBC | 1 | 9.500000 |
| 3 | IGHV1-69 | IgM MBC | 1 | 0.300000 |
| 4 | IGHV1-8 | IgG MBC | 1 | 8.800000 |
| 5 | IGHV1-8 | IgM MBC | 3 | 2.833333 |
| 6 | IGHV3-11 | IgM MBC | 1 | 1.000000 |
| 7 | IGHV3-15 | IgG MBC | 6 | 10.116667 |
| 8 | IGHV3-15 | IgM MBC | 1 | 4.700000 |
| 9 | IGHV3-23 | IgM MBC | 1 | 3.400000 |
| 10 | IGHV3-23D | IgG MBC | 1 | 7.500000 |
| 11 | IGHV3-30 | IgG MBC | 2 | 11.900000 |
| 12 | IGHV3-30-3 | IgM MBC | 1 | 0.000000 |
| 13 | IGHV3-30-5 | IgG MBC | 1 | 8.800000 |
| 14 | IGHV3-33 | IgG MBC | 1 | 9.800000 |
| 15 | IGHV3-48 | IgG MBC | 1 | 8.200000 |
| 16 | IGHV3-48 | IgM MBC | 1 | 0.700000 |
| 17 | IGHV3-7 | IgM MBC | 1 | 4.500000 |
| 18 | IGHV3-74 | IgM MBC | 1 | 7.100000 |
| 19 | IGHV3-9 | IgG MBC | 6 | 10.150000 |
| 20 | IGHV4-30-4 | IgG MBC | 2 | 9.350000 |
| 21 | IGHV4-31 | IgM MBC | 1 | 1.700000 |
| 22 | IGHV4-34 | IgG MBC | 4 | 10.775000 |
| 23 | IGHV4-34 | IgM MBC | 7 | 3.271429 |
| 24 | IGHV4-38-2 | IgG MBC | 3 | 13.166667 |
| 25 | IGHV4-38-2 | IgM MBC | 2 | 0.500000 |
| 26 | IGHV4-39 | IgG MBC | 3 | 7.700000 |
| 27 | IGHV4-39 | IgM MBC | 4 | 1.100000 |
| 28 | IGHV4-4 | IgG MBC | 1 | 5.900000 |
| 29 | IGHV4-59 | IgG MBC | 2 | 16.200000 |
| 30 | IGHV4-59 | IgM MBC | 1 | 3.200000 |
| 31 | IGHV4-61 | IgG MBC | 3 | 11.500000 |
| 32 | IGHV4-61 | IgM MBC | 3 | 1.466667 |
| 33 | IGHV5-51 | IgG MBC | 1 | 11.100000 |
| 34 | IGHV5-51 | IgM MBC | 1 | 4.400000 |

TABLE 22
Mutation Rates (%) by VK Segment and Cell Subset
A tibble: 17 × 4
Groups: V.Segment [?]

| | V.Segment | Cell.Subset | Count | Mean.V.Mutation.Rate |
|---|---|---|---|---|
| | <fctr> | <fctr> | <int> | <dbl> |
| 1 | IGKV1-12 | IgG MBC | 1 | 7.800000 |
| 2 | IGKV1-39 | IgM MBC | 3 | 3.233333 |
| 3 | IGKV1-5 | IgG MBC | 7 | 7.257143 |
| 4 | IGKV1-5 | IgM MBC | 4 | 2.375000 |
| 5 | IGKV1-8 | IgG MBC | 1 | 8.300000 |
| 6 | IGKV1D-33 | IgG MBC | 1 | 9.400000 |
| 7 | IGKV2-28 | IgM MBC | 1 | 3.400000 |
| 8 | IGKV2-30 | IgG MBC | 5 | 5.400000 |
| 9 | IGKV2D-29 | IgG MBC | 1 | 5.200000 |
| 10 | IGKV3-11 | IgM MBC | 1 | 0.000000 |
| 11 | IGKV3-15 | IgG MBC | 3 | 6.833333 |
| 12 | IGKV3-15 | IgM MBC | 1 | 2.000000 |
| 13 | IGKV3-20 | IgG MBC | 4 | 3.800000 |
| 14 | IGKV3-20 | IgM MBC | 5 | 2.760000 |
| 15 | IGKV3D-20 | IgM MBC | 1 | 3.900000 |
| 16 | IGKV4-1 | IgG MBC | 4 | 6.650000 |
| 17 | IGKV4-1 | IgM MBC | 4 | 2.650000 |

TABLE 23
Mutation Rates (%) by VL Segment and Cell Subset
A tibble: 14 × 4
Groups: V.Segment [?]

| | V.Segment | Cell.Subset | Count | Mean.V.Mutation.Rate |
|---|---|---|---|---|
| | <fctr> | <fctr> | <int> | <dbl> |
| 1 | IGLV1-44 | IgM MBC | 2 | 3.900000 |
| 2 | IGLV1-47 | IgG MBC | 1 | 3.400000 |
| 3 | IGLV1-47 | IgM MBC | 1 | 5.500000 |
| 4 | IGLV1-51 | IgM MBC | 2 | 1.350000 |
| 5 | IGLV2-11 | IgM MBC | 1 | 0.700000 |
| 6 | IGLV2-14 | IgG MBC | 1 | 6.700000 |
| 7 | IGLV2-23 | IgM MBC | 1 | 1.400000 |
| 8 | IGLV2-8 | IgM MBC | 1 | 10.800000 |
| 9 | IGLV3-21 | IgM MBC | 1 | 0.000000 |
| 10 | IGLV3-9 | IgG MBC | 2 | 4.100000 |
| 11 | IGLV4-69 | IgG MBC | 1 | 5.400000 |
| 12 | IGLV4-69 | IgM MBC | 1 | 2.100000 |
| 13 | IGLV6-57 | IgM MBC | 3 | 8.366667 |
| 14 | IGLV8-61 | IgM MBC | 1 | 0.000000 |

TABLE 24
IgM MBC VH Segments Paired by Light Chain Type; NA represents
paired sequence not found.

| | Chain.Light | | |
|---|---|---|---|
| V.Segment.Heavy | Kappa | Lambda | <NA> |
| IGHV1-46 | 1 | 0 | 0 |
| IGHV1-69 | 1 | 0 | 0 |
| IGHV1-8 | 3 | 0 | 0 |
| IGHV3-11 | 0 | 1 | 0 |
| IGHV3-15 | 0 | 0 | 1 |
| IGHV3-23 | 0 | 0 | 1 |
| IGHV3-30-3 | 0 | 0 | 1 |
| IGHV3-48 | 0 | 0 | 1 |
| IGHV3-7 | 0 | 0 | 1 |
| IGHV3-74 | 0 | 0 | 1 |
| IGHV4-31 | 0 | 1 | 0 |
| IGHV4-34 | 3 | 3 | 1 |
| IGHV4-38-2 | 1 | 1 | 0 |
| IGHV4-39 | 1 | 1 | 2 |
| IGHV4-59 | 1 | 0 | 0 |
| IGHV4-61 | 1 | 2 | 0 |
| IGHV5-51 | 0 | 1 | 0 |
| <NA> | 0 | 0 | 0 |

TABLE 25
IgG MBC VH Segments Paired by Light Chain Type; NA represents
paired sequence not found.

| | Chain.Light | | |
|---|---|---|---|
| V.Segment.Heavy | Kappa | Lambda | <NA> |
| IGHV1-2 | 0 | 0 | 2 |
| IGHV1-8 | 0 | 0 | 1 |
| IGHV3-15 | 2 | 2 | 2 |
| IGHV3-23D | 1 | 0 | 0 |
| IGHV3-30 | 0 | 0 | 2 |
| IGHV3-30-5 | 1 | 0 | 0 |
| IGHV3-33 | 1 | 0 | 0 |
| IGHV3-48 | 0 | 0 | 1 |
| IGHV3-9 | 0 | 1 | 5 |
| IGHV4-30-4 | 1 | 0 | 1 |
| IGHV4-34 | 4 | 0 | 0 |
| IGHV4-38-2 | 2 | 0 | 1 |
| IGHV4-39 | 0 | 0 | 3 |
| IGHV4-4 | 1 | 0 | 0 |
| IGHV4-59 | 2 | 0 | 0 |
| IGHV4-61 | 3 | 0 | 0 |
| IGHV5-51 | 1 | 0 | 0 |
| <NA> | 0 | 0 | 0 |

TABLE 26
IgM MBC VH Segments Paired by Light Chain V Segment

| | V.Segment.Light | | | | | |
|---|---|---|---|---|---|---|
| V.Seg- | IGKV1- | IGKV1- | IGKV2- | IGKV3- | IGKV3- | IGKV4- |
| ment.Heavy | 39 | 5 | 28 | 11 | 15 | 1 |
| IGHV1-46 | 0 | 0 | 0 | 0 | 1 | 0 |
| IGHV1-69 | 0 | 0 | 0 | 1 | 0 | 0 |
| IGHV1-8 | 1 | 2 | 0 | 0 | 0 | 0 |
| IGHV3-11 | 0 | 0 | 0 | 0 | 0 | 0 |
| IGHV4-31 | 0 | 0 | 0 | 0 | 0 | 0 |
| IGHV4-34 | 0 | 0 | 0 | 0 | 0 | 3 |
| IGHV4-38-2 | 0 | 0 | 0 | 0 | 0 | 1 |
| IGHV4-39 | 0 | 0 | 1 | 0 | 0 | 0 |
| IGHV4-59 | 0 | 1 | 0 | 0 | 0 | 0 |
| IGHV4-61 | 0 | 1 | 0 | 0 | 0 | 0 |
| IGHV5-51 | 0 | 0 | 0 | 0 | 0 | 0 |
| | V.Segment.Light | | | | | |
| V.Seg- | IGLV1- | IGLV1- | IGLV1- | IGLV2- | IGLV2- | IGLV3- |
| ment.Heavy | 44 | 47 | 51 | 11 | 23 | 21 |
| IGHV1-46 | 0 | 0 | 0 | 0 | 0 | 0 |
| IGHV1-69 | 0 | 0 | 0 | 0 | 0 | 0 |
| IGHV1-8 | 0 | 0 | 0 | 0 | 0 | 0 |
| IGHV3-11 | 0 | 0 | 0 | 0 | 0 | 0 |
| IGHV4-31 | 0 | 0 | 0 | 0 | 1 | 0 |
| IGHV4-34 | 0 | 1 | 1 | 0 | 0 | 0 |
| IGHV4-38-2 | 0 | 0 | 0 | 1 | 0 | 0 |
| IGHV4-39 | 1 | 0 | 0 | 0 | 0 | 0 |
| IGHV4-59 | 0 | 0 | 0 | 0 | 0 | 0 |
| IGHV4-61 | 0 | 0 | 0 | 0 | 0 | 1 |
| IGHV5-51 | 1 | 0 | 0 | 0 | 0 | 0 |

| | V.Segment.Light | | |
|---|---|---|---|
| V.Segment.Heavy | IGLV4-69 | IGLV6-57 | IGLV8-61 |
| IGHV1-46 | 0 | 0 | 0 |
| IGHV1-69 | 0 | 0 | 0 |
| IGHV1-8 | 0 | 0 | 0 |
| IGHV3-11 | 1 | 0 | 0 |
| IGHV4-31 | 0 | 0 | 0 |
| IGHV4-34 | 0 | 1 | 0 |
| IGHV4-38-2 | 0 | 0 | 0 |
| IGHV4-39 | 0 | 0 | 0 |
| IGHV4-59 | 0 | 0 | 0 |
| IGHV4-61 | 0 | 0 | 1 |
| IGHV5-51 | 0 | 0 | 0 |

TABLE 27
IgG MBC VH Segments Paired by Light Chain V Segment

| | V.Segment.Light | | | | | |
|---|---|---|---|---|---|---|
| V.Seg- | IGKV1- | IGKV1- | IGKV1- | IGKV2- | IGKV2D- | IGKV3- |
| ment.Heavy | 12 | 5 | 8 | 30 | 29 | 15 |
| IGHV3-15 | 0 | 0 | 0 | 2 | 0 | 0 |
| IGHV3-23D | 0 | 0 | 0 | 0 | 0 | 0 |
| IGHV3-30-5 | 0 | 0 | 0 | 0 | 1 | 0 |
| IGHV3-33 | 0 | 0 | 1 | 0 | 0 | 0 |
| IGHV3-9 | 0 | 0 | 0 | 0 | 0 | 0 |
| IGHV4-30-4 | 0 | 0 | 0 | 0 | 0 | 1 |
| IGHV4-34 | 0 | 3 | 0 | 0 | 0 | 0 |
| IGHV4-38-2 | 0 | 0 | 0 | 0 | 0 | 0 |
| IGHV4-4 | 0 | 0 | 0 | 0 | 0 | 0 |
| IGHV4-59 | 0 | 0 | 0 | 0 | 0 | 2 |
| IGHV4-61 | 0 | 3 | 0 | 0 | 0 | 0 |
| IGHV5-51 | 1 | 0 | 0 | 0 | 0 | 0 |

| | V.Segment.Light | | | |
|---|---|---|---|---|
| V.Segment.Heavy | IGKV3-20 | IGKV4-1 | IGLV3-9 | IGLV4-69 |
| IGHV3-15 | 0 | 0 | 2 | 0 |
| IGHV3-23D | 1 | 0 | 0 | 0 |
| IGHV3-30-5 | 0 | 0 | 0 | 0 |
| IGHV3-33 | 0 | 0 | 0 | 0 |
| IGHV3-9 | 0 | 0 | 0 | 1 |
| IGHV4-30-4 | 0 | 0 | 0 | 0 |
| IGHV4-34 | 1 | 0 | 0 | 0 |
| IGHV4-38-2 | 0 | 2 | 0 | 0 |
| IGHV4-4 | 1 | 0 | 0 | 0 |
| IGHV4-59 | 0 | 0 | 0 | 0 |
| IGHV4-61 | 0 | 0 | 0 | 0 |
| IGHV5-51 | 0 | 0 | 0 | 0 |

TABLE 28
Average Difference in Heavy and Light Chain V Segment Mutation
Rates by Person, Chain, and Cell Subset

| | cDNA.plate.Heavy | Cell.Subset.Heavy | Chain.Light | Avg.Mut.Rate.Diff | StdDev.Mut.Rate.Diff |
|---|---|---|---|---|---|
| 1 | Kali-077 | IgG MBC | Kappa | 3.6500000 | 0.4949747 |
| 2 | Kali-080 | IgG MBC | Kappa | 3.4600000 | 1.8063776 |
| 3 | Kali-080 | IgG MBC | Lambda | 2.0000000 | NA |
| 4 | Kali-080 | IgM MBC | Kappa | 0.1500000 | 0.2121320 |
| 5 | Kali-080 | IgM MBC | Lambda | −0.2333333 | 0.4041452 |
| 6 | Kali-102 | IgG MBC | Kappa | 7.5000000 | 0.0000000 |
| 7 | Kali-102 | IgM MBC | Kappa | 0.5000000 | 2.7513633 |
| 8 | Kali-102 | IgM MBC | Lambda | −0.0500000 | 0.4949747 |
| 9 | Kali-110 | IgG MBC | Kappa | 3.3600000 | 2.6548070 |
| 10 | Kali-110 | IgM MBC | Kappa | 2.1500000 | 3.4648232 |
| 11 | Kali-121 | IgG MBC | Lambda | 2.2500000 | 0.4949747 |
| 12 | Kali-121 | IgM MBC | Kappa | 0.3000000 | 0.5656854 |
| 13 | Kali-121 | IgM MBC | Lambda | −0.4000000 | 0.9899495 |
| 14 | Kali-125 | IgM MBC | Kappa | 2.9333333 | 4.3247351 |
| 15 | Kali-138 | IgG MBC | Kappa | 10.7333333 | 1.4047538 |
| 16 | Kali-147 | IgG MBC | Kappa | 5.0500000 | 2.0506097 |
| 17 | Kali-147 | IgM MBC | Lambda | −0.2333333 | 1.8823744 |

### REFERENCES

Achtman, A. H., Khan, M., MacLennan, I. C., and Langhome, J. (2003). Plasmodium chabaudi chabaudi infection in mice induces strong B cell responses and striking but temporary changes in splenic cell distribution. J Immunol 171, 317-324.
al-Yaman, F., Genton, B., Kramer, K. J., Chang, S. P., Hui, G. S., Baisor, M., and Alpers, M. P. (1996). Assessment of the role of naturally acquired antibody levels to Plasmodium falciparum merozoite surface protein-1 in protecting Papua New Guinean children from malaria morbidity. The American journal of tropical medicine and hygiene 54, 443-448.
Anderson, S. M., Tomayko, M. M., Ahuja, A., Haberman, A. M., and Shlomchik, M. J. (2007). New markers for murine memory B cells that define mutated and unmutated subsets. J Exp Med 204, 2103-2114.
Arama, C., Skinner, J., Doumtabe, D., Portugal, S., Tran, T. M., Jain, A., Traore, B., Doumbo, O. K., Davies, D. H., Troye-Blomberg, M., et al. (2015). Genetic Resistance to Malaria Is Associated With Greater Enhancement of Immunoglobulin (Ig)M Than IgG Responses to a Broad Array of Plasmodium falciparum Antigens. Open Forum Infect Dis 2, ofv118.
Benson, M. J., Elgueta, R., Schpero, W., Molloy, M., Zhang, W., Usherwood, E., and Noelle, R. J. (2009). Distinction of the memory B cell response to cognate antigen versus bystander inflammatory signals. J Exp Med 206, 2013-2025.
Bemasconi, N. L., Traggiai, E., and Lanzavecchia, A. (2002). Maintenance of serological memory by polyclonal activation of human memory B cells. Science 298, 2199-2202.
Blackman, M. J., Heidrich, H. G., Donachie, S., McBride, J. S., and Holder, A. A. (1990). A single fragment of a malaria merozoite surface protein remains on the parasite during red cell invasion and is the target of invasion-inhibiting antibodies. J Exp Med 172, 379-382.
Bortnick, A., Chernova, I., Quinn, W. J., 3rd, Mugnier, M., Cancro, M. P., and Allman, D. (2012). Long-lived bone marrow plasma cells are induced early in response to T cell-independent or T cell-dependent antigens. J Immunol 188, 5389-5396.
Boyle, M. J., Reiling, L., Feng, G., Langer, C., Osier, F. H., Aspeling-Jones, H., Cheng, Y. S., Stubbs, J., Tetteh, K. K., Conway, D. J., et al. (2015). Human antibodies fix complement to inhibit Plasmodium falciparum invasion of erythrocytes and are associated with protection against malaria. Immunity 42, 580-590.
Branch, O. H., Udhayakumar, V., Hightower, A. W., Oloo, A. J., Hawley, W. A., Nahlen, B. L., Bloland, P. B., Kaslow, D. C., and Lal, A. A. (1998). A longitudinal investigation of IgG and IgM antibody responses to the merozoite surface protein-1 19-kiloDalton domain of Plasmodium falciparum in pregnant women and infants: associations with febrile illness, parasitemia, and anemia. The American journal of tropical medicine and hygiene 58, 211-219.
Butler, N. S., Moebius, J., Pewe, L. L., Traore, B., Doumbo, O. K., Tygrett, L. T., Waldschmidt, T. J., Crompton, P. D., and Harty, J. T. (2012). Therapeutic blockade of PD-L1 and LAG-3 rapidly clears established blood-stage Plasmodium infection. Nat Immunol 13, 188-195.
Chan, T. D., and Brink, R. (2012). Affinity-based selection and the germinal center response. Immunol Rev 247, 11-23.
Clark, E. H., Silva, C. J., Weiss, G. E., Li, S., Padilla, C., Crompton, P. D., Hernandez, J. N., and Branch, O. H. (2012). Plasmodium falciparum malaria in the Peruvian Amazon, a region of low transmission, is associated with immunologic memory. Infect Immun 80, 1583-1592.
Cohen, S., Mc, G. I., and Carrington, S. (1961). Gamma-globulin and acquired immunity to human malaria. Nature 192, 733-737.
Couper, K. N., Phillips, R. S., Brombacher, F., and Alexander, J. (2005). Parasite-specific IgM plays a significant role in the protective immune response to asexual erythrocytic stage Plasmodium chabaudi AS infection. Parasite Immunol 27, 171-180.
Crompton, P. D., Traore, B., Kayentao, K., Doumbo, S., Ongoiba, A., Diakite, S. A., Krause, M. A., Doumtabe, D., Kone, Y., Weiss, G., et al. (2008). Sickle cell trait is associated with a delayed onset of malaria: implications for time-to-event analysis in clinical studies of malaria. J Infect Dis 198, 1265-1275.
Dodoo, D., Aikins, A., Kusi, K. A., Lamptey, H., Remarque, E., Milligan, P., Bosomprah, S., Chilengi, R., Osei, Y. D., Akanmori, B. D., and Theisen, M. (2008). Cohort study of the association of antibody levels to AMA1, MSP119, MSP3 and GLURP with protection from clinical malaria in Ghanaian children. Malaria journal 7, 142.
Dogan, I., Bertocci, B., Vilmont, V., Delbos, F., Megret, J., Storck, S., Reynaud, C. A., and Weill, J. C. (2009). Multiple layers of B cell memory with different effector functions. Nat Immunol 10, 1292-1299. Gitlin, A. D., von Boehmer, L., Gazumyan, A., Shulman, Z., Oliveira, T. Y., and Nussenzweig, M. C. (2016). Independent Roles of Switching and Hypermutation in the Development and Persistence of B Lymphocyte Memory. Immunity.
Hirunpetcharat, C., Tian, J. H., Kaslow, D. C., van Rooijen, N., Kumar, S., Berzofsky, J. A., Miller, L. H., and Good, M. F. (1997). Complete protective immunity induced in mice by immunization with the 19-kilodalton carboxyl-terminal fragment of the merozoite surface protein-1 (MSP1[19]) of Plasmodium yoelii expressed in Saccharomyces cerevisiae: correlation of protection with antigen-specific antibody titer, but not with effector CD4+ T cells. J Immunol 159, 3400-3411.
Ise, W., Inoue, T., McLachlan, J. B., Kometani, K., Kubo, M., Okada, T., and Kurosaki, T. (2014). Memory B cells contribute to rapid Bc16 expression by memory follicular helper T cells. Proc Natl Acad Sci USA 111, 11792-11797.
Kadekoppala, M., and Holder, A. A. (2010). Merozoite surface proteins of the malaria parasite: the MSP1 complex and the MSP7 family. Int J Parasitol 40, 1155-1161.
Kaji, T., Ishige, A., Hikida, M., Taka, J., Hijikata, A., Kubo, M., Nagashima, T., Takahashi, Y., Kurosaki, T., Okada, M., et al. (2012). Distinct cellular pathways select germline-encoded and somatically mutated antibodies into immunological memory. J Exp Med 209, 2079-2097.
Kaminski, D. A., Wei, C., Qian, Y., Rosenberg, A. F., and Sanz, I. (2012). Advances in human B cell phenotypic profiling. Front Immunol 3, 302.
Klein, U., Kuppers, R., and Rajewsky, K. (1997). Evidence for a large compartment of IgM-expressing memory B cells in humans. Blood 89, 1288-1298.
Klein, U., Rajewsky, K., and Kuppers, R. (1998). Human immunoglobulin (Ig)M+IgD+ peripheral blood B cells expressing the CD27 cell surface antigen carry somatically mutated variable region genes: CD27 as a general marker for somatically mutated (memory) B cells. J Exp Med 188, 1679-1689.
Kolhatkar, N. S., Brahmandam, A., Thouvenel, C. D., Becker-Herman, S., Jacobs, H. M., Schwartz, M. A., Allenspach, E. J., Khim, S., Panigrahi, A. K., Luning Prak, E. T., et al. (2015). Altered BCR and TLR signals promote enhanced positive selection of autoreactive transitional B cells in Wiskott-Aldrich syndrome. J Exp Med 212, 1663-1677.
Kurosaki, T., Kometani, K., and Ise, W. (2015). Memory B cells. Nat Rev Immunol 15, 149-159.
Malleret, B., Claser, C., Ong, A. S., Suwanarusk, R., Sriprawat, K., Howland, S. W., Russell, B., Nosten, F., and Renia, L. (2011). A rapid and robust tri-color flow cytometry assay for monitoring malaria parasite development. Sci Rep 1, 118.
McHeyzer-Williams, L. J., Milpied, P. J., Okitsu, S. L., and McHeyzer-Williams, M. G. (2015). Class-switched memory B cells remodel BCRs within secondary germinal centers. Nat Immunol 16, 296-305.
Moss, D. K., Remarque, E. J., Faber, B. W., Cavanagh, D. R., Arnot, D. E., Thomas, A. W., and Holder, A. A. (2012). Plasmodium falciparum 19-kilodalton merozoite surface protein 1 (MSP1)-specific antibodies that interfere with parasite growth in vitro can inhibit MSP1 processing, merozoite invasion, and intracellular parasite development. Infect Immun 80, 1280-1287.
Nduati, E. W., Ng, D. H., Ndungu, F. M., Gardner, P., Urban, B. C., and Langhorne, J. (2010). Distinct kinetics of memory B-cell and plasma-cell responses in peripheral blood following a blood-stage Plasmodium chabaudi infection in mice. PLoS One 5, e15007.
Ndungu, F. M., Cadman, E. T., Coulcher, J., Nduati, E., Couper, E., Macdonald, D. W., Ng, D., and Langhorne, J. (2009). Functional memory B cells and long-lived plasma cells are generated after a single Plasmodium chabaudi infection in mice. PLoS Pathog 5, e1000690.
Ndungu, F. M., Lundblom, K., Rono, J., Illingworth, J., Eriksson, S., and Farnert, A. (2013). Long-lived Plasmodium falciparum specific memory B cells in naturally exposed Swedish travelers. Eur J Immunol 43, 2919-2929.
Ndungu, F. M., Olotu, A., Mwacharo, J., Nyonda, M., Apfeld, J., Mramba, L. K., Fegan, G. W., Bejon, P., and Marsh, K. (2012). Memory B cells are a more reliable archive for historical antimalarial responses than plasma antibodies in no-longer exposed children. Proc Natl Acad Sci USA 109, 8247-8252.
Obukhanych, T. V., and Nussenzweig, M. C. (2006). T-independent type II immune responses generate memory B cells. J Exp Med 203, 305-310.
Pape, K. A., Taylor, J. J., Maul, R. W., Gearhart, P. J., and Jenkins, M. K. (2011). Different B cell populations mediate early and late memory during an endogenous immune response. Science 331, 1203-1207.
Richard, K., Pierce, S. K., and Song, W. (2008). The agonists of TLR4 and 9 are sufficient to activate memory B cells to differentiate into plasma cells in vitro but not in vivo. J Immunol 181, 1746-1752.
Riley, E. M., Allen, S. J., Wheeler, J. G., Blackman, M. J., Bennett, S., Takacs, B., Schonfeld, H. J., Holder, A. A., and Greenwood, B. M. (1992). Naturally acquired cellular and humoral immune responses to the major merozoite surface antigen (PfMSP1) of Plasmodium falciparum are associated with reduced malaria morbidity. Parasite Immunol 14, 321-337.
Robbiani, D. F., Deroubaix, S., Feldhahn, N., Oliveira, T. Y., Callen, E., Wang, Q., Jankovic, M., Silva, I. T., Rommel, P. C., Bosque, D., et al. (2015). Plasmodium Infection Promotes Genomic Instability and AID-Dependent B Cell Lymphoma. Cell 162, 727-737.
Schwartz, M. A., Kolhatkar, N. S., Thouvenel, C., Khim, S., and Rawlings, D. J. (2014). CD4+ T cells and CD40 participate in selection and homeostasis of peripheral B cells. J Immunol 193, 3492-3502.
Seifert, M., Przekopowitz, M., Taudien, S., Lollies, A., Ronge, V., Drees, B., Lindemann, M., Hillen, U., Engler, H., Singer, B. B., and Kuppers, R. (2015). Functional capacities of human IgM memory B cells in early inflammatory responses and secondary germinal center reactions. Proc Natl Acad Sci USA 112, E546-555.
Tangye, S. G., and Good, K. L. (2007). Human IgM+CD27+B cells: memory B cells or “memory” B cells? J Immunol 179, 13-19.
Tarlinton, D., and Good-Jacobson, K. (2013). Diversity among memory B cells: origin, consequences, and utility. Science 341, 1205-1211.
Taylor, J. J., Martinez, R. J., Titcombe, P. J., Barsness, L. O., Thomas, S. R., Zhang, N., Katzman, S. D., Jenkins, M. K., and Mueller, D. L. (2012a). Deletion and anergy of polyclonal B cells specific for ubiquitous membrane-bound self-antigen. J Exp Med 209, 2065-2077.
Taylor, J. J., Pape, K. A., and Jenkins, M. K. (2012b). A germinal center-independent pathway generates unswitched memory B cells early in the primary response. J Exp Med 209, 597-606.
Tiller, T., Busse, C. E., and Wardemann, H. (2009). Cloning and expression of murine Ig genes from single B cells. J Immunol Methods 350, 183-193.
Tomayko, M. M., Steinel, N. C., Anderson, S. M., and Shlomchik, M. J. (2010). Cutting edge:
Hierarchy of maturity of murine memory B cell subsets. J Immunol 185, 7146-7150.
Toyama, H., Okada, S., Hatano, M., Takahashi, Y., Takeda, N., Ichii, H., Takemori, T., Kuroda, Y., and
Tokuhisa, T. (2002). Memory B cells without somatic hypermutation are generated from Bc16-deficient B cells. Immunity 17, 329-339.
Von Eschen, K. B., and Rudbach, J. A. (1974). Immunological responses of mice to native protoplasmic polysaccharide and lipopolysaccharide: functional separation of the two signals required to stimulate a secondary antibody response. J Exp Med 140, 1604-1614.
Weill, J. C., Weller, S., and Reynaud, C. A. (2009). Human marginal zone B cells. Annu Rev Immunol 27, 267-285.
Weisel, Florian J., Zuccarino-Catania, Griselda V., Chikina, M., and Shlomchik, Mark J. (2016). A Temporal Switch in the Germinal Center Determines Differential Output of Memory B and Plasma Cells. Immunity 44, 116-130 Weiss, G. E., Ndungu, F. M., McKittrick, N., Li, S., Kimani, D., Crompton, P. D., Marsh, K., and Pierce, S. K. (2012). High efficiency human memory B cell assay and its application to studying Plasmodium falciparum-specific memory B cells in natural infections. J Immunol Methods 375, 68-74. Weiss, G. E., Traore, B., Kayentao, K., Ongoiba, A., Doumbo, S., Doumtabe, D., Kone, Y., Dia, S., Guindo, A., Traore, A., et al. (2010). The Plasmodium falciparum-specific human memory B cell compartment expands gradually with repeated malaria infections. PLoS Pathog 6, el000912.
Wipasa, J., Suphavilai, C., Okell, L. C., Cook, J., Conan, P. H., Thaikla, K., Liewsaree, W., Riley, E. M., and Hafalla, J. C. (2010). Long-lived antibody and B Cell memory responses to the human malaria parasites, Plasmodium falciparum and Plasmodium vivax. PLoS Pathog 6, e1000770.
Yates, J. L., Racine, R., McBride, K. M., and Winslow, G. M. (2013). T cell-dependent IgM memory B cells generated during bacterial infection are required for IgG responses to antigen challenge. J Immunol 191, 1240-1249.
Zuccarino-Catania, G. V., Sadanand, S., Weisel, F. J., Tomayko, M. M., Meng, H., Kleinstein, S. H., Good-Jacobson, K. L., and Shlomchik, M. J. (2014). CD80 and PD-L2 define functionally distinct memory B cell subsets that are independent of antibody isotype. Nat Immunol 15, 631637.

What is claimed:

1.-91. (canceled)

92. A recombinant antigen-binding polypeptide comprising an antigen-binding domain of an IgM memory B cell receptor.

93. The recombinant antigen-binding polypeptide of claim 92, wherein the IgM memory B cell receptor antigen-binding domain is comprised in a non-IgM isotype antibody framework.

94. The recombinant antigen-binding polypeptide of claim 92, wherein the IgM memory B cell receptor antigen-binding domain is a human IgM memory B cell receptor antigen binding domain.

95. The recombinant antigen-binding polypeptide of claim 93, wherein the non-IgM isotype antibody framework is an IgG antibody framework.

96. The recombinant antigen-binding domain polypeptide of claim 92, wherein the recombinant antigen-binding polypeptide comprises an scFv polypeptide, a single-domain antibody construct, a chimeric antibody construct or a bispecific antibody construct.

97. The recombinant antigen-binding polypeptide of claim 92, wherein the polypeptide binds its antigen with a KD of 10-6 M or lower.

98. The recombinant antigen-binding polypeptide of claim 92, wherein the variable light chain sequence, variable heavy chain sequence, or both has one to eight somatic mutations relative to a variable heavy chain sequence or variable light chain sequence from a naïve B cell.

99. The recombinant antigen-binding polypeptide of claim 92, wherein the IgM memory B cell receptor antigen-binding domain specifically binds an antigen comprised or expressed by an infectious organism.

100. The recombinant antigen-binding polypeptide of claim 99, wherein the infectious organism is P. falciparum.

101. The recombinant antigen-binding polypeptide of claim 100, wherein the antigen is P. falciparum merozoite surface protein 1 (MSP1) or apical membrane antigen 1 (AMA).

102. A method of sorting antigen-specific IgM memory B cells comprising:
contacting a biological sample obtained from a subject having had prior exposure to an antigen of interest with an agent comprising the antigen or a portion thereof; and
sorting a cell population comprising IgM memory B cells based on binding to the agent comprising the antigen.

103. The method of claim 102, further comprising sorting the population comprising antigen-specific IgM memory B cells using an agent specific for CD21, an agent specific for CD27, an agent specific for IgM isotype, or any combination thereof to isolate a population of IgM memory B cells specific for the antigen.

104. The method of claim 102, wherein the agent comprising the antigen comprises a multimer of the antigen.

105. The method of claim 104, wherein the agent comprising the antigen comprises a dimer, trimer or tetramer of the antigen.

106. The method of claim 102, further comprising a step of cloning one or more B cell receptors (BCRs) of the cell population comprising IgM memory B cells, or antigen binding domains thereof, and expressing the one or more BCRs or antigen-binding domains thereof as one or more recombinant antigen-binding polypeptides.

107. A pharmaceutical composition comprising a composition of claim 92, and a pharmaceutically acceptable carrier.

108. A vaccine composition comprising a composition of claim 92.

109. A method of treating a subject in need of treatment for a disease caused by an infectious organism, the method comprising administering a composition of claim 92 to the subject, wherein the antigen-binding polypeptide specifically binds an antigen comprised by the infectious organism.

110. A method of sorting Plasmodium-specific IgM memory B cells (MBCs), comprising:
generating B cell tetramers specific for blood or liver stage Plasmodium antigens;
providing the B cell tetramers to a biological sample obtained from a subject infected with malaria; and
sorting the Plasmodium-specific IgM MBCs based on binding to the tetramers.

111. The method of claim 110, further comprising a step of cloning Plasmodium-specific IgM MBC B cell receptors (BCRs) from tetramer-bound MBCs and expressing a BCR as a recombinant antigen-binding polypeptide.


SEQUENCE LISTING (154 sequences)

SEQ ID NO: 1 - DNA, 343 bp, Artificial Sequence
  Description of Artificial Sequence Synthetic polynucleotide
caggtgcagctgcagcagcctggggctgagctggtaaagcctggggcttcagtgaagttgtcctgcaaggcttctggctacactttcatcaactactggatgcactgggtgaagcagaggcctggacaaggccttgagtggattggaatgattcatcctaaaagtggtagcactaacttcaatgagaagttcaagagcaaggccacactgactgtagacaaatcctccagcacagcctacatgcaagtcagcagcctgacatctgaggactctgcggtctattattgtgcaaaggaagtcatggactactggggtcaaggaacctcagtcaccgtctcctcag

SEQ ID NO: 2 - DNA, 340 bp, Artificial Sequence
  Description of Artificial Sequence Synthetic polynucleotide
gacattgtgatgactcagtctccatcctccctggctatgtcagtaggacagaaggtcactatgaactgcaagtccagtcagagccttttaaatagtagaaatcaaaagaattttttggcctggtaccaacagaaaccaggacagtctcctaaacttctggtatactttgcatccactagggactctggggtccctgatcgcttcataggcagtggatctgggacagatttcactcttaccatcagcagtgtgcaggctgaagacctggcagattacttctgtcagcaacattatagcactccgtacacgttcggaggggggaccaagctggaaataaaac

SEQ ID NO: 3 - DNA, 355 bp, Artificial Sequence
  Description of Artificial Sequence Synthetic polynucleotide
gatgtacagcttcaggagtcaggacctggcctcgtgaaaccttctcagtctctgtctctcacctgttctgtcattggttattccatcaccagtggttattactggaactgggtccggcagtttccaggaaacaaactggaatggatgtcctacataaactacgatggtaacaataactacaacccttctctcaaaaatcgaatctccatcactcgtgacacatctaagaaccagtttttcctgaagttgaattctgtgactactgaggacacagccacatattactgtgcaagagggaggtttccttatgctttggactactggggtcaaggaacctcagtcaccgtctcctcag

SEQ ID NO: 4 - DNA, 322 bp, Artificial Sequence
  Description of Artificial Sequence Synthetic polynucleotide
gacatccagatgactcagtctccagcctccctatctgtctctgtgggagaaactgtcaccatcacatgtcgagcaagtgagaatatttacagtaatttagcatggtatcagcagaaacagggaaaatctcctcagctcctggtctatgaaacaacaaagttaagagatggtgtgtcatcaaggttcagtggcagtggatcaggcacacagtatttcctcaagatcaacaacctgcagtctgaagattttgggagttattactgtcaacatttttgggggagtccgtggacgttcggtgggggcaccaagctggaaatcaaac

SEQ ID NO: 5 - DNA, 360 bp, Artificial Sequence
  Description of Artificial Sequence Synthetic polynucleotide
gaggtgcagctggtggagtctggtggaggattggtgcagcctaaaggatcattgaaactctcatgtgccgcctctggtttcaccttcaatacctatgccatgcactgggtccgccaggctccaggaaagggtttggaatgggttgctcgcataagaagtaaaagtagtaattatgcaacatattatgccgattcagtgaaagacagattcaccatctccagagatgattcacaaagcatgctctatctgcaaatgaacaacctgaaaactgaggacacagccatgtattactgtgtgagagagagttgggacctctggtttgcttactggggccaagggactctggtcactgtctctgca

SEQ ID NO: 6 - DNA, 337 bp, Artificial Sequence
  Description of Artificial Sequence Synthetic polynucleotide
aacattatgatgacacagtcgccatcatctctggctgtgtctgcaggagaaaaggtcactatgagctgtaagtccagtcaaagtgttttatacagttcaaatcagaagaactacttggcctggtaccagcagaaaccagggcagtctcctaaattgctgatctactgggcatccactagggaatctggtgtccctgatcgcttcacaggcagtggatctgggacagattttactcttaccatcagcagtgtacaagctgaagacctggcagtttattactgtcatcaatacctctcctcgtggacgttcggtggaggcaccaagctggaaatcaaac

SEQ ID NO: 7 - DNA, 360 bp, Artificial Sequence
  Description of Artificial Sequence Synthetic polynucleotide
gaggtgcagctgcagcagtctggacctgagctggtgaagcctggggcttcagtgaagataccctgcaaggcttctggatacacattcactgactacaacatggactgggtgaagcagagccatggaaagagccttgagtggattggagatattaatcctaacaatggtggtactatctacaaccagaagttcaagggcaaggccacattgactgtagacaagtcctccagcacagcctacatggagctccgcagcctgacatctgaggacactgcagtctattactgtgcaagaaggagattacgacgtgagggggactttgactactggggccaaggcaccactctcacagtctcctca

SEQ ID NO: 8 - DNA, 322 bp, Artificial Sequence
  Description of Artificial Sequence Synthetic polynucleotide
gacattgtgatgactcagtctcaaaaattcatgtccacatcagtaggagacagggtcagcgtcacctgcaaggccagtcagaatgtgaatactaatgtagcctggtatcaacagaaaccagggcaatctcctaaagcactgatttactcggcatccttccggtacagtggagtccctgatcgcttcacaggcagtggatctgggacagatttcactctcaccatcagcaatgtgcagtctgaagacttggcagagtatttctgtcagcaatataacagctatcctctcacgttcggaggggggaccaagctggaaataaaac

SEQ ID NO: 9 - DNA, 355 bp, Artificial Sequence
  Description of Artificial Sequence Synthetic polynucleotide
gatgtacagcttcaggagtcaggacctggcctcgtgaaaccttctcagtctctgtctctcacctgctctgtcactggctactccatcaccagtggttattactggaactggatccggcagtttccaggaaacaaactggaatggatgggctacataagctacgatggtagcaataactacaacccatctctcaaaaatcgaatctccatcactcgtgacacatctaagaaccagtttttcctgaagttgaattctgtgactactgaggacacagccacatattactgtgcaagagggaagggatcctatgctatggactactggggtcaaggaacctcagtcaccgtctcctcag

SEQ ID NO: 10 - DNA, 322 bp, Artificial Sequence
  Description of Artificial Sequence Synthetic polynucleotide
gacatccagatgactcagtctccagcctccctatctgtatctgtgggagaaactgtcaccatcacatgtcgagcaagtgagaatatttacagtaatttagcatggtatcagcagaaacagggaaaatctcctcagctcctggtctatgttgcaacaaacttagcagatggtgtgccatcaaggttcagtggcagtggatcaggcacacagtattccctcaagatcaacagcctgcagtctgaagattttgggagttattactgtcaacatttttggggtactccattcacgttcggctcggggacaaagttggaaataaaac

SEQ ID NO: 11 - DNA, 355 bp, Artificial Sequence
  Description of Artificial Sequence Synthetic polynucleotide
caggtgcagctgaagcagtctggagctgagctggtgaagcctggggcttcagtgaagatgtcctgcaaggcttctggctacaccttcactgactactatataaactggttgaaacagaggcctggacagggccttgagtggattggaaagattggtcctggaagtggtagtacttactacaatgagaagttcaaggacaaggccacactgactgcagacaaatcctccagcacagcctacatgcagctcagcagcctgacatctgaggactctgcagtctatttctgtacaagaacctactatagtaattactttgactactggggccaaggcaccactctcacagtctcctcag

SEQ ID NO: 12 - DNA, 322 bp, Artificial Sequence
  Description of Artificial Sequence Synthetic polynucleotide
agtattgtgatgacccagactcccaaattcctgcttgtatcagcaggagacagggttaccataacctgcaaggccagtcagagtgtgaataataatgtagcttggtaccaacagaagccagggcagtctcctaaactgctgatattttatgcatccaatcgctacactggagtccctgatcgcttcactggcagtggatatgggacggatttcaccttcaccatcaacactgtgcaggctgaagacctggcagtttatttctgtcagcaggattatagttctccgaacacgttcggaggggggaccaagctggaaataaaac

SEQ ID NO: 13 - DNA, 346 bp, Artificial Sequence
  Description of Artificial Sequence Synthetic polynucleotide
caggtgcagctgcagcagcctggggctgaactggtgaagcctggggcttcagtgaagttgtcctgtaaggcttctggctacactttcaccttctactggatgcactgggtgaagcagaggcctggacaaggccttgagtggattggaatgattcatcctaatagtggtagtactaactacaatgagaagttcaagagcaaggccacactgactgtagacaaatcctccagcacagcctacatgcaactcagcagcctgacatctgaggactctgcggtctattactgtgcaagagggggggactttgactactggggccaaggcaccactctcacagtctcctcag

SEQ ID NO: 14 - DNA, 334 bp, Artificial Sequence
  Description of Artificial Sequence Synthetic polynucleotide
gacattgtgctgacccaatctccaccttctttggctgtgtctctagggcagagggccaccatctcctgcagagccagcgaaagtgttgataattttggcattaattttatgaactggttccaacagaaaccaggacagccacccaaactcctcatctatgctgcatccaaccaaggatccggggtccctgccaggtttagtggcagtgggtctgggacagacttcagcctcaacatccatcctatggaggaggatgatactgcaatgtatttctgtcagcaaagtaaggaggttcctcggacgttcggtggaggcaccaagctggaaatcaaac

SEQ ID NO: 15 - DNA, 367 bp, Artificial Sequence
  Description of Artificial Sequence Synthetic polynucleotide
gatgtacagcttcaggagtcaggacctggcctcgtgaaaccttctcagtctctgtctctcacctgctctgtcactggctactccatcaccagtggttattactggaactggatccggcagtttccaggaaacaacctggaatggatgggctacataaactacgatggtagcaataactacaatccttctctcaaaaatcgaatctccatcactcgtgacacatctaagaaccagtttttcctgaagttgaattctgtgactagtgaggacacaggcacgtattactgtgcaagaggggcctacaatagtaactgggggggtgctatggactgctggggtcaaggaacctcagtcaccgtctcctcag

SEQ ID NO: 16 - DNA, 322 bp, Artificial Sequence
  Description of Artificial Sequence Synthetic polynucleotide
gacatccagatgactcagtctccagcctccctatctgtatctgtgggagaaattgtcaccatcacatgtcgagcaagtgagaatatttacagtaatttagcatggtatcagcagaaacagggaaaatctcctcacctcctggtctatgctgcaacaaagttagcagctggtgtgccatcaaggttcagtggcagtggatcaggcacgcagtattccctcaagatcaacagcctgcagtctgaagattttgggagttattactgtcaacatttttggggtattcccccgacgttcggtggaggcaccaagctggaaatcaaac

SEQ ID NO: 17 - DNA, 346 bp, Artificial Sequence
  Description of Artificial Sequence Synthetic polynucleotide
caggtgcagctgcagcagcctggggctgagctggtaaagccaggggcttcagtgaagttgtcctgcagggcttctggctacactttcaccagctactggatgcactgggtgaagcagaggcctggacaaggccttgagtggattggaatgattcatcctaaaagtggtagtattaattacaatgagaagttcaagagcaaggccacactgactgtagacaaatcctccagcacagcctacatgcaactcagcagcctgacatctgaggactctgcggtctattactgtgcaagaggtggggactttgactactggggccagggcaccactctcacagtctcctcag

SEQ ID NO: 18 - DNA, 334 bp, Artificial Sequence
  Description of Artificial Sequence Synthetic polynucleotide
gacattgtgctgacccaatctccagcttctttggctgtgtctctagggcagagggccaccatctcctgcagagccagcgaaagtgttgataattatggcattagttttatgaactggttccaacagaaaccaggacagccacccaaactcctcatctatgctgcatccaaccaaggatccggggtccctgccaggtttagtggcagtgggtctgggacagatttcagcctcaacatccatcctatggaggaggatgatactgcaatgtatttctgtcagcaaagtagggaaattcctcggacgttcggtggaggcaccaagctggaaatcaaac

SEQ ID NO: 19 - DNA, 358 bp, Artificial Sequence
  Description of Artificial Sequence Synthetic polynucleotide
gatgtacagcttcaggagtcaggacctggcctcgtgaaaccttctcagtctctgtctctcacctgctctgtcactggctactccatcaccagtggttattactggaactggatccggcagtttccaggaaacaaactggaatggatgggctacataagctacgatggtagcaataactacaacccatctctcaaaaatcgaatctccatcactcgtgacacatctaagaaccagtttttcctgaagttgaattctgtgactactgaggacacagccacatattactgtgcaagggaaagtccgggctactatgctatggactactggggtcaaggaacctcagtcaccgtctcctcag

SEQ ID NO: 20 - DNA, 322 bp, Artificial Sequence
  Description of Artificial Sequence Synthetic polynucleotide
gacatccagatgactcagtctccagcctccctatctgtatctgtgggagaaactgtcaccatcacatgtcgagcaagtgagaatatttacagtaatttagcatggtatcagcagaaacagggaaaatctcctcagctcctggtctatgctgcaacaaacttagcagatggtgtgccatcaaggttcagtggcagtggatcaggcacacagtattccctcaagatcaacagcctgcagtctgaagattttgggagttattactgtcaacatttttggggtactccgttcacgttcggtgctgggaccaagctggagctgaaac

SEQ ID NO: 21 - DNA, 346 bp, Artificial Sequence
  Description of Artificial Sequence Synthetic polynucleotide
caggtgcagctgcagcagcctggggctgagctggtaaagcctggggcttcagtgaagttgtcctgcaaggcttctggctacactttcaccagctactggatgcactgggtgaagcagaggcctggacaaggccttgagtggattggaatgattcatcctaatagtggtagtactaactacaatgagaagttcaagagcaaggccacactgactgtagacaaatcctccagcacagcctacatgcaactcagcagcctgacatctgaggactctgcggtctattactgtgcaagaggaggtgactttgactactggggccaaggcaccactctcacagtctcctcag

SEQ ID NO: 22 - DNA, 334 bp, Artificial Sequence
  Description of Artificial Sequence Synthetic polynucleotide
gacattgtgctgacccaatctccagcttctttggctgtgtctctagggcagagggccaccatctcctgcagagccagcgaaagtgttgataattatggcattagttttatgaactggttccaacagaaaccaggacagccacccaaactcctcatctatgctgcatccaaccaaggatccggggtccctgccaggtttagtggcagtgggtctgggacagacttcagcctcaacatccatcctatggaggaggatgatactgcaatgtatttctgtcagcaaagtaaggaggttcctcggacgttcggtggaggcaccaagctggaaatcaaac

SEQ ID NO: 23 - DNA, 355 bp, Artificial Sequence
  Description of Artificial Sequence Synthetic polynucleotide
gatgtacagcttcaggagtcaggacctggcctcgtgaaaccttctcagtctctgtctctcacctgctctgtcactggctactccatcaccagtggttattactggaactggatccggcagtttccaggaaacaaactggaatggatgggctacataagctacgatggtagcaataactacaacccatctctcaaaaatcgaatctccatcactcgtgacacatctaagaaccagtttttcctgaagttgaattctgtgactactgaggacacagccacatattactgtgcaagagagactgggacgaggtactttgactactggggccaaggcaccactctcacagtctcctcag

SEQ ID NO: 24 - DNA, 322 bp, Artificial Sequence
  Description of Artificial Sequence Synthetic polynucleotide
gacatccagatgactcagtctccagcctccctatctgtatctgtgggagaaactgtcaccatcacatgtcgagcaagtgagaatatttacagtaatttagcatggtatcagcagaaacagggaaaatctcctcagctcctggtctatgctgcaacaaacttagcagatggtgtgccatcaaggttcagtggcagtggatcaggcacacagtattccctcaagatcaacagcctgcagtctgaagattttgggagttattactgtcaacatttttggggtactccgtacacgttcggaggggggaccaagctggaaataaaac

SEQ ID NO: 25 - DNA, 272 bp, Artificial Sequence
  Description of Artificial Sequence Synthetic polynucleotide
caggtgcagctgcaggagtcgggcccaggactggtgaagccttcacagaccctgtccctcacctgcactgtctctggtggctccatcagcagtagtggttactactggagctggatccgccagcacccagcgaagggcctggagtggattgggtacatctattacagtgggagcacctatcacaacccgtccctcaagagtcgagttaccatatcagtaggcacgtctaagaaccagttctccctgaagctgagctctgtgactgccgcgga

SEQ ID NO: 26 - protein, 123 aa, Artificial Sequence
  Description of Artificial Sequence Synthetic polypeptide
QVQLQESGPGLVKPSQTLSLTCTVSGGSISSSGYYWSWIRQHPAKGLEWIGYIYYSGSTYHNPSLKSRVTISVGTSKNQFSLKLSSVTAADTAVYYCARGYFSGTYSGAFDIWGQGTMVTVSS

SEQ ID NO: 27 - protein, 10 aa, Artificial Sequence
  Description of Artificial Sequence Synthetic peptide
GGSISSSGYY

SEQ ID NO: 28 - protein, 7 aa, Artificial Sequence
  Description of Artificial Sequence Synthetic peptide
IYYSGST

SEQ ID NO: 29 - protein, 15 aa, Artificial Sequence
  Description of Artificial Sequence Synthetic peptide
ARGYFSGTYSGAFDI

SEQ ID NO: 30 - DNA, 320 bp, Artificial Sequence
  Description of Artificial Sequence Synthetic polynucleotide
cagtctgccctgactcagcctgcctccgtgtctgggtctcctggacagtcgatcaccatctcctgcactggaaccagcagtgatgttgggagttataaccttgtctcctggtaccaacagcacccaggcaaagcccccaaactcatggtttatgagggcagtaaacggccctcagggctttctaatcgcttctctggctccaagtctggcaacacggcctccctgacaatctctgggctccaggctgaagacgaggctgattattactgctgctcatatgcaggtagtagcacttgggtgttcggcggagggaccaaact

SEQ ID NO: 31 - protein, 110 aa, Artificial Sequence
  Description of Artificial Sequence Synthetic polypeptide
QSALTQPASVSGSPGQSITISCTGTSSDVGSYNLVSWYQQHPGKAPKLMVYEGSKRPSGLSNRFSGSKSGNTASLTISGLQAEDEADYYCCSYAGSSTWVFGGGTKLTVL

SEQ ID NO: 32 - protein, 9 aa, Artificial Sequence
  Description of Artificial Sequence Synthetic peptide
SSDVGSYNL

SEQ ID NO: 33 - protein, 3 aa, Artificial Sequence
  Description of Artificial Sequence Synthetic peptide
EGS

SEQ ID NO: 34 - protein, 10 aa, Artificial Sequence
  Description of Artificial Sequence Synthetic peptide
CSYAGSSTWV

SEQ ID NO: 35 - DNA, 271 bp, Artificial Sequence
  Description of Artificial Sequence Synthetic polynucleotide
caggtgcagctggtggagtctgggggaggcttggtcaagcctggagggtccctgagactctcctgtgcagcctctggattcaccttcagtgactactacatgagctggatccgccaggctccagggaaggggctggagtggatttcatacattagtagtagtggtagtaccatatactacggagactctgtgaagggccgattcaccatctccagggacaacgccaagaactcactgtatctacaaatgaacagcctgagagccgaggaca

SEQ ID NO: 36 - protein, 120 aa, Artificial Sequence
  Description of Artificial Sequence Synthetic polypeptide
QVQLVESGGGLVKPGGSLRLSCAASGFTFSDYYMSWIRQAPGKGLEWISYISSSGSTIYYGDSVKGRFTISRDNAKNSLYLQMNSLRAEDTAVYYCARERGSGSYWVDYWGQGTLVTVSS

SEQ ID NO: 37 - protein, 8 aa, Artificial Sequence
  Description of Artificial Sequence Synthetic peptide
GFTFSDYY

SEQ ID NO: 38 - protein, 8 aa, Artificial Sequence
  Description of Artificial Sequence Synthetic peptide
ISSSGSTI

SEQ ID NO: 39 - protein, 13 aa, Artificial Sequence
  Description of Artificial Sequence Synthetic peptide
ARERGSGSYWVDY

SEQ ID NO: 40 - DNA, 316 bp, Artificial Sequence
  Description of Artificial Sequence Synthetic polynucleotide
cagcttgtgctgactcaatcgccctctgcctctgcctccctgggagcctcggtcaagctcacctgcactctgagcagtgggcacagcaactacgccatcgcatggcatcagcagcagccagagaagggccctcggtgcttgatgaaggttaacagtgatggcagccacagcaagggggacgggattcctgatcgcttctcaggctccagctctggggctgagcgctacctcaccatctccagcctccagtctgaggatgaggctgactattactgtcagacctggaccactggcattcgggtattcggcggaggga

SEQ ID NO: 41 - protein, 111 aa, Artificial Sequence
  Description of Artificial Sequence Synthetic polypeptide
QLVLTQSPSASASLGASVKLTCTLSSGHSNYAIAWHQQQPEKGPRCLMKVNSDGSHSKGDGIPDRFSGSSSGAERYLTISSLQSEDEADYYCQTWTTGIRVFGGGTKLTVL

SEQ ID NO: 42 - protein, 7 aa, Artificial Sequence
  Description of Artificial Sequence Synthetic peptide
SGHSNYA

SEQ ID NO: 43 - protein, 7 aa, Artificial Sequence
  Description of Artificial Sequence Synthetic peptide
VNSDGSH

SEQ ID NO: 44 - protein, 9 aa, Artificial Sequence
  Description of Artificial Sequence Synthetic peptide
QTWTTGIRV

SEQ ID NO: 45 - DNA, 268 bp, Artificial Sequence
  Description of Artificial Sequence Synthetic polynucleotide
gaggtgcagctggtggagtctgggggaggcttggtaaagcctggggggtcccttagactctcctgtgttgcctctggattcactttcgataacgcctggatgagctgggtccgccaggctccagggaaggggctggagtgggttggccgtattaaaagtaaaagtgatggtgtgacaacggactacgccgcacacgtgaaaggcagattcacgatctcaagagacgaatcaaaaactcctctatatctgcaaatgaacagcctgagag

SEQ ID NO: 46 - protein, 121 aa, Artificial Sequence
  Description of Artificial Sequence Synthetic polypeptide
EVQLVESGGGLVKPGGSLRLSCVASGFTFDNAWMSWVRQAPGKGLEWVGRIKSKSDGVTTDYAAHVKGRFTISRDESKTPLYLQMNSLRVEDTAMYYCTTGGNQYSFFDSWGRGTLVTVSS

SEQ ID NO: 47 - protein, 8 aa, Artificial Sequence
  Description of Artificial Sequence Synthetic peptide
GFTFDNAW

SEQ ID NO: 48 - protein, 10 aa, Artificial Sequence
  Description of Artificial Sequence Synthetic peptide
IKSKSDGVTT

SEQ ID NO: 49 - protein, 12 aa, Artificial Sequence
  Description of Artificial Sequence Synthetic peptide
TTGGNQYSFFDS

SEQ ID NO: 50 - DNA, 314 bp, Artificial Sequence
  Description of Artificial Sequence Synthetic polynucleotide
tcctatgagctgacacagccactctcagtgtcagtggccctgggacagacggccaagattacctgtgggggagacaacattggaagaaagaatgtgcactggtaccagcagaagccaggccaggcccctgtcttggtcatctataaggatcgctaccggccctctgggatccctgagcgattctctggctccaactcggggaacacggccaccctgaccatcaacagagcccaaggcggggatgacgctgactatttctgtcaggtgtgggacagtagcgctgcgggggtcctattcggcggagggaccaagct

SEQ ID NO: 51 - protein, 108 aa, Artificial Sequence
  Description of Artificial Sequence Synthetic polypeptide
SYELTQPLSVSVALGQTAKITCGGDNIGRKNVHWYQQKPGQAPVLVIYKDRYRPSGIPERFSGSNSGNTATLTINRAQGGDDADYFCQVWDSSAAGVLFGGGTKLTVL

SEQ ID NO: 52 - protein, 6 aa, Artificial Sequence
  Description of Artificial Sequence Synthetic peptide
NIGRKN

SEQ ID NO: 53 - protein, 3 aa, Artificial Sequence
  Description of Artificial Sequence Synthetic peptide
KDR

SEQ ID NO: 54 - protein, 11 aa, Artificial Sequence
  Description of Artificial Sequence Synthetic peptide
QVWDSSAAGVL

SEQ ID NO: 55 - DNA, 272 bp, Artificial Sequence
  Description of Artificial Sequence Synthetic polynucleotide
caggtgcagctgcaggagtcgggcccaggactggtgaagccttcggagaccctgtccctcacctgcagtgtctctggtgactccatcaatttttactactggaactggatccggcagtccccagggaagggactggagtggattgcgtatgtgtctaaccgtggtgacagtacgaagtataacccctcactcgagagtcgagtcaccatttcaagagagccgtccaagcgccagtcctccctgaaactgaactctgtgaccgccgcggacac

SEQ ID NO: 56 - protein, 120 aa, Artificial Sequence
  Description of Artificial Sequence Synthetic polypeptide
QVQLQESGPGLVKPSETLSLTCSVSGDSINFYYWNWIRQSPGKGLEWIAYVSNRGDSTKYNPSLESRVTISREPSKRQSSLKLNSVTAADTAVYYCALWSSYFRGYFDYWGQGILVTVSS

SEQ ID NO: 57 - protein, 9 aa, Artificial Sequence
  Description of Artificial Sequence Synthetic peptide
GDSINFYYW

SEQ ID NO: 58 - protein, 7 aa, Artificial Sequence
  Description of Artificial Sequence Synthetic peptide
SNRGDST

SEQ ID NO: 59 - protein, 13 aa, Artificial Sequence
  Description of Artificial Sequence Synthetic peptide
ALWSSYFRGYFDY

SEQ ID NO: 60 - DNA, 317 bp, Artificial Sequence
  Description of Artificial Sequence Synthetic polynucleotide
gaaatagtgatgacgcagtctccagccaccctgtctgtgtctccaggggaaagagtcaccctctcctgcagggccagtcagagtgtgagcaccaatttagcctggtaccagcagaggcctggccaggctcccaggctcctcatctatgcttcttccaccagggccactggtatcccagccaggttcagtggcggtgggtctgggacagagttcactctcaccatcagcagcctgcagtctgaagattttgcagtttattactgtcagcagtatggtcactggcctccttacactttcggccctgggaccaaagtgga

SEQ ID NO: 61 - protein, 108 aa, Artificial Sequence
  Description of Artificial Sequence Synthetic polypeptide
EIVMTQSPATLSVSPGERVTLSCRASQSVSTNLAWYQQRPGQAPRLLIYASSTRATGIPARFSGGGSGTEFTLTISSLQSEDFAVYYCQQYGHWPPYTFGPGTKVDIK

SEQ ID NO: 62 - protein, 6 aa, Artificial Sequence
  Description of Artificial Sequence Synthetic peptide
QSVSTN

SEQ ID NO: 63 - protein, 3 aa, Artificial Sequence
  Description of Artificial Sequence Synthetic peptide
ASS

SEQ ID NO: 64 - protein, 10 aa, Artificial Sequence
  Description of Artificial Sequence Synthetic peptide
QQYGHWPPYT

SEQ ID NO: 65 - DNA, 272 bp, Artificial Sequence
  Description of Artificial Sequence Synthetic polynucleotide
caggtgcagctacagcagtggggcgcaggactgttgaagccttcggagaccctgtccctcacgtgcgctgtctatggtgggtccttcagtggttactactggacttggatccgccagcccccagggaagggactggagtggattggagaaatcaataatagtggaaaaaccaactacaacccgtccctcaaaagtcgagtcagcatttcaatagacacgtccaagaaccagttttccctgaaggtgacttctgtgaccgccgcggacacagc

SEQ ID NO: 66 - protein, 120 aa, Artificial Sequence
  Description of Artificial Sequence Synthetic polypeptide
QVQLQQWGAGLLKPSETLSLTCAVYGGSFSGYYWTWIRQPPGKGLEWIGEINNSGKTNYNPSLKSRVSISIDTSKNQFSLKVTSVTAADTAVYYCARGPQQHLEPPFDYWGHGTLVTVSS

SEQ ID NO: 67 - protein, 8 aa, Artificial Sequence
  Description of Artificial Sequence Synthetic peptide
GGSFSGYY

SEQ ID NO: 68 - protein, 7 aa, Artificial Sequence
  Description of Artificial Sequence Synthetic peptide
INNSGKT

SEQ ID NO: 69 - protein, 14 aa, Artificial Sequence
  Description of Artificial Sequence Synthetic peptide
ARGPQQHLEPPFDY

SEQ ID NO: 70 - DNA, 316 bp, Artificial Sequence
  Description of Artificial Sequence Synthetic polynucleotide
cagtctgtgctgactcagccaccctcagcgtctgggacccccgggcaaagggtcaccatctcttgttctggaagcaactccaacatcgcgactaattatgtgtgctggtaccagcaatacccaggaacggcccccaaacccctcatctacaggactgatcagcggccctcaggggtccctgaccgattctctggctccaagtctggcacctcagcctccctggccatcagtgggctccggtccgaggatgaggctgattattattgtgcaacatgggatgacagcctgagtgcctgggtgttcggcggagggacca

SEQ ID NO: 71 - protein, 110 aa, Artificial Sequence
  Description of Artificial Sequence Synthetic polypeptide
QSVLTQPPSASGTPGQRVTISCSGSNSNIATNYVCWYQQYPGTAPKPLIYRTDQRPSGVPDRFSGSKSGTSASLAISGLRSEDEADYYCATWDDSLSAWVFGGGTKLTVL

SEQ ID NO: 72 - protein, 8 aa, Artificial Sequence
  Description of Artificial Sequence Synthetic peptide
NSNIATNY

SEQ ID NO: 73 - protein, 3 aa, Artificial Sequence
  Description of Artificial Sequence Synthetic peptide
RTD

SEQ ID NO: 74 - protein, 11 aa, Artificial Sequence
  Description of Artificial Sequence Synthetic peptide
ATWDDSLSAWV

SEQ ID NO: 75 - DNA, 293 bp, Artificial Sequence
  Description of Artificial Sequence Synthetic polynucleotide
caggttcagctggtgcagtctgggactgaagtgagggagcctggggcctcagtgaaggtctcctgcaaggcttctggatacaccttcaccaactatgatatcaactgggtgcgacaggccacaggacaagggcttgagtgggtgggatggatgaaccctaatagtggtgagacaggctatgcacaggagttccagggcagaatcaccattactagggacacctccataagcacaatttacatggagttgagcagcctgacatctgaggacacggccgtatattactgtgccag

SEQ ID NO: 76 - protein, 125 aa, Artificial Sequence
  Description of Artificial Sequence Synthetic polypeptide
QVQLVQSGTEVREPGASVKVSCKASGYTFTNYDINWVRQATGQGLEWVGWMNPNSGETGYAQEFQGRITITRDTSISTIYMELSSLTSEDTAVYYCARGGFCTSTSCYYHYMDVWGKGTTVTVSS

SEQ ID NO: 77 - protein, 8 aa, Artificial Sequence
  Description of Artificial Sequence Synthetic peptide
GYTFTNYD

SEQ ID NO: 78 - protein, 8 aa, Artificial Sequence
  Description of Artificial Sequence Synthetic peptide
MNPNSGET

SEQ ID NO: 79 - protein, 18 aa, Artificial Sequence
  Description of Artificial Sequence Synthetic peptide
ARGGFCTSTSCYYHYMDV

SEQ ID NO: 80 - DNA, 322 bp, Artificial Sequence
  Description of Artificial Sequence Synthetic polynucleotide
gacatccagatgacccagtctccttccaccctgtctgcatctgtaggagacagagtcaccatcacttgtcgggccagtcagagtgttaatagctggttggcctggtatcagcagaaaccagggaaagcccctaaggtcctgatctataaggcaactagtttagaaagtggggtcccatcaaggttcagcggcagtgaatctgggacagaattcactctcaccatcagcagcctgcaggctgatgattttgcaacttattactgccaacagtataatgattttccgtacacttttggccgggggaccaagctggagatcaaac

SEQ ID NO: 81 - protein, 107 aa, Artificial Sequence
  Description of Artificial Sequence Synthetic polypeptide
DIQMTQSPSTLSASVGDRVTITCRASQSVNSWLAWYQQKPGKAPKVLIYKATSLESGVPSRFSGSESGTEFTLTISSLQADDFATYYCQQYNDFPYTFGRGTKLEIK

SEQ ID NO: 82 - protein, 6 aa, Artificial Sequence
  Description of Artificial Sequence Synthetic peptide
QSVNSW

SEQ ID NO: 83 - protein, 3 aa, Artificial Sequence
  Description of Artificial Sequence Synthetic peptide
KAT

SEQ ID NO: 84 - protein, 9 aa, Artificial Sequence
  Description of Artificial Sequence Synthetic peptide
QQYNDFPYT

SEQ ID NO: 85 - DNA, 270 bp, Artificial Sequence
  Description of Artificial Sequence Synthetic polynucleotide
caggtgcagctacagcagtggggcgcaggactgttgaagcctgcggagaccctgtccctcacttgcgctgtctatggtgggtccttcactggtcactactggacctggatccgtcagccccctggtaaggggccggaatggattggggaaatcaatcatcgtggaggcaccgactacaacccgtccctcaagagtcgagtcaccatttcactagacacgtccaagaaccaggtgtccctgaaactgagcgctgtgaccgccgtagacacg

SEQ ID NO: 86 - protein, 120 aa, Artificial Sequence
  Description of Artificial Sequence Synthetic polypeptide
QVQLQQWGAGLLKPAETLSLTCAVYGGSFTGHYWTWIRQPPGKGPEWIGEINHRGGTDYNPSLKSRVTISLDTSKNQVSLKLSAVTAVDTAVYYCARGHGRYYYSYLDSWAQGTLVTVSS

SEQ ID NO: 87 - protein, 8 aa, Artificial Sequence
  Description of Artificial Sequence Synthetic peptide
GGSFTGHY

SEQ ID NO: 88 - protein, 7 aa, Artificial Sequence
  Description of Artificial Sequence Synthetic peptide
INHRGGT

SEQ ID NO: 89 - protein, 14 aa, Artificial Sequence
  Description of Artificial Sequence Synthetic peptide
ARGHGRYYYSYLDS

SEQ ID NO: 90 - DNA, 319 bp, Artificial Sequence
  Description of Artificial Sequence Synthetic polynucleotide
gacatccagatgacccagtctccttccaccctgtctgcagctgtaggagaccgagtcaccatcacttgccgggccagtcaggctattagtccctgggtggcctggtatcagcagaaaccagggaaagccccttcactccttatctatcaggcgtctactttacaaagtgcggtcccattaaggttcagcggcagtggatctgggacagacttcactctcaccatcagcagcctgcagcctgaggattttgcaacttatttctgccaacagtatggtcgctattccacttttggccaggggaccaagctggagatcaaac

SEQ ID NO: 91 - protein, 106 aa, Artificial Sequence
  Description of Artificial Sequence Synthetic polypeptide
DIQMTQSPSTLSAAVGDRVTITCRASQAISPWVAWYQQKPGKAPSLLIYQASTLQSAVPLRFSGSGSGTDFTLTISSLQPEDFATYFCQQYGRYSTFGQGTKLEIK

SEQ ID NO: 92 - protein, 6 aa, Artificial Sequence
  Description of Artificial Sequence Synthetic peptide
QAISPW

SEQ ID NO: 93 - protein, 3 aa, Artificial Sequence
  Description of Artificial Sequence Synthetic peptide
QAS

SEQ ID NO: 94 - protein, 8 aa, Artificial Sequence
  Description of Artificial Sequence Synthetic peptide
QQYGRYST

SEQ ID NO: 95 - DNA, 272 bp, Artificial Sequence
  Description of Artificial Sequence Synthetic polynucleotide
caggtgcagctacagcagtggggcgcaggactgttgaagcctgcggagaccctgtccctcacctgcgctgtctatggtgggtccttcactggttactactggacctggatccgtcagccccctggtaaggggctggaatggattggggagatcaatcatcgtggaggcaccgactacaatccgtccctcaagagtcgagtcaccatttctcttgacacgtccaagaaccaggtgtccctgaaactccgctctgcgaccgccgtagacacggc

SEQ ID NO: 96 - protein, 120 aa, Artificial Sequence
  Description of Artificial Sequence Synthetic polypeptide
QVQLQQWGAGLLKPAETLSLTCAVYGGSFTGYYWTWIRQPPGKGLEWIGEINHRGGTDYNPSLKSRVTISLDTSKNQVSLKLRSATAVDTAVYYCARGHGRYYYSYLNLWAQGTLVTVSS

SEQ ID NO: 97 - protein, 8 aa, Artificial Sequence
  Description of Artificial Sequence Synthetic peptide
GGSFTGYY

SEQ ID NO: 98 - protein, 7 aa, Artificial Sequence
  Description of Artificial Sequence Synthetic peptide
INHRGGT

SEQ ID NO: 99 - protein, 14 aa, Artificial Sequence
  Description of Artificial Sequence Synthetic peptide
ARGHGRYYYSYLNL

SEQ ID NO: 100 - DNA, 319 bp, Artificial Sequence
  Description of Artificial Sequence Synthetic polynucleotide
gacatccagatgacccagtctccttccaccctgtctgcatctgtaggagacagagtcaccatcacttgccgggccagtcaggctattagtccctggttggcctggtatcagcagaaaccagggagagcccctaaactcctgatctatcaggcgtccactttacaaagtgcggtcccatcaagattcagcggcagtggatctgggacagaattcactctcaccatcaccagcctgcagcctgaagattttgcaacttattactgccaacagtatggtcgttattccacttttggccaggggaccaagctggagatcaaac

SEQ ID NO: 101 - protein, 106 aa, Artificial Sequence
  Description of Artificial Sequence Synthetic polypeptide
DIQMTQSPSTLSASVGDRVTITCRASQAISPWLAWYQQKPGRAPKLLIYQASTLQSAVPSRFSGSGSGTEFTLTITSLQPEDFATYYCQQYGRYSTFGQGTKLEIK

SEQ ID NO: 102 - protein, 6 aa, Artificial Sequence
  Description of Artificial Sequence Synthetic peptide
QAISPW

SEQ ID NO: 103 - protein, 3 aa, Artificial Sequence
  Description of Artificial Sequence Synthetic peptide
QAS

SEQ ID NO: 104 - protein, 8 aa, Artificial Sequence
  Description of Artificial Sequence Synthetic peptide
QQYGRYST

SEQ ID NO: 105 - DNA, 272 bp, Artificial Sequence
  Description of Artificial Sequence Synthetic polynucleotide
caggtgcagctgcaggagtcgggcccaggactggtgaagccttcacagaccctgtccctcacctgcactgtctctggtggctccatcagcagtggtagttactactggagctggatccggcagcccgccgggaagggactggagtggattgggcgtatctataccagtgggagcaccaactacaacccctccctcaagagtcgagtcaccatatcagtagacacgtccaagaaccagttctccctgaagctgagctctgtgaccgccgcaga

SEQ ID NO: 106 - protein, 124 aa, Artificial Sequence
  Description of Artificial Sequence Synthetic polypeptide
QVQLQESGPGLVKPSQTLSLTCTVSGGSISSGSYYWSWIRQPAGKGLEWIGRIYTSGSTNYNPSLKSRVTISVDTSKNQFSLKLSSVTAADTAVYYCARVMVRGVIGSYGMDVWGQGTTVTVSS

SEQ ID NO: 107 - protein, 10 aa, Artificial Sequence
  Description of Artificial Sequence Synthetic peptide
GGSISSGSYY

SEQ ID NO: 108 - protein, 7 aa, Artificial Sequence
  Description of Artificial Sequence Synthetic peptide
IYTSGST

SEQ ID NO: 109 - protein, 16 aa, Artificial Sequence
  Description of Artificial Sequence Synthetic peptide
ARVMVRGVIGSYGMDV

SEQ ID NO: 110 - DNA, 312 bp, Artificial Sequence
  Description of Artificial Sequence Synthetic polynucleotide
tcctatgtgctgactcagccaccctcggtgtcagtggccccaggacagacggccaggattacctgtgggggaaacaacattggaagtaaaagtgtgcactggtaccagcagaagccaggccaggcccctgtgctggtcgtctatgatgatagcgaccggccctcagggatccctgagcgattctctggctccaactctgggaacacggccaccctgaccatcagcagggtcgaagccggggatgaggccgactattactgtcaggtgtgggatagtagtagtgatcatgaggtgttcggcggagggaccaag

SEQ ID NO: 111 - protein, 108 aa, Artificial Sequence
  Description of Artificial Sequence Synthetic polypeptide
SYVLTQPPSVSVAPGQTARITCGGNNIGSKSVHWYQQKPGQAPVLVVYDDSDRPSGIPERFSGSNSGNTATLTISRVEAGDEADYYCQVWDSSSDHEVFGGGTKLTVL

SEQ ID NO: 112 - protein, 6 aa, Artificial Sequence
  Description of Artificial Sequence Synthetic peptide
NIGSKS

SEQ ID NO: 113 - protein, 3 aa, Artificial Sequence
  Description of Artificial Sequence Synthetic peptide
DDS

SEQ ID NO: 114 - protein, 11 aa, Artificial Sequence
  Description of Artificial Sequence Synthetic peptide
QVWDSSSDHEV

SEQ ID NO: 115 - DNA, 273 bp, Artificial Sequence
  Description of Artificial Sequence Synthetic polynucleotide
gaggtgcagctgttggagtctgggggagccttggtacagcctggggggtccctgagactgtcctgtgtaacctctggattcacctttagctcctatgccatgacctgggtccgccaggctccagggaaggggctggaatgggtctcaggtattagttccggtggctttatcacatactacgcagactccgtgaagggccgcttcaccatctccagagacaattccaagaacacagtgtatttgcaaatgaacagcctgagagccgaggacacg

SEQ ID NO: 116 - protein, 121 aa, Artificial Sequence
  Description of Artificial Sequence Synthetic polypeptide
EVQLLESGGALVQPGGSLRLSCVTSGFTFSSYAMTWVRQAPGKGLEWVSGISSGGFITYYADSVKGRFTISRDNSKNTVYLQMNSLRAEDTALYYCAKGMGSNIYVGFDYWGQGTLVTVSS

SEQ ID NO: 117 - protein, 8 aa, Artificial Sequence
  Description of Artificial Sequence Synthetic peptide
GFTFSSYA

SEQ ID NO: 118 - protein, 8 aa, Artificial Sequence
  Description of Artificial Sequence Synthetic peptide
ISSGGFIT

SEQ ID NO: 119 - protein, 14 aa, Artificial Sequence
  Description of Artificial Sequence Synthetic peptide
AKGMGSNIYVGFDY

SEQ ID NO: 120 - DNA, 317 bp, Artificial Sequence
  Description of Artificial Sequence Synthetic polynucleotide
gaaattgtgttgacgcagtctccaggcaccctgtctttgtctccaggggaaagagccaccctctcctgcagggccagtcagattgtaagcagcaactacttagcctggtaccagcagaaacctggccaggctcccaggctcctcatctttggtgcatccagcagggccactggcatcccagacaggttcagtggcagtgggtctgacacagacttcactctcaccatccgcagactggagtctgaagattttgcagtgtattactgtcaccagtatggtagctcaccggggacgttcggccaagggaccaaggtgga

SEQ ID NO: 121 - protein, 108 aa, Artificial Sequence
  Description of Artificial Sequence Synthetic polypeptide
EIVLTQSPGTLSLSPGERATLSCRASQIVSSNYLAWYQQKPGQAPRLLIFGASSRATGIPDRFSGSGSDTDFTLTIRRLESEDFAVYYCHQYGSSPGTFGQGTKVEIK

SEQ ID NO: 122 - protein, 7 aa, Artificial Sequence
  Description of Artificial Sequence Synthetic peptide
QIVSSNY

SEQ ID NO: 123 - protein, 3 aa, Artificial Sequence
  Description of Artificial Sequence Synthetic peptide
GAS

SEQ ID NO: 124 - protein, 9 aa, Artificial Sequence
  Description of Artificial Sequence Synthetic peptide
HQYGSSPGT

SEQ ID NO: 125 - DNA, 271 bp, Artificial Sequence
  Description of Artificial Sequence Synthetic polynucleotide
caggtgcagctgcaggagtcgggcccaggactggtgaagccttcggagaccctgtccctcacctgctctgtctctggtggctccatcagtaattcctacgtgagctggatccggcagcccccagggaagggactggagtggattgggtatatctattacagtgggggcaccaactacaacccctcccttaagagtcgagtcaccatttcagtagacacgtccaagaaccagttctccctgaagctgagctccgtgaccgctgcggacacgg

SEQ ID NO: 126 - protein, 115 aa, Artificial Sequence
  Description of Artificial Sequence Synthetic polypeptide
QVQLQESGPGLVKPSETLSLTCSVSGGSISNSYVSWIRQPPGKGLEWIGYIYYSGGTNYNPSLKSRVTISVDTSKNQFSLKLSSVTAADTAVYYCARGKIYFDYWGQGTLVTVSS

SEQ ID NO: 127 - protein, 8 aa, Artificial Sequence
  Description of Artificial Sequence Synthetic peptide
GGSISNSY

SEQ ID NO: 128 - protein, 7 aa, Artificial Sequence
  Description of Artificial Sequence Synthetic peptide
IYYSGGT

SEQ ID NO: 129 - protein, 9 aa, Artificial Sequence
  Description of Artificial Sequence Synthetic peptide
ARGKIYFDY

SEQ ID NO: 130 - DNA, 317 bp, Artificial Sequence
  Description of Artificial Sequence Synthetic polynucleotide
gacatccagatgacccagtctccttccaccctgtctgcatccgtaggagacagagtcaccatcacttgccgggccagtcagagtattagtagttggttggcctggtatcagctgaaaccagggaaggcccctaaactcctgatttataaggcgtccagtttagaaagtggggtcccatcgagattcagcggcagtggatctgggacagaattcactctcaccatcagcagcctgcagcctggtgattttgcaacttattactgccaacagtataatagttatgctttggcgttcggccaagggaccaaggtggagat

SEQ ID NO: 131 - protein, 107 aa, Artificial Sequence
  Description of Artificial Sequence Synthetic polypeptide
DIQMTQSPSTLSASVGDRVTITCRASQSISSWLAWYQLKPGKAPKLLIYKASSLESGVPSRFSGSGSGTEFTLTISSLQPGDFATYYCQQYNSYALAFGQGTKVEIK

SEQ ID NO: 132 - protein, 6 aa, Artificial Sequence
  Description of Artificial Sequence Synthetic peptide
QSISSW

SEQ ID NO: 133 - protein, 3 aa, Artificial Sequence
  Description of Artificial Sequence Synthetic peptide
KAS

SEQ ID NO: 134 - protein, 9 aa, Artificial Sequence
  Description of Artificial Sequence Synthetic peptide
QQYNSYALA

SEQ ID NO: 135 - DNA, 296 bp, Artificial Sequence
  Description of Artificial Sequence Synthetic polynucleotide
cagctgcagctgcaggagtcgggcccaggactggtgaaaccttcggagaccctgtccctcacctgcactgtctctggtggctccatcagcagtagtctttactactggggctggatccgccagcccccagggaagggactggagtggattgggaatatctattatagtgggatcacctattacaacccgtccctcacaagtcgagtcaccatatccgtagacacgtccaaggaccagttctccctgaagctgagctctgtgaccgtcgcagacacggctgtgtattactgtgcgcg

SEQ ID NO: 136 - protein, 126 aa, Artificial Sequence
  Description of Artificial Sequence Synthetic polypeptide
QLQLQESGPGLVKPSETLSLTCTVSGGSISSSLYYWGWIRQPPGKGLEWIGNIYYSGITYYNPSLTSRVTISVDTSKDQFSLKLSSVTVADTAVYYCAREILTGDPSVGGDPFDYWGQGTLVTVSS

SEQ ID NO: 137 - protein, 10 aa, Artificial Sequence
  Description of Artificial Sequence Synthetic peptide
GGSISSSLYY

SEQ ID NO: 138 - protein, 7 aa, Artificial Sequence
  Description of Artificial Sequence Synthetic peptide
IYYSGIT

SEQ ID NO: 139 - protein, 18 aa, Artificial Sequence
  Description of Artificial Sequence Synthetic peptide
AREILTGDPSVGGDPFDY

SEQ ID NO: 140 - DNA, 331 bp, Artificial Sequence
  Description of Artificial Sequence Synthetic polynucleotide
cagtctgtgctgacgcagccgccctcagtgtctgcggccccaggacagaaggtcaccatctcctgctctggaagcagctccaacattgggaataattatgtttcttggtatcgacaactcccaggaacagcccccaaactcctcgtctatgaaagtaataagcgaccctcagggattcctgaccgattctctggctccaagtctgccacgtcagccaccctgggcatcaccggactccagactggggacgaggccgattattactgcggaacatgggataccagcctgagtgctgtggtattcggcggagggaccaaactgaccgtcctag

SEQ ID NO: 141 - protein, 110 aa, Artificial Sequence
  Description of Artificial Sequence Synthetic polypeptide
QSVLTQPPSVSAAPGQKVTISCSGSSSNIGNNYVSWYRQLPGTAPKLLVYESNKRPSGIPDRFSGSKSATSATLGITGLQTGDEADYYCGTWDTSLSAVVFGGGTKLTVL

SEQ ID NO: 142 - protein, 8 aa, Artificial Sequence
  Description of Artificial Sequence Synthetic peptide
SSNIGNNY

SEQ ID NO: 143 - protein, 3 aa, Artificial Sequence
  Description of Artificial Sequence Synthetic peptide
ESN

SEQ ID NO: 144 - protein, 11 aa, Artificial Sequence
  Description of Artificial Sequence Synthetic peptide
GTWDTSLSAVV

SEQ ID NO: 145 - DNA, 273 bp, Artificial Sequence
  Description of Artificial Sequence Synthetic polynucleotide
caggtgcagctgcaggagtcgggcccaggactggtgaagccttcgcagaccctgtccctcacctgcactgtctctaatttccccattgccagttcttactactggaactggatccgccagtcgccagaaaagggactggaatggattggaagtgtgtattttagtggcagcacccactctaatccgtctttcgcgagtcgagtgagcatgtcggtggacacctccaagagccaattcaccctcaagttgacctctctgtccgccgcggacaca

SEQ ID NO: 146 - protein, 121 aa, Artificial Sequence
  Description of Artificial Sequence Synthetic polypeptide
QVQLQESGPGLVKPSQTLSLTCTVSNFPIASSYYWNWIRQSPEKGLEWIGSVYFSGSTHSNPSFASRVSMSVDTSKSQFTLKLTSLSAADTAVYYCAKGDTSRLATNFDDWGPGIQVIVSS

SEQ ID NO: 147 - protein, 9 aa, Artificial Sequence
  Description of Artificial Sequence Synthetic peptide
NFPIASSYY

SEQ ID NO: 148 - protein, 7 aa, Artificial Sequence
  Description of Artificial Sequence Synthetic peptide
VYFSGST

SEQ ID NO: 149 - protein, 14 aa, Artificial Sequence
  Description of Artificial Sequence Synthetic peptide
AKGDTSRLATNFDD

SEQ ID NO: 150 - DNA, 319 bp, Artificial Sequence
  Description of Artificial Sequence Synthetic polynucleotide
gacatcgtgatgacccagtctccagagaccctgcctgtgtctctgggcgagagggccaccatcaactgcaagtccagccagactcttttatttacctccaacaataaggactacgtagcttggtaccagcagaaaccaggacagcctcctaagttgctcatttactgggcatctacccgggaatccggggtccctgaccgcttcagtggcagcgggtctgggacagatttcactctcaccatcaacagcctgcaggctgaagatgtggcggtttattattgtcagcaataccttactactcctcttaccttcggcggag

SEQ ID NO: 151 - protein, 113 aa, Artificial Sequence
  Description of Artificial Sequence Synthetic polypeptide
DIVMTQSPETLPVSLGERATINCKSSQTLLFTSNNKDYVAWYQQKPGQPPKLLIYWASTRESGVPDRFSGSGSGTDFTLTINSLQAEDVAVYYCQQYLTTPLTFGGGTKVDIR

SEQ ID NO: 152 - protein, 12 aa, Artificial Sequence
  Description of Artificial Sequence Synthetic peptide
QTLLFTSNNKDY

SEQ ID NO: 153 - protein, 3 aa, Artificial Sequence
  Description of Artificial Sequence Synthetic peptide
WAS

SEQ ID NO: 154 - protein, 9 aa, Artificial Sequence
  Description of Artificial Sequence Synthetic peptide
QQYLTTPLT